(19) United States
(12) Patent Application Publication
(10) Pub. No.: US 2005/0216075 A1
(43) Pub. Date: Sep. 29, 2005

(54) Materials and devices of enhanced electromagnetic transparency

(76) Inventors: Xingwu Wang, Wellsville, NY (US); Howard J. Greenwald, Rochester, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/045,790

(22) Filed: Jan. 28, 2005

Related U.S. Application Data
(63) Continuation-in-part of application No. 10/974,412, filed on Oct. 27, 2004.
(63) Continuation-in-part of application No. 11/029,187, filed on Jan. 4, 2005.
Application No. 11/029,187 is a continuation-in-part of application No. 10/923,579, filed on Aug. 20, 2004.
Application No. 10/923,579 is a continuation-in-part of application No. 10/914,691, filed on Aug. 9, 2004.
Application No. 10/923,579 is a continuation-in-part of application No. 10/887,521, filed on Jul. 7, 2004.
Application No. 10/923,579 is a continuation-in-part of application No. 10/867,517, filed on Jun. 14, 2004.
Application No. 10/923,579 is a continuation-in-part of application No. 10/810,916, filed on Mar. 26, 2004, now Pat. No. 6,846,985.
Application No. 10/923,579 is a continuation-in-part of application No. 10/808,618, filed on Mar. 24, 2004.
Application No. 10/923,579 is a continuation-in-part of application No. 10/786,198, filed on Feb. 25, 2004.
Application No. 10/923,579 is a continuation-in-part of application No. 10/780,045, filed on Feb. 17, 2004.
Application No. 10/923,579 is a continuation-in-part of application No. 10/747,472, filed on Dec. 29, 2003.
Application No. 10/923,579 is a continuation-in-part of application No. 10/744,543, filed on Dec. 22, 2003.
Application No. 10/923,579 is a continuation-in-part of application No. 10/442,420, filed on May 21, 2003, now Pat. No. 6,914,412.
Application No. 10/923,579 is a continuation-in-part of application No. 10/409,505, filed on Apr. 8, 2003, now Pat. No. 6,815,609.
(60) Provisional application No. 60/559,555, filed on Apr. 5, 2004.

(51) Int. Cl. A61F 2/06

(52) U.S. Cl. 623/1.15; 623/1.46; 174/35 MS; 424/422

(57) ABSTRACT

Materials, devices and methods are described for making and using devices of enhanced electromagnetic transparency. Desirable embodiments include for example, nanomagnetic compositions that provide series and/or parallel resonances that act to diminish induced current and/or voltage in devices and thereby alter electromagnetic penetration. Devices, including medical implants, such as stents, may be formed or modified in a variety of protective conformations. Such conformations include, for example, the addition or formulation with layer(s) of protective material or with of discrete components such as multiple capacitors and inductors.

### CROSS-REFERENCE TO RELATED APPLICATIONS

This application is a continuation-in-part of applicant's U.S. patent application Ser. No. 10/974,412, filed Oct. 27, 2004 and entitled “Stent and MRI Imaging Process,” and a continuation-in-part of applicant's U.S. patent application Ser. No. 11/029,187, filed on Jan. 4, 2005 and entitled “Novel Composition,” which in turn was a continuation-in-part of applicant's U.S. patent application Ser. No. 10/923,579, filed on Aug. 20, 2004, which in turn was a continuation-in-part of each of applicants' copending patent application Ser. Nos. 10/914,691 (filed on Aug. 9, 2004), 10/887,521 (filed on Jul. 7, 2004), 10,867,517 (filed on Jun. 14, 2004), 10/810,916 (filed on Mar. 26, 2004), 10/808,618 (filed on Mar. 24, 2004), 10/786,198 (filed on Feb. 25, 2004), 10/780,045 (filed on Feb. 17, 2004), 10/747,472 (filed on Dec. 29, 2003), 10/744,543 (filed on Dec. 22, 2003), 10/442,420 (filed on May 21, 2003), and 10/409,505 (filed on Apr. 8, 2003). The entire disclosure of each of these patent applications is hereby incorporated by reference into this specification.

This patent application also claims priority based upon provisional patent application 60/559,555, filed on Apr. 5, 2004, the entire disclosure of which is hereby incorporated by reference into this specification.

### FIELD OF THE INVENTION

The invention relates to electromagnetic energy transmission and more specifically to materials and devices that can alter electromagnetic energy transmission for imaging and other purposes.

### BACKGROUND OF THE INVENTION

Applicants have been awarded several United States patents that describe nanomagnetic material. These include U.S. Pat. No. 6,506,972 (“Magnetically shielded conductor”), U.S. Pat. No. 6,673,999 (“Magnetically shielded assembly”), U.S. Pat. No. 6,713,671 (“Magnetically shielded assembly”), U.S. Pat. No. 6,765,144 (“Magnetic resonance imaging coated assembly”), U.S. Pat. No. 6,768,053 (“Optical fiber assembly”), and U.S. Pat. No. 6,815,609 (“Nanomagnetic composition”). The entire disclosure of each of these United States patents is hereby incorporated by reference into this specification.

In addition, applicants have published several United States patent applications that relate to nanomagnetic material including, published United States patent applications US20040210289 (“Novel nanomagnetic particles”), US20040211580 (“Magnetically shielded assembly”), US20040225213 (“Magnetic resonance imaging coated assembly”), US20040226603 (“Optical fiber assembly”), 20040230271 (“Magnetically shielded assembly”), US20040249428 (“Magnetically shielded assembly”), US20040254419 (“Therapeutic assembly”), and US20040256131 (“Nanomagnetically shielded assembly”). The entire disclosure of each of these published United States patent applications

It is an object of this invention to provide improved compositions that comprise such nanomagnetic material and one or more other materials.

### SUMMARY OF THIS INVENTION

Embodiments provide materials that affect electromagnetic transparency of devices. A representative embodiment is a material that is on, in, near to, and/or that comprises a device and that acts to make the device more transparent to electromagnetic energy. The material may, for example have the bulk property of induced current cancellation and/or induced voltage cancellation. Discrete circuit resonance elements may be used in addition to and/or instead of materials with such bulk properties. Preferred embodiments that employ nanomagnetic materials are provided as examples of representative materials, their conformations and use. Further details of materials useful for embodiments, and representative methods of their preparation and use are provided to show some of the ways that a skilled artisan may implement embodiments of the invention, after reading and understanding the specification and are not meant to be limiting in any way. A skilled artisan may, for example, select from a variety of alternative materials, as will be appreciated by a worker in this field.

### BRIEF DESCRIPTION OF THE DRAWINGS

Applicants' inventions will be described by reference to the specification and the drawings, in which like numerals refer to like elements, and wherein:

FIG. 1 is a schematic illustration, not drawn to scale, of a coated substrate assembly 10 comprised of a substrate 12 and, disposed thereon, a coating 14 comprised of a multiplicity of nanomagnetic particles 16;

FIGS. 2 and 3 schematically illustrate the porosity of the side of coating 14, and the top of the coating 14, depicted in FIG. 1;

FIG. 4 is a schematic illustration of a coated stent assembly 100;

FIG. 5 is a partial schematic view of a coated stent assembly 200;

FIG. 6 is a schematic of one preferred sputtering process;

FIG. 7 is a partial schematic of one preferred particle collection process;

FIG. 8 is a schematic of a plasma deposition process;

FIG. 9 is a schematic of one preferred forming process;

FIGS. 10, 11, 12, 13, and 14 are schematic illustrations of preferred particles of the invention;

FIG. 15 is a phase diagram showing various compositions that may contain moieties E, F, and G;

FIG. 16 is a cross-sectional view of a preferred stent of this invention;

FIG. 17 is a cross-sectional view of a coated strut 1020 of the stent of FIG. 16;

FIG. 18 shows the effect on the coated strut 1020 when a patient is exposed to an electromagnetic field 1090;

FIG. 19 is a cross-sectional view of another coated strut 1021;

FIG. 20 shows the effect on the coated strut 1021 when a patient is exposed to an electromagnetic field 1090;

FIG. 21 is a cross-sectional view of another coated strut 1023;

FIG. 22 shows the effect on the coated strut 1023 when a patient is exposed to an electromagnetic field 1090;

FIG. 23 is a cross-sectional view of a coated strut 1027;

FIG. 24 is a schematic illustration of an inorganic tubular mineral composition;

FIG. 25 is a sectional view of the inorganic tubular mineral composition of FIG. 24;

FIG. 26 is a schematic view of an inorganic tubular mineral composition comprised of nanomagnetic material on the exterior surfaces of the tubules;

FIG. 27 is a schematic view of an inorganic tubular mineral composition comprised of nanomagnetic material on the interior surfaces of the tubules;

FIG. 28 is a schematic diagram of a flexed inorganic tubules comprised of a film of nanomagnetic material on its exterior surface;

FIG. 29 is a flow diagram illustrating the adaptive shielding capabilities of the nanonomagnetic coatings of this invention;

FIG. 30 is a graph illustrating how the susceptibility of the nanomagnetic coatings of the invention varies in the presence of an alternating current electromagnetic field;

FIG. 31 is a schematic of one preferred stent assembly of this invention;

FIG. 32 is a graph of the input electromagnetic wave, and the output electromagnetic wave, depicted in the stent assembly of FIG. 31;

FIG. 33 is a sectional view of strut of one preferred stent of the invention;

FIG. 34 is a schematic sectional view of one preferred coated substrate; and

FIG. 35 is an equivalent circuit representing the electrical phenomena that occur when the substrate of FIG. 34 is subjected to an MRI field.

### DESCRIPTION OF THE PREFERRED EMBODIMENTS

In the first portion of this specification, the properties of applicants' preferred nanomagnetic material are described. In the second portion of this specification, applicants will describe a preferred process for preparing such nanomagnetic material. In the third part of this specification, applicants will describe certain preferred devices that comprise the preferred nanomagnetic material. In the fourth part of this specification, applicants will describe a composition comprised of such nanomagnetic material and one or more minerals.

The Magnetic Permeability of the Nanomagnetic Material

In one preferred embodiment, the nanomagnetic material of this invention has a magnetic permeability of from about 0.7 to about 2.0. As used in this specification, the term “magnetic permeability” refers to “ . . . a property of materials modifying the action of magnetic poles placed therein and modifying the magnetic induction resulting when the material is subjected to a magnetic field of magnetizing force. The permeability of a substance may be defined as the ratio of the magnetic induction in the substance to the magnetizing field to which it is subjected. The permeability of a vacuum is unity.” See, e.g., page F-102 of -Robert E. Weast et al.'s “Handbook of Chemistry and Physics,” 63^{rd} Edition (CRC Press, Inc., Boca Raton, Fla., 1982-1983 edition). Reference may also be had, e.g., to U.S. Pat. No. 4,007,066 (material having a high magnetic permeability), U.S. Pat. No. 4,340,770 (enhancement of the magnetic permeability in glass metal shielding), U.S. Pat. No. 4,482,397 (method for improving the magnetic permeability of grain oriented silicon steel), U.S. Pat. No. 4,702,935 (high magnetic permeability alloy film), U.S. Pat. No. 4,725,490 (high magnetic permeability composites containing fibers with ferrite fill), U.S. Pat. No. 5,073,211 (method for manufacturing steel article having high magnetic permeability and low coercive force), U.S. Pat. No. 5,099,518 (electrical conductor of high magnetic permeability material), U.S. Pat. No. 5,645,774 (method for establishing a target magnetic permeability in a ferrite), U.S. Pat. No. 5,691,645 (process for determining intrinsic magnetic permeability of elongated ferromagnetic elements), U.S. Pat. No. 5,691,645 (process for determining intrinsic magnetic permeability of elongated ferromagnetic elements), U.S. Pat. No. 6,020,741 (wellbore imaging using magnetic permeability measurements), U.S. Pat. No. 6,176,944 (method for making low magnetic permeability cobalt sputter targets), U.S. Pat. No. 6,190,516 (high magnetic flux sputter targets with varied magnetic permeability in selected regions), U.S. Pat. No. 6,233,126 (thin film magnetic head having low magnetic permeability layer), U.S. Pat. No. 6,472,836 (magnetic permeability position detector), and the like. The entire disclosure of each of these United States patents is hereby incorporated by reference into this specification.

Reference may also be had to page 1399 of Sybil P. Parker's “McGraw-Hill Dictionary of Scientific and Technical Terms,” Fourth Edition (McGraw Hill Book Company, New York, 1989). As is disclosed on this page 1399, permeability is “ . . . a factor, characteristic of a material, that is proportional to the magnetic induction produced in a material divided by the magnetic field strength; it is a tensor when these quantities are not parallel.

Nanomagnetic Particles in the Nanomagnetic Material

In one embodiment of this invention, there is provided a multiplicity of nanomagnetic particles that may be in the form of a film, a powder, a solution, etc. This multiplicity of nanomagnetic particles is hereinafter referred to as a collection of nanomagnetic particles.

The collection of nanomagnetic particles of this embodiment of the invention is generally comprised of at least about 0.05 weight percent of such nanomagnetic particles and, preferably, at least about 5 weight percent of such nanomagnetic particles. In one embodiment, such collection is comprised of at least about 50 weight percent of such magnetic particles. In another embodiment, such collection consists essentially of such nanomagnetic particles.

When the collection of nanomagnetic particles consists essentially of nanomagnetic particles, the term “compact” will be used to refer to such collection of nanomagnetic particles.

Particle Size of the Nanomagnetic Particles

In general, the nanomagnetic particles of this invention are smaller than about 100 nanometers. In one embodiment, these nano-sized particles have a particle size distribution such that at least about 90 weight percent of the particles have a maximum dimension in the range of from about 1 to about 100 nanometers.

In one embodiment, the average size of the nanomagnetic particles is preferably less than about 50 nanometers. In one embodiment, the nanomagnetic particles have an average size of less than about 20 nanometers. In another embodiment, the nanomagnetic particles have an average size of less than about 15 nanometers. In yet another embodiment, such average size is less than about 11 nanometers. In yet another embodiment, such average size is less than about 3 nanometers.

Coherence Length of the Nanomagnetic Particles

As is used in this specification, the term “coherence length” refers to the distance between adjacent nanomagnetic moieties, and it has the meaning set forth in applicants' published international patent document W003061755A2, the entire disclosure of which is hereby incorporated by reference into this specification. As is disclosed in such published international patent document, “Referring to FIG. 38, and in the preferred embodiment depicted therein, it will be seen that A moieties 5002, 5004, and 5006 are separated from each other either at the atomic level and/or at the nanometer level. The A moieties may be, e.g., A atoms, clusters of A atoms, A compounds, A solid solutions, etc; regardless of the form of the A moiety, it has the magnetic properties described hereinabove . . . Thus, referring . . . to FIG. 38, the normalized magnetic interaction between adjacent A moieties 5002 and 5004, and also between 5004 and 5006, is preferably described by the formula M=exp(−x/L), wherein M is the normalized magnetic interaction, exp is the base of the natural logarithm (and is approximately equal to 2.71828), x is the distance between adjacent A moieties, and L is the coherence length . . . . In one embodiment, and referring again to FIG. 38, x is preferably measured from the center 5001 of A moiety 5002 to the center 5002 of A moiety 5004; and x is preferably equal to from about 0.00001×L to about 100×L . . . . In one embodiment, the ratio of x/L is at least 0.5 and, preferably, at least 1.”

With regard to the term “coherence length,” reference also may be had to U.S. Pat. No. 4,411,959 (which discloses that “ . . . the spherical particle diameter, .phi., preferably is to exceed the Ginzburg-Landau coherence lengths, .xi.GL, to avoid any significant degradation of Tc. The spacing between adjacent particles is to be much less than .xi.GL to ensure strong coupling while the diameter of voids between dense-packed spheres should be comparable to .xi.GL in order to ensure maximum flux pinning . . . ”), U.S. Pat. No. 5,098,178 (which discloses that “In addition, the anisotropic shrinkage of the Sol-Gel during polymerization is utilized to increase the concentration of the superconducting inclusions 22 so that the average particle distance . . . between the superconducting inclusions 22 approaches the coherence length as much as possible. An average particle distance comparable to the coherence length between the super conducting inclusions 22 is necessary in order to achieve significant enhancement through the proximity effect and high critical currents for the matrix 10.”), U.S. Pat. No. 5,998,336 (“The ceramic particles 2 have physical dimensions larger than the superconducting coherence length of the ceramic. Typically, the coherence length of high T_{c} ceramic materials is 1.5 nm.”), U.S. Pat. No. 6,420,318 (“The particles 22 preferably have dimensions larger than the superconducting coherence length of the superconducting material.”), and the like. The entire disclosure of each of these United States patents is hereby incorporated by reference into this specification. The coherence length (L) between adjacent magnetic particles is, on average, preferably from about 10 to about 200 nanometers and, more preferably, from about 50 to about 150 nanometers. In one preferred embodiment, the coherence length (L) between adjacent nanomagnetic particles is from about 75 to about 125 nanometers.

In one embodiment, x is preferably equal to from about 0.00001 times L to about 100 times L. In one embodiment, the ratio of x/L is at least 0.5 and, preferably, at least 1.5.

Ratio of the Coherence Length Between Nanomagnetic Particles to Their Particle Size

In one preferred embodiment, the ratio of the coherence length between adjacent nanomagnetic particles to their particle size is at least 2 and, preferably, at least 3. In one aspect of this embodiment, such ratio is at least 4. In another aspect of this embodiment, such ratio is at least 5.

The Saturation Magnetization of the Nanomagnetic Particles of the Invention

The nanomagnetic particles of this invention preferably have a saturation magnetization (“magnetic moment”) of from about 2 to about 3,000 electromagnetic units (emu) per cubic centimeter of material. As is known to those skilled in the art, saturation magnetization is the maximum possible magnetization of a material. Reference may be had, e.g., to U.S. Pat. No. 3,901,741 (saturation magnetization of cobalt, samarium, and gadolinium alloys), U.S. Pat. No. 4,134,779 (iron-boron solid solution alloys having high saturation magnetization), U.S. Pat. No. 4,390,853 (microwave transmission devices having high saturation magnetization and low magnetostriction), U.S. Pat. No. 4,532,979 (iron-boron solid solution alloys having high saturation magnetization and low magnetostriction), U.S. Pat. No. 4,631,613 (thin film head having improved saturation magnetization), U.S. Pat. Nos. 4,705,613, 4,782,416 (magnetic head having two legs of predetermined saturation magnetization for a recording medium to be magnetized vertically), U.S. Pat. No. 4,894,360 (method of using a ferromagnetic material having a high permeability and saturation magnetization at low temperatures), U.S. Pat. No. 5,543,070 (magnetic recording powder having low curie temperature and high saturation magnetization), U.S. Pat. No. 5,761,011 (magnetic head having a magnetic shield film with a lower saturation magnetization than a magnetic response film of an MR element), U.S. Pat. No. 5,922,442 (magnetic recording medium having a cobalt/chromium alloy interlayer of a low saturation magnetization), U.S. Pat. No. 6,492,035 (magneto-optical recording medium with intermediate layer having a controlled saturation magnetization), and the like. The entire disclosure of each of these United States patents is hereby incorporated by reference into this specification. As will be apparent to those skilled in the art, especially upon studying the aforementioned patents, the saturation magnetization of thin films is often higher than the saturation magnetization of bulk objects.

Saturation magnetization may be measured by conventional means. Reference may be had, e.g., to U.S. Pat. No. 5,068,519 (magnetic document validator employing remanence and saturation measurements), U.S. Pat. Nos. 5,581,251, 6,666,930, 6,506,264 (ferromagnetic powder), U.S. Pat. Nos. 4,631,202, 4,610,911, 5,532,095, and the like. The entire disclosure of each of these United States patents is hereby incorporated by reference into this specification.

In one embodiment, the saturation magnetization of the nanomagnetic particles of this invention is preferably measured by a SQUID (superconducting quantum interference device). Reference may be had, e.g., to U.S. Pat. No. 5,423,223 (fatigue detection in steel using squid mangetometry), U.S. Pat. No. 6,496,713 (ferromagnetic foreign body detection with background canceling), U.S. Pat. Nos. 6,418,335, 6,208,884 (noninvasive room temperature instrument to measure magnetic susceptibility variations in body tissue), U.S. Pat. No. 5,842,986 (ferromagnetic foreign body screening method), U.S. Pat. Nos. 5,471,139, 5,408,178, and the like. The entire disclosure of each of these United States patents is hereby incorporated by reference into this specification.

In one preferred embodiment, the saturation magnetization of the nanomagnetic particle of this invention is at least 100 electromagnetic units (emu) per cubic centimeter and, more preferably, at least about 200 electromagnetic units (emu) per cubic centimeter. In one aspect of this embodiment, the saturation magnetization of such nanomagnetic particles is at least about 1,000 electromagnetic units per cubic centimeter.

In another embodiment, the nanomagnetic material of this invention is present in the form a film with a saturization magnetization of at least about 2,000 electromagnetic units per cubic centimeter and, more preferably, at least about 2,500 electromagnetic units per cubic centimeter. In this embodiment, the nanomagnetic material in the film preferably has the formula A₁A₂(B)ₓC₁(C₂)_{y}, wherein y is 1, the C moieties are oxygen and nitrogen, respectively, and the A moieties and the B moiety are as described elsewhere in this specification.

Without wishing to be bound to any particular theory, applicants believe that the saturation magnetization of their nanomagnetic particles may be varied by varying the concentration of the “magnetic” moiety A in such particles, and/or the concentrations of moieties B and/or C.

In one embodiment, in order to achieve the degree of saturation magnetization, the nanomagnetic particles used typically comprise one or more of iron, cobalt, nickel, gadolinium, and samarium atoms. Thus, e.g., typical nanomagnetic materials include alloys of iron and nickel (permalloy), cobalt, niobium, and zirconium (CNZ), iron, boron, and nitrogen, cobalt, iron, boron, and silica, iron, cobalt, boron, and fluoride, and the like. These and other materials are described in a book by J. Douglas Adam et al. entitled “Handbook of Thin Film Devices” (Academic Press, San Diego, Calif., 2000). Chapter 5 of this book, beginning at page 185, describes “magnetic films for planar inductive components and devices;” and Tables 5.1 and 5.2 in this chapter describe many magnetic materials.

The Coercive Force of the Nanomagnetic Particles

In one preferred embodiment, the nanomagnetic particles of this invention have a coercive force of from about 0.01 to about 5,000 Oersteds. The term coercive force refers to the magnetic field, H, which must be applied to a magnetic material in a symmetrical, cyclically magnetized fashion, to make the magnetic induction, B, vanish; this term often is referred to as magnetic coercive force. Reference may be had, e.g., to U.S. Pat. Nos. 3,982,276, 4,003,813 (method of making a magnetic oxide film with a high coercive force), U.S. Pat. No. 4,045,738 (variable reluctance speed sensor using a shielded high coercive force rare earth magnet), U.S. Pat. Nos. 4,061,824, 4,115,159 (method of increasing the coercive force of pulverized rare earth-cobalt alloys) U.S. Pat. No. 4,277,552 (toner containing high coercive force magnetic powder), U.S. Pat. No. 4,396,441 (permanent magnet having ultra-high coercive force), U.S. Pat. No. 4,465,526 (high coercive force permanent magnet), U.S. Pat. No. 4,481,045 (high-coercive-force permanent magnet), U.S. Pat. No. 4,485,163 (triiron tetroxide having specified coercive force), U.S. Pat. No. 4,675,170 (preparation of finely divided acicular hexagonal ferrites having a high coercive force), U.S. Pat. Nos. 4,741,953, 4,816,933 (magnetic recording medium of particular coercive force), U.S. Pat. No. 4,863,530 (Fe—Pt—Nb magnet with ultra-high coercive force), U.S. Pat. Nos. 4,939,210, 5,073,211 (method for manufacturing steel article having high magnetic permeability and low coercive force), U.S. Pat. No. 5,211,770 (magnetic recording powder having a high coercive force at room temperatures and a low curie point), U.S. Pat. No. 5,329,413 (magnetoresistive sensor magnetically coupled with high-coercive force film at two end regions), U.S. Pat. No. 5,596,555 (magnetooptical recording medium having magnetic layers that satisfy predetermined coercive force relationships), U.S. Pat. No. 5,686,137 (method of providing hexagonal ferrite magnetic powder with enhanced coercive force stability), U.S. Pat. No. 5,742,458 (giant magnetoresistive material film which includes a free layer, a pinned layer, and a coercive force increasing layer), U.S. Pat. Nos. 5,967,223, 6,189,791 (magnetic card reader and method for determining the coercive force of a magnetic card therein), U.S. Pat. Nos. 6,257,512, 6,295,186, 6,637,653 (method of measuring coercive force of a magnetic card), U.S. Pat. No. 6,449,122 (thin-film magnetic head including soft magnetic film exhibiting high saturation magnetic flux density and low coercive force), U.S. Pat. No. 6,496,338 (spin-valve magnetoresistive sensor including a first antiferromagnetic layer for increasing a coercive force), U.S. Pat. No. 6,667,119 (magnetic recording medium comprising magnetic layers, the coercive force thereof specifically related to saturation magnetic flux density), U.S. Pat. No. 6,687,009 (magnetic head with conductors formed on end layers of a multilayer film having magnetic layer coercive force difference), and the like. The entire disclosure of each of these United States patents is hereby incorporated by reference into this specification.

In one embodiment, the nanomagnetic particles have a coercive force of from about 0.01 to about 3,000 Oersteds. In yet another embodiment, the nanomagnetic particles have a coercive force of from about 0.1 to about 10.

The Phase Transition Temperature of the Nanomagnetic Particles

In one embodiment of this invention, the nanomagnetic particles have a phase transition temperature is from about 40 degrees Celsius to about 200 degrees Celsius. As used herein, the term phase transition temperature refers to temperature in which the magnetic order of a magnetic particle transitions from one magnetic order to another. Thus, for example, when a magnetic particle transitions from the ferromagnetic order to the paramagnetic order, the phase transition temperature is the Curie temperature. Thus, e.g., when the magnetic particle transitions from the anti-ferromagnetic order to the paramagnetic order, the phase transition temperature is known as the Neel temperature.

For a discussion of phase transition temperature, reference may be had, e.g., to U.S. Pat. No. 4,804,274 (method and apparatus for determining phase transition temperature using laser attenuation), U.S. Pat. No. 5,758,968 (optically based method and apparatus for detecting a phase transition temperature of a material of interest), U.S. Pat. Nos. 5,844,643, 5,933,565 (optically based method and apparatus for detecting a phase transition temperature of a material of interest), U.S. Pat. No. 6,517,235 (using refractory metal silicidation phase transition temperature points to control and/or calibrate RTP low temperature operation), and the like. The entire disclosure of each of these United States patents is hereby incorporated by reference into this specification.

For a discussion of Curie temperature, reference may be had, e.g., to U.S. Pat. No. 3,736,500 (liquid identification using magnetic particles having a preselected Curie temperature), U.S. Pat. No. 4,229,234 (passivated, particulate high Curie temperature magnetic alloys), U.S. Pat. Nos. 4,771,238, 4,778,867 (ferroelectric copolymers of vinylidene fluoride and trifluoroethyelene), U.S. Pat. No. 5,108,191 (method and apparatus for determining Curie temperatures of ferromagnetic materials), U.S. Pat. No. 5,229,219 (magnetic recording medium having a Curie temperature up to 180 degrees C.), U.S. Pat. No. 5,325,343 (magneto-optical recording medium having two RE-TM layers with the same Curie temperature), U.S. Pat. No. 5,420,728 (recording medium with several recording layers having different Curie temperatures), U.S. Pat. No. 5,487,046 (magneto-optical recording medium having two magnetic layers with the same Curie temperature), U.S. Pat. No. 5,543,070 (magnetic recording powder having low Curie temperature and high saturation magnetization), U.S. Pat. Nos. 5,563,852, 601,742 (heating device for an internal combustion engine with PTC elements having different Curie temperatures), U.S. Pat. No. 5,679,474 (overwritable optomagnetic recording medium having a layer with a Curie temperature that varies in the thickness direction), U.S. Pat. No. 5,764,601 (magneto-optical recording medium with a readout layer of varying composition and Curie temperature), U.S. Pat. Nos. 5,949,743, 6,125,083 (magneto-optical recording medium containing a middle layer with a lower Curie temperature than the other layers), U.S. Pat. No. 6,731,111 (magnetic ink containing magnetic powders with different Curie temperatures), and the like. The entire disclosure of each of these United States patents is hereby incorporated by reference into this specification.

As used herein, the term “Curie temperature” refers to the temperature marking the transition between ferromagnetism and paramagnetism, or between the ferroelectric phase and paraelectric phase. This term is also sometimes referred to as the “Curie point.”

As used herein, the term “Neel temperature” refers to a temperature, characteristic of certain metals, alloys, and salts, below which spontaneous magnetic ordering takes place so that they become antiferromagnetic, and above which they are paramagnetic; this is also known as the Neel point. Reference may be had, e.g., to U.S. Pat. Nos. 3,845,306; 3,883,892; 3,946,372; 3,971,843; 4,103,315; 4,396,886; 5,264,980; 5,492,720; 5,756,191; 6,083,632; 6,181,533, 3,883,892, 3,845,306; 6,020,060; 6,083,632, 4,396,886, 4,438,462; 4,621,030; 5,923,504; 6,020,060; 6,146,752; 6,483,674; 6,631,057; 6,534,204; 6,534,205; 6,754,720; and the like. The entire disclosure of each of these United States patents is hereby incorporated by reference into this specification.

Neel temperature is also discussed at page F-92 of the “Handbook of Chemistry and Physics,” 63^{rd} Edition (CRC Press, Inc., Boca Raton, Fla., 1982-1983). As is disclosed on such page, ferromagnetic materials are “those in which the magnetic moments of atoms or ions tend to assume an ordered but nonparallel arrangement in zero applied field, below a characteristic temperature called the Neel point. In the usual case, within a magnetic domain, a substantial net magnetization results form the antiparallel alignment of neighboring nonequivalent subslattices. The macroscopic behavior is similar to that in ferromagnetism. Above the Neel point, these materials become paramagnetic.”

Without wishing to be bound to any particular theory, applicants believe that the phase temperature of their nanomagnetic particles can be varied by varying the ratio of the A, B, and C moieties described hereinabove as well as the particle sizes of the nanoparticles.

In one embodiment, the phase transition temperature of the nanomagnetic particles of is higher than the temperature needed to kill cancer cells but lower than the temperature needed to kill normal cells. As is disclosed in, e.g., U.S. Pat. No. 4,776,086 (the entire disclosure of which is hereby incorporated by reference into this specification), “The use of elevated temperatures, i.e., hyperthermia, to repress tumors has been under continuous investigation for many years. When normal human cells are heated to 41°-43° C., DNA synthesis is reduced and respiration is depressed. At about 45° C., irreversible destruction of structure, and thus function of chromosome associated proteins, occurs. Autodigestion by the cell's digestive mechanism occurs at lower temperatures in tumor cells than in normal cells. In addition, hyperthermia induces an inflammatory response which may also lead to tumor destruction. Cancer cells are more likely to undergo these changes at a particular temperature. This may be due to intrinsic differences, between normal cells and cancerous cells. More likely, the difference is associated with the lop pH (acidity), low oxygen content and poor nutrition in tumors as a consequence of decreased blood flow. This is confirmed by the fact that recurrence of tumors in animals, after hyperthermia, is found in the tumor margins; probably as a consequence of better blood supply to those areas.”

In one embodiment of this invention, the phase transition temperature of the nanomagnetic particles is less than about 50 degrees Celsius and, preferably, less than about 46 degrees Celsius. In one aspect of this embodiment, such phase transition temperature is less than about 45 degrees Celsius.

The Diverse Atomic Nature of the Nanomagnetic Particles

In one embodiment, the nanomagnetic particles are depicted by the formula A₁A₂(B)ₓC₁(C₂)_{y}, wherein each of A₁ and A₂ are separate magnetic A moieties, as described below; B is as defined elsewhere in this specification; x is an integer from 0 to 1; each of C₁ and C₂ is as descried elsewhere in this specification; and y is an integer from 0 to 1.

The composition of these preferred nanomagnetic particles may be depicted by a phase diagram such as, e.g., the phase diagram depicted in FIGS. 37 et seq. of U.S. Pat. No. 6,765,144, the entire disclosure of which is hereby incorporated by reference into this specification. As is disclosed in such United States patent, “Referring to FIG. 37, and in the preferred embodiment depicted therein, a phase diagram 5000 is presented. As is illustrated by this phase diagram 5000, the nanomagnetic material used in the composition of this invention preferably is comprised of one or more of moieties A, B, and C . . . . The moiety A depicted in phase diagram 5000 is comprised of a magnetic element selected from the group consisting of a transition series metal, a rare earth series metal, or actinide metal, a mixture thereof, and/or an alloy thereof . . . . As is known to those skilled in the art, the transition series metals include chromium, manganese, iron, cobalt, nickel. One may use alloys or iron, cobalt and nickel such as, e.g., iron-aluminum, iron-carbon, iron-chromium, iron-cobalt, iron-nickel, iron nitride (Fe3 N), iron phosphide, iron-silicon, iron-vanadium, nickel-cobalt, nickel-copper, and the like. One may use alloys of manganese such as, e.g., manganese-aluminum, manganese-bismuth, MnAs, MnSb, MnTe, manganese-copper, manganese-gold, manganese-nickel, manganese-sulfur and related compounds, manganese-antimony, manganese-tin, manganese-zinc, Heusler alloy, and the like. One may use compounds and alloys of the iron group, including oxides of the iron group, halides of the iron group, borides of the transition elements, sulfides of the iron group, platinum and palladium with the iron group, chromium compounds, and the like.”

U.S. Pat. No. 6,765,144 also discloses that: “One may use a rare earth and/or actinide metal such as, e.g., Ce, Pr, Nd, Pm, Sm, Eu, Gd, Tb, Dy, Ho, Er, Tm, Yb, Lu, La, mixtures thereof, and alloys thereof. One may also use one or more of the actinides such as, e.g., Th, Pa, U, Np, Pu, Am, Cm, Bk, Cf. Es, Fm, Md, No, Lr, Ac, and the like . . . . These moieties, compounds thereof, and alloys thereof are well known and are described, e.g., in the aforementioned text of R. S. Tebble et al. entitled “Magnetic Materials . . . . In one preferred embodiment, moiety A is selected from the group consisting of iron, nickel, cobalt, alloys thereof, and mixtures thereof. In this embodiment, the moiety A is magnetic, i.e., it has a relative magnetic permeability of from about 1 to about 500,000 . . . . ”

U.S. Pat. No. 6,765,144 also discloses that “The moiety A also preferably has a saturation magnetization of from about 1 to about 36,000 Gauss, and a coercive force of from about 0.01 to about 5,000 Oersteds . . . . The moiety A may be present in the nanomagnetic material either in its elemental form, as an alloy, in a solid solution, or as a compound . . . It is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.).”

U.S. Pat. No. 6,765,144 also discloses that “In addition to moiety A, it is preferred to have moiety B be present in the nanomagnetic material. In this embodiment, moieties A and B are admixed with each other. The mixture may be a physical mixture, it may be a solid solution, it may be comprised of an alloy of the A/B moieties, etc.

In one embodiment, the magnetic material A is dispersed within nonmagnetic material B. This embodiment is depicted schematically in FIG. 38.”

U.S. Pat. No. 6,765,144 also discloses that “Referring to FIG. 38, and in the preferred embodiment depicted therein, it will be seen that A moieties 5002, 5004, and 5006 are separated from each other either at the atomic level and/or at the nanometer level. The A moieties may be, e.g., A atoms, clusters of A atoms, A compounds, A solid solutions, etc; regardless of the form of the A moiety, it has the magnetic properties described hereinabove . . . . In the embodiment depicted in FIG. 38, each A moiety produces an independent magnetic moment. The coherence length (L) between adjacent A moieties is, on average, from about 0.1 to about 100 nanometers and, more preferably, from about 1 to about 50 nanometers . . . . the normalized magnetic interaction between adjacent A moieties 5002 and 5004, and also between 5004 and 5006, is preferably described by the formula M=exp(−x/L), wherein M is the normalized magnetic interaction, exp is the base of the natural logarithm (and is approximately equal to 2.71828), x is the distance between adjacent A moieties, and L is the coherence length.”

U.S. Pat. No. 6,765,144 also discloses that “In one embodiment, and referring again to FIG. 38, x is preferably measured from the center 5001 of A moiety 5002 to the center 5003 of A moiety 5004; and x is preferably equal to from about 0.00001×L to about 100×L . . . . In one embodiment, the ratio of x/L is at least 0.5 and, preferably, at least 1.5.”

U.S. Pat. No. 6,765,144 also discloses that “Referring again to FIG. 37, the nanomagnetic material may be comprised of 100 percent of moiety A, provided that such moiety A has the required normalized magnetic interaction (M). Alternatively, the nanomagnetic material may be comprised of both moiety A and moiety B . . . . When moiety B is present in the nanomagnetic material, in whatever form or forms it is present, it is preferred that it be present at a mole ratio (by total moles of A and B) of from about 1 to about 99 percent and, preferably, from about 10 to about 90 percent . . . . The B moiety, in whatever form it is present, is nonmagnetic, i.e., it has a relative magnetic permeability of 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like . . . . In one embodiment, and without wishing to be bound to any particular theory, it is believed that B moiety provides plasticity to the nanomagnetic material that it would not have but for the presence of B . . . . ”

U.S. Pat. No. 6,765,144 also discloses that “The use of the B material allows one to produce a coated substrate with a springback angle of less than about 45 degrees. As is known to those skilled in the arty all materials have a finite modulus of elasticity; thus, plastic deformations followed by some elastic recovery when the load is removed. In bending, this recovery is called springback. See, e.g., page 462 of S. Kalparjian's “Manufacturing Engineering and Technology,” Third Edition (Addison Wesley Publishing Company, New York, N.Y., 1995) . . . . FIG. 39 illustrates how springback is determined in accordance with this invention. Referring to FIG. 39, a coated substrate 5010 is subjected to a force in the direction of arrow 5012 that bends portion 5014 of the substrate to an angle 5016 of 45 degrees, preferably in a period of less than about 10 seconds. Thereafter, when the force is released, the bent portion 5014 springs back to position 5018. The springback angle 5020 is preferably less than 45 degrees and, preferably, is less than about 10 degrees.”

U.S. Pat. No. 6,765,144 also discloses that “Referring again to FIG. 37, and in one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, and the like . . . . It is preferred, when the C moiety is present, that it be present in a concentration of from about 1 to about 90 mole percent, based upon the total number of moles of the A moiety and/or the B moiety and C moiety in the composition.”

In one embodiment, the aforementioned moiety A is preferably comprised of a magnetic element selected from the group consisting of a transition series metal, a rare earth series metal, or actinide metal, a mixture thereof, and/or an alloy thereof. In one embodiment, the moiety A is iron. In another embodiment, moiety A is nickel. In yet another embodiment, moiety A is cobalt. In yet another embodiment, moiety A is gadolinium. In another embodiment, the A moiety is selected from the group consisting of samarium, holmium, neodymium, and one or more other member of the Lanthanide series of the periodic table of elements.

In one preferred embodiment, two or more A moieties are present, as atoms. In one aspect of this embodiment, the magnetic susceptibilities of the atoms so present are both positive.

In one embodiment, two or more A moieties are present, at least one of which is iron. In one aspect of this embodiment, both iron and cobalt atoms are present.

When both iron and cobalt are present, it is preferred that from about 10 to about 90 mole percent of iron be present by mole percent of total moles of iron and cobalt present in the ABC moiety. In another embodiment, from about 50 to about 90 mole percent of iron is present. In yet another embodiment, from about 60 to about 90 mole percent of iron is present. In yet another embodiment, from about 70 to about 90 mole percent of iron is present.

In one preferred embodiment, moiety A is selected from the group consisting of iron, nickel, cobalt, alloys thereof, and mixtures thereof.

The moiety A may be present in the nanomagnetic material either in its elemental form, as an alloy, in a solid solution, or as a compound.

In one embodiment, it is preferred at least about 1 mole percent of moiety A be present in the nanomagnetic material (by total moles of A, B, and C), and it is more preferred that at least 10 mole percent of such moiety A be present in the nanomagnetic material (by total moles of A, B, and C). In one embodiment, at least 60 mole percent of such moiety A is present in the nanomagnetic material, (by total moles of A, B, and C.)

In one embodiment, the nanomagnetic material has the formula A₁A₂(B)ₓC₁(C₂)_{y}, wherein each of A₁ and A₂ are separate magnetic A moieties, as described above; B is as defined elsewhere in this specification; x is an integer from 0 to 1; each of C₁ and C₂ is as descried elsewhere in this specification; and y is an integer from 0 to 1.

In this embodiment, there are always two distinct A moieties, such as, e.g., nickel and iron, iron and cobalt, etc. The A moieties may be present in equimolar amounts; or they may be present in non-equimolar amount.

In one aspect of this embodiment, either or both of the A₁ and A₂ moieties are radioactive. Thus, e.g., either or both of the A₁ and A₂ moieties may be selected from the group consisting of radioactive cobalt, radioactive iron, radioactive nickel, and the like. These radioactive isotopes are well known. Reference may be had, e.g., to U.S. Pat. Nos. 3,894,584; 3,936,440 (method of labeling complex metal chelates with radioactive metal isotopes); U.S. Pat. Nos. 4,031,387; 4,282,092; 4,572,797; 4,642,193; 4,659,512; 4,704,245; 4,758,874 (minimization of radioactive material deposition in water-cooled nuclear reactors); U.S. Pat. No. 4,950,449 (inhibition of radioactive cobalt deposition); U.S. Pat. No. 4,647,585 (method for separating cobalt, nickel, and the like from alloys), U.S. Pat. Nos. 4,759,900; 4,781,198 (biopsy tracer needle); U.S. Pat. No. 4,876,449; 5,035,858; 5,196,113; 5,205,167; 5,222,065; 5,241,060 (base moiety-labeled detectable nucleotide); U.S. Pat. No. 6,314,153; and the like. The entire disclosure of each of these United States patents is hereby incorporated by reference into this specification.

In one preferred embodiment, at least one of the A₁ and A₂ moieties is radioactive cobalt. This radioisotope is discussed, e.g., in U.S. Pat. No. 3,936,440, the entire disclosure of which is hereby incorporated by reference into this specification.

In one embodiment, at least one of the A₁ and A₂ is radioactive iron. This radioisotope is also well known as is evidenced, e.g., by U.S. Pat. No. 4,459,356, the entire disclosure of which is also hereby incorporated by reference into this specification. Thus, and as is disclosed in such patent, “In accordance with the present invention, a radioactive stain composition is developed as a result of introduction of a radionuclide (e.g., radioactive iron isotope 59 Fe, which is a strong gamma emitter having peaks of 1.1 and 1.3 MeV) into BPS to form ferrous BPS . . . . In order to prepare the radioactive stain composition, sodium bathophenanthroline sulfonate (BPS), ascorbic acid and Tris buffer salts were obtained from Sigma Chemical Co. (St. Louis, Mo.). Enzymes grade acrylamide, N,N′ methylenebisacrylamide and N,N,N′,N′-tetramethylethylenediamine (TEMED) are products of and were obtained from Eastman Kodak Co. (Rochester, N.Y.). Sodium dodecylsulfate (SDS) was obtained from Pierce Chemicals (Rockford, Ill.). The radioactive isotope (59 FeCl3 in 0.05M HCl, specific activity 15.6 mC/mg) was purchased from New England Nuclear (Boston, Mass.), but was diluted to 10 ml with 0.5N HCl to yield an approximately 0.1 mM Fe(III) solution.”

In the nanomagnetic particles, there may be, but need not be, a B moiety (such as, e.g., aluminum). There preferably are at least two C moieties such as, e.g., oxygen and nitrogen. The A moieties, in combination, comprise at least about 80 mole percent of such a composition; and they preferably comprise at least 90 mole percent of such composition.

When two C moieties are present, and when the two C moieties are oxygen and nitrogen, they preferably are present in a mole ratio such that from about 10 to about 90 mole percent of oxygen is present, by total moles of oxygen and nitrogen. It is preferred that at least about 60 mole percent of oxygen be present. In one embodiment, at least about 70 mole percent of oxygen is so present. In yet another embodiment, at least 80 mole percent of oxygen is so present.

One may measure the surface of the nanomagnetic material, measuring the first 8.5 nanometers of material. When such surface is measured, it is preferred that at least 50 mole percent of oxygen, by total moles of oxygen and nitrogen, be present in such surface. It is preferred that at least about 60 mole percent of oxygen be present. In one embodiment, at least about 70 mole percent of oxygen is so present. In yet another embodiment, at least 80 mole percent of oxygen is so present.

Without wishing to be bound to any particular theory, applicants believe that the presence of two distinct A moieties in their composition, and two distinct C moieties (such as, e.g., oxygen and nitrogen), provides better magnetic properties for applicants' nanomagnetic materials.

The B moiety, in one embodiment, in whatever form it is present, is preferably nonmagnetic, i.e., it has a relative magnetic permeability of about 1.0; without wishing to be bound to any particular theory, applicants believe that the B moiety acts as buffer between adjacent A moieties. One may use, e.g., such elements as silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, and the like.

In one embodiment, the B moiety has a relative magnetic permeability that is about equal to 1 plus the magnetic susceptibility. The relative magnetic susceptilities of silicon, aluminum, boron, platinum, tantalum, palladium, yttrium, zirconium, titanium, calcium, beryllium, barium, silver, gold, indium, lead, tin, antimony, germanium, gallium, tungsten, bismuth, strontium, magnesium, zinc, copper, cesium, cerium, hafnium, iodine, iridium, lanthanum, lithium, lutetium, manganese, molybdenum, potassium, sodium, strontium, praseodymium, rhenium, rhodium, rubidium, ruthenium, scandium, selenium, tantalum, technetium, tellurium, chromium, thallium, thorium, thulium, titanium, vanadium, zinc, yttrium, ytterbium, zirconium, and the like. Reference may be had, e.g., to pages E-118 through E 123 of the aforementioned CRC Handbook of Chemistry and Physics.

In one embodiment, the nanomagnetic particles may be represented by the formula AₓB_{y}C_{z} wherein x+y+z is equal to 1. In this embodiment the ratio of x/y is at least 0.1 and preferably at least 0.2; and the ratio of z/x is from 0.001 to about 0.5.

In one preferred embodiment, the B material is aluminum and the C material is nitrogen, whereby an AlN moiety is formed. Without wishing to be bound to any particular theory, applicants believe that aluminum nitride (and comparable materials) are both electrically insulating and thermally conductive, thus providing a excellent combination of properties for certain end uses.

In one embodiment, the nanomagnetic material is comprised of moiety A, moiety C, and optionally moiety B. The moiety C is preferably selected from the group consisting of elemental oxygen, elemental nitrogen, elemental carbon, elemental fluorine, elemental chlorine, elemental hydrogen, and elemental helium, elemental neon, elemental argon, elemental krypton, elemental xenon, elemental fluorine, elemental sulfur, elemental hydrogen, elemental helium, the elemental chlorine, elemental bromine, elemental iodine, elemental boron, elemental phosphorus, and the like. In one aspect of this embodiment, the C moiety is selected from the group consisting of elemental oxygen, elemental nitrogen, and mixtures thereof.

In one embodiment, the C moiety is chosen from the group of elements that, at room temperature, form gases by having two or more of the same elements combine. Such gases include, e.g., hydrogen, the halide gases (fluorine, chlorine, bromine, and iodine), inert gases (helium, neon, argon, krypton, xenon, etc.), etc.

In one embodiment, the C moiety is chosen from the group consisting of oxygen, nitrogen, and mixtures thereof. In one aspect of this embodiment, the C moiety is a mixture of oxygen and nitrogen, wherein the oxygen is present at a concentration from about 10 to about 90 mole percent, by total moles of oxygen and nitrogen.

It is preferred, when the C moiety (or moieties) is present, that it be present in a concentration of from about 1 to about 90 mole percent, based upon the total number of moles of the A moiety and/or the B moiety and the C moiety in the composition. In one embodiment, the C moiety is both oxygen and nitrogen.

The molar ratio of A/(A and B and C) generally is from about 1 to about 99 molar percent and, preferably, from about 10 to about 90 molar percent. In one embodiment, such molar ratio is from about 30 to about 60 molar percent.

The molar ratio of B/(A plus B plus C) generally is from about 1 to about 99 mole percent and, preferably, from about 10 to about 40 mole percent.

The molar ratio of C/(A plus B plus C) generally is from about 1 to about 99 mole percent and, preferably, from about 10 to about 50 mole percent.

In one embodiment, the B moiety is added to the nanomagnetic A moiety, preferably with a B/A molar ratio of from about 5:95 to about 95:5 (see FIG. 3). In one aspect of this embodiment, the resistivity of the mixture of the B moiety and the A moiety is from about 1 micro-ohm-cm to about 10,000 micro-ohm-cm.

In one particularly preferred embodiment, the A moiety is iron, the B moiety is aluminum, and the molar ratio of A/B is about 70:30; the resistivity of this mixture is about 8 micro-ohms-cm.

The Squareness of the Nanomagnetic Particles of the Invention

As is known to those skilled in the art, the squareness of a magnetic material is the ratio of the residual magnetic flux and the saturation magnetic flux density. Reference may be had, e.g., to U.S. Pat. Nos. 6,627,313, 6,517,934, 6,458,452, 6,391,450, 6,350,505, 6,248,437, 6,194,058, 6,042,937, 5,998,048, 5,645,652, and the like. The entire disclosure of such United States patents is hereby incorporated by reference into this specification. Reference may also be had to page 1802 of the McGraw-Hill Dictionary of Scientific and Technical Terms, Fourth Edition (McGraw-Hill Book Company, New York, N.Y., 1989). At such page 1802, the “squareness ratio” is defined as “The magnetic induction at zero magnetizing force divided by the maximum magnetic indication, in a symmetric cyclic magnetization of a material.”

In one embodiment, the squareness of applicants' nanomagnetic particles is from about 0.05 to about 1.0. In one aspect of this embodiment, such squareness is from about 0.1 to about 0.9. In another aspect of this embodiment, the squareness is from about 0.2 to about 0.8. In applications where a large residual magnetic moment is desired, the squareness is preferably at least about 0.8.

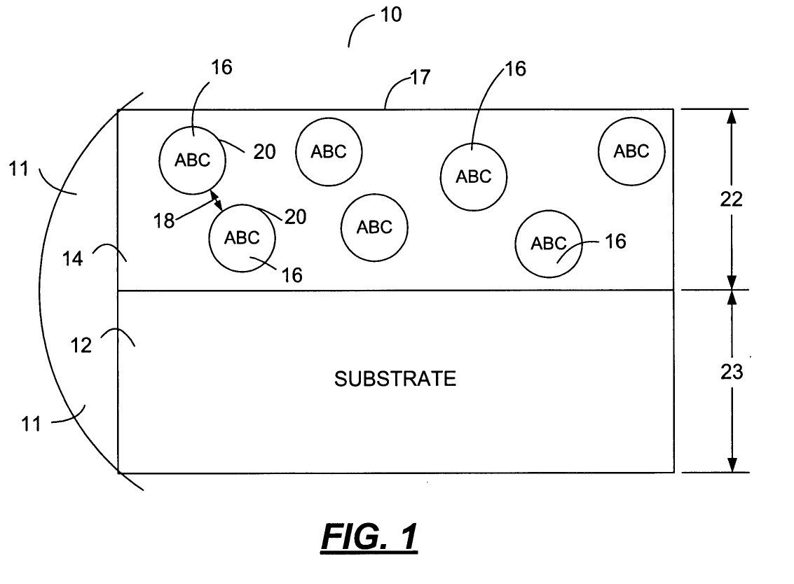

FIG. 1 is a schematic illustration, not drawn to scale, of a coated substrate assembly 10 comprised of a substrate 12 and, disposed thereon, a coating 14 comprised of a multiplicity of nanomagnetic particles 16. Similar coated substrate assemblies are illustrated and described in applicants' United States patents. Reference may be had, e.g., to U.S. Pat. No. 6,506,972 (magnetically shielded conductor), U.S. Pat. No. 6,700,472 (magnetic thin film inductors), U.S. Pat. No. 6,713,671 (magnetically shielded assembly), U.S. Pat. No. 6,765,144 (magnetic resonance imaging coated assembly), and the like. The entire disclosure of each of these United States patents is hereby incorporated by reference into this specification.

Referring to FIG. 1, and to the preferred embodiment depicted therein, it will be seen that the nanomagnetic particles 16 are preferably comprised of the “ABC” atoms described elsewhere in this specification. With regard to such “ABC” particles, the term “coherence length” refers to the smallest distance 18 between the surfaces 20 of any particles 16 that are adjacent to each other. In one aspect of this embodiment, it is preferred that such coherence length, with regard to such ABC particles, be less than about 100 nanometers and, preferably, less than about 50 nanometers. In one embodiment, such coherence length is less than about 20 nanometers. It is preferred that, regardless of the coherence length used, it be at least 2 times as great as the maximum dimension of the particles 16.

The Mass Density of the Nanomagnetic Particles

In one embodiment, the nanomagnetic material preferably has a mass density of at least about 0.001 grams per cubic centimeter; in one aspect of this embodiment, such mass density is at least about 1 gram per cubic centimeter. As used in this specification, the term mass density refers to the mass of a give substance per unit volume. See, e.g., page 510 of the aforementioned “McGraw-Hill Dictionary of Scientific and Technical Terms.” In another embodiment, the material has a mass density of at least about 3 grams per cubic centimeter. In another embodiment, the nanomagnetic material has a mass density of at least about 4 grams per cubic centimeter.

The Thickness of the Coating 14

Referring again to FIG. 1, and to the preferred embodiment depicted therein, the coating 14 may be comprised of one layer of material, two layers of material, or three or more layers of material. Regardless of the number of coating layers used, it is preferred that the total thickness 22 of the coating 14 be at least about 400 nanometers and, preferably, be from about 400 to about 4,000 nanometers. In one embodiment, thickness 22 is from about 600 to about 1,000 nanometers. In another embodiment, thickness 22 is from about 750 to about 850 nanometers.

In the embodiment depicted, the substrate 12 has a thickness 23 that is substantially greater than the thickness 22. As will be apparent, the coated substrate 10 is not drawn to scale.

In general, the thickness 22 is preferably less than about 5 percent of thickness 23 and, more preferably, less than about 2 percent. In one embodiment, the thickness 22 is no greater than about 1.5 percent of the thickness 23.

The Flexibility of Coated Substrate 10

Referring to FIG. 1, and in one preferred embodiment thereof, substrate 12 is a conductor that preferably has a resistivity at 20 degrees Centigrade of from about 1 to about 100-microohom-centimeters. In this embodiment, disposed above the conductor 12 is a film 14 comprised of nanomagnetic particles 16 that preferably have a maximum dimension of from about 10 to about 100 nanometers. The film 114 also preferably has a saturation magnetization of from about 200 to about 26,000 Gauss and a thickness of less than about 2 microns.

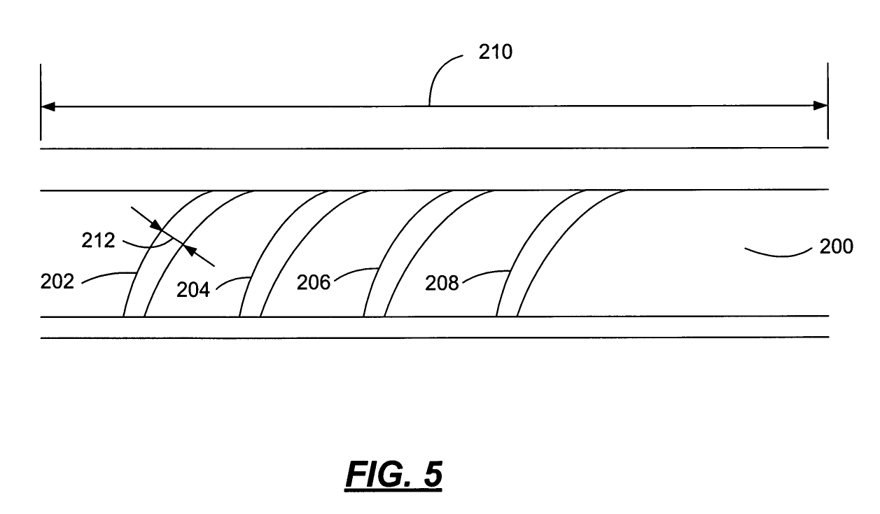

In one aspect of this embodiment, conductor assembly 10 is flexible, having a bend radius of less than 2 centimeters. Reference may be had, e.g., to U.S. Pat. No. 6,506,972, the entire disclosure of which is hereby incorporated by reference into this specification. A similar device is depicted in FIG. 5 of U.S. Pat. No. 6,713,671; the entire disclosure of such United States patent is hereby incorporated by reference into this specification.

As used in this specification, the term flexible refers to an assembly that can be bent to form a circle with a radius of less than 2 centimeters without breaking. Put another way, the bend radius of the coated assembly is preferably less than 2 centimeters. Reference may be had, e.g., to U.S. Pat. Nos. 4,705,353, 5,946,439, 5,315,365, 4,641,917, 5,913,005, and the like. The entire disclosure of each of these United States patents is hereby incorporated by reference into this specification.

Without wishing to be bound to any particular theory, applicants believe that the use of nanomagnetic particles in their coatings and their articles of manufacture allows one to produce a flexible device that otherwise could not be produced were not the materials so used nano-sized (less than 100 nanometers).

In another embodiment, not shown, the assembly 10 is not flexible.

The Morphological Density of the Coating 14

In one preferred embodiment, and referring to FIG. 1, the coating 14 has a morphological density of at least about 98 percent. In the embodiment depicted, the coating 14 has a thickness 22 of from about 400 to about 2,000 nanometers and, in one embodiment, has a thickness 22 of from about 600 to about 1200 nanometers.

As is known to those skilled in the art, the morphological density of a coating is a function of the ratio of the dense coating material on its surface to the pores on its surface; and it is usually measured by scanning electron microscopy. By way of illustration, e.g., published United States patent application US 2003/0102222A1 contains a FIG. 3A that is a scanning electron microscope (SEM) image of a coating of “long” single-walled carbon nanotubes on a substrate. Referring to this SEM image, it will be seen that the white areas are the areas of the coating where pores occur.

The technique of making morphological density measurements also is described, e.g., in a M.S. thesis by Raymond Lewis entitled “Process study of the atmospheric RF plasma deposition system for oxide coatings” that was deposited in the Scholes Library of Alfred University, Alfred, N.Y. in 1999 (call Number TP2 a75 1999 vol 1., no. 1.).

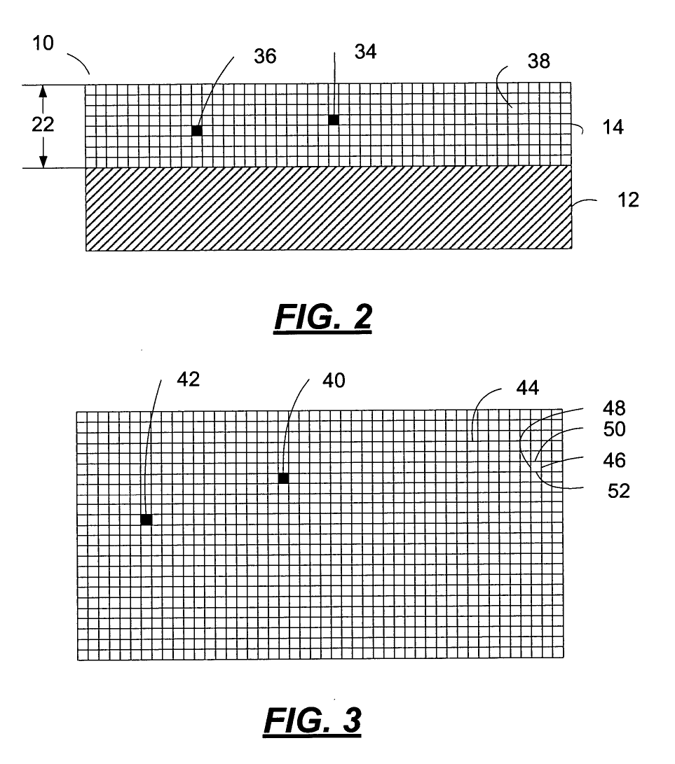

The scanning electron microscope (SEM) images obtained in making morphological density measurements can be divided into a matrix., as is illustrated in FIGS. 2 and 3 which schematically illustrate the porosity of the side of coating 14, and the top of the coating 14. The SEM image depicted shows two pores 34 and 36 in the cross-sectional area 38, and it also shows two pores 40 and 42 in the top 44. As will be apparent, the SEM image can be divided into a matrix whose adjacent lines 46/48, and adjacent lines 50/52 define a square portion with a surface area of 100 square nanometers (10 nanometers×10 nanometers). Each such square portion that contains a porous area is counted, as is each such square portion that contains a dense area. The ratio of dense areas/porous areas, ×100, is preferably at least 98. Put another way, the morphological density of the coating 14 is at least 98 percent. In one embodiment, the morphological density of the coating 14 is at least about 99 percent. In another embodiment, the morphological density of the coating 14 is at least about 99.5 percent.

One may obtain such high morphological densities by atomic size deposition, i.e., the particles sizes deposited on the substrate are atomic scale. The atomic scale particles thus deposited often interact with each other to form nano-sized moieties that are less than 100 nanometers in size.

The Surface Roughness of the Coating 14

In one embodiment, the coating 14 (see FIG. 1) has an average surface roughness of less than about 100 nanometers and, more preferably, less than about 10 nanometers. As is known to those skilled in the art, the average surface roughness of a thin film is preferably measured by an atomic force microscope (AFM). Reference may be had, e.g., to U.S. Pat. No. 5,420,796 (method of inspecting planarity of wafer surface), U.S. Pat. Nos. 6,610,004, 6,140,014, 6,548,139, 6,383,404, 6,586,322, 5,832,834, and 6,342,277. The entire disclosure of each of these United States patents is hereby incorporated by reference into this specification.

Alternatively, or additionally, one may measure surface roughness by a laser interference technique. This technique is well known. Reference may be had, e.g., to U.S. Pat. No. 6,285,456 (dimension measurement using both coherent and white light interferometers), U.S. Pat. Nos. 6,136,410, 5,843,232 (measuring deposit thickness), U.S. Pat. No. 4,151,654 (device for measuring axially symmetric aspherics), and the like. The entire disclosure of these United States patents are hereby incorporated by reference into this specification.

Hydrophobic and Hydrophilic Coatings

By varying the surface roughness of the coating 14 (see FIG. 1), one may make the surface 17 of such coating either hydrophobic or hydrophilic.

As is known to those skilled in the art, a hydrophobic material is antagonistic to water and incapable of dissolving in water. Inasmuch as the average water droplet has a minimum cross-sectional dimension of at least about 3 nanometers, the water droplets will tend not to bond to a coated surface 17 which, has a surface roughness of, e.g., 1 nanometer.

One may vary the average surface roughness of coated surface 17 by varying the pressure used in the sputtering process described elsewhere in this specification. In general, the higher the gas pressure used, the rougher the surface.

If, on the other hand, one modifies the sputtering process to allow a surface roughness of at about, e.g., 20 nanometers, the water droplets then have an opportunity to bond to the surface 17 which, in this embodiment, will tend to be hydrophilic.

Durable Properties of the Coated Substrate 10

In one embodiment, the coated substrate of this invention has durable magnetic properties that do not vary upon extended exposure to a saline solution. If the magnetic moment of a coated substrate is measured at “time zero” (i.e., prior to the time it has been exposed to a saline solution), and then the coated substrate is then immersed in a saline solution comprised of 7.0 mole percent of sodium chloride and 93 mole percent of water, and if the substrate/saline solution is maintained at atmospheric pressure and at temperature of 98.6 degrees Fahrenheit for 6 months, the coated substrate, upon removal from the saline solution and drying, will be found to have a magnetic moment that is within plus or minus 5 percent of its magnetic moment at time zero.

In another embodiment, the coated substrate of this invention has durable mechanical properties when tested by the saline immersion test described above.

Thus, e.g., the substrate 12, prior to the time it is coated with coating 14, has a certain flexural strength, and a certain spring constant.

The flexural strength is the strength of a material in bending, i.e., its resistance to fracture. As is disclosed in ASTM C-790, the flexural strength is a property of a solid material that indicates its ability to withstand a flexural or transverse load. As is known to those skilled in the art, the spring constant is the constant of proportionality k which appears in Hooke's law for springs. Hooke's law states that: F=−kx, wherein F is the applied force and x is the displacement from equilibrium. The spring constant has units of force per unit length.

Means for measuring the spring constant of a material are well known to those skilled in the art. Reference may be had, e.g., to U.S. Pat. No. 6,360,589 (device and method for testing vehicle shock absorbers), U.S. Pat. No. 4,970,645 (suspension control method and apparatus for vehicle), U.S. Pat. Nos. 6,575,020, 4,157,060, 3,803,887, 4,429,574, 6,021,579, and the like. The entire disclosure of each of these United States patents is hereby incorporated by reference into this specification.

Referring again to FIG. 1, the flexural strength of the uncoated substrate 10 preferably differs from the flexural strength of the coated substrate 10 by no greater than about 5 percent. Similarly, the spring constant of the uncoated substrate 10 differs from the spring constant of the coated substrate 10 by no greater than about 5 percent.

In one embodiment, the coating 14 biocompatible with biological organisms. As used herein, the term biocompatible refers to a coating whose chemical composition does not change substantially upon exposure to biological fluids. Thus, when the coating 14s immersed in a 7.0 mole percent saline solution for 6 months maintained at a temperature of 98.6 degrees Fahrenheit, its chemical composition (as measured by, e.g., energy dispersive X-ray analysis [EDS, or EDAX]) is substantially identical to its chemical composition at “time zero.”

The Susceptibility of the Coated Substrate 10

In one preferred embodiment (see FIG. 1), the coated substrate 10 has a direct current (d.c.) magnetic susceptibility within a specified range. As is known to those skilled in the art, magnetic susceptibility is the ratio of the magnetization of a material to the magnetic field strength; it is a tensor when these two quantities are not parallel; otherwise it is a simple number. Reference may be had, e.g., to U.S. Pat. No. 3,614,618 (magnetic susceptibility tester), U.S. Pat. No. 3,644,823 (nulling coil for magnetic susceptibility logging), U.S. Pat. No. 3,758,848 (method and system with voltage cancellation for measuring the magnetic susceptibility of a subsurface earth formation), U.S. Pat. No. 3,879,658 (apparatus for measuring magnetic susceptibility), U.S. Pat. No. 3,980,076 (method for measuring externally of the human body magnetic susceptibility changes), U.S. Pat. No. 4,277,750 (induction probe for the measurement of magnetic susceptibility), U.S. Pat. No. 4,662,359 (use of magnetic susceptibility probes in the treatment of cancer), U.S. Pat. No. 4,985,165 (material having a predeterminable magnetic susceptibility), U.S. Pat. No. 5,300,886 (method to enhance the susceptibility of MRI for magnetic susceptibility effects), U.S. Pat. No. 6,208,884 (noninvasive room temperature instrument to measure magnetic susceptibiolity variations in body tissue), U.S. Pat. No. 6,477,398 (resonant magnetic susceptibility imaging), and the like. The entire disclosure of each of these United States patents is hereby incorporated by reference into this specification.

In one aspect of this embodiment, and referring again to FIG. 1, the substrate 12 is a stent that is comprised of wire mesh constructed in such a manner as to define a multiplicity of openings. The mesh material is preferably a metal or metal alloy, such as, e.g., stainless steel, Nitinol (an alloy of nickel and titanium), niobium, copper, etc.

Typically the materials used in stents tend to cause current flow when exposed to a radio frequency field. When the field is a nuclear magnetic resonance field, it generally has a direct current component, and a radio-frequency component. For MRI (magnetic resonance imaging) purposes, a gradient component is added for spatial resolution.

The material or materials used to make the stent itself have certain magnetic properties such as, e.g., magnetic susceptibility. Thus, e.g., niobium has a magnetic susceptibility of 1.95×10⁻⁶ centimeter-gram-second units. Nitonol has a magnetic susceptibility of from about 2.5 to about 3.8×10⁻⁶ centimeter-gram-second units. Copper has a magnetic susceptibility of from −5.46 to about −6.16×10⁻⁶ centimeter-gram-second units.

The total magnetic susceptibility of an object is equal to the mass of the object times its susceptibility. Thus, assuming an object has equal parts of niobium, Nitinol, and copper, its total susceptibility would be equal to (+1.95+3.15−5.46)×10⁻⁶ cgs, or about 0.36×10⁻⁶ cgs.

In a more general case, where the masses of niobium, Nitinol, and copper are not equal in the object, the susceptibility, in c.g.s. units, would be equal to 1.95 Mn+3.15 Mni−5.46 Mc, wherein Mn is the mass of niobium, Mni is th mass of Nitinol, and Mc is the mass of copper.

Referring again to FIG. 1, and in one preferred embodiment thereof, the coated substrate assembly 10 preferably materials that will provide the desired mechanical properties generally do not have desirable magnetic and/or electromagnetic properties. In an ideal situation, the stent 500 will produce no loop currents and no surface eddy currents when exposed to magnetic resonance imaging (MRI) radiation and, in such situation, has an effective zero magnetic susceptibility. Put another way, ideally the direct current magnetic susceptibility of an ideal coated substrate that is exposed to MRI radiation should be about 0.

A d.c. (“direct current”) magnetic susceptibility of precisely zero is often difficult to obtain. In general, it is sufficient if the d.c. susceptibility of the coated substrate 10 is plus or minus 1×10⁻³ centimeter-gram-seconds (cgs) and, more preferably, plus or minus 1×10⁻⁴ centimeter-gram-seconds. In one embodiment, the d.c. susceptibility of the coated substrate 10 is equal to plus or minus 1×10⁻⁵ centimeter-gram-seconds. In another embodiment, the d.c. susceptibility of the coated substrate 10 is equal to plus or minus 1×10⁻⁶ centimeter-gram-seconds.

In one embodiment, and referring again to FIG. 1, the coated substrate assembly 10 is in contact with biological tissue 11. In FIG. 1, only a portion of the biological tissue 11 actually contiguous with assembly 10 is shown for the sake of simplicity of representation. In such an embodiment, it is preferred that such biological tissue 11 be taken into account when determining the net susceptibility of the assembly, and that such net susceptibility of the assembly 10 in contact with bodily fluid is plus or minus plus or minus 1×10⁻³ centimeter-gram-seconds (cgs), or plus or minus 1×10⁻⁴ centimeter-gram-seconds, or plus or minus 1×10⁻⁵ centimeter-gram-seconds, or plus or minus 1×10⁻⁶ centimeter-gram-seconds. In this embodiment, the materials comprising the nanomagnetic coating 14 on the substrate 12 are chosen to have susceptibility values that, in combination with the susceptibility values of the other components of the assembly, and of the bodily fluid, will yield the desired values.

The prior art has heretofore been unable to provide such an implantable stent that will have the desired degree of net magnetic susceptibility. Applicants' invention allows one to compensate for the deficiencies of the current stents, and/or of the current stents in contact with bodily fluid, by canceling the undesirable effects due to their magnetic susceptibilities, and/or by compensating for such undesirable effects.

When different objects are subjected to an electromagnetic field (such as an MRI field), they will exhibit different magnetic responses at different field strengths. Thus, e.g., copper, at a d.c. field strength of 1.5 Tesla, changes its magnetization as a function of the composite field strength (including the d.c. field strength, the r.f. field strength, and the gradient field strength) at a rate (defined by delta-magnetization/delta composite field strength) that is decreasing. With regard to the r.f. field and the gradient field, it should be understood that the order of magnitude of these fields is relatively small compared to the d.c. field, which is usually about 1.5 Tesla. The slope of the graph of magnetization versus field strength for copper is negative; this negative slope indicates that copper, in response to the applied fields, is opposing the applied fields. Because the applied fields (including r.f. fields, and the gradient fields), are required for effective MRI imaging, the response of the copper to the applied fields tends to block the desired imaging. The d.c. susceptibility of copper is equal to the mass of the copper present in the device 10 times its magnetic susceptibility.

By comparison to copper, the ideal magnetization response of a composite assembly (such as, e.g., assembly 10) will be a line whose slope is substantially zero. As used herein, the term “substantially zero” includes a slope will produce an effective magnetic susceptibility of from about 1×10⁻⁷ to about 1×10⁻⁸ centimeters-gram-second (cgs).

One means of correcting negative slope the graph for copper is by coating the copper with a coating which produces a magnetization response with a positive slope so that the composite material produces the desired effective magnetic susceptibility of from about 1×10⁻⁷ to about 1×10⁻⁸ centimeters-gram-second (cgs) units. In order to do so, the following equation must be satisfied: (magnetic susceptibility of the uncoated device)(mass of uncoated device)+(magnetic susceptibility of copper)(mass of copper)=from about 1×10⁻⁷ to about 1×10⁻⁸ centimeters-gram-second (cgs).

In one embodiment, the desired correction for the slope of the copper graph may be obtained by coating the copper with a coating comprised of both nanomagnetic material and nanodielectric material.

In one aspect of this embodiment, the nanomagnetic material preferably has an average particle size of less than about 20 nanometers and a saturation magnetization of from 10,000 to about 26,000 Gauss. In another aspect of this embodiment, the nanomagnetic material used is iron. In another aspect of this embodiment, the nanomagnetic material used is FeAlN. In yet another aspect of this embodiment, the nanomagnetic material is FeAl. Other suitable materials will be apparent to those skilled in the art and include, e.g., nickel, cobalt, magnetic rare earth materials and alloys, thereof, and the like.

In this embodiment, the nanodielectric material used preferably has a resistivity at 20 degrees Centigrade of from about 1×10⁻⁵ ohm-centimeters to about 1×10¹³ ohm-centimeters.

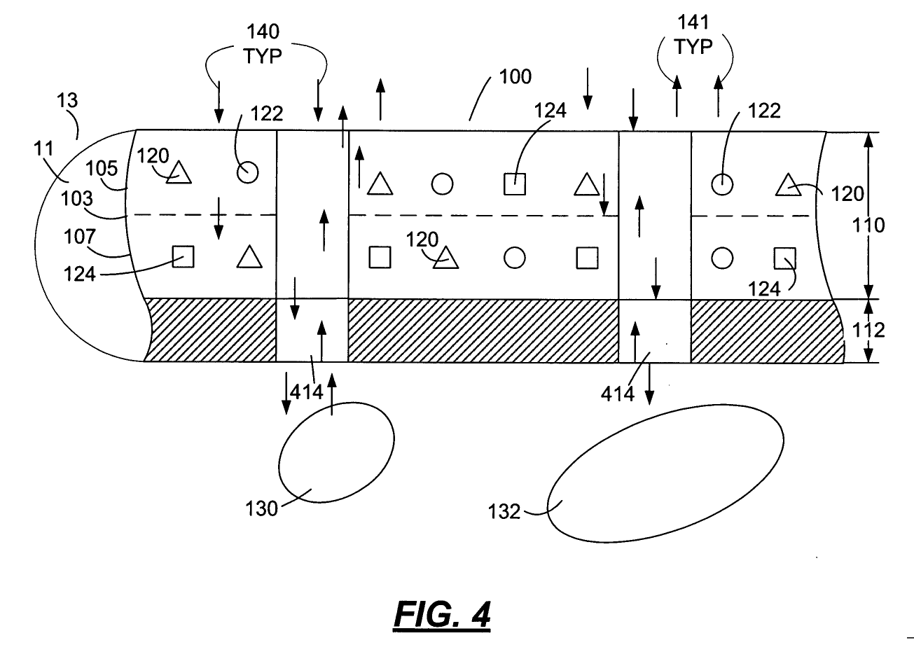

Referring again to FIG. 4, and in the preferred embodiment depicted therein, a coated stent assembly 100 that is comprised of a stent 104 on which is disposed a coating 103 is illustrated. The coating 103 is comprised of nanomagnetic material 120 that is preferably homogeneously dispersed within nanodielectric material 122, which acts as an insulating matrix. In general, the amount of nanodielectric material 122 in coating 103 exceeds the amount of nanomagnetic material 120 in such coating 103.

In one embodiment, the coating 103 is comprised of at least about 70 mole percent of such nanodielectric material (by total moles of nanomagnetic material and nanodielectric material). In another embodiment, the coating 103 is comprised of less than about 20 mole percent of the nanomagnetic material 120, by total moles of nanomagnetic material and nanodielectric material. In one embodiment, the nanodielectric material used is aluminum nitride.

Referring again to FIG. 4, one may optionally include nanoconductive material 424 in the coating 103. This nanoconductive material 124 generally has a resistivity at 20 degrees Centigrade of from about 1×10⁻⁶ ohm-centimeters to about 1×10⁻⁵ ohm-centimeters; and it generally has an average particle size of less than about 100 nanometers. In one aspect of this embodiment, the nanoconductive material used is aluminum.

Referring again to FIG. 4, and in the embodiment depicted, it will be seen that two layers 105/107 are preferably used to obtain the desired correction. In one embodiment, three or more such layers are used. Regardless of the number of such layers 105/107 used, it is preferred that the thickness 110 of coating 103 be from about 400 to about 4000 nanometers

In the embodiment depicted in FIG. 4, the direct current susceptibility of the assembly depicted is equal to the sum of the (mass)×(susceptibility) for each individual layer 105/107 and for the substrate 104.

As will be apparent, it may be difficult with only one layer of coating material to obtain the desired correction for the material comprising the stent assembly 400. With a multiplicity of layers comprising the coating 103, which may have the same and/or different thicknesses, and/or the same and/or different masses, and/or the same and/or different compositions, and/or the same and/or different magnetic susceptibilities, more flexibility is provided in obtaining the desired correction.

Without wishing to be bound to any particular theory, applicants believe that, in the assembly 100 depicted in FIG. 4, each of the different species 120/122/124 within the coatings 105/107 retains its individual magnetic characteristics. These species are preferably not alloyed with each other; when such species are alloyed with each other, each of the species does not retain its individual magnetic characteristics.

An alloy, as that term is used in this specification, is a substance having magnetic properties and consisting of two or more elements, which usually are metallic elements. The bonds in the alloy are usually metallic bonds, and thus the individual elements in the alloy do not retain their individual magnetic properties because of the substantial “crosstalk” between the elements via the metallic bonding process.

By comparison, e.g., materials that are covalently bond to each other are more likely to retain their individual magnetic characteristics; it is such materials whose behavior is illustrated in FIG. 4. Each of the “magnetically distinct” materials may be, e.g., a material in elemental form, a compound, an alloy, etc.

In one embodiment, and referring again to FIG. 4, one may mix “positively magnetized materials” with “negatively magnetized materials” to obtain the desired degree of net magnetization. As is known to those skilled in the art, the positively magnetized species include, e.g., those species that exhibit paramagnetism, superparamagnetism, ferromagnetism, and/or ferrimagnetism.

Paramagnetism is a property exhibited by substances which, when placed in a magnetic field, are magnetized parallel to the field to an extent proportional to the field (except at very low temperatures or in extremely large magnetic fields). Paramagnetic materials are well known to those skilled in the art. Reference may be had, e.g., to U.S. Pat. No. 5,578,922 (paramagnetic material in solution), U.S. Pat. No. 4,704,871 (magnetic refrigeration apparatus with belt of paramagnetic material), U.S. Pat. No. 4,243,939 (base paramagnetic material containing ferromagnetic impurity), U.S. Pat. No. 3,917,054 (articles of paramagnetic material), U.S. Pat. No. 3,796,4999 (paramagnetic material disposed in a gas mixture), and the like. The entire disclosure of each of these United States patents is hereby incorporated by reference into this specification.

Superparamagnetic materials are also well known to those skilled in the art. Reference may be had, e.g., to U.S. Pat. No. 5,238,811, the entire disclosure of which is hereby incorporated by reference into this specification, it is disclosed (at column 5) that: “In one embodiment, the superparamagnetic material used is a substance which has a particle size smaller than that of a ferromagnetic material and retains no residual magnetization after disappearance of the external magnetic field. The superparamagnetic material and ferromagnetic material are quite different from each other in their hysteresis curve, susceptibility, Mesbauer effect, etc. Indeed, ferromagnetic materials are most suited for the conventional assay methods since they require that magnetic micro-particles used for labeling be efficiently guided even when a weak magnetic force is applied.

The preparation of these superparamagnetic materials is discussed at columns 6 et seq. of U.S. Pat. No. 5,238,811, wherein it is disclosed that: “The ferromagnetic substances can be selected appropriately, for example, from various compound magnetic substances such as magnetite and gamma-ferrite, metal magnetic substances such as iron, nickel and cobalt, etc. The ferromagnetic substances can be converted into ultramicro particles using conventional methods excepting a mechanical grinding method, i.e., various gas phase methods and liquid phase methods. For example, an evaporation-in-gas method, a laser heating evaporation method, a coprecipitation method, etc. can be applied. The ultramicro particles produced by the gas phase methods and liquid phase methods contain both superparamagnetic particles and ferromagnetic particles in admixture, and it is therefore necessary to separate and collect only those particles which show superparamagnetic property. For the separation and collection, various methods including mechanical, chemical and physical methods can be applied, examples of which include centrifugation, liquid chromatography, magnetic filtering, etc. The particle size of the superparamagnetic ultramicro particles may vary depending upon the kind of the ferromagnetic substance used but it must be below the critical size of single domain particles. Preferably, it is not larger than 10 nm when the ferromagnetic substance used is magnetite or gamma-ferrite and it is not larger than 3 nm when pure iron is used as a ferromagnetic substance, for example.”

Ferromagnetic materials may also be used as the positively magnetized species. As is known to those skilled in the art, ferromagnetism is a property, exhibited by certain metals, alloys, and compounds of the transition (iron group), rare-earth, and actinide elements, in which the internal magnetic moments spontaneously organize in a common direction; this property gives rise to a permeability considerably greater than that of a cuum, and also to magnetic hysteresis. Reference may be had, e.g., to U.S. Pat. Nos. 6,475,650; 6,299,990; 6,690,287 (ferromagnetic material having improved impedance matching); U.S. Pat. No. 6,366,083 (crud layer containing ferromagnetic material on nuclear fuel rods); U.S. Pat. No. 6,011,674 (magnetoresistance effect multilayer film with ferromagnetic film sublayers of different ferromagnetic material compositions); U.S. Pat. No. 5,648,015 (process for preparing ferromagnetic materials); U.S. Pat. Nos. 5,382,304; 5,272,238 (organo-ferromagnetic material); U.S. Pat. No. 5,247,054 (organic polymer ferromagnetic material); U.S. Pat. No. 5,030,371 (acicular ferromagnetic material consisting essentially of iron-containing chromium dioxide); U.S. Pat. No. 4,917,736 (passive ferromagnetic material); U.S. Pat. No. 4,863,715 (contrast agent comprising particles of ferromagnetic material); U.S. Pat. No. 4,835,510 (magnetoresistive element of ferromagnetic material); U.S. Pat. No. 4,739,294 (amorphous and non-amorphous ferromagnetic material); U.S. Pat. No. 4,289,937 (fine grain ferromagnetic material); U.S. Pat. No. 4,023,412 (the Curie point of a ferromagnetic material); U.S. Pat. No. 4,015,030 (stabilized ferromagnetic material); U.S. Pat. No. 4,004,997 (a polymerizable composition containing a magnetized powdered ferromagnetic material); U.S. Pat. No. 3,851,375 (sintered oxidic ferromagnetic material); U.S. Pat. No. 3,850,706 (ferromagnetic materials comprised of transition metals); and the like. The entire disclosure of each of these United States patents is hereby incorporated by reference into this specification.

Ferrimagnetic materials may also be used as the positively magnetized specifies. As is known to those skilled in the art, ferrimagnetism is a type of magnetism in which the magnetic moments of neighboring ions tend to align nonparallel, usually antiparallel, to each other, but the moments are of different magnitudes, so there is an appreciable, resultant magnetization. Reference may be had, e.g., to U.S. Pat. No. 6,538,919; 6,056,890 (ferrimagnetic materials with temperature stability); U.S. Pat. Nos. 4,649,495; 4,062,920 (lithium-containing ferrimagnetic materials); U.S. Pat. Nos. 4,059,664; 3,947,372 (ferromagnetic material); U.S. Pat. No. 3,886,077 (garnet structure ferromagnetic material); U.S. Pat. Nos. 3,765,021; 3,670,267; and the like. The entire disclosure of each of these United States patents is hereby incorporated by reference into this specification.

By way of yet further illustration, and not limitation, some suitable positively magnetized species include, e.g., iron; iron/aluminum; iron/aluminum oxide; iron/aluminum nitride; iron/tantalum nitride; iron/tantalum oxide; nickel; nickel/cobalt; cobalt/iron; cobalt; samarium; gadolinium; neodymium; mixtures thereof; nano-sized particles of the aforementioned mixtures, where super-paramagnetic properties are exhibited; and the like.

By way of yet further illustration, other suitable positively magnetized species are listed in the “CRC Handbook of Chemistry and Physics,” 63^{rd} Edition (CRC Press, Inc., Boca-Raton, Fla., 1982-1983). As is discussed on pages E-118 to E-123 of such CRC Handbook, materials with positive susceptibility include, e.g., aluminum, americium, cerium (beta form), cerium (gamma form), cesium, compounds of cobalt, dysprosium, compounds of dysprosium, europium, compounds of europium, gadolium, compounds of gadolinium, hafnium, compounds of holmium, iridium, compounds of iron, lithium, magnesium, manganese, molybdenum, neodymium, niobium, osmium, palladium, plutonium, potassium, praseodymium, rhodium, rubidium, ruthenium, samarium, sodium, strontium, tantalum, technicium; terbium, thorium, thulium, titanium, tungsten, uranium, vanadium, ytterbium, yttrium, and the like.

In addition to using positively magnetized species in coating 103 (see FIG. 4), one may also use negatively magnetized species. The negatively magnetized species include those materials with negative susceptibilities that are listed on such pages E-118 to E-123 of the CRC Handbook. By way of illustration and not limitation, such species include, e.g.: antimony; argon; arsenic; barium; beryllium; bismuth; boron; calcium; carbon (dia); chromium; copper; gallium; germanium; gold; indium; krypton; lead; mercury; phosphorous; selenium; silicon; silver; sulfur; tellurium; thallium; tin (gray); xenon; zinc; and the link.

Many diamagnetic materials also are suitable negatively magnetized species. As is known to those skilled in the art, diamagnetism is that property of a material that is repelled by magnets. The term “diamagnetic susceptibility” refers to the susceptibility of a diamagnetic material, which is always negative. Diamagnetic materials are well known to those skilled in the art. Reference may be had, e.g., to U.S. Pat. No. 6,162,364 (diamagnetic objects); U.S. Pat. No. 6,159,271 (diamagnetic liquid); U.S. Pat. No. 5,408,178 (diamagnetic and paramagnetic objects); U.S. Pat. No. 5,315,997 (method of magnetic resonance imaging using diamagnetic contrast); U.S. Pat. Nos. 5,162,301; 5,047,392 (diamagnetic colloids); U.S. Pat. Nos. 5,043,101; 5,026,681 (diamagnetic colloid pumps); U.S. Pat. No. 4,908,347 (diamagnetic flux shield); U.S. Pat. Nos. 4,778,594; 4,735,796; 4,590,922; 4,290,070; 3,899,758; 3,864,824; 3,815,963 (pseudo-diamagnetic suspension); U.S. Pat. Nos. 3,597,022; 3,572,273; and the like. The entire disclosure of each of these United States patents is hereby incorporated by reference into this specification.

By way of further illustration, the diamagnetic material used may be an organic compound with a negative susceptibility. Referring to pages E-123 to pages E-134 of the aforementioned CRC Handbook, such compounds include, e.g.: alanine; allyl alcohol; amylamine; aniline; asparagines; aspartic acid; butyl alcohol; cholesterol; coumarin; diethylamine; erythritol; eucalyptol; fructose; galactose; glucose; D-glucose; glutamic acid; glycerol; glycine; leucine; isoleucine; mannitol; mannose; and the like.

Referring again to FIG. 4, when a positively magnetized species is mixed with a negatively magnetized species, and assuming that each species retains its magnetic properties, the resulting magnetic properties exhibit substantially zero magnetization. In this embodiment, one must insure that the positively magnetized species does not lose its magnetic properties, as often happens when one material is alloyed with another. The magnetic properties of alloys and compounds containing different species are known, and thus it readily ascertainable whether the different species that make up such alloys and/or compounds have retained their unique magnetic characteristics.

Without wishing to be bound to any particular theory, applicants believe that, when a positively magnetized species is mixed with a negatively magnetized species, and assuming that each species retains its magnetic properties, the desired magnetization plot (substantially zero slope) will be achieved when the volume of the positively magnetized species times its positive susceptibility is substantially equal to the volume of the negatively magnetized species times its negative susceptibility For this relationship to hold, however, each of the positively magnetized species and the negatively magnetized species must retain the distinctive magnetic characteristics when mixed with each other.

Thus, for example, if element A has a positive magnetic susceptibility, and element B has a negative magnetic susceptibility, the alloying of A and B in equal proportions may not yield a zero magnetization compact.

Without wishing to be bound to any particular theory, nano-sized particles, or micro-sized particles (with a size of at least about 0.5 nanometers) tend to retain their magnetic properties as long as they remain in particulate form. On the other hand, alloys of such materials often do not retain such properties.

Nullification of the Susceptibility Contribution Due to the Substrate

As will be apparent by reference, e.g., to FIG. 4, when the substrate 104 is a copper stent, the copper substrate 104 depicted therein has a negative susceptibility, the coating 103 depicted therein preferably has a positive susceptibility, and the coated substrate 100 thus has a substantially zero susceptibility. As will also be apparent, some substrates (such niobium, nitinol, stainless steel, etc.) have positive susceptibilities. In such cases, and in one preferred embodiment, the coatings should preferably be chosen to have a negative susceptibility so that, under the conditions of the MRI radiation (or of any other radiation source used), the net susceptibility of the coated object is still substantially zero. As will be apparent, the contribution of each of the materials in the coating(s) is a function of the mass of such material and its magnetic susceptibility.

The magnetic susceptibilities of various substrate materials are well known. Reference may be had, e.g., to pages E-118 to E-123 of the “Handbook of Chemistry and Physics,” 63rd edition (CRC Press, Inc., Boca Raton, Fla., 1974).

Once the susceptibility of the substrate 104 material is determined, one can use the following equation: χ_{sub}+χ_{coat}=0, wherein χ_{sub} is the susceptibility of the substrate, and χ_{coat} is the susceptibility of the coating, when each of these is present in a 1/1 ratio. As will be apparent, the aforementioned equation is used when the coating and substrate are present in a 1/1 ratio. When other ratios are used other than a 1/1 ratio, the volume percent of each component (or its mass) must be taken into consideration in accordance with the equation: (volume percent of substrate×susceptibility of the substrate)+(volume percent of coating×susceptibility of the coating)=0. One may use a comparable formula in which the weight percent of each component is substituted for the volume percent, if the susceptibility is measured in terms of the weight percent.

By way of illustration, and in one embodiment, the uncoated substrate 104 may either comprise or consist essentially of niobium, which has a susceptibility of +195.0×10⁻⁶ centimeter-gram seconds at 298 degrees Kelvin.

In another embodiment, the substrate 104 may contain at least 98 molar percent of niobium and less than 2 molar percent of zirconium. Zirconium has a susceptibility of −122×0×10⁻⁶ centimeter-gram seconds at 293 degrees Kelvin. As will be apparent, because of the predominance of niobium, the net susceptibility of the uncoated substrate will be positive.

The substrate may comprise Nitinol. Nitinol is a paramagnetic alloy, an intermetallic compound of nickel and titanium; the alloy preferably contains from 50 to 60 percent of nickel, and it has a permeability value of about 1.002. The susceptibility of Nitinol is positive.

Nitinols with nickel content ranging from about 53 to 57 percent are known as “memory alloys” because of their ability to “remember” or return to a previous shape upon being heated which is an alloy of nickel and titanium, in an approximate 1/1 ratio. The susceptibility of Nitinol is positive.

The substrate 104 may comprise tantalum and/or titanium, each of which has a positive susceptibility. See, e.g., the CRC handbook cited above.

When the uncoated substrate has a positive susceptibility, the coating to be used for such a substrate should have a negative susceptibility. Referring again to said CRC handbook, it will be seen that the values of negative susceptibilities for various elements are −9.0 for beryllium, −280.1 for bismuth (s), −10.5 for bismuth (l), −6.7 for boron, −56.4 for bromine (l), −73.5 for bromine(g), −19.8 for cadmium(s), −18.0 for cadmium(l), −5.9 for carbon(dia), −6.0 for carbon (graph), −5.46 for copper(s), −6.16 for copper(l), −76.84 for germanium, −28.0 for gold(s), −34.0 for gold(l), −25.5 for indium, −88.7 for iodine(s), −23.0 for lead(s), −15.5 for lead(l), −19.5 for silver(s), −24.0 for silver(l), −15.5 for sulfur(alpha), −14.9 for sulfur(beta), −15.4 for sulfur(l), −39.5 for tellurium(s), −6.4 for tellurium(l), −37.0 for tin(gray), −31.7 for tin(gray), −4.5 for tin(l), −11.4 for zinc(s), −7.8 for zinc(l), and the like. As will be apparent, each of these values is expressed in units equal to the number in question×10⁻⁶ centimeter-gram seconds at a temperature at or about 293 degrees Kelvin. As will also be apparent, those materials which have a negative susceptibility value are often referred to as being diamagnetic.

By way of further reference, a listing of organic compounds that are diamagnetic is presented on pages E123 to E134 of the aforementioned “Handbook of Chemistry and Physics,” 63rd edition (CRC Press, Inc., Boca Raton, Fla., 1974).

In one embodiment, and referring again to the aforementioned “Handbook of Chemistry and Physics,” 63rd edition (CRC Press, Inc., Boca Raton, Fla., 1974), one or more of the following magnetic materials described below are preferably incorporated into the coating.

The desired magnetic materials, in this embodiment, preferably have a positive susceptibility, with values ranging from +1×10⁻⁶ centimeter-gram seconds at a temperature at or about 293 degrees Kelvin, to about 1×10⁷ centimeter-gram seconds at a temperature at or about 293 degrees Kelvin.

Thus, by way of illustration and not limitation, one may use materials such as Alnicol (see page E-112 of the CRC handbook), which is an alloy containing nickel, aluminum, and other elements such as, e.g., cobalt and/or iron. Thus, e.g., one my use silicon iron (see page E113 of the CRC handbook), which is an acid resistant iron containing a high percentage of silicon. Thus, e.g., one may use steel (see page 117 of the CRC handbook). Thus, e.g., one may use elements such as dyprosium, erbium, europium, gadolinium, hafnium, holmium, manganese, molybdenum, neodymium, nickel-cobalt, alloys of the above, and compounds of the above such as, e.g., their oxides, nitrides, carbonates, and the like.

Nullification of the Reactance of the Uncoated Substrate 104

In one preferred embodiment, and referring again to FIG. 4, the uncoated substrate 104 has an effective inductive reactance at a d.c. field of 1.5 Tesla that exceeds its capacitive reactance, whereas the coating 103 has a capacitive reactance that exceeds its inductive reactance. The coated (composite) substrate 100 706 has a net reactance that is preferably substantially zero.

As will be apparent, the effective inductive reactance of the uncoated stent 104 may be due to a multiplicity of factors including, e.g., the positive magnetic susceptibility of the materials which it is comprised of it, the loop currents produced, the surface eddy produced, etc. Regardless of the source(s) of its effective inductive reactance, it can be “corrected” by the use of one or more coatings which provide, in combination, an effective capacitive reactance that is equal to the effective inductive reactance.

Imaging of Restenosis

Referring again to FIG. 4, and in the embodiment depicted, plaque particles 130,132 are disposed on the inside of substrate 104. When the net reactance of the coated substrate 104 is essentially zero, the imaging field 140 can pass substantially unimpeded through the coating 103 and the substrate 104 and interact with the plaque particles 130/132 to produce imaging signals 141.

The imaging signals 141 are able to pass back through the substrate 104 and the coating 103 because the net reactance is substantially zero. Thus, these imaging signals are able to be received and processed by the MRI apparatus.

Thus, by the use of applicants' technology, one may negate the negative substrate effect and, additionally, provide pathways for the image signals to interact with the desired object to be imaged (such as, e.g., the plaque particles) and to produce imaging signals that are capable of escaping the substrate assembly and being received by the MRI apparatus.

Referring again to FIG. 4, and in one preferred embodiment, when an MRI field is present, the entire assembly 13, including the biological material 130/132, preferably presents a direct current magnetic susceptibility that is plus or minus 1×10⁻³ centimeter-gram-seconds (cgs) and, more preferably, plus or minus 1×10⁻⁴ centimeter-gram-seconds. In one embodiment, the d.c. susceptibility of the assembly 13 is equal to plus or minus 1×10⁻⁵ centimeter-gram-seconds. In another embodiment, the d.c. susceptibility of the assembly 13 is equal to plus or minus 1×10⁻⁶ centimeter-gram-seconds.

Referring again to FIG. 4, each of the components of assembly 13 has its own value of magnetic susceptibility. Thus, the biological material 130/132 has a magnetic susceptibility of S₁. Thus, the substrate 104 has a magnetic susceptibility of S₂. Thus, the coating 103 has a magnetic susceptibility of S₃.

Each of the components of the assembly 13 makes a contribution to the total magnetic susceptibility of such assembly, depending upon (a) whether its magnetic susceptibility is positive or negative, (b) the amount of its positive or negative susceptibility value, and (c) the percentage of the total mass that the individual component represents.

In determining the total susceptibility of the assembly 13, one can first determine the product of Mc and Sc, wherein Mc is the weight fraction of that component (the weight of that component divided by the total weight of all components in the assembly 6000).

In one preferred process, the McSc values for the nanomagnetic material 120 are chosen to, when appropriate, correct for the total McSc values of all of the other components (including the biological material 130/132) such that, after such correction(s), the total susceptibility of the assembly 13 is plus or minus 1×10⁻³ centimeter-gram-seconds (cgs) and, more preferably, plus or minus 1×10−4 centimeter-gram-seconds. In one embodiment, the d.c. susceptibility of the assembly 13 is equal to plus or minus 1×10⁻⁵ centimeter-gram-seconds. In another embodiment, the d.c. susceptibility of the assembly 13 is equal to plus or minus 1×10−6 centimeter-gram-seconds.

As will be apparent, there may be other materials/components in the assembly 13 whose values of positive or negative susceptibility, and/or their mass, may be chosen such that the total magnetic susceptibility of the assembly is plus or minus 1×10⁻³ centimeter-gram-seconds (cgs) and, more preferably, plus or minus 1×10−4 centimeter-gram-seconds. Similarly, the configuration of the substrate may be varied in order to vary its magnetic susceptibility properties and/or other properties.

Cancellation of the Positive Susceptibility of a Nitinol Stent

In one preferred embodiment, illustrated in FIG. 5, a stent 200 constructed form Nitinol is comprised of struts 202, 204, 206, and 208 coated with a layer of elemental bismuth. As is known to those skilled in the art, Nitinol is a paramagnetic alloy that was developed by the Naval Ordnance Laboratory; it is an intermetallic compound of nickel and titanium. See, e.g., page 552 of George S. Brady et al.'s “Materials Handbook,” Thirteenth Edition (McGraw-Hill Company, New York, N.Y., 1991).

Referring again to FIG. 5, and to the preferred embodiment depicted therein, the stent 200 is preferably cylindrical with a diameter (not shown) of less than 1 centimeter and a length 210 of about 3 centimeters. Each strut, such as strut 202, is preferably arcuate, having an effective diameter 212 of less than about 1 millimeter.

As is known to those skilled in the art, the magnetic permeability of the Nitinol material is about 1.003; and its susceptibility is about 0.03 centimeter-grams-seconds (cgs). In order to nullify the susceptibility, one can introduce a diamagnetic material, such as bismuth, that has a negative susceptibility. In one embodiment, a bismuth coating with a thickness of form about 10 to about 20 microns is deposited upon each of the struts 202.

Thus, and as will be apparent from the discussions presented in other parts of this specification, the susceptibility for these struts 202 becomes substantially zero, whereby there is no substantial direct current (d.c.) susceptibility distortion in the MRI field.

As used herein, the term “substantially zero” refers to a net susceptibility of from about 0.9 to about 1.1.

As will be apparent, when applicant's nanomagnetic coating 103 is added to such stent 210, the amount and type of the coating is chosen such that the net susceptibility for the struts is still preferably substantially zero,

As will be also be apparent, susceptibility varies with both direct current and alternating current. It is desired that, with the composite coating 103 described hereinabove, the susceptibility at a direct current field of about 1.5 Tesla (which is also the slope of the plot of magnetization versus the applied magnetic field), should preferably be from about 0.9 to about 1.1.

Incorporation by Reference of U.S. Pat. No. 6,713,671

United States patent application U.S. Ser. No. 10/303,264 (and also U.S. Pat. No. 6,713,671) discloses a shielded assembly comprised of a substrate and, disposed above a substrate, a shield comprising from about 1 to about 99 weight percent of a first nanomagnetic material, and from about 99 to about 1 weight percent of a second material with a resistivity of from about 1 microohm-centimeter to about 1×1025 microohm centimeters; the nanomagnetic material comprises nanomagnetic particles, and these nanomagnetic particles respond to an externally applied magnetic field by realigning to the externally applied field. Such a shielded assembly and/or the substrate thereof and/or the shield thereof may be used in the processes, compositions, and/or constructs of this invention.

As is disclosed in U.S. Pat. No. 6,713,671, the entire disclosure of which is hereby incorporated by reference into this specification, in one embodiment the substrate used may be, e.g., comprised of one or more conductive material(s) that have a resistivity at 20 degrees Centigrade of from about 1 to about 100 microohm-centimeters. Thus, e.g., the conductive material(s) may be silver, copper, aluminum, alloys thereof, mixtures thereof, and the like.

In one embodiment, the substrate consists consist essentially of such conductive material. Thus, e.g., it is preferred not to use, e.g., copper wire coated with enamel in this embodiment.

In the first step of the process preferably used to make this embodiment of the invention, (see step 40 of FIG. 1 of U.S. Pat. No. 6,713,671), conductive wires are coated with electrically insulative material. Suitable insulative materials include nano-sized silicon dioxide, aluminum oxide, cerium oxide, yttrium-stabilized zirconia, silicon carbide, silicon nitride, aluminum nitride, and the like. In general, these nano-sized particles will have a particle size distribution such that at least about 90 weight percent of the particles have a maximum dimension in the range of from about 10 to about 100 nanometers.

In such process, the coated conductors may be prepared by conventional means such as, e.g., the process described in U.S. Pat. No. 5,540,959, the entire disclosure of which is hereby incorporated by reference into this specification. Alternatively, one may coat the conductors by means of the processes disclosed in a text by D. Satas on “Coatings Technology Handbook” (Marcel Dekker, Inc., New York, N.Y., 1991). As is disclosed in such text, one may use cathodic arc plasma deposition (see pages 229 et seq.), chemical vapor deposition (see pages 257 et seq.), sol-gel coatings (see pages 655 et seq.), and the like.

FIG. 2 of U.S. Pat. No. 6,713,671 is a sectional view of the coated conductors 14/16. In the embodiment depicted in such FIG. 2, it will be seen that conductors 14 and 16 are separated by insulating material 42. In order to obtain the structure depicted in such FIG. 2, one may simultaneously coat conductors 14 and 16 with the insulating material so that such insulators both coat the conductors 14 and 16 and fill in the distance between them with insulation.

Referring again to such FIG. 2 of U.S. Pat. No. 6,713,671, the insulating material 42 that is disposed between conductors 14/16, may be the same as the insulating material 44/46 that is disposed above conductor 14 and below conductor 16. Alternatively, and as dictated by the choice of processing steps and materials, the insulating material 42 may be different from the insulating material 44 and/or the insulating material 46. Thus, step 48 of the process of such FIG. 2 describes disposing insulating material between the coated conductors 14 and 16. This step may be done simultaneously with step 40; and it may be done thereafter.

Referring again to such FIG. 2, and to the preferred embodiment depicted therein, the insulating material 42, the insulating material 44, and the insulating material 46 each generally has a resistivity of from about 1,000,000,000 to about 10,000,000,000,000 ohm-centimeters.

Referring again to FIG. 2 of U.S. Pat. No. 6,713,671, after the insulating material 42/44/46 has been deposited, and in one embodiment, the coated conductor assembly is preferably heat treated in step 50. This heat treatment often is used in conjunction with coating processes in which the heat is required to bond the insulative material to the conductors 14/16.

The heat-treatment step may be conducted after the deposition of the insulating material 42/44/46, or it may be conducted simultaneously therewith. In either event, and when it is used, it is preferred to heat the coated conductors 14/16 to a temperature of from about 200 to about 600 degrees Centigrade for from about 1 minute to about 10 minutes.

Referring again to FIG. 1A of U.S. Pat. No. 6,713,671 and in step 52 of the process, after the coated conductors 14/16 have been subjected to heat treatment step 50, they are allowed to cool to a temperature of from about 30 to about 100 degrees Centigrade over a period of time of from about 3 to about 15 minutes.

One need not invariably heat treat and/or cool. Thus, referring to such FIG. 1A, one may immediately coat nanomagnetic particles onto to the coated conductors 14/16 in step 54 either after step 48 and/or after step 50 and/or after step 52.

Referring again to FIG. 1A of U.S. Pat. No. 6,713,671 in step 54, nanomagnetic materials are coated onto the previously coated conductors 14 and 16. This is best shown in FIG. 2 of such patent, wherein the nanomagnetic particles are identified as particles 24.

In general, and as is known to those skilled in the art, nanomagnetic material is magnetic material which has an average particle size less than 100 nanometers and, preferably, in the range of from about 2 to 50 nanometers. Reference may be had, e.g., to U.S. Pat. No. 5,889,091 (rotationally free nanomagnetic material), U.S. Pat. Nos. 5,714,136, 5,667,924, and the like. The entire disclosure of each of these United States patents is hereby incorporated by reference into this specification.

In general, the thickness of the layer of nanomagnetic material deposited onto the coated conductors 14/16 is less than about 5 microns and generally from about 0.1 to about 3 microns.

Referring again to FIG. 2 of U.S. Pat. No. 6,713,671, after the nanomagnetic material is coated in step 54, the coated assembly may be optionally heat-treated in step 56. In this optional step 56, it is preferred to subject the coated conductors 14/16 to a temperature of from about 200 to about 600 degrees Centigrade for from about 1 to about 10 minutes.

In one embodiment, illustrated in FIG. 3 of U.S. Pat. No. 6,713,671, one or more additional insulating layers 43 are coated onto the assembly depicted in FIG. 2 of such patent. This is conducted in optional step 58 (see FIG. 1A of such patent).

FIG. 4 of U.S. Pat. No. 6,713,671 is a partial schematic view of the assembly 11 of FIG. 2 of such patent, illustrating the current flow in such assembly. Referring again to FIG. 4 of U.S. Pat. No. 6,713,671, it will be seen that current flows into conductor 14 in the direction of arrow 60, and it flows out of conductor 16 in the direction of arrow 62. The net current flow through the assembly 11 is zero; and the net Lorentz force in the assembly 11 is thus zero. Consequently, even high current flows in the assembly 11 do not cause such assembly to move.

Referring again to FIG. 4 of U.S. Pat. No. 6,713,671 conductors 14 and 16 are substantially parallel to each other. As will be apparent, without such parallel orientation, there may be some net current and some net Lorentz effect.

In the embodiment depicted in such FIG. 4, and in one preferred aspect thereof, the conductors 14 and 16 preferably have the same diameters and/or the same compositions and/or the same length.

Referring again to FIG. 4 of U.S. Pat. No. 6,713,671, the nanomagnetic particles 24 are present in a density sufficient so as to provide shielding from magnetic flux lines 64. Without wishing to be bound to any particular theory, applicant believes that the nanomagnetic particles 24 trap and pin the magnetic lines of flux 64.

In order to function optimally, the nanomagnetic particles 24 preferably have a specified magnetization. As is known to those skilled in the art, magnetization is the magnetic moment per unit volume of a substance. Reference may be had, e.g., to U.S. Pat. Nos. 4,169,998, 4,168,481, 4,166,263, 5,260,132, 4,778,714, and the like. The entire disclosure of each of these United States patents is hereby incorporated by reference into this specification.

Referring again to FIG. 4 of U.S. Pat. No. 6,713,671, the entire disclosure of which is hereby incorporated by reference into this specification, the layer of nanomagnetic particles 24 preferably has a saturation magnetization, at 25 degrees Centigrade, of from about 1 to about 36,000 Gauss, or higher. In one embodiment, the saturation magnetization at room temperature of the nanomagnetic particles is from about 500 to about 10,000 Gauss. For a discussion of the saturation magnetization of various materials, reference may be had, e.g., to U.S. Pat. Nos. 4,705,613, 4,631,613, 5,543,070, 3,901,741 (cobalt, samarium, and gadolinium alloys), and the like. The entire disclosure of each of these United States patents is hereby incorporated by reference into this specification.

In one embodiment, it is preferred to utilize a thin film with a thickness of less than about 2 microns and a saturation magnetization in excess of 20,000 Gauss. The thickness of the layer of nanomagnetic material is measured from the bottom surface of the layer that contains such material to the top surface of such layer that contains such material; and such bottom surface and/or such top surface may be contiguous with other layers of material (such as insulating material) that do not contain nanomagnetic particles.

Thus, e.g., one may make a thin film in accordance with the procedure described at page 156 of Nature, Volume 407, Sep. 14, 2000, that describes a multilayer thin film has a saturation magnetization of 24,000 Gauss.

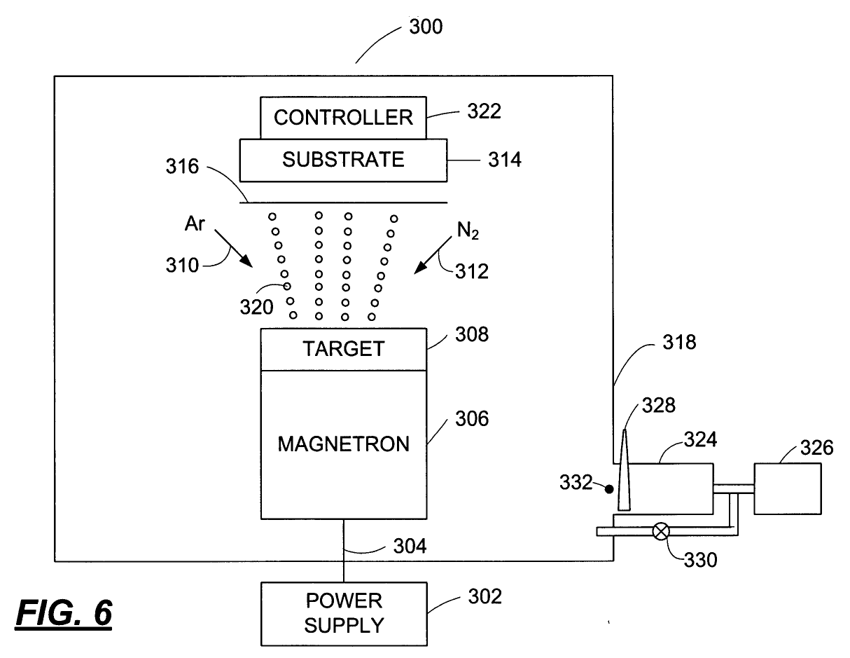

Referring again to FIG. 6 of U.S. Pat. No. 6,713,671, the film 104 is adapted to reduce the magnetic field strength at point 108 (which is disposed less than 1 centimeter above film 104) by at least about 50 percent. Thus, if one were to measure the magnetic field strength at point 108, and thereafter measure the magnetic field strength at point 110 (which is disposed less than 1 centimeter below film 104), the latter magnetic field strength would be no more than about 50 percent of the former magnetic field strength. Put another way, the film 104 has a magnetic shielding factor of at least about 0.5.

Referring again to FIG. 6 of U.S. Pat. No. 6,713,671, in one embodiment, the film 104 has a magnetic shielding factor of at least about 0.9, i.e., the magnetic field strength at point 110 is no greater than about 10 percent of the magnetic field strength at point 108. Thus, e.g., the static magnetic field strength at point 108 can be, e.g., one Tesla, whereas the static magnetic field strength at point 110 can be, e.g., 0.1 Tesla. Furthermore, the time-varying magnetic field strength of a 100 milliTesla would be reduced to about 10 milliTesla of the time-varying field.

An MRI Imaging Process

In one embodiment of the invention, best illustrated in FIG. 4, a coated stent 100 is imaged by an MRI imaging process. As will be apparent to those skilled in the art, the process depicted in FIG. 4 can be used with reference to other medical devices such as, e.g., a coated brachytherapy seed.

In the first step of this process, the coated stent 100 is contacted with the radio-frequency, direct current, and gradient fields normally associated with MRI imaging processes; these fields are discussed elsewhere in this specification. They are depicted as an MRI imaging signal 140 in FIG. 4

In the second step of this process, the MRI imaging signal 140 penetrates the coated stent 100 and interacts with material disposed on the inside of such stent, such as, e.g., plaque particles 130 and 132. This interaction produces a signal best depicted as arrow 141 in FIG. 4.

In one embodiment, the signal 440 is substantially unaffected by its passage through the coated stent 100. Thus, in this embodiment, the radio-frequency field that is disposed on the outside of the coated stent 100 is substantially the same as the radio-frequency field that passes through and is disposed on the inside of the coated stent 100.

By comparison, when the stent (not shown) is not coated with the coatings of this invention, the characteristics of the signal 140 are substantially varied by its passage through the uncoated stent. Thus, with such uncoated stent, the radio-frequency signal that is disposed on the outside of the stent (not shown) differs substantially from the radio-frequency field inside of the uncoated stent (not shown). In some cases, because of substrate effects, substantially none of such radio-frequency signal passes through the uncoated stent (not shown).

In the third step of this process, and in one embodiment thereof, the MRI field(s) interact with material disposed on the inside of coated stent 100 such as, e.g., plaque particles 130 and 132. This interaction produces a signal 141 by means well known to those in the MRI imaging art.

In the fourth step of the preferred process of this invention, the signal 141 passes back through the coated stent 100 in a manner such that it is substantially unaffected by the coated stent 100. Thus, in this embodiment, the radio-frequency field that is disposed on the inside of the coated stent 100 is substantially the same as the radio-frequency field that passes through and is disposed on the outside of the coated stent 100.

By comparison, when the stent (not shown) is not coated with the coatings of this invention, the characteristics of the signal 141 are substantially varied by its passage through the uncoated stent. Thus, with such uncoated stent, the radio-frequency signal that is disposed on the inside of the stent (not shown) differs substantially from the radio-frequency field outside of the uncoated stent (not shown). In some cases, because of substrate effects, substantially none of such signal 141 passes through the uncoated stent (not shown).

A Process for Preparation of an Iron-Containing Thin Film

In one preferred embodiment of the invention, a sputtering technique is used to prepare an AlFe thin film or particles, as well as comparable thin films containing other atomic moieties, or particles, such as, e.g., elemental nitrogen, and elemental oxygen. Conventional sputtering techniques may be used to prepare such films by sputtering. See, for example, R. Herrmann and G. Brauer, “D.C.- and R.F. Magnetron Sputtering,” in the “Handbook of Optical Properties: Volume I—Thin Films for Optical Coatings,” edited by R. E. Hummel and K. H. Guenther (CRC Press, Boca Raton, Fla., 1955). Reference also may be had, e.g., to M. Allendorf, “Report of Coatings on Glass Technology Roadmap Workshop,” Jan. 18-19, 2000, Livermore, Calif.; and also to U.S. Pat. No. 6,342,134, “Method for producing piezoelectric films with rotating magnetron sputtering system.” The entire disclosure of each of these prior art documents is hereby incorporated by reference into this specification.

One may utilize conventional sputtering devices in this process. By way of illustration and not limitation, a typical sputtering system is described in U.S. Pat. No. 5,178,739, the entire disclosure of which is hereby incorporated by reference into this specification. As is disclosed in this patent, “ . . . a sputter system 10 includes a vacuum chamber 20, which contains a circular end sputter target 12, a hollow, cylindrical, thin, cathode magnetron target 14, a RF coil 16 and a chuck 18, which holds a semiconductor substrate 19. The atmosphere inside the vacuum chamber 20 is controlled through channel 22 by a pump (not shown). The vacuum chamber 20 is cylindrical and has a series of permanent, magnets 24 positioned around the chamber and in close proximity therewith to create a multiple field configuration near the interior surface 15 of target 12. Magnets 26, 28 are placed above end sputter target 12 to also create a multipole field in proximity to target 12. A singular magnet 26 is placed above the center of target 12 with a plurality of other magnets 28 disposed in a circular formation around magnet 26. For convenience, only two magnets 24 and 28 are shown. The configuration of target 12 with magnets 26, 28 comprises a magnetron sputter source 29 known in the prior art, such as the Torus-10E system manufactured by K. Lesker, Inc. A sputter power supply 30 (DC or RF) is connected by a line 32 to the sputter target 12. A RF supply 34 provides power to RF coil 16 by a line 36 and through a matching network 37. Variable impedance 38 is connected in series with the cold end 17 of coil 16. A second sputter power supply 39 is connected by a line 40 to cylindrical sputter target 14. A bias power supply 42 (DC or RF) is connected by a line 44 to chuck 18 in order to provide electrical bias to substrate 19 placed thereon, in a manner well known in the prior art.”

By way of yet further illustration, other conventional sputtering systems and processes are described in U.S. Pat. No. 5,569,506 (a modified Kurt Lesker sputtering system), U.S. Pat. No. 5,824,761 (a Lesker Torus 10 sputter cathode), U.S. Pat. Nos. 5,768,123, 5,645,910, 6,046,398 (sputter deposition with a Kurt J. Lesker Co. Torus 2 sputter gun), U.S. Pat. Nos. 5,736,488, 5,567,673, 6,454,910, and the like. The entire disclosure of each of these United States patents is hereby incorporated by reference into this specification.

By way of yet further illustration, one may use the techniques described in a paper by Xingwu Wang et al. entitled “Technique Devised for Sputtering AlN Thin Films,” published in “the Glass Researcher,” Volume 11, No. 2 (Dec. 12, 2002).

In one preferred embodiment, a magnetron sputtering technique is utilized, with a Lesker Super System III system The vacuum chamber of this system is preferably cylindrical, with a diameter of approximately one meter and a height of approximately 0.6 meters. The base pressure used is from about 0.001 to 0.0001 Pascals. In one aspect of this process, the target is a metallic FeAl disk, with a diameter of approximately 0.1 meter. The molar ratio between iron and aluminum used in this aspect is approximately 70/30. Thus, the starting composition in this aspect is almost non-magnetic. See, e.g., page 83 (FIG. 3.1aii) of R. S. Tebble et al.'s “Magnetic Materials” (Wiley-Interscience, New York, N.Y., 1969); this Figure discloses that a bulk composition containing iron and aluminum with at least 30 mole percent of aluminum (by total moles of iron and aluminum) is substantially non-magnetic.

In this aspect, to fabricate FeAl films, a DC power source is utilized, with a power level of from about 150 to about 550 watts (Advanced Energy Company of Colorado, model MDX Magnetron Drive). The sputtering gas used in this aspect is argon, with a flow rate of from about 0.0012 to about 0.0018 standard cubic meters per second. To fabricate FeAlN films in this aspect, in addition to the DC source, a pulse-forming device is utilized, with a frequency of from about 50 to about 250 MHz (Advanced Energy Company, model Sparc-le V). One may fabricate FeAl0 films in a similar manner but using oxygen rather than nitrogen.

In this aspect, a typical argon flow rate is from about (0.9 to about 1.5)×10⁻³ standard cubic meters per second; a typical nitrogen flow rate is from about (0.9 to about 1.8)×10⁻³ standard cubic meters per second; and a typical oxygen flow rate is from about. (0.5 to about 2)×10⁻³ standard cubic meters per second. During fabrication, the pressure typically is maintained at from about 0.2 to about 0.4 Pascals. Such a pressure range has been found to be suitable for nanomagnetic materials fabrications. In one embodiment, it is preferred that both gaseous nitrogen and gaseous oxygen are present during the sputtering process.

In this aspect, the substrate used may be either flat or curved. A typical flat substrate is a silicon wafer with or without a thermally grown silicon dioxide layer, and its diameter is preferably from about 0.1 to about 0.15 meters. A typical curved substrate is an aluminum rod or a stainless steel wire, with a length of from about 0.10 to about 0.56 meters and a diameter of from (about 0.8 to about 3.0)×10⁻³ meters The distance between the substrate and the target is preferably from about 0.05 to about 0.26 meters.

In this aspect, in order to deposit a film on a wafer, the wafer is fixed on a substrate holder. The substrate may or may not be rotated during deposition. In one embodiment, to deposit a film on a rod or wire, the rod or wire is rotated at a rotational speed of from about 0.01 to about 0.1 revolutions per second, and it is moved slowly back and forth along its symmetrical axis with a maximum speed of about 0.01 meters per second.

In this aspect, to achieve a film deposition rate on the flat wafer of 5×10⁻¹⁰ meters per second, the power required for the FeAl film is 200 watts, and the power required for the FeAlN film is 500 watts The resistivity of the FeAlN film is approximately one order of magnitude larger than that of the metallic FeAl film. Similarly, the resistivity of the FeAl0 film is about one order of magnitude larger than that of the metallic FeAl film.

Iron containing magnetic materials, such as FeAl, FeAlN and FeAlO, FeAlNO, FeCoAlNO, and the like, may be fabricated by sputtering. The magnetic properties of those materials vary with stoichiometric ratios, particle sizes, and fabrication conditions; see, e.g., R. S. Tebble and D. J. Craik, “Magnetic Materials”, pp. 81-88, Wiley-Interscience, New York, 1969 As is disclosed in this reference, when the iron molar ratio in bulk FeAl materials is less than 70 percent or so, the materials will no longer exhibit magnetic properties.

However, it has been discovered that, in contrast to bulk materials, a thin film material often exhibits different properties.

A Preferred Sputtering Process

On Dec. 29, 2003, applicants filed U.S. patent application Ser. No. 10/747,472, for “Nanoelectrical Compositions.” The entire disclosure of this United States patent application is hereby incorporated by reference into this specification.

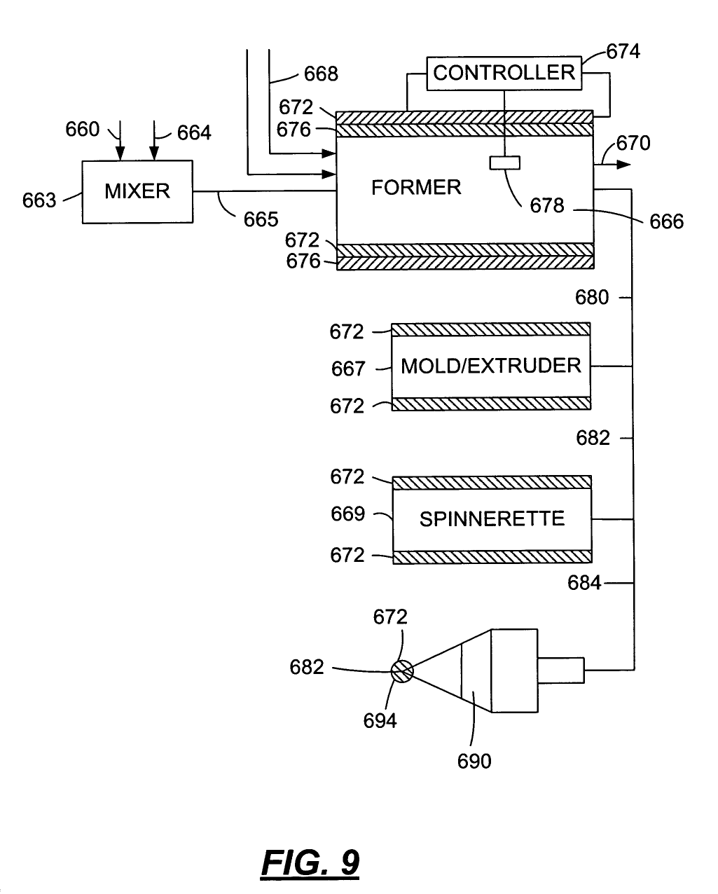

U.S. Ser. No. 10/747,472, at pages 10-15 thereof (and by reference to its FIG. 9), described the “ . . . preparation of a doped aluminum nitride assembly.” This portion of U.S. Ser. No. 10/747,472 is specifically incorporated by reference into this specification. It is also described below, by reference to FIG. 6, which is similar to the FIG. 9 of U.S. Ser. No. 10/747,472 but utilizes different reference numerals.

The system depicted in FIG. 6 may be used to prepare an assembly comprised of moieties A, B, and C that are described elsewhere in this specification. FIG. 5 will be described hereinafter with reference to one of the preferred ABC moieties, i.e., aluminum nitride doped with magnesium.

FIG. 6 is a schematic of a deposition system 300 comprised of a power supply 302 operatively connected via line 304 to a magnetron 306. Disposed on top of magnetron 306 is a target 308. The target 308 is contacted by gas 310 and gas 312, which cause sputtering of the target 308. The material so sputtered contacts substrate 314 when allowed to do so by the absence of shutter 316.

In one preferred embodiment, the target 308 is mixture of aluminum and magnesium atoms in a molar ratio of from about 0.05 to about 0.5 Mg/(Al+Mg). In one aspect of this embodiment, the ratio of Mg/(Al+Mg) is from about 0.08 to about 0.12. These targets are commercially available and are custom made by companies such as, e.g., Kurt Lasker and Company of Pittsburgh, Pa.

The power supply 302 preferably provides pulsed direct current. Generally, power supply 302 provides power in excess of 300 watts, preferably in excess of 500 watts, and more preferably in excess of 1,000 watts. In one embodiment, the power supplied by power supply 302 is from about 1800 to about 2500 watts.

The power supply preferably provides rectangular-shaped pulses with a duration (pulse width) of from about 10 nanoseconds to about 100 nanoseconds. In one embodiment, the pulse width is from about 20 to about 40 nanoseconds.

In between adjacent pulses, preferably substantially no power is delivered. The time between adjacent pulses is generally from about 1 microsecond to about 10 microseconds and is generally at least 100 times greater than the pulse width. In one embodiment, the repetition rate of the rectangular pulses is preferably about 150 kilohertz.

One may use a conventional pulsed direct current (d.c.) power supply. Thus, e.g., one may purchase such a power supply from Advanced Energy Company of Colorado, and/or from ENI Company of Rochester, N.Y.

The pulsed d.c. power from power supply 302 is delivered to a magnetron 306, that creates an electromagnetic field near target 308. In one embodiment, a magnetic field has a magnetic flux density of from about 0.01 Tesla to about 0.1 Tesla.

As will be apparent, because the energy provided to magnetron 306 preferably comprises intermittent pulses, the resulting magnetic fields produced by magnetron 306 will also be intermittent. Without wishing to be bound to any particular theory, applicants believe that the use of such intermittent electromagnetic energy yields better results than those produced by continuous radio-frequency energy.

Referring again to FIG. 6, it will be seen that the process depicted therein preferably is conducted within a vacuum chamber 318 in which the base pressure is from about 1×10⁻⁸ Torr to about 0.000005 Torr. In one embodiment, the base pressure is from about 0.000001 to about 0.000003 Torr.

The temperature in the vacuum chamber 318 generally is ambient temperature prior to the time sputtering occurs.

In one aspect of the embodiment illustrated in FIG. 6, argon gas is fed via line 310, and nitrogen gas is fed via line 312 so that both impact target 308, preferably in an ionized state. In another embodiment of the invention, argon gas, nitrogen gas, and oxygen gas are fed via target 312.

The argon gas, and the nitrogen gas, are fed at flow rates such that the flow rate of the argon gas divided by the flow rate of the nitrogen gas preferably is from about 0.6 to about 1.2. In one aspect of this embodiment, such ratio of argon to nitrogen is from about 0.8 to about 0.95. Thus, for example, the flow rate of the argon may be 20 standard cubic centimeters per minute, and the flow rate of the nitrogen may be 23 standard cubic feet per minute.

The argon gas, and the nitrogen gas, contact a target 308 that is preferably immersed in an electromagnetic field. This field tends to ionize the argon and the nitrogen, providing ionized species of both gases. It is such ionized species that bombard target 308.

In one embodiment, target 308 may be, e.g., pure aluminum. In one preferred embodiment, however, target 308 is aluminum doped with minor amounts of one or more of the aforementioned moieties B.

In the latter embodiment, the moieties B are preferably present in a concentration of from about 1 to about 40 molar percent, by total moles of aluminum and moieties B. It is preferred to use from about 5 to about 30 molar percent of such moieties B.

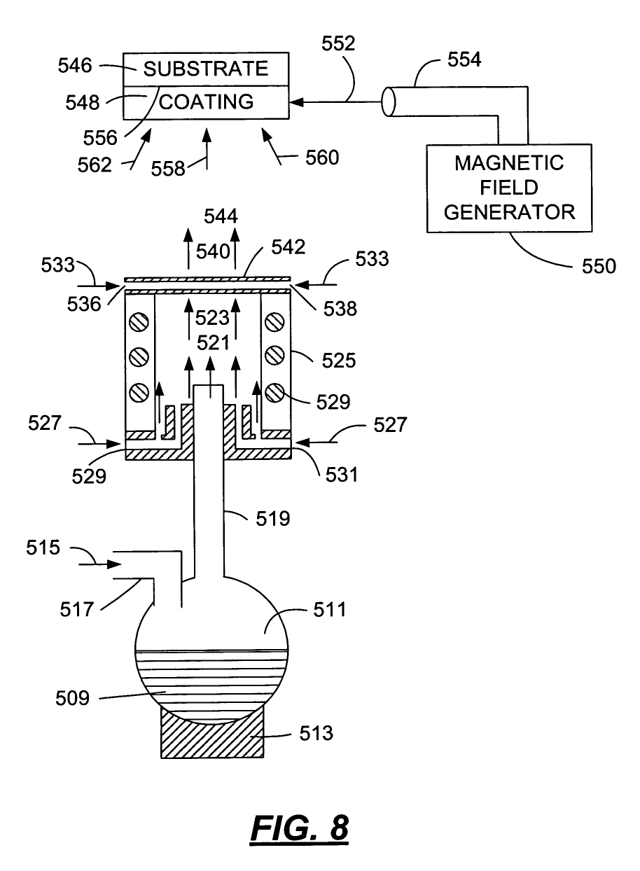

The ionized argon gas, and the ionized nitrogen gas, after impacting the target 308, creates a multiplicity of sputtered particles 320. In the embodiment illustrated in FIG. 8 the shutter 316 prevents the sputtered particles from contacting substrate 314.

When the shutter 316 is removed, however, the sputtered particles 320 can contact and coat the substrate 314.

In one embodiment, illustrated in FIG. 6 the temperature of substrate 314 is controlled by controller 322 that can heat the substrate (by means such as a conduction heater or an infrared heater) and/or cool the substrate (by means such as liquid nitrogen or water).

The sputtering operation increases the pressure within the region of the sputtered particles 320. In general, the pressure within the area of the sputtered particles 320 is at least 100 times, and preferably 1000 times, greater than the base pressure.

Referring again to FIG. 6 a cryo pump 324 is preferably used to maintain the base pressure within vacuum chamber 318. In the embodiment depicted, a mechanical pump (dry pump) 326 is operatively connected to the cryo pump 324. Atmosphere from chamber 318 is removed by dry pump 326 at the beginning of the evacuation. At some point, shutter 328 is removed and allows cryo pump 324 to continue the evacuation. A valve 330 controls the flow of atmosphere to dry pump 326 so that it is only open at the beginning of the evacuation.

It is preferred to utilize a substantially constant pumping speed for cryo pump 324, i.e., to maintain a constant outflow of gases through the cryo pump 324. This may be accomplished by sensing the gas outflow via sensor 332 and, as appropriate, varying the extent to which the shutter 328 is open or partially closed.

Without wishing to be bound to any particular theory, applicants believe that the use of a substantially constant gas outflow rate insures a substantially constant deposition of sputtered nitrides.

Referring again to FIG. 6, and in one embodiment thereof, it is preferred to clean the substrate 314 prior to the time it is utilized in the process. Thus, e.g., one may use detergent to clean any grease or oil or fingerprints off the surface of the substrate. Thereafter, one may use an organic solvent such as acetone, isopropyl alcohol, toluene, etc.

In one embodiment, the cleaned substrate 314 is presputtered by suppressing sputtering of the target 308 and sputtering the surface of the substrate 314.

As will be apparent to those skilled in the art, the process depicted in FIG. 6 may be used to prepare coated substrates 314 comprised of moieties other than doped aluminum nitride.

A Preferred Process for Preparing Nanomagnetic Particles

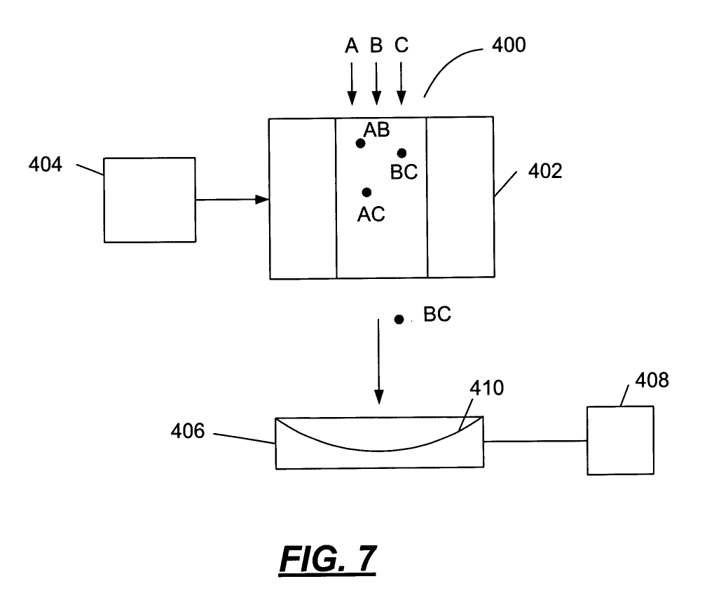

In one embodiment, illustrated in FIG. 7, a substrate is cooled so that nanomagnetic particles are collected on such substrate. Referring to FIG. 7, and in the preferred embodiment depicted therein, a precursor 400 that preferably contains moieties A, B, and C (which are described elsewhere in this specification) are charged to reactor 402.

The reactor 402 may be a plasma reactor. Plasma reactors are described in applicants' U.S. Pat. No. 5,100,868 (process for preparing superconducting films), U.S. Pat. No. 5,120,703 (process for preparing oxide superconducting films by radio-frequency generated aerosol-plasma deposition in atmosphere), U.S. Pat. No. 5,157,015 (process for preparing superconducting films by radio-frequency generated aerosol-plasma deposition in atmosphere), U.S. Pat. No. 5,213,851 (process for preparing ferrite films by radio-frequency generated aerosol plasma deposition in atmosphere), U.S. Pat. No. 5,260,105 (aerosol plasma deposition of films for electrochemical cells), U.S. Pat. No. 5,364,562 (aerosol plasma deposition of insulating oxide powder), U.S. Pat. No. 5,366,770 (aerosol plasma deposition of films for electronic cells), and the like. The entire disclosure of each of these United States patents is hereby incorporated by reference into this specification.

The reactor 402 may be sputtering reactor 300 depicted in FIG. 6.

Referring again to FIG. 7, it will be seen that an energy source 4045 is preferably used in order to cause reaction between moieties A, B, and C. The energy source 404 may be an electromagnetic energy source that supplies energy to the reactor 400. In one embodiment, there are at least two species of moiety A present, and at least two species of moiety C present. The two preferred moiety C species are oxygen and nitrogen.

Within reactor 402 moieties A, B, and C are preferably combined into a metastable state. This metastable state is then caused to travel towards collector 406. Prior to the time it reaches the collector 406, the ABC moiety is formed, either in the reactor 3 and/or between the reactor 402 and the collector 406.

In one embodiment, collector 406 is preferably cooled with a chiller 408 so that its surface 410 is at a temperature below the temperature at which the ABC moiety interacts with surface 410; the goal is to prevent bonding between the ABC moiety and the surface 410. In one embodiment, the surface 410 is at a temperature of less than about 30 degrees Celsius. In another embodiment, the temperature of surface 410 is at the liquid nitrogen temperature, i.e., about 77 degrees Kelvin.

After the ABC moieties have been collected by collector 406, they are removed from surface 410.

FIG. 8 is a schematic illustration of one process of the invention that may be used to make nanomagnetic material. This FIG. 8 is similar in many respects to the FIG. 1 of U.S. Pat. No. 5,213,851, the entire disclosure of which is hereby incorporated by reference into this specification.

Referring to FIG. 8, and in the preferred embodiment depicted therein, it is preferred that the reagents charged into misting chamber 511 will be sufficient, in one embodiment, to form a nano-sized ferrite in the process. The term ferrite, as used in this specification, refers to a material that exhibits ferromagnetism. Ferromagnetism is a property, exhibited by certain metals, alloys, and compounds of the transition (iron group) rare earth and actinide elements, in which the internal magnetic moments spontaneously organize in a common direction; ferromagnetism gives rise to a permeability considerably greater than that of vacuum and to magnetic hysteresis. See, e.g., page 706 of Sybil B. Parker's “McGraw-Hill Dictionary of Scientific and Technical Terms,” Fourth Edition (McGraw-Hill Book Company, New York, N.Y., 1989).

As will be apparent to those skilled in the art, in addition to making nano-sized ferrites by the process depicted in FIG. 8, one may also make other nano-sized materials such as, e.g., nano-sized nitrides and/or nano-sized oxides containing moieties A, B, and C, as is described elsewhere in this specification. For the sake of simplicity of description, and with regard to FIG. 8, a discussion will be had regarding the preparation of ferrites, it being understood that, e.g., other materials may also be made by such process.

Referring again to FIG. 8, and to the production of ferrites by such process, in one embodiment, the ferromagnetic material contains Fe₂O₃. See, for example, U.S. Pat. No. 3,576,672 of Harris et al., the entire disclosure of which is hereby incorporated by reference into this specification. As will be apparent, the corresponding nitrides also may be made.

In one embodiment, the ferromagnetic material contains garnet. Pure iron garnet has the formula M₃Fe₅O₁₂; see, e.g., pages 65-256 of Wilhelm H. Von Aulock's “Handbook of Microwave Ferrite Materials” (Academic Press, New York, 1965). Garnet ferrites are also described, e.g., in U.S. Pat. No. 4,721,547, the disclosure of which is hereby incorporated by reference into this specification. As will be apparent, the corresponding nitrides also may be made.

In another embodiment, the ferromagnetic material contains a spinel ferrite. Spinel ferrites usually have the formula MFe₂O₄, wherein M is a divalent metal ion and Fe is a trivalent iron ion. M is typically selected from the group consisting of nickel, zinc, magnesium, manganese, and like. These spinel ferrites are well known and are described, for example, in U.S. Pat. Nos. 5,001,014, 5,000,909, 4,966,625, 4,960,582, 4,957,812, 4,880,599, 4,862,117, 4,855,205, 4,680,130, 4,490,268, 3,822,210, 3,635,898, 3,542,685, 3,421,933, and the like. The disclosure of each of these patents is hereby incorporated by reference into this specification. Reference may also be had to pages 269-406 of the Von Aulock book for a discussion of spinel ferrites. As will be apparent, the corresponding nitrides also may be made.

In yet another embodiment, the ferromagnetic material contains a lithium ferrite. Lithium ferrites are often described by the formula (Li_{0.5}Fe_{0.5})2+(Fe₂)3+O₄. Some illustrative lithium ferrites are described on pages 407-434 of the aforementioned Von Aulock book and in U.S. Pat. Nos. 4,277,356, 4,238,342, 4,177,438, 4,155,963, 4,093,781, 4,067,922, 3,998,757, 3,767,581, 3,640,867, and the like. The disclosure of each of these patents is hereby incorporated by reference into this specification. As will be apparent, the corresponding nitrides also may be made.

In yet another embodiment, the ferromagnetic material contains a hexagonal ferrite. These ferrites are well known and are disclosed on pages 451-518 of the Von Aulock book and also in U.S. Pat. Nos. 4,816,292, 4,189,521, 5,061,586, 5,055,322, 5,051,201, 5,047,290, 5,036,629, 5,034,243, 5,032,931, and the like. The disclosure of each of these patents is hereby incorporated by reference into this specification. As will be apparent, the corresponding nitrides also may be made.

In yet another embodiment, the ferromagnetic material contains one or more of the moieties A, B, and C disclosed in the phase diagram disclosed elsewhere in this specification and discussed elsewhere in this specification.

Referring again to FIG. 8, and in the preferred embodiment depicted therein, it will be appreciated that the solution 509 will preferably comprise reagents necessary to form the required magnetic material. For example, in one embodiment, in order to form the spinel nickel ferrite of the formula NiFe₂O₄, the solution should contain nickel and iron, which may be present in the form of nickel nitrate and iron nitrate. By way of further example, one may use nickel chloride and iron chloride to form the same spinel. By way of further example, one may use nickel sulfate and iron sulfate.

It will be apparent to skilled chemists that many other combinations of reagents, both stoichiometric and nonstoichiometric, may be used in applicants' process to make many different magnetic materials.

In one preferred embodiment, the solution 509 contains the reagent needed to produce a desired ferrite in stoichiometric ratio. Thus, to make the NiFe₂O₄ ferrite in this embodiment, one mole of nickel nitrate may be charged with every two moles of iron nitrate.

In one embodiment, the starting materials are powders with purities exceeding 99 percent.

In one embodiment, compounds of iron and the other desired ions are present in the solution in the stoichiometric ratio.

In one preferred embodiment, ions of nickel, zinc, and iron are present in a stoichiometric ratio of 0.5/0.5/2.0, respectively. In another preferred embodiment, ions of lithium and iron are present in the ratio of 0.5/2.5. In yet another preferred embodiment, ions of magnesium and iron are present in the ratio of 1.0/2.0. In another embodiment, ions of manganese and iron are present in the ratio 1.0/2.0. In yet another embodiment, ions of yttrium and iron are present in the ratio of 3.0/5.0. In yet another embodiment, ions of lanthanum, yttrium, and iron are present in the ratio of 0.5/2.5/5.0. In yet another embodiment, ions of neodymium, yttrium, gadolinium, and iron are present in the ratio of 1.0/1.07/0.93/5.0, or 1.0/1.1/0.9/5.0, or 1/1.12/0.88/5.0. In yet another embodiment, ions of samarium and iron are present in the ratio of 3.0/5.0. In yet another embodiment, ions of neodymium, samarium, and iron are present in the ratio of 0.1/2.9/5.0, or 0.25/2.75/5.0, or 0.375/2.625/5.0. In yet another embodiment, ions of neodymium, erbium, and iron are present in the ratio of 1.5/1.5/5.0. In yet another embodiment, samarium, yttrium, and iron ions are present in the ratio of 0.51/2.49/5.0, or 0.84/2.16/5.0, or 1.5/1.5/5.0. In yet another embodiment, ions of yttrium, gadolinium, and iron are present in the ratio of 2.25/0.75/5.0, or 1.5/1.5/5.0, or 0.75/2.25/5.0. In yet another embodiment, ions of terbium, yttrium, and iron are present in the ratio of 0.8/2.2/5.0, or 1.0/2.0/5.0. In yet another embodiment, ions of dysprosium, aluminum, and iron are present in the ratio of 3/x/5-x, when x is from 0 to 1.0. In yet another embodiment, ions of dysprosium, gallium, and iron are also present in the ratio of 3/x/5-x. In yet another embodiment, ions of dysprosium, chromium, and iron are also present in the ratio of 3/x/5-x.

The ions present in the solution, in one embodiment, may be holmium, yttrium, and iron, present in the ratio of z/3-z/5.0, where z is from about 0 to 1.5.

The ions present in the solution may be erbium, gadolinium, and iron in the ratio of 1.5/1.5/5.0. The ions may be erbium, yttrium, and iron in the ratio of 1.5/1.5/1.5, or 0.5/2.5/5.0.

The ions present in the solution may be thulium, yttrium, and iron, in the ratio of 0.06/2.94/5.0.

The ions present in the solution may be ytterbium, yttrium, and iron, in the ratio of 0.06/2.94/5.0.

The ions present in the solution may be lutetium, yttrium, and iron in the ratio of y/3-y/5.0, wherein y is from 0 to 3.0.

The ions present in the solution may be iron, which can be used to form Fe₆O₈ (two formula units of Fe₃O₄). The ions present may be barium and iron in the ratio of 1.0/6.0, or 2.0/8.0. The ions present may be strontium and iron, in the ratio of 1.0/12.0. The ions present may be strontium, chromium, and iron in the ratio of 1.0/1.0/10.0, or 1.0/6.0/6.0. The ions present may be suitable for producing a ferrite of the formula (Meₓ)₃+Ba₁₋ₓFe₁₂O₁₉, wherein Me is a rare earth selected from the group consisting of lanthanum, promethium, neodymium, samarium, europium, and mixtures thereof.

The ions present in the solution may contain barium, either lanthanum or promethium, iron, and cobalt in the ratio of 1-a/a/12-a/a, wherein a is from 0.0 to 0.8.

The ions present in the solution may contain barium, cobalt, titanium, and iron in the ratio of 1.0/b/b/12-2b, wherein b is from 0.0 to 1.6.

The ions present in the solution may contain barium, nickel or cobalt or zinc, titanium, and iron in the ratio of 1.0/c/c/12-2c, wherein c is from 0.0 to 1.5.

The ions present in the solution may contain barium, iron, iridium, and zinc in the ratio of 1.0/12-2d/d/d, wherein d is from 0.0 to 0.6.

The ions present in the solution may contain barium, nickel, gallium, and iron in the ratio of 1.0/2.0/7.0/9.0, or 1.0/2.0/5.0/11.0. Alternatively, the ions may contain barium, zinc, gallium or aluminum, and iron in the ratio of 1.0/2.0/3.0/13.0.

Each of these ferrites is well known to those in the ferrite art and is described, e.g., in the aforementioned Von Aulock book.

The ions described above are preferably available in solution 509 in water-soluble form, such as, e.g., in the form of water-soluble salts. Thus, e.g., one may use the nitrates or the chlorides or the sulfates or the phosphates of the cations. Other anions which form soluble salts with the cation(s) also may be used.

Alternatively, one may use salts soluble in solvents other than water. Some of these other solvents which may be used to prepare the material include nitric acid, hydrochloric acid, phosphoric acid, sulfuric acid, and the like. As is well known to those skilled in the art, many other suitable solvents may be used; see, e.g., J. A. Riddick et al., “Organic Solvents, Techniques of Chemistry,” Volume II, 3rd edition (Wiley-Interscience, New York, N.Y., 1970).

In one preferred embodiment, where a solvent other than water is used, each of the cations is present in the form of one or more of its oxides. For example, one may dissolve iron oxide in nitric acid, thereby forming a nitrate. For example, one may dissolve zinc oxide in sulfuric acid, thereby forming a sulfate. One may dissolve nickel oxide in hydrochloric acid, thereby forming a chloride. Other means of providing the desired cation(s) will be readily apparent to those skilled in the art.

In general, as long as the desired cation(s) are present in the solution, it is not significant how the solution was prepared.

In general, one may use commercially available reagent grade materials. Thus, by way of illustration and not limitation, one may use the following reagents available in the 1988-1989 Aldrich catalog (Aldrich Chemical Company, Inc., Milwaukee, Wis.): barium chloride, catalog number 31,866-3; barium nitrate, catalog number 32,806-5; barium sulfate, catalog number 20,276-2; strontium chloride hexhydrate, catalog number 20,466-3; strontium nitrate, catalog number 20,449-8; yttrium chloride, catalog number 29,826-3; yttrium nitrate tetrahydrate, catalog number 21,723-9; yttrium sulfate octahydrate, catalog number 20,493-5. This list is merely illustrative, and other compounds that can be used will be readily apparent to those skilled in the art. Thus, any of the desired reagents also may be obtained from the 1989-1990 AESAR catalog (Johnson Matthey/AESAR Group, Seabrook, N.H.), the 1990/1991 Alfa catalog (Johnson Matthey/Alfa Products, Ward Hill, Mass.), the Fisher 88 catalog (Fisher Scientific, Pittsburgh, Pa.), and the like.

As long as the metals present in the desired ferrite material are present in solution 509 in the desired stoichiometry, it does not matter whether they are present in the form of a salt, an oxide, or in another form. In one embodiment, however, it is preferred to have the solution contain either the salts of such metals, or their oxides.

The solution 509 of the compounds of such metals preferably will be at a concentration of from about 0.01 to about 1,000 grams of said reagent compounds per liter of the resultant solution. As used in this specification, the term liter refers to 1,000 cubic centimeters.

In one embodiment, it is preferred that solution 509 have a concentration of from about 1 to about 300 grams per liter and, preferably, from about 25 to about 170 grams per liter. It is even more preferred that the concentration of said solution 9 be from about 100 to about 160 grams per liter. In an even more preferred embodiment, the concentration of said solution 509 is from about 140 to about 160 grams per liter.

In one preferred embodiment, aqueous solutions of nickel nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel nitrate, zinc nitrate, and iron nitrate with purities of at least 99.9 percent are mixed in the molar ratio of 0.5:0.5:2 and then dissolved in distilled water to form a solution with a concentration of 150 grams per liter.

In one preferred embodiment, aqueous solutions of zinc nitrate, and iron nitrate with purities of at least 99.9 percent are mixed in the molar ratio of 1:2 and then dissolved in distilled water to form a solution with a concentration of 150 grams per liter.

In one preferred embodiment, aqueous solutions of nickel chloride, and iron chloride with purities of at least 99.9 percent are mixed in the molar ratio of 1:2 and then dissolved in distilled water to form a solution with a concentration of 150 grams per liter.

In one preferred embodiment, aqueous solutions of nickel chloride, zinc chloride, and iron chloride with purities of at least 99.9 percent are mixed in the molar ratio of 0.5:0.5:2 and then dissolved in distilled water to form a solution with a concentration of 150 grams per liter.

In one preferred embodiment, aqueous solutions of zinc chloride, and iron chloride with purities of at least 99.9 percent are mixed in the molar ratio of 1:2 and then dissolved in distilled water to form a solution with a concentration of 150 grams per liter.

In one embodiment, mixtures of chlorides and nitrides may be used. Thus, for example, in one preferred embodiment, the solution is comprised of both iron chloride and nickel nitrate in the molar ratio of 2.0/1.0.

Referring again to FIG. 8, and to the preferred embodiment depicted therein, the solution 509 in misting chamber 511 is preferably caused to form into an aerosol, such as a mist.

The term aerosol, as used in this specification, refers to a suspension of ultramicroscopic solid or liquid particles in air or gas, such as smoke, fog, or mist. See, e.g., page 15 of “A dictionary of mining, mineral, and related terms,” edited by Paul W. Thrush (U.S. Department of the Interior, Bureau of Mines, 1968), the disclosure of which is hereby incorporated by reference into this specification.

As used in this specification, the term mist refers to gas-suspended liquid particles which have diameters less than 10 microns.

The aerosol/mist consisting of gas-suspended liquid particles with diameters less than 10 microns may be produced from solution 509 by any conventional means that causes sufficient mechanical disturbance of said solution. Thus, one may use mechanical vibration. In one preferred embodiment, ultrasonic means are used to mist solution 9. As is known to those skilled in the art, by varying the means used to cause such mechanical disturbance, one can also vary the size of the mist particles produced.

As is known to those skilled in the art, ultrasonic sound waves (those having frequencies above 20,000 hertz) may be used to mechanically disturb solutions and cause them to mist. Thus, by way of illustration, one may use the ultrasonic nebulizer sold by the DeVilbiss Health Care, Inc. of Somerset, Pa.; see, e.g., the “Instruction Manual” for the “Ultra-Neb 99 Ultrasonic Nebulizer, publication A-850-C (published by DeVilbiss, Somerset, Pa., 1989).

In the embodiment shown in FIG. 8, the oscillators of ultrasonic nebulizer 513 are shown contacting an exterior surface of misting chamber 511. In this embodiment, the ultrasonic waves produced by the oscillators are transmitted via the walls of the misting chamber 511 and effect the misting of solution 509.

In another embodiment, not shown, the oscillators of ultrasonic nebulizer 513 are in direct contact with solution 509.

In one embodiment, it is preferred that the ultrasonic power used with such machine is in excess of one watt and, more preferably, in excess of 10 watts. In one embodiment, the power used with such machine exceeds about 50 watts.

During the time solution 509 is being caused to mist, it is preferably contacted with carrier gas to apply pressure to the solution and mist. It is preferred that a sufficient amount of carrier gas be introduced into the system at a sufficiently high flow rate so that pressure on the system is in excess of atmospheric pressure. Thus, for example, in one embodiment wherein chamber 511 has a volume of about 200 cubic centimeters, the flow rate of the carrier gas was from about 100 to about 150 milliliters per minute.

In one embodiment, the carrier gas 515 is introduced via feeding line 517 at a rate sufficient to cause solution 509 to mist at a rate of from about 0.5 to about 20 milliliters per minute. In one embodiment, the misting rate of solution 9 is from about 1.0 to about 3.0 milliliters per minute.

Substantially any gas that facilitates the formation of plasma may be used as carrier gas 515. Thus, by way of illustration, one may use oxygen, air, argon, nitrogen, mixtures thereof and the like; in one embodiment, a mixture of oxygen and nitrogen is used. It is preferred that the carrier gas used be a compressed gas under a pressure in excess 760 millimeters of mercury. In this embodiment, the use of the compressed gas facilitates the movement of the mist from the misting chamber 511 to the plasma region 521.

The misting container 511 may be any reaction chamber conventionally used by those skilled in the art and preferably is constructed out of such acid-resistant materials such as glass, plastic, and the like.

The mist from misting chamber 511 is fed via misting outlet line 519 into the plasma region 521 of plasma reactor 525. In plasma reactor 525, the mist is mixed with plasma generated by plasma gas 527 and subjected to radio frequency radiation provided by a radio-frequency coil 529.

The plasma reactor 525 provides energy to form plasma and to cause the plasma to react with the mist. Any of the plasmas reactors well known to those skilled in the art may be used as plasma reactor 525. Some of these plasma reactors are described in J. Mort et al.'s “Plasma Deposited Thin Films” (CRC Press Inc., Boca Raton, Fla., 1986); in “Methods of Experimental Physics,” Volume 9—Parts A and B, Plasma Physics (Academic Press, New York, 1970/1971); and in N. H. Burlingame's “Glow Discharge Nitriding of Oxides,” Ph.D. thesis (Alfred University, Alfred, N.Y., 1985), available from University Microfilm International, Ann Arbor, Mich.

In one preferred embodiment, the plasma reactor 525 is a “model 56 torch” available from the TAFA Inc. of Concord, N.H. It is preferably operated at a frequency of about 4 megahertz and an input power of 30 kilowatts.

Referring again to FIG. 8, and to the preferred embodiment depicted therein, it will be seen that into feeding lines 529 and 531 is fed plasma gas 527. As is known to those skilled in the art, a plasma can be produced by passing gas into a plasma reactor. A discussion of the formation of plasma is contained in B. Chapman's “Glow Discharge Processes” (John Wiley & Sons, New York, 1980)

In one preferred embodiment, the plasma gas used is a mixture of argon and oxygen. In another embodiment, the plasma gas is a mixture of nitrogen and oxygen. In yet another embodiment, the plasma gas is pure argon or pure nitrogen.

When the plasma gas is pure argon or pure nitrogen, it is preferred to introduce into the plasma reactor at a flow rate of from about 5 to about 30 liters per minute.

When a mixture of oxygen and either argon or nitrogen is used, the concentration of oxygen in the mixture preferably is from about 1 to about 40 volume percent and, more preferably, from about 15 to about 25 volume percent. When such a mixture is used, the flow rates of each gas in the mixture should be adjusted to obtain the desired gas concentrations. Thus, by way of illustration, in one embodiment that uses a mixture of argon and oxygen, the argon flow rate is 15 liters per minute, and the oxygen flow rate is 40 liters per minute.

In one embodiment, auxiliary oxygen 533 is fed into the top of reactor 25, between the plasma region 521 and the flame region 540, via lines 536 and 538. In this embodiment, the auxiliary oxygen is not involved in the formation of plasma but is involved in the enhancement of the oxidation of the ferrite material.

Radio frequency energy is applied to the reagents in the plasma reactor 525, and it causes vaporization of the mist.

In general, the energy is applied at a frequency of from about 100 to about 30,000 kilohertz. In one embodiment, the radio frequency used is from about 1 to 20 megahertz. In another embodiment, the radio frequency used is from about 3 to about 5 megahertz.

As is known to those skilled in the art, such radio frequency alternating currents may be produced by conventional radio frequency generators. Thus, by way of illustration, said TAPA Inc. “model 56 torch” may be attached to a radio frequency generator rated for operation at 35 kilowatts which manufactured by Lepel Company (a division of TAFA Inc.) and which generates an alternating current with a frequency of 4 megahertz at a power input of 30 kilowatts. Thus, e.g., one may use an induction coil driven at 2.5-5.0 megahertz that is sold as the “PLASMOC 2” by ENI Power Systems, Inc. of Rochester, N.Y.

The use of these type of radio-frequency generators is described in the Ph.D. theses entitled (1) “Heat Transfer Mechanisms in High-Temperature Plasma Processing of Glasses,” Donald M. McPherson (Alfred University, Alfred, N.Y., January, 1988) and (2) the aforementioned Nicholas H. Burlingame's “Glow Discharge Nitriding of Oxides.”

The plasma vapor 523 formed in plasma reactor 525 is allowed to exit via the aperture 542 and can be visualized in the flame region 540. In this region, the plasma contacts air that is at a lower temperature than the plasma region 521, and a flame is visible. A theoretical model of the plasma/flame is presented on pages 88 et seq. of said McPherson thesis.

The vapor 544 present in flame region 540 is propelled upward towards substrate 546. Any material onto which vapor 544 will condense may be used as a substrate. Thus, by way of illustration, one may use nonmagnetic materials such alumina, glass, gold-plated ceramic materials, and the like. In one embodiment, substrate 46 consists essentially of a magnesium oxide material such as single crystal magnesium oxide, polycrystalline magnesium oxide, and the like.

In another embodiment, the substrate 546 consists essentially of zirconia such as, e.g., yttrium stabilized cubic zirconia.

In another embodiment, the substrate 546 consists essentially of a material selected from the group consisting of strontium titanate, stainless steel, alumina, sapphire, and the like.

The aforementioned listing of substrates is merely meant to be illustrative, and it will be apparent that many other substrates may be used. Thus, by way of illustration, one may use any of the substrates mentioned in M. Sayer's “Ceramic Thin Films . . . ” article, supra. Thus, for example, in one embodiment it is preferred to use one or more of the substrates described on page 286 of “Superconducting Devices,” edited by S. T. Ruggiero et al. (Academic Press, Inc., Boston, 1990).

One advantage of this embodiment of applicants' process is that the substrate may be of substantially any size or shape, and it may be stationary or movable. Because of the speed of the coating process, the substrate 546 may be moved across the aperture 542 and have any or all of its surface be coated.

As will be apparent to those skilled in the art, in the embodiment depicted in FIG. 8, the substrate 546 and the coating 548 are not drawn to scale but have been enlarged to the sake of ease of representation.

Referring again to FIG. 8, the substrate 546 may be at ambient temperature. Alternatively, one may use additional heating means to heat the substrate prior to, during, or after deposition of the coating.

Referring again to FIG. 8, and in one preferred embodiment, a heater (not shown) is used to heat the substrate to a temperature of from about 100 to about 800 degrees centigrade.

In one aspect of this embodiment, temperature sensing means (not shown) may be used to sense the temperature of the substrate and, by feedback means (not shown), adjust the output of the heater (not shown). In one embodiment, not shown, when the substrate 46 is relatively near flame region 40, optical pyrometry measurement means (not shown) may be used to measure the temperature near the substrate.

In one embodiment, a shutter (not shown) is used to selectively interrupt the flow of vapor 544 to substrate 546. This shutter, when used, should be used prior to the time the flame region has become stable; and the vapor should preferably not be allowed to impinge upon the substrate prior to such time.

The substrate 546 may be moved in a plane that is substantially parallel to the top of plasma chamber 525. Alternatively, or additionally, it may be moved in a plane that is substantially perpendicular to the top of plasma chamber 525. In one embodiment, the substrate 46 is moved stepwise along a predetermined path to coat the substrate only at certain predetermined areas.

In one embodiment, rotary substrate motion is utilized to expose as much of the surface of a complex-shaped article to the coating. This rotary substrate motion may be effectuated by conventional means. See, e.g., “Physical Vapor Deposition,” edited by Russell J. Hill (Temescal Division of The BOC Group, Inc., Berkeley, Calif., 1986).

The process of this embodiment of the invention allows one to coat an article at a deposition rate of from about 0.01 to about 10 microns per minute and, preferably, from about 0.1 to about 1.0 microns per minute, with a substrate with an exposed surface of 35 square centimeters. One may determine the thickness of the film coated upon said reference substrate material (with an exposed surface of 35 square centimeters) by means well known to those skilled in the art.

The film thickness can be monitored in situ, while the vapor is being deposited onto the substrate. Thus, by way of illustration, one may use an IC-6000 thin film thickness monitor (also referred to as “deposition controller”) manufactured by Leybold Inficon Inc. of East Syracuse, N.Y.

The deposit formed on the substrate may be measured after the deposition by standard profilometry techniques. Thus, e.g., one may use a DEKTAK Surface Profiler, model number 900051 (available from Sloan Technology Corporation, Santa Barbara, Calif.).

In general, at least about 80 volume percent of the particles in the as-deposited film are smaller than about 1 micron. It is preferred that at least about 90 percent of such particles are smaller than 1 micron. Because of this fine grain size, the surface of the film is relatively smooth.

In one preferred embodiment, the as-deposited film is post-annealed.

It is preferred that the generation of the vapor in plasma rector 525 be conducted under substantially atmospheric pressure conditions. As used in this specification, the term “substantially atmospheric” refers to a pressure of at least about 600 millimeters of mercury and, preferably, from about 600 to about 1,000 millimeters of mercury. It is preferred that the vapor generation occur at about atmospheric pressure. As is well known to those skilled in the art, atmospheric pressure at sea level is 760 millimeters of mercury.

The process of this invention may be used to produce coatings on a flexible substrate such as, e.g., stainless steel strips, silver strips, gold strips, copper strips, aluminum strips, and the like. One may deposit the coating directly onto such a strip. Alternatively, one may first deposit one or more buffer layers onto the strip(s). In other embodiments, the process of this invention may be used to produce coatings on a rigid or flexible cylindrical substrate, such as a tube, a rod, or a sleeve.

Referring again to FIG. 8, and in the embodiment depicted therein, as the coating 548 is being deposited onto the substrate 546, and as it is undergoing solidification thereon, it is preferably subjected to a magnetic field produced by magnetic field generator 550.

In this embodiment, it is preferred that the magnetic field produced by the magnetic field generator 550 have a field strength of from about 2 Gauss to about 40 Tesla.

It is preferred to expose the deposited material for at least 10 seconds and, more preferably, for at least 30 seconds, to the magnetic field, until the magnetic moments of the nano-sized particles being deposited have been substantially aligned.

As used herein, the term “substantially aligned” means that the inductance of the device being formed by the deposited nano-sized particles is at least 90 percent of its maximum inductance. One may determine when such particles have been aligned by, e.g., measuring the inductance, the permeability, and/or the hysteresis loop of the deposited material.

Thus, e.g., one may measure the degree of alignment of the deposited particles with an impedance meter, a inductance meter, or a SQUID.

In one embodiment, the degree of alignment of the deposited particles is measured with an inductance meter. One may use, e.g., a conventional conductance meter such as, e.g., the conductance meters disclosed in U.S. Pat. Nos. 4,779,462, 4,937,995, 5,728,814 (apparatus for determining and recording injection does in syringes using electrical inductance), U.S. Pat. Nos. 6,318,176, 5,014,012, 4,869,598, 4,258,315 (inductance meter), U.S. Pat. No. 4,045,728 (direct reading inductance meter), U.S. Pat. Nos. 6,252,923, 6,194,898, 6,006,023 (molecular sensing apparatus), U.S. Pat. No. 6,048,692 (sensors for electrically sensing binding events for supported molecular receptors), and the like. The entire disclosure of each of these United States patents is hereby incorporated by reference into this specification.

When measuring the inductance of the coated sample, the inductance is preferably measured using an applied wave with a specified frequency. As the magnetic moments of the coated samples align, the inductance increases until a specified value; and it rises in accordance with a specified time constant in the measurement circuitry.

In one embodiment, the deposited material is contacted with the magnetic field until the inductance of the deposited material is at least about 90 percent of its maximum value under the measurement circuitry. At this time, the magnetic particles in the deposited material have been aligned to at least about 90 percent of the maximum extent possible for maximizing the inductance of the sample.

By way of illustration and not limitation, a metal rod with a diameter of 1 micron and a length of 1 millimeter, when uncoated with magnetic nano-sized particles, might have an inductance of about 1 nanohenry. When this metal rod is coated with, e.g., nano-sized ferrites, then the inductance of the coated rod might be 5 nanohenries or more. When the magnetic moments of the coating are aligned, then the inductance might increase to 50 nanohenries, or more. As will be apparent to those skilled in the art, the inductance of the coated article will vary, e.g., with the shape of the article and also with the frequency of the applied electromagnetic field.

One may use any of the conventional magnetic field generators known to those skilled in the art to produce such as magnetic field. Thus, e.g., one may use one or more of the magnetic field generators disclosed in U.S. Pat. Nos. 6,503,364, 6,377,149 (magnetic field generator for magnetron plasma generation), U.S. Pat. No. 6,353,375 (magnetostatic wave device), U.S. Pat. No. 6,340,888 (magnetic field generator for MRI), U.S. Pat. Nos. 6,336,989, 6,335,617 (device for calibrating a magnetic field generator), U.S. Pat. Nos. 6,313,632, 6,297,634, 6,275,128, 6,246,066 (magnetic field generator and charged particle beam irradiator), U.S. Pat. No. 6,114,929 (magnetostatic wave device), U.S. Pat. No. 6,099,459 (magnetic field generating device and method of generating and applying a magnetic field), U.S. Pat. Nos. 5,795,212, 6,106,380 (deterministic magnetorheological finishing), U.S. Pat. No. 5,839,944 (apparatus for deterministic magnetorheological finishing), U.S. Pat. No. 5,971,835 (system for abrasive jet shaping and polishing of a surface using a magnetorheological fluid), U.S. Pat. Nos. 5,951,369, 6,506,102 (system for magnetorheological finishing of substrates), U.S. Pat. Nos. 6,267,651, 6,309,285 (magnetic wiper), and the like. The entire disclosure of each of these United States patents is hereby incorporated by reference into this specification.

In one embodiment, the magnetic field is 1.8 Tesla or less. In this embodiment, the magnetic field can be applied with, e.g., electromagnets disposed around a coated substrate.

For fields greater than about 2 Tesla, one may use superconducting magnets that produce fields as high as 40 Tesla. Reference may be had, e.g., to U.S. Pat. No. 5,319,333 (superconducting homogeneous high field magnetic coil), U.S. Pat. Nos. 4,689,563, 6,496,091 (superconducting magnet arrangement), U.S. Pat. No. 6,140,900 (asymmetric superconducting magnets for magnetic resonance imaging), U.S. Pat. No. 6,476,700 (superconducting magnet system), U.S. Pat. No. 4,763,404 (low current superconducting magnet), U.S. Pat. No. 6,172,587 (superconducting high field magnet), U.S. Pat. No. 5,406,204, and the like. The entire disclosure of each of these United States patents is hereby incorporated by reference into this specification.

In one embodiment, no magnetic field is applied to the deposited coating while it is being solidified. In this embodiment, as will be apparent to those skilled in the art, there still may be some alignment of the magnetic domains in a plane parallel to the surface of substrate as the deposited particles are locked into place in a matrix (binder) deposited onto the surface.

In one embodiment, depicted in FIG. 8, the magnetic field 552 is preferably delivered to the coating 548 in a direction that is substantially parallel to the surface 556 of the substrate 546. In another embodiment, not shown, the magnetic field 558 is delivered in a direction that is substantially perpendicular to the surface 556. In yet another embodiment, the magnetic field 560 is delivered in a direction that is angularly disposed vis-à-vis surface 556 and may form, e.g., an obtuse angle (as in the case of field 62). As will be apparent, combinations of these magnetic fields may be used.

FIG. 9 is a flow diagram of another process that may be used to make the nanomagnetic compositions of this invention. Referring to FIG. 9, and to the preferred process depicted therein, it will be seen that nano-sized ferromagnetic material(s), with a particle size less than about 100 nanometers, is preferably charged via line 660 to mixer 63. It is preferred to charge a sufficient amount of such nano-sized material(s) so that at least about 10 weight percent of the mixture formed in mixer 663 is comprised of such nano-sized material. In one embodiment, at least about 40 weight percent of such mixture in mixer 663 is comprised of such nano-sized material. In another embodiment, at least about 50 weight percent of such mixture in mixer 663 is comprised of such nano-sized material.

In one embodiment, one or more binder materials are charged via line 664 to mixer 662. In one embodiment, the binder used is a ceramic binder. These ceramic binders are well known. Reference may be had, e.g., to pages 172-197 of James S. Reed's “Principles of Ceramic Processing,” Second Edition (John Wiley & Sons, Inc., New York, N.Y., 1995). As is disclosed in the Reed book, the binder may be a clay binder (such as fine kaolin, ball clay, and bentonite), an organic colloidal particle binder (such as microcrystalline cellulose), a molecular organic binder (such as natural gums, polysaccharides, lignin extracts, refined alginate, cellulose ethers, polyvinyl alcohol, polyvinylbutyl, polymethyl methacrylate, polyethylene glycol, paraffin, and the like.). etc.

In one embodiment, the binder is a synthetic polymeric or inorganic composition. Thus, and referring to George S. Brady et al.'s “Materials Handbook,” (McGraw-Hill, Inc., New York, N.Y. 1991), the binder may be acrylonitrile-butadiene-styrene (see pages 5-6), an acetal resin (see pages 6-7), an acrylic resin (see pages 10-12), an adhesive composition (see pages 14-18), an alkyd resin (see page 27-28), an allyl plastic (see pages 31-32), an amorphous metal (see pages 53-54), a biocompatible material (see pages 95-98), boron carbide (see page 106), boron nitride (see page 107), camphor (see page 135), one or more carbohydrates (see pages 138-140), carbon steel (see pages 146-151), casein plastic (see page 157), cast iron (see pages 159-164), cast steel (see pages 166-168), cellulose (see pages 172-175), cellulose acetate (see pages 175-177), cellulose nitrate (see pages 177), cement (see page 178-180), ceramics (see pages 180-182), cermets (see pages 182-184), chlorinated polyethers (see pages 191-191), chlorinated rubber (see pages 191-193), cold-molded plastics (see pages 220-221), concrete (see pages 225-227), conductive polymers and elastomers (see pages 227-228), degradable plastics (see pages 261-262), dispersion-strengthened metals (see pages 273-274), elastomers (see pages 284-290), enamel (see pages 299-301), epoxy resins (see pages 301-302), expansive metal (see page 313), ferrosilicon (see page 327), fiber-reinforced plastics (see pages 334-335), fluoroplastics (see pages 345-347), foam materials (see pages 349-351), fusible alloys (see pages 362-364), glass (see pages 376-383), glass-ceramic materials (see pages 383-384), gypsum (see pages 406-407), impregnated wood (see pages 422-423), latex (see pages 456-457), liquid crystals (see page 479). lubricating grease (see pages 488-492), magnetic materials (see pages 505-509), melamine resin (see pages 5210-521), metallic materials (see pages 522-524), nylon (see pages 567-569), olefin copolymers (see pages 574-576), phenol-formaldehyde resin (see pages 615-617), plastics (see pages 637-639), polyarylates (see pages 647-648), polycarbonate resins (see pages 648), polyester thermoplastic resins (see pages 648-650), polyester thermosetting resins (see pages 650-651), polyethylenes (see pages 651-654), polyphenylene oxide (see pages 644-655), polypropylene plastics (see pages 655-656), polystyrenes (see pages 656-658), proteins (see pages 666-670), refractories (see pages 691-697), resins (see pages 697-698), rubber (see pages 706-708), silicones (see pages 747-749), starch (see pages 797-802), superalloys (see pages 819-822), super polymers (see pages 823-825), thermoplastic elastomers (see pages 837-839), urethanes (see pages 874-875), vinyl resins (see pages 885-888), wood (see pages 912-916), mixtures thereof, and the like.

Referring again to FIG. 9, one may charge to line 664 either one or more of these “binder material(s)” and/or the precursor(s) of these materials that, when subjected to the appropriate conditions in former 666, will form the desired mixture of nanomagnetic material and binder.

Referring again to FIG. 9, and in the preferred process depicted therein, the mixture within mixer 63 is preferably stirred until a substantially homogeneous mixture is formed. Thereafter, it may be discharged via line 665 to former 66.

One process for making a fluid composition comprising nanomagnetic particles is disclosed in U.S. Pat. No. 5,804,095, “Magnetorheological Fluid Composition,”, of Jacobs et al; the disclosure of this patent is incorporated herein by reference. In this patent, there is disclosed a process comprising numerous material handling steps used to prepare a nanomagnetic fluid comprising iron carbonyl particles. One suitable source of iron carbonyl particles having a median particle size of 3.1 microns is the GAF Corporation.

The process of Jacobs et al, is applicable to the present invention, wherein such nanomagnetic fluid further comprises a polymer binder, thereby forming a nanomagnetic paint. In one embodiment, the nanomagnetic paint is formulated without abrasive particles of cerium dioxide. In another embodiment, the nanomagnetic fluid further comprises a polymer binder, and aluminum nitride is substituted for cerium dioxide.

There are many suitable mixing processes and apparatus for the milling, particle size reduction, and mixing of fluids comprising solid particles. For example, e.g., iron carbonyl particles or other ferromagnetic particles of the paint may be further reduced to a size on the order of 100 nanometers or less, and/or thoroughly mixed with a binder polymer and/or a liquid solvent by the use of a ball mill, a sand mill, a paint shaker holding a vessel containing the paint components and hard steel or ceramic beads; a homogenizer (such as the Model Ytron Z made by the Ytron Quadro Corporation of Chesham, United Kingdom, or the Microfluidics M700 made by the MFIC Corporation of Newton, Mass.), a powder dispersing mixer (such as the Ytron Zyclon mixer, or the Ytron Xyclon mixer, or the Ytron PID mixer by the Ytron Quadro Corporation); a grinding mill (such as the Model F10 Mill by the Ytron Quadro Corporation); high shear mixers (such as the Ytron Y mixer by the Ytron Quadro Corporation), the Silverson Laboratory Mixer sold by the Silverson Corporation of East Longmeadow, Mass., and the like. The use of one or more of these apparatus in series or in parallel may produce a suitably formulated nanomagnetic paint.

Referring again to FIG. 9, the former 666 is preferably equipped with an input line 68 and an exhaust line 670 so that the atmosphere within the former can be controlled. One may utilize an ambient atmosphere, an inert atmosphere, pure nitrogen, pure oxygen, mixtures of various gases, and the like. Alternatively, or additionally, one may use lines 668 and 670 to afford subatmospheric pressure, atmospheric pressure, or superatomspheric pressure within former 666.

In the embodiment depicted, former 666 is also preferably comprised of an electromagnetic coil 672 that, in response from signals from controller 674, can control the extent to which, if any, a magnetic field is applied to the mixture within the former 666 (and also within the mold 667 and/or the spinnerette 669).

The controller 674 is also adapted to control the temperature within the former 666 by means of heating/cooling assembly.

Referring again to FIG. 8, and in one preferred embodiment, a heater (not shown) is used to heat the substrate 546 to a temperature of from about 100 to about 800 degrees centigrade.

In one aspect of this embodiment, temperature sensing means (not shown) may be used to sense the temperature of the substrate 546 and, by feedback means (not shown), adjust the output of the heater (not shown). In one embodiment, not shown, when the substrate 546 is relatively near flame region 540, optical pyrometry measurement means (not shown) may be used to measure the temperature near the substrate.

In one embodiment, a shutter (not shown) is used to selectively interrupt the flow of vapor 544 to substrate 546. This shutter, when used, should be used prior to the time the flame region has become stable; and the vapor should preferably not be allowed to impinge upon the substrate prior to such time.

The substrate 546 may be moved in a plane that is substantially parallel to the top of plasma chamber 525. Alternatively, or additionally, it may be moved in a plane that is substantially perpendicular to the top of plasma chamber 525. In one embodiment, the substrate 546 is moved stepwise along a predetermined path to coat the substrate only at certain predetermined areas.

In one embodiment, rotary substrate motion is utilized to expose as much of the surface of a complex-shaped article to the coating. This rotary substrate motion may be effectuated by conventional means. See, e.g., “Physical Vapor Deposition,” edited by Russell J. Hill (Temescal Division of The BOC Group, Inc., Berkeley, Calif., 1986).

The process of this embodiment of the invention allows one to coat an article at a deposition rate of from about 0.01 to about 10 microns per minute and, preferably, from about 0.1 to about 1.0 microns per minute, with a substrate with an exposed surface of 35 square centimeters. One may determine the thickness of the film coated upon said reference substrate material (with an exposed surface of 35 square centimeters) by means well known to those skilled in the art.

The film thickness can be monitored in situ, while the vapor is being deposited onto the substrate. Thus, by way of illustration, one may use an IC-6000 thin film thickness monitor (also referred to as “deposition controller”) manufactured by Leybold Inficon Inc. of East Syracuse, N.Y.

The deposit formed on the substrate may be measured after the deposition by standard profilometry techniques. Thus, e.g., one may use a DEKTAK Surface Profiler, model number 900051 (available from Sloan Technology Corporation, Santa Barbara, Calif.).

In general, at least about 80 volume percent of the particles in the as-deposited film are smaller than about 1 micron. It is preferred that at least about 90 percent of such particles are smaller than 1 micron. Because of this fine grain size, the surface of the film is relatively smooth.

In one preferred embodiment, the as-deposited film is post-annealed.

It is preferred that the generation of the vapor in plasma rector 525 be conducted under substantially atmospheric pressure conditions. As used in this specification, the term “substantially atmospheric” refers to a pressure of at least about 600 millimeters of mercury and, preferably, from about 600 to about 1,000 millimeters of mercury. It is preferred that the vapor generation occur at about atmospheric pressure. As is well known to those skilled in the art, atmospheric pressure at sea level is 760 millimeters of mercury.

The process of this invention may be used to produce coatings on a flexible substrate such as, e.g., stainless steel strips, silver strips, gold strips, copper strips, aluminum strips, and the like. One may deposit the coating directly onto such a strip. Alternatively, one may first deposit one or more buffer layers onto the strip(s). In other embodiments, the process of this invention may be used to produce coatings on a rigid or flexible cylindrical substrate, such as a tube, a rod, or a sleeve.

Referring again to FIG. 8, and in the embodiment depicted therein, as the coating 548 is being deposited onto the substrate 546, and as it is undergoing solidification thereon, it is preferably subjected to a magnetic field produced by magnetic field generator 550.

In this embodiment, it is preferred that the magnetic field produced by the magnetic field generator 550 have a field strength of from about 2 Gauss to about 40 Tesla.

Substrates with Composite Coatings Disposed Thereon

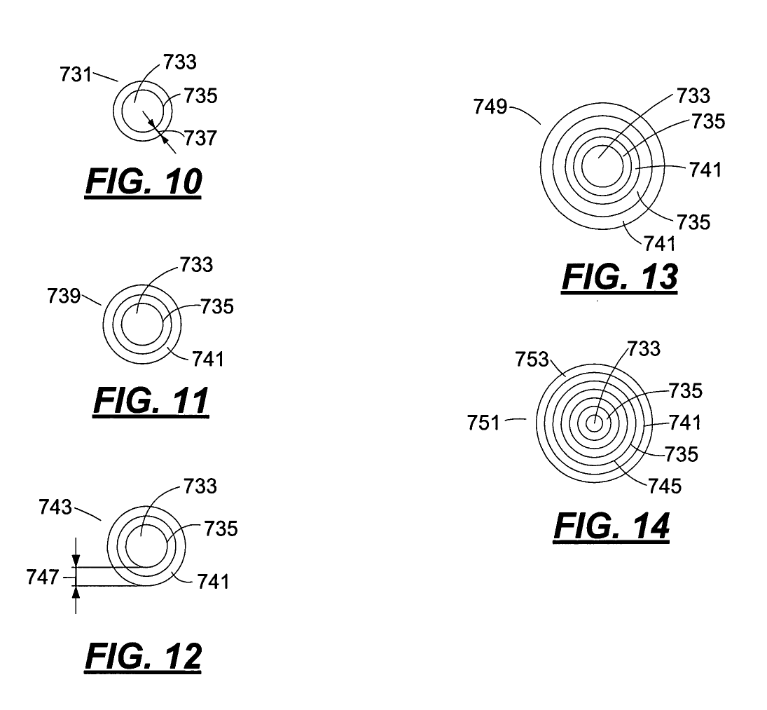

FIGS. 10-14 are sectional views of coated substrates wherein the coatings comprise two more discrete layers of different materials.

FIG. 10 is a sectional view one preferred coated assembly 731 that is comprised of a conductor 733 and, disposed around such conductor 733, a layer of nanomagnetic material 735.

In the embodiment depicted in FIG. 10, the layer 735 of nanomagnetic material preferably has a thickness of at least 150 nanometers and, more preferably, at least about 200 nanometers. In one embodiment, the thickness of layer 735 is from about 500 to about 1,000 nanometers.

FIG. 11 is a schematic sectional view of a magnetically shielded assembly 739 that is similar to assembly 731 but differs therefrom in that a layer 741 of nanoelectrical material is disposed around layer 735.

The layer of nanoelectrical material 741 preferably has a thickness of from about 0.5 to about 2 microns. In this embodiment, the nanoelectrical material comprising layer 741 has a resistivity of from about 1 to about 100 microohm-centimeters. As is known to those skilled in the art, when nanoelectrical material is exposed to electromagnetic radiation, and in particular to an electric field, it will shield the substrate over which it is disposed from such electrical field. Reference may be had, e.g., to International patent publication WO9820719 in which reference is made to U.S. Pat. No. 4,963,291; each of these patents and patent applications is hereby incorporated by reference into this specification.

As is disclosed in U.S. Pat. No. 4,963,291, one may produce electromagnetic shielding resins comprised of electroconductive particles, such as iron, aluminum, copper, silver and steel in sizes ranging from 0.5 to 0.50 microns. The entire disclosure of this United States patent is hereby incorporated by reference into this specification.

The nanoelectrical particles used in this aspect of the invention preferably have a particle size within the range of from about 1 to about 100 microns, and a resistivity of from about 1.6 to about 100 microohm-centimeters. In one embodiment, such nanoelectrical particles comprise a mixture of iron and aluminum. In another embodiment, such nanoelectrical particles consist essentially of a mixture of iron and aluminum.

It is preferred that, in such nanoelectrical particles, and in one embodiment, at least 9 moles of aluminum are present for each mole of iron. In another embodiment, at least about 9.5 moles of aluminum are present for each mole of iron. In yet another embodiment, at least 9.9 moles of aluminum are present for each mole of iron.

In one embodiment, and referring again to FIG. 13, the layer 741 of nanoelectrical material has a thermal conductivity of from about 1 to about 4 watts/centimeter-degree Kelvin.

In one embodiment, not shown, in either or both of layers 735 and 741 there is present both the nanoelectrical material and the nanomagnetic material One may produce such a layer 735 and/or 741 by simultaneously depositing the nanoelectrical particles and the nanomagnetic particles with, e.g., sputtering technology such as, e.g., the sputtering technology described elsewhere in this specification.

FIG. 12 is a sectional schematic view of a magnetically shielded assembly 743 that differs from assembly 731 in that it contains a layer 745 of nanothermal material disposed around the layer 735 of nanomagnetic material. The layer 745 of nanothermal material preferably has a thickness of less than 2 microns and a thermal conductivity of at least about 150 watts/meter-degree Kelvin and, more preferably, at least about 200 watts/meter-degree Kelvin. It is preferred that the resistivity of layer 745 be at least about 10¹⁰ microohm-centimeters and, more preferably, at least about 10¹² microohm-centimeters. In one embodiment, the resistivity of layer 745 is at least about 10¹³ microohm centimeters. In one embodiment, the nanothermal layer is comprised of AlN.

In one embodiment, depicted in FIG. 12, the thickness 747 of all of the layers of material coated onto the conductor 733 is preferably less than about 20 microns.

In FIG. 13, a sectional view of an assembly 749 is depicted that contains, disposed around conductor 733, layers of nanomagnetic material 735, nanoelectrical material 741, nanomagnetic material 735, and nanoelectrical material 741.

In FIG. 14, a sectional view of an assembly 751 is depicted that contains, disposed around conductor 733, a layer 735 of nanomagnetic material, a layer 741 of nanoelectrical material, a layer 735 of nanomagnetic material, a layer 745 of nanothermal material, and a layer 735 of nanomagnetic material. Optionally disposed in layer 753 is antithrombogenic material that is biocompatible with the living organism in which the assembly 751 is preferably disposed.

In the embodiments depicted in FIGS. 10 through 14, the coatings 735, and/or 741, and/or 745, and/or 753, are disposed around a conductor 733. In one embodiment, the conductor so coated is preferably part of medical device, preferably an implanted medical device (such as, e.g., a pacemaker). In another embodiment, in addition to coating the conductor 733, or instead of coating the conductor 733, the actual medical device itself is coated.

Preparation of Coatings Comprised of Nanoelectrical Material

In this portion of the specification, coatings comprised of nanoelectrical material will be described. In accordance with one aspect of this invention, there is provided a nanoelectrical material with an average particle size of less than 100 nanometers, a surface area to volume ratio of from about 0.1 to about 0.05l/nanometer, and a relative dielectric constant of less than about 1.5.

The nanoelectrical particles of this aspect of the invention have an average particle size of less than about 100 nanometers. In one embodiment, such particles have an average particle size of less than about 50 nanometers. In yet another embodiment, such particles have an average particle size of less than about 10 nanometers.

The nanoelectrical particles of this invention have surface area to volume ratio of from about 0.1 to about 0.05l/nanometer.

When the nanoelectrical particles of this invention are agglomerated into a cluster, or when they are deposited onto a substrate, the collection of particles preferably has a relative dielectric constant of less than about 1.5. In one embodiment, such relative dielectric constant is less than about 1.2.

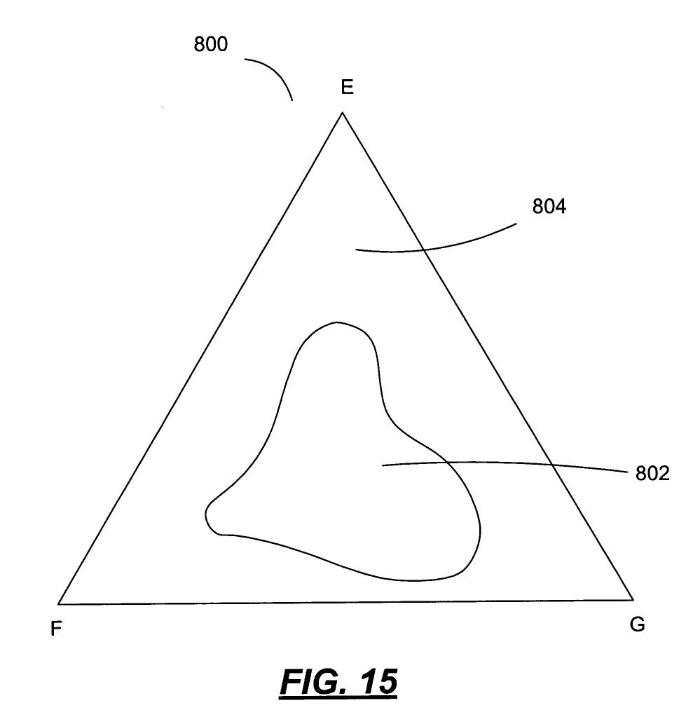

In one embodiment, the nanoelectrical particles of this invention are preferably comprised of aluminum, magnesium, and nitrogen atoms. This embodiment is illustrated in FIG. 15.

FIG. 15 illustrates a phase diagram 800 comprised of moieties E, F, and G. Moiety E is preferably selected from the group consisting of aluminum, copper, gold, silver, and mixtures thereof. It is preferred that the moiety E have a resistivity of from about 2 to about 100 microohm-centimeters. In one preferred embodiment, moiety E is aluminum with a resistivity of about 2.824 microohm-centimeters. As will apparent, other materials with resistivities within the desired range also may be used.

Referring again to FIG. 15, moiety G is selected from the group consisting of nitrogen, oxygen, and mixtures thereof. In one embodiment, C is nitrogen, A is aluminum, and aluminum nitride is present as a phase in the system.

Referring again to FIG. 15, and in one embodiment, moiety F is preferably a dopant that is present in a minor amount in the preferred aluminum nitride. In general, less than about 50 percent (by weight) of the F moiety is present, by total weight of the doped aluminum nitride. In one aspect of this embodiment, less than about 10 weight percent of the F moiety is present, by total weight of the doped aluminum nitride.

The F moiety may be, e.g., magnesium, zinc, tin, indium, gallium, niobium, zirconium, strontium, lanthanum, tungsten, mixtures thereof, and the like. In one embodiment, F is selected from the group consisting of magnesium, zinc, tin, and indium. In another especially preferred embodiment, the F moiety is magnesium.

Referring again to FIG. 15, and when E is aluminum, F is magnesium, and G is nitrogen, it will be seen that regions 802 and 804 correspond to materials which have a low relative dielectric constant (less than about 1.5), and a high relative dielectric constant (greater than about 1.5), respectively.

A Preferred Drug Delivery Assembly

In this section of the specification, applicants will describe a medical device with improved drug delivery capabilities. This medical device is similar to the medical device disclosed in published United States patent application 2004/0030379, the entire disclosure of which is hereby incorporated by reference into this specification. However, because applicants use an improved form of magnetic particles in the device, applicants device provides superior magnetic performance and, additionally, superior MRI imageability.

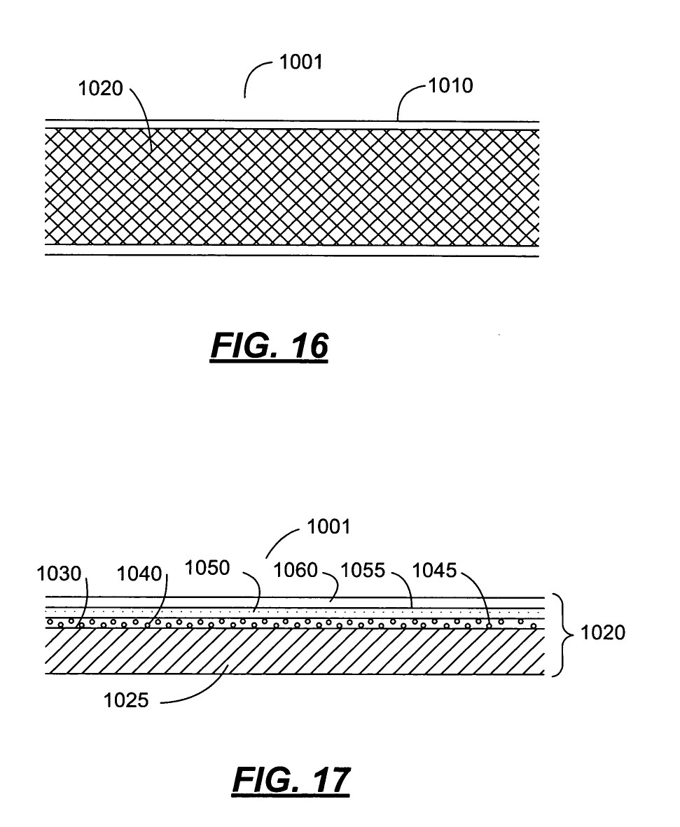

The medical system described in this section of the specification is preferably a stent 1010 (see FIG. 16) comprised of wire like struts 1020 (also see FIG. 16). As is disclosed in paragraph 22 of published United States patent application 2004/0030379, “The system of the present invention comprises (1) a medical device having a coating containing a biologically active material, and (2) a source of electromagnetic energy or a source for generating an electromagnetic field. The present invention can facilitate and/or modulate the delivery of the biologically active material from the medical device. The release of the biologically active material from the medical device is facilitated or modulated by the electromagnetic energy source or field. To utilize the system of the present invention, the practitioner may implant the coated medical device using regular procedures. After implantation, the patient is exposed to an extracorporal or external electromagnetic energy source or field to facilitate the release of the biologically active material from the medical device. The delivery of the biologically active material is on-demand, i.e., the material is not delivered or released from the medical device until a practitioner determines that the patient is in need of the biologically active material. The coating of the medical device of the present invention further comprises particles comprising a magnetic material, i.e., magnetic particles . . . ”

One embodiment of the medical device 1001 (see FIG. 16) is illustrated in FIG. 17, which shows a cross-sectional view of a coated strut 1020 of the stent.

In the embodiment depicted in FIG. 17, the coated strut 1020 comprises a strut 1025 having a surface 1030. The coated strut 1020 has a composite coating that comprises a first coating layer 1040 that contains a biologically active material 1045; in one embodiment, this first coating layer 1040 also contains polymeric material.

Referring again to FIG. 17, a second coating layer 1050 comprising nanomagnetic particles 1055 is disposed over the first coating layer 1040. This second coating layer 1055, in one embodiment, also includes polymeric material.

Referring again to FIG. 17, and in the preferred embodiment depicted, a third coating layer or sealing layer 1060 is disposed on top of the second coating layer 1050.

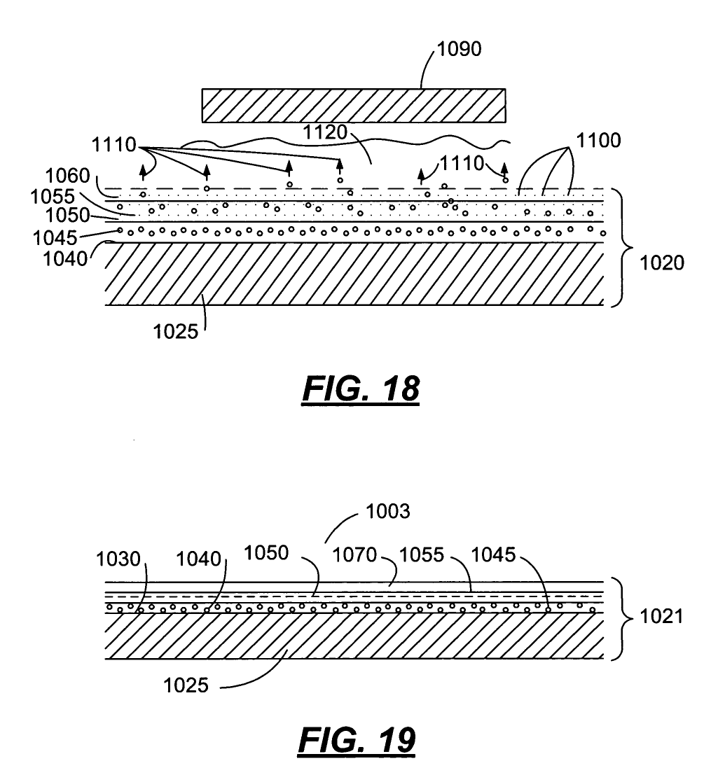

FIG. 18 is similar to FIG. 2B of United States published patent application 2004/0030379; and it illustrates the effect of exposing a patient (not shown), who is implanted with a stent having struts 1020 shown in FIG. 17, to an electromagnetic energy source or field 1090. When such a field 1090 is applied, the magnetic particles 1055 move out of the second coating layer 1050 in the direction of upward arrow 1110. This movement disrupts the sealing layer 1160 and forms channels 1100 in such sealing layer 1060.

Referring again to FIG. 18, it will be seen that the size of the channels 1100 formed generally depends on the size of the magnetic particles 1055 used. The biologically active material 1045 can then be released from the coating through the disrupted sealing layer 1060 into the surrounding tissue 1120. The duration of exposure to the field and the strength of the electromagnetic field 1090 determine the rate of delivery of the biologically active material 1045.

FIG. 19 illustrates another coated stent 1003; this Figure is similar to FIG. 3A of United States published patent application 2004/0030379. Referring to FIG. 19, and in the preferred embodiment depicted therein, it will be seen that, in this embodiment, the coated strut 1021 contains a coating comprised of a first coating layer 1040 comprising a biologically active material 1045 and preferably a polymeric material disposed over the surface 1030 of the strut 1025. A second coating layer or sealing layer 1070 comprising magnetic particles 1055 and a polymeric material is disposed on top of the first coating layer 1040.

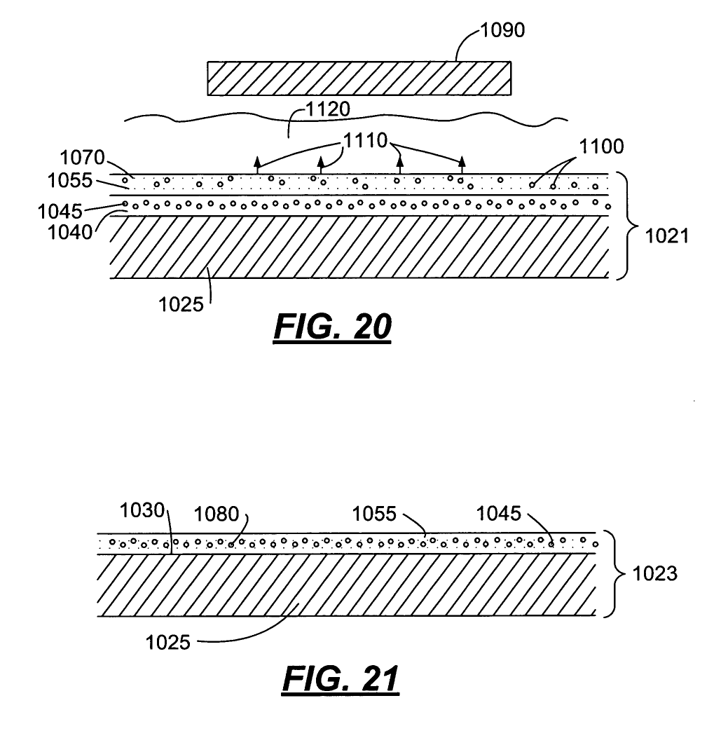

FIG. 20 illustrates the effect of exposing a patient (not shown) who is implanted with a stent having struts 1021 shown in FIG. 19 to an electromagnetic field 1090; this Figure is similar to FIG. 3B of United States published patent application 2004/0030379. Referring to FIG. 20 when such a field 1090 is applied, the magnetic particles 1055 move through the sealing layer 1070 as shown by the upward arrow 1110, and they create channels 1100 in the sealing layer 1070. The biologically active material 1045 in the underlying first coating layer 1040 is allowed to travel through the channels 1100 in the sealing layer 1070 and be released to the surrounding tissue 1120. Since the biologically active material 1045 is in a separate first coating layer 1040 and must migrate through the second coating layer or the sealing layer 1070, the release of the biologically active material 1045 is controlled after formation of the channels 1100.

FIG. 21 is similar to FIG. 4A of published United States patent application 2004/0030379, and it shows another embodiment of a coated stent strut 1023. In this embodiment, the coating comprises a coating layer 1080 comprising a biologically active material 1045, magnetic particles 1055, and a polymeric material.

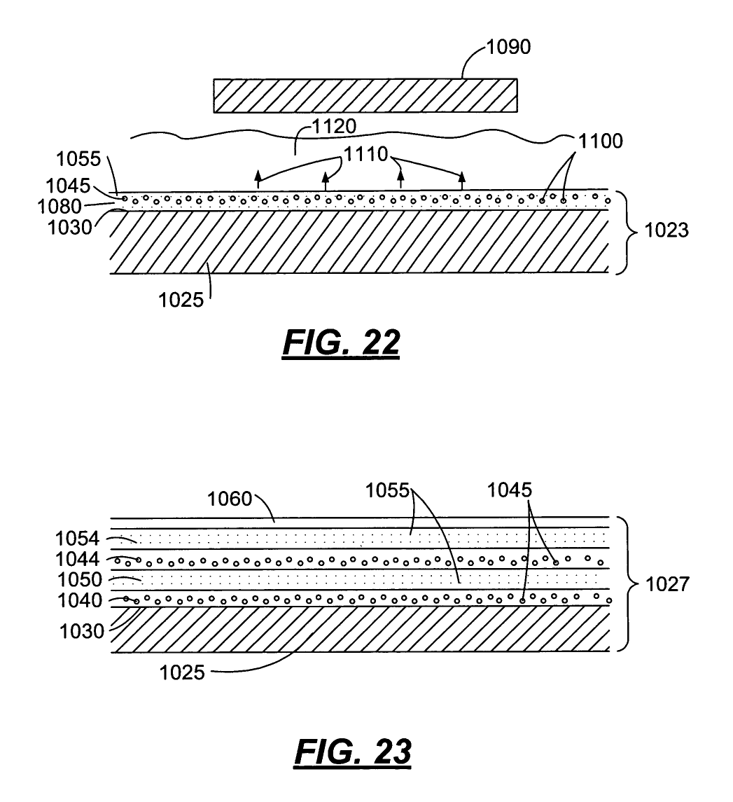

FIG. 22, which is similar to FIG. 4B of published United States patent application 2004/0030379, illustrates the effect of exposing a patient (not shown) who is implanted with a stent having struts 1023 to an electromagnetic field 1090. The field 1090 is applied, the magnetic particles 1055 move through the layer 1080 as shown by the arrow 1110 and create channels in the coating layer 1080. The biologically active material 1045 can then be released to the surrounding tissue 1120.

In another embodiment, and referring to FIGS. 16 and 23, the medical device 1001 of the present invention may be a stent having struts coated with a coating comprising more than one coating layer containing a magnetic material. FIG. 23 illustrates such a coated strut 1027. The coating comprises a first coating layer 1040 containing a polymeric material and a biologically active material 1045 which is disposed on the surface 1030 of a strut 1025. A second coating layer 1050 comprising a polymeric material and magnetic particles 1055 is disposed over the first coating layer 1040. A third coating layer 1044 comprising a polymeric material and a biologically active material 1045 is disposed over the second coating layer 1050. A fourth coating layer 1054 comprising a polymeric material and magnetic particles 1055 is disposed over this third layer 1044. Finally a sealing layer 1060 of a polymeric material is disposed over the fourth coating layer 1054. The permeability of the coating layers may be different from layer to layer so that the release of the biologically active material from each layer can differ. Also, the magnetic susceptibility of the magnetic particles may differ from layer to layer. The magnetic susceptibility may be varied using different concentrations or percentages of magnetic particles in the coating layers. The magnetic susceptibility of the magnetic particles may also be varied by changing the size and type of material used for the magnetic particles. When the magnetic susceptibility of the magnetic particles differs from layer to layer, different excitation intensity and/or frequency are required to activate the magnetic particles in each layer.

Referring again to FIG. 23, (and also to paragraph 27 at page 3 of published United States patent application 2004/0030379), the nanomagnetic particles preferably used in the embodiment depicted in FIG. 23 may be coated with a biologically active material and then incorporated into a coating for the medical device. In one embodiment, the biologically active material is a nucleic acid molecule. The nucleic acid coated nanomagnetic magnetic particles may be formed by painting, dipping, or spraying the magnetic particles with a solution comprising the nucleic acid. The nucleic acid molecules may adhere to the nanomagnetic particles via adsorption. Also the nucleic acid molecules may be linked to the magnetic particles chemically, via linking agents, covalent bonds, or chemical groups that have affinity for charged molecules. Application of an external electromagnetic field can cause the adhesion between the biologically active material and the magnetic particle to break, thereby allowing for release of the biologically active material.

In another embodiment, and referring to such FIGS. 16-23, the magnetic particles may be molded into or coated onto a non-metallic medical device, including a bio-absorb able medical device. The magnetic properties of the preferred nanomagnetic particles allow the non-metallic implant to be extracorporally imaged, vibrated, or moved. In specific embodiments, the nanomagnetic particles are painted, dipped or sprayed onto the outer surface of the device. The nanomagnetic particles may also be suspended in a curable coating, such as a UV curable epoxy, or they may be electrostatically sprayed onto the medical device and subsequently coated with a UV or heat curable polymeric material.

Additionally, and in some embodiments, the movement of the magnetic particles that occurs when the patient implanted with the coated device is exposed to an external electromagnetic field, releases mechanical energy into the surrounding tissue in which the medical device is implanted and triggers histamine production by the surrounding tissues. The histamine has a protective effect in preventing the formation of scar tissues in the vicinity at which the medical device is implanted.

In one embodiment, the movement of the preferred nanomagnetic particles creates a sufficient amount of heat to kill cells by hyperthermia. This embodiment is described elsewhere in this specification, wherein nanomagnetic particles with specified Curie temperatures that preferentially kill cancer cells when heated are described.

In one preferred embodiment, the application of the external electromagnetic field 9090 activates the biologically active material in the coating of the medical device. A biologically active material that may be used in this embodiment may be a thermally sensitive substance that is coupled to nitric oxide, e.g., nitric oxide adducts, which prevent and/or treat adverse effects associated with use of a medical device in a patient, such as restenosis and damaged blood vessel surface. The nitric oxide is attached to a carrier molecule and suspended in the polymer of the coating, but it is only biologically active after a bond breaks, thereby releasing the smaller nitric oxide molecule in the polymer and eluting into the surrounding tissue. Typical nitric oxide adducts include, e.g., nitroglycerin, sodium nitroprusside, S-nitroso-proteins, S-nitroso-thiols, long carbon-chain lipophilic S-nitrosothiols, S-nitrosodithiols, iron-nitrosyl compounds, thionitrates, thionitrites, sydnonimines, furoxans, organic nitrates, and nitrosated amino acids, preferably mono- or poly-nitrosylated proteins, particularly polynitrosated albumin or polymers or aggregates thereof. The albumin is preferably human or bovine, including humanized bovine serum albumin. Such nitric oxide adducts are disclosed in U.S. Pat. No. 6,087,479 to Stamler et al., the entire disclosure of which is incorporated herein by reference into this specification.

In one embodiment, the application of the electromagnetic field 1090 effects a chemical change in the polymer coating, thereby allowing for faster release of the biologically active material from the coating.

Paragraphs 32-35 of published United States patent application 2004/0030379 are applicable to the devices of the instant invention. They are presented herein in their entireties.

“B. Drug Release Modulation Employing a Mechanical Vibrational Energy Source”

“Another embodiment of the present invention is a system for delivering a biologically active material to a body of a patient that comprises a mechanical vibrational energy source and an insertable medical device comprising a coating containing the biologically active material. The coating can optionally contain magnetic particles. After the device is implanted in a patient, the biologically active material can be delivered to the patient on-demand or when the material is needed by the patient. To deliver the biologically active material, the patient is exposed to an extracorporal or external mechanical vibrational energy source. The mechanical vibrational energy source includes various sources which cause vibration such as sonic or ultrasonic energy. Exposure to such energy source causes disruption in the coating that allows for the biologically active material to be released from the coating and delivered to body tissue.”

“Moreover, in certain embodiments, the biologically active material contained in the coating of the medical device is in a modified form. The modified biologically active material has a chemical moiety bound to the biologically active material. The chemical bond between the moiety and the biologically active material is broken by the mechanical vibrational energy. Since the biologically active material is generally smaller than the modified biologically active material, it is more easily released from the coating. Examples of such modified biologically active materials include the nitric oxide adducts described above.”

“In another embodiment, the coating comprises at least a coating layer containing a polymeric material whose structural properties are changed by mechanical vibrational energy. Such change facilitates release of the biologically active material which is contained in the same coating layer or another coating layer.”

Paragraphs 36, 37, 38, 39, 40, and 41 of published United States patent application 2004/0030379 are also applicable to the medical devices of this invention. They are presented below in their entireties.

“C. Materials Suitable for the Invention 1. Suitable Medical Devices”

“The medical devices of the present invention are insertable into the body of a patient. Namely, at least a portion of such medical devices may be temporarily inserted into or semi-permanently or permanently implanted in the body of a patient. Preferably, the medical devices of the present invention comprise a tubular portion which is insertable into the body of a patient. The tubular portion of the medical device need not to be completely cylindrical. For instance, the cross-section of the tubular portion can be any shape, such as rectangle, a triangle, etc., not just a circle.”

“The medical devices suitable for the present invention include, but are not limited to, stents, surgical staples, catheters, such as central venous catheters and arterial catheters, guidewires, balloons, filters (e.g., vena cava filters), cannulas, cardiac pacemaker leads or lead tips, cardiac defibrillator leads or lead tips, implantable vascular access ports, stent grafts, vascular grafts or other grafts, interluminal paving system, intra-aortic balloon pumps, heart valves, cardiovascular sutures, total artificial hearts and ventricular assist pumps.”

“Medical devices which are particularly suitable for the present invention include any kind of stent for medical purposes, which are known to the skilled artisan. Suitable stents include, for example, vascular stents such as self-expanding stents and balloon expandable stents. Examples of self-expanding stents useful in the present invention are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 4,733,665 issued to Palmaz, U.S. Pat. No. 4,800,882 issued to Gianturco, U.S. Pat. No. 4,886,062 issued to Wiktor and U.S. Pat. No. 5,449,373 issued to Pinchasik et al. A bifurcated stent is also included among the medical devices suitable for the present invention.”

“The medical devices suitable for the present invention may be fabricated from polymeric and/or metallic materials. Examples of such polymeric materials include polyurethane and its copolymers, silicone and its copolymers, ethylene vinyl-acetate, poly(ethylene terephthalate), thermoplastic elastomer, polyvinyl chloride, polyolephines, cellulosics, polyamides, polyesters, polysulfones, polytetrafluoroethylenes, acrylonitrile butadiene styrene copolymers, acrylics, polyactic acid, polyclycolic acid, polycaprolactone, polyacetal, poly(lactic acid), polylactic acid-polyethylene oxide copolymers, polycarbonate cellulose, collagen and chitins. Examples of suitable metallic materials include metals and alloys based on titanium (e.g., nitinol, nickel titanium alloys, thermo-memory alloy materials), stainless steel, platinum, tantalum, nickel-chrome, certain cobalt alloys including cobalt-chromium-nickel alloys (e.g., Elgiloy® and Phynox®) and gold/platinum alloy. Metallic materials also include clad composite filaments, such as those disclosed in WO 94/16646.”

Paragraphs 42-47 of published United States patent application 2004/0030379 describes the magnetic particles used in the device of such application. In applicants' preferred device, the magnetic particles of such device are replaced with certain nanomagnetic particles described elsewhere in this specification These nanomagnetic particles preferably have the properties described below.

The nanomagnetic particles are usually in to form of a coating a nanomagnetic material comprised of such particles. An assembly comprised of a device, wherein said device comprises a substrate and, disposed over such substrate, nanomagnetic material and magnetoresistive material, wherein the nanomagnetic material has a saturation magnetization of from about 2 to about 3000 electromagnetic units per cubic centimeter. The nanomagnetic particles generally have an average particle size of less than about 100 nanometers, wherein the average coherence length between adjacent nanomagnetic particles is less than 100 nanometers.

In one embodiment, the nanomagnetic material has an average particle size of less than about 20 nanometers and a phase transition temperature of less than about 200 degrees Celsius.

In one embodiment, the average particle size of such nanomagnetic particles is less than about 15 nanometers. In another embodiment, the nanomagnetic material has a saturation magnetization of at least 2,000 electromagnetic units per cubic centimeter.

In yet another embodiment, the nanomagnetic material has a saturation magnetization of at least 2,500 electromagnetic units per cubic centimeter.

In yet another embodiment, the nanomagnetic, the particles of nanomagnetic material have a squareness of from about 0.05 to about 1.0.

In yet another embodiment, the nanomagnetic, the particles of nanomagnetic material are at least triatomic, being comprised of a first distinct atom, a second distinct atom, and a third distinct atom. In one aspect of this embodiment, the first distinct atom is an atom selected from the group consisting of atoms of actinium, americium, berkelium, californium, cerium, chromium, cobalt, curium, dysprosium, einsteinium, erbium, europium, fermium, gadolinium, holmium, iron, lanthanum, lawrencium, lutetium, manganese, mendelevium, nickel, neodymium, neptunium, nobelium, plutonium, praseodymium, promethium, protactinium, samarium, terbium, thorium, thulium, uranium, and ytterbium. In another aspect of this embodiment, the distinct atom is a cobalt atom.

In yet another embodiment, the particles of nanomagnetic material are comprised of atoms of cobalt and atoms of iron.

In yet another embodiment, such first distinct atom is a radioactive cobalt atom.

In yet another embodiment, the particles of nanomagnetic material are comprised of a said first distinct atom, said second distinct atom, said third distinct atom, and a fourth distinct atom. In one aspect of this embodiment, the particles of nanomagnetic material are comprised of a fifth distinct atom.

In yet another embodiment, such particles of nanomagnetic material have a squareness of from about 0.1 to about 0.9. In one aspect of this embodiment, such particles of nanomagnetic material have a squareness is from about 0.2 to about 0.8.

In yet another embodiment, the nanomagnetic particles have an average size of less of less than about 3 nanometers. In yet another embodiment, the nanomagnetic particles have an average size of less than about 15 nanometers. In yet another embodiment, the nanomagnetic particles have an average size is less than about 11 nanometers.

In yet another embodiment, the nanomagnetic particles have a phase transition temperature of less than 46 degrees Celsius. In yet another embodiment, the nanomagnetic particles have a phase transition temperature of less than about 50 degrees Celsius.

In yet another embodiment, the nanomagnetic material has a coercive force of from about 0.1 to about 10 Oersteds.

In yet another embodiment, the nanomagnetic particles have a relative magnetic permeability of from about 1.5 to about 2,000.

In yet another embodiment, the nanomagnetic particles have a saturation magnetization of at least 100 electromagnetic units per cubic centimeter. In one aspect of this embodiment, the particles of nanomagnetic material have a saturation magnetization of at least about 200 electromagnetic units (emu) per cubic centimeter. In yet another aspect of this embodiment, the particles of nanomagnetic material have a saturation magnetization of at least about 1,000 electromagnetic units per cubic centimeter.

In yet another embodiment, the nanomagnetic particles have a coercive force of from about 0.01 to about 5,000 Oersteds. In one aspect of this embodiment, such particles of nanomagnetic material have a coercive force of from about 0.01 to about 3,000 Oersteds.

In yet another embodiment, the nanomagnetic particles have a relative magnetic permeability of from about 1 to about 500,000. In one aspect of this embodiment, such particles have a relative magnetic permeability of from about 1.5 to about 260,000.

In yet another embodiment, the nanomagnetic particles have a mass density of at least about 0.001 grams per cubic centimeter. In one aspect of this embodiment, such particles of nanomagnetic material have a mass density of at least about 1 gram per cubic centimeter. In another aspect of this embodiment, such particles of nanomagnetic material have a mass density of at least about 3 grams per cubic centimeter. In yet another aspect of this embodiment, such particles of nanomagnetic material have a mass density of at least about 4 grams per cubic centimeter.

In yet another embodiment, the second distinct atom of such nanomagnetic particles has a relative magnetic permeability of about 1.0. In one aspect of this embodiment, such second distinct atom is an atom selected from the group consisting of aluminum, antimony, barium, beryllium, boron, bismuth, calcium, gallium, germanium, gold, indium, lead, magnesium, palladium, platinum, silicon, silver, strontium, tantalum, tin, titanium, tungsten, yttrium, zirconium, magnesium, and zinc.

In yet another embodiment, the nanomagnetic particles are comprised of a third distinct atom that is an atom selected from the group consisting of argon, bromine, carbon, chlorine, fluorine, helium, helium, hydrogen, iodine, krypton, oxygen, neon, nitrogen, phosphorus, sulfur, and xenon. In one aspect of this embodiment, the third distinct atom is nitrogen.

In yet another embodiment, the nanomagnetic particles are represented by the formula AxByCz, wherein A is said first distinct atom, B is said second distinct atom, C is said third distinct atom, and x+y+z is equal to 1. In one aspect of this embodiment, such nanomagnetic particles are comprised of atoms of oxygen. In another aspect of this embodiment, the nanomagnetic particles are comprised of atoms of iron which optionally me be radioactive. In another aspect of this embodiment, such nanomagnetic particles are comprised of atoms of cobalt which, optionally, may be radioactive.

In yet another embodiment, the particles of nanomagnetic material are present in the form of a coating with a thickness of from about 400 to about 2000 nanometers. In one aspect of this embodiment, the coating has a thickness of from about 600 to about 1200 nanometers. In another aspect of this embodiment, the coating has a morphological density of at least about 98 percent, preferably at least about 99 percent, and more preferably at least about 99.5 percent. In another aspect of this embodiment, such coating has an average surface roughness of less than about 100 nanometers, and preferably of less than about 10 nanometers. In another aspect of this embodiment, such coating is biocompatible. In another aspect of this embodiment, such coating is hydrophobic. In yet another aspect of this embodiment, such coating is hydrophilic.

Paragraphs 48, through 72 of published United States patent application 2004/0030379 describe biologically active material that may be used in the device of this invention. This paragraphs are presented below in their entireties.

“3. Biologically Active Material”

“The term ‘biologically active material’ encompasses therapeutic agents, such as drugs, and also genetic materials and biological materials. The genetic materials mean DNA or RNA, including, without limitation, of DNA/RNA encoding a useful protein stated below, anti-sense DNA/RNA, intended to be inserted into a human body including viral vectors and non-viral vectors. Examples of DNA suitable for the present invention include DNA encoding . . . anti-sense RNA . . . tRNA or rRNA to replace defective or deficient endogenous molecules . . . angiogenic factors including growth factors, such as acidic and basic fibroblast growth factors, vascular endothelial growth factor, epidermal growth factor, transforming growth factor α and β, platelet-derived endothelial growth factor, platelet derived growth factor, tumor necrosis factor α, hepatocyte growth factor and insulin like growth factor . . . cell cycle inhibitors including CD inhibitors . . . thymidine kinase (“TK”) and other agents useful for interfering with cell proliferation, and . . . the family of bone morphogenic proteins (“BMP's”) as explained below. Viral vectors include adenoviruses, gutted adenoviruses, adeno-associated virus, retroviruses, alpha virus (Semliki Forest, Sindbis, etc.), lentiviruses, herpes simplex virus, ex vivo modified cells (e.g., stem cells, fibroblasts, myoblasts, satellite cells, pericytes, cardiomyocytes, skeletal myocytes, macrophage), replication competent viruses (e.g., ONYX-015), and hybrid vectors. Non-viral vectors include artificial chromosomes and mini-chromosomes, plasmid DNA vectors (e.g., pCOR), cationic polymers (e.g., polyethyleneimine, polyethyleneimine (PEI)) graft copolymers (e.g., polyether-PEI and polyethylene oxide-PEI), neutral polymers PVP, SP1017 (SUPRATEK), lipids or lipoplexes, nanoparticles and microparticles with and without targeting sequences such as the protein transduction domain (PTD).”

“The biological materials include cells, yeasts, bacteria, proteins, peptides, cytokines and hormones. Examples for peptides and proteins include growth factors (FGF, FGF-1, FGF-2, VEGF, Endothelial Mitogenic Growth Factors, and epidermal growth factors, transforming growth factor α and β, platelet derived endothelial growth factor, platelet derived growth factor, tumor necrosis factor α, hepatocyte growth factor and insulin like growth factor), transcription factors, protein kinases, CD inhibitors, thymidine kinase, and bone morphogenic proteins (BMP's), such as BMP-2, BMP-3, BMP-4, BMP-5, BMP-6 (Vgr-1), BMP-7 (OP-1), BMP-8. BMP-9, BMP-10, BMP-11, BMP-12, BMP-13, BMP-14, BMP-15, and BMP-16. Currently preferred BMP's are BMP-2, BMP-3, BMP-4, BMP-5, BMP-6, BMP-7. Alternatively or in addition, molecules capable of inducing an upstream or downstream effect of a BMP can be provided. Such molecules include any of the “hedgehog” proteins, or the DNA's encoding them. These dimeric proteins can be provided as homodimers, heterodimers, or combinations thereof, alone or together with other molecules. Cells can be of human origin (autologous or allogeneic) or from an animal source (xenogeneic), genetically engineered, if desired, to deliver proteins of interest at the transplant site. The delivery media can be formulated as needed to maintain cell function and viability. Cells include whole bone marrow, bone marrow derived mono-nuclear cells, progenitor cells (e.g., endothelial progentitor cells) stem cells (e.g., mesenchymal, hematopoietic, neuronal), pluripotent stem cells, fibroblasts, macrophage, and satellite cells.” “Biologically active material also includes non-genetic therapeutic agents, such as: . . . anti-thrombogenic agents such as heparin, heparin derivatives, urokinase, and PPack (dextrophenylalanine proline arginine chloromethylketone); . . . anti-proliferative agents such as enoxaprin, angiopeptin, or monoclonal antibodies capable of blocking smooth muscle cell proliferation, hirudin, and acetylsalicylic acid, amlodipine and doxazosin; . . . anti-inflammatory agents such as glucocorticoids, betamethasone, dexamethasone, prednisolone, corticosterone, budesonide, estrogen, sulfasalazine, and mesalamine; . . . immunosuppressants such as sirolimus (RAPAMYCIN), tacrolimus, everolimus and dexamethasone, . . . antineoplastic/antiproliferative/anti-miotic agents such as paclitaxel, 5-fluorouracil, cisplatin, vinblastine, vincristine, epothilones, methotrexate, azathioprine, halofuginone, adriamycin, actinomycin and mutamycin; cladribine; endostatin, angiostatin and thymidine kinase inhibitors, and its analogs or derivatives; . . . anesthetic agents such as lidocaine, bupivacaine, and ropivacaine; . . . anti-coagulants such as D-Phe-Pro-Arg chloromethyl keton, an RGD peptide-containing compound, heparin, antithrombin compounds, platelet receptor antagonists, anti-thrombin antibodies, anti-platelet receptor antibodies, aspirin (aspirin is also classified as an analgesic, antipyretic and anti-inflammatory drug), dipyridamole, protamine, hirudin, prostaglandin inhibitors, platelet inhibitors and tick antiplatelet peptides; . . . vascular cell growth promotors such as growth factors, Vascular Endothelial Growth Factors (FEGF, all types including VEGF-2), growth factor receptors, transcriptional activators, and translational promotors; vascular cell growth inhibitors such as antiproliferative agents, growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; . . . cholesterol-lowering agents; vasodilating agents; and agents which interfere with endogenous vasoactive mechanisms; . . . anti-oxidants, such as probucol; . . . antibiotic agents, such as penicillin, cefoxitin, oxacillin, tobranycin . . . angiogenic substances, such as acidic and basic fibroblast growth factors, estrogen including estradiol (E2), estriol (E3) and 17-Beta Estradiol; and . . . drugs for heart failure, such as digoxin, beta-blockers, angiotensin-converting enzyme (ACE) inhibitors including captopril and enalopril.”

“Also, the biologically active materials of the present invention include trans-retinoic acid and nitric oxide adducts. A biologically active material may be encapsulated in micro-capsules by the known methods.”

Paragraphs 73 through 82 of published United States patent application 1004/0030379 describe coating compositions that may be used in the device of the instant invention; and they are reproduced in their entireties below.

“4. Coating Compositions . . . The coating compositions suitable for the present invention can be applied by any method to a surface of a medical device to form a coating. Examples of such methods are painting, spraying, dipping, rolling, electrostatic deposition and all modern chemical ways of immobilization of bio-molecules to surfaces.”

“The coating composition used in the present invention may be a solution or a suspension of a polymeric material and/or a biologically active material and/or magnetic particles in an aqueous or organic solvent suitable for the medical device which is known to the skilled artisan. A slurry, wherein the solid portion of the suspension is comparatively large, can also be used as a coating composition for the present invention. Such coating composition may be applied to a surface, and the solvent may be evaporated, and optionally heat or ultraviolet (UV) cured.”

“The solvents used to prepare coating compositions include ones which can dissolve the polymeric material into solution and do not alter or adversely impact the therapeutic properties of the biologically active material employed. For example, useful solvents for silicone include tetrahydrofuran (THF), chloroform, toluene, acetone, isooctane, 1,1,1-trichloroethane, dichloromethane, and mixture thereof.”

“A coating of a medical device of the present invention may consist of various combinations of coating layers. For example, the first layer disposed over the surface of the medical device can contain a polymeric material and a first biologically active material. The second coating layer, that is disposed over the first coating layer, contains magnetic particles and optionally a polymeric material. The second coating layer protects the biologically active material in the first coating layer from exposure during implantation and prior to delivery. Preferably, the second coating layer is substantially free of a biologically active material.”

“Another layer, i.e. sealing layer, which is free of magnetic particles, can be provided over the second coating layer. Further, there may be another coating layer containing a second biologically active material disposed over the second coating layer. The first and second biologically active materials may be identical or different. When the first and second biologically active material are identical, the concentration in each layer may be different. The layer containing the second biologically active material may be covered with yet another coating layer containing magnetic particles. The magnetic particles in two different layers may have an identical or a different average particle size and/or an identical or a different concentrations. The average particle size and concentration can be varied to obtain a desired release profile of the biologically active material. In addition, the skilled artisan can choose other combinations of those coating layers.”

“Alternatively, the coating of a medical device of the present invention may comprise a layer containing both a biologically active material and magnetic particles. For example, the first coating layer may contain the biologically active material and magnetic particles, and the second coating layer may contain magnetic particles and be substantially free of a biologically active material. In such embodiment, the average particle size of the magnetic particles in the first coating layer may be different than the average particle size of the magnetic particles in the second coating layer. In addition, the concentration of the magnetic particles in the first coating layer may be different than the concentration of the magnetic particles in the second coating layer. Also, the magnetic susceptibility of the magnetic particles in the first coating layer may be different than the magnetic susceptibility of the magnetic particles in the second coating layer.”

“The polymeric material should be a material that is biocompatible and avoids irritation to body tissue. Examples of the polymeric materials used in the coating composition of the present invention include, but not limited to, polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate, styrene-isobutylene copolymers and blends and copolymers thereof. Also, other examples of such polymers include polyurethane (BAYHDROL®, etc.) fibrin, collagen and derivatives thereof, polysaccharides such as celluloses, starches, dextrans, alginates and derivatives, hyaluronic acid, and squalene. Further examples of the polymeric materials used in the coating composition of the present invention include other polymers which can be used include ones that can be dissolved and cured or polymerized on the medical device or polymers having relatively low melting points that can be blended with biologically active materials. Additional suitable polymers include, thermoplastic elastomers in general, polyolefins, polyisobutylene, ethylene-alphaolefin copolymers, acrylic polymers and copolymers, vinyl halide polymers and copolymers such as polyvinyl chloride, polyvinyl ethers such as polyvinyl methyl ether, polyvinylidene halides such as polyvinylidene fluoride and polyvinylidene chloride, polyacrylonitrile, polyvinyl ketones, polyvinyl aromatics such as polystyrene, polyvinyl esters such as polyvinyl acetate, copolymers of vinyl monomers, copolymers of vinyl monomers and olefins such as ethylene-methyl methacrylate copolymers, acrylonitrile-styrene copolymers, ABS (acrylonitrile-butadiene-styrene) resins, ethylene-vinyl acetate copolymers, polyamides such as Nylon 66 and polycaprolactone, alkyd resins, polycarbonates, polyoxymethylenes, polyimides, epoxy resins, rayon-triacetate, cellulose, cellulose acetate, cellulose butyrate, cellulose acetate butyrate, cellophane, cellulose nitrate, cellulose propionate, cellulose ethers, carboxymethyl cellulose, collagens, chitins, polylactic acid, polyglycolic acid, polylactic acid-polyethylene oxide copolymers, EPDM (etylene-propylene-diene) rubbers, fluorosilicones, polyethylene glycol, polysaccharides, phospholipids, and combinations of the foregoing. Preferred is polyacrylic acid, available as HYDROPLUS® (Boston Scientific Corporation, Natick, Mass.), and described in U.S. Pat. No. 5,091,205, the disclosure of which is hereby incorporated herein by reference. In a most preferred embodiment of the invention, the polymer is a copolymer of polylactic acid and polycaprolactone.”

“More preferably for medical devices which undergo mechanical challenges, e.g. expansion and contraction, the polymeric materials should be selected from elastomeric polymers such as silicones (e.g. polysiloxanes and substituted polysiloxanes), polyurethanes, thermoplastic elastomers, ethylene vinyl acetate copolymers, polyolefin elastomers, and EPDM rubbers. Because of the elastic nature of these polymers, the coating composition adheres better to the surface of the medical device when the device is subjected to forces, stress or mechanical challenge.”

“The amount of the polymeric material present in the coatings can vary based on the application for the medical device. One skilled in the art is aware of how to determine the desired amount and type of polymeric material used in the coating. For example, the polymeric material in the first coating layer may be the same as or different than the polymeric material in the second coating layer. The thickness of the coating is not limited, but generally ranges from about 25 μm to about 0.5 mm. Preferably, the thickness is about 30 μm to 100 μm.”

Paragraphs 84 through 92 of published United States patent application 2004/0030379 describes certain energy sources which may be used in conjunction with the medical devices of this invention. These paragraphs are presented below in their entireties.

“5. Electromagnetic Sources . . . An external electromagnetic source or field may be applied to the patient having an implanted coated medical device using any method known to skilled artisan. In the method of the present invention, the electromagnetic field is oscillated. Examples of devices which can be used for applying an electromagnetic field include a magnetic resonance imaging (“MRI”) apparatus. Generally, the magnetic field strength suitable is within the range of about 0.50 to about 5 Tesla (Webber per square meter). The duration of the application may be determined based on various factors including the strength of the magnetic field, the magnetic substance contained in the magnetic particles, the size of the particles, the material and thickness of the coating, the location of the particles within the coating, and desired releasing rate of the biologically active material.”

“In an MRI system, an electromagnetic field is uniformly applied to an object under inspection. At the same time, a gradient magnetic field, superposing the electromagnetic field, is applied to the same. With the application of these electromagnetic fields, the object is applied with a selective excitation pulse of an electromagnetic wave with a resonance frequency which corresponds to the electromagnetic field of a specific atomic nucleus. As a result, a magnetic resonance (MR) is selectively excited. A signal generated is detected as an MR signal. See U.S. Pat. No. 4,115,730 to Mansfield, U.S. Pat. No. 4,297,637 to Crooks et al., and U.S. Pat. No. 4,845,430 to Nakagayashi. For the present invention, among the functions of the MRI apparatus, the function to create an electromagnetic field is useful for the present invention. The implanted medical device of the present can be located as usually done for MRI imaging, and then an electromagnetic field is created by the MRI apparatus to facilitate release of the biologically active material. The duration of the procedure depends on many factors, including the desired releasing rate and the location of the inserted medical device. One skilled in the art can determine the proper cycle of the electromagnetic field, proper intensity of the electromagnetic field, and time to be applied in each specific case based on experiments using an animal as a model.

“In addition, one skilled in the art can determine the excitation source frequency of the electromagnetic energy source. For example, the electromagnetic field can have an excitation source frequency in the range of about 1 Hertz to about 300 kilohertz. Also, the shape of the frequency can be of different types. For example, the frequency can be in the form of a square pulse, ramp, saw tooth, sine, triangle, or complex. Also, each form can have a varying duty cycle.”

“6. Mechanical Vibrational Energy Source . . . . The mechanical vibrational energy source includes various sources which cause vibration such as ultrasound energy. Examples of suitable ultrasound energy are disclosed in U.S. Pat. No. 6,001,069 to Tachibana et al. and U.S. Pat. No. 5,725,494 to Brisken, PCT publications WO00/16704, WO00/18468, WO00/00095, WO00/07508 and WO99/33391, which are all incorporated herein by reference. Strength and duration of the mechanical vibrational energy of the application may be determined based on various factors including the biologically active material contained in the coating, the thickness of the coating, structure of the coating and desired releasing rate of the biologically active material.”

“Various methods and devices may be used in connection with the present invention. For example, U.S. Pat. No. 5,895,356 discloses a probe for transurethrally applying focused ultrasound energy to produce hyper thermal and thermo therapeutic effect in diseased tissue. U.S. Pat. No. 5,873,828 discloses a device having an ultrasonic vibrator with either a microwave or radio frequency probe. U.S. Pat. No. 6,056,735 discloses an ultrasonic treating device having a probe connected to a ultrasonic transducer and a holding means to clamp a tissue. Any of those methods and devices can be adapted for use in the method of the present invention.”

“Ultrasound energy application can be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely to an outer surface of the patient body proximal to the inserted medical device.”

“The duration of the procedure depends on many factors, including the desired releasing rate and the location of the inserted medical device. The procedure may be performed in a surgical suite where the patient can be monitored by imaging equipment. Also, a plurality of probes can be used simultaneously. One skilled in the art can determine the proper cycle of the ultrasound, proper intensity of the ultrasound, and time to be applied in each specific case based on experiments using an animal as a model.”

“In addition, one skilled in the art can determine the excitation source frequency of the mechanical vibrational energy source. For example, the mechanical vibrational energy source can have an excitation source frequency in the range of about 1 Hertz to about 300 kilohertz. Also, the shape of the frequency can be of different types. For example, the frequency can be in the form of a square pulse, ramp, saw tooth, sine, triangle, or complex. Also, each form can have a varying duty cycle.”

Paragraphs 93 through 97 of published United States patent application 2004/0030379 describe processes for treating body tissue that may be used in conjunction with the medical device of this invention. These paragraphs are presented below in their entireties.”

“D. Treatment of Body Tissue With the Invention . . . . The present invention provides a method of treatment to reduce or prevent the degree of restenosis or hyperplasia after vascular intervention such as angioplasty, stenting, atherectomy and grafting. All forms of vascular intervention are contemplated by the invention, including, those for treating diseases of the cardiovascular and renal system. Such vascular intervention include, renal angioplasty, percutaneous coronary intervention (PCI), percutaneous transluminal coronary angioplasty (PTCA); carotid percutaneous transluminal angioplasty (PTA); coronary by-pass grafting, angioplasty with stent implantation, peripheral percutaneous transluminal intervention of the iliac, femoral or popliteal arteries, carotid and cranial vessels, surgical intervention using impregnated artificial grafts and the like. Furthermore, the system described in the present invention can be used for treating vessel walls, portal and hepatic veins, esophagus, intestine, ureters, urethra, intracerebrally, lumen, conduits, channels, canals, vessels, cavities, bile ducts, or any other duct or passageway in the human body, either in-born, built in or artificially made. It is understood that the present invention has application for both human and veterinary use.”

“The present invention also provides a method of treatment of diseases and disorders involving cell over proliferation, cell migration, and enlargement. Diseases and disorders involving cell over proliferation that can be treated or prevented include but are not limited to malignancies, premalignant conditions (e.g., hyperplasia, metaplasia, dysplasia), benign tumors, hyperproliferative disorders, benign dysproliferative disorders, etc. that may or may not result from medical intervention. For a review of such disorders, see Fishman et al., 1985, Medicine, 2d Ed., J. B. Lippincott Co., Philadelphia.”

“Whether a particular treatment of the invention is effective to treat restenosis or hyperplasia of a body lumen can be determined by any method known in the art, for example but not limited to, those methods described in this section. The safety and efficiency of the proposed method of treatment of a body lumen may be tested in the course of systematic medical and biological assays on animals, toxicological analyses for acute and systemic toxicity, histological studies and functional examinations, and clinical evaluation of patients having a variety of indications for restenosis or hyperplasia in a body lumen.”

“The efficacy of the method of the present invention may be tested in appropriate animal models, and in human clinical trials, by any method known in the art. For example, the animal or human subject may be evaluated for any indicator of restenosis or hyperplasia in a body lumen that the method of the present invention is intended to treat. The efficacy of the method of the present invention for treatment of restenosis or hyperplasia can be assessed by measuring the size of a body lumen in the animal model or human subject at suitable time intervals before, during, or after treatment. Any change or absence of change in the size of the body lumen can be identified and correlated with the effect of the treatment on the subject. The size of the body lumen can be determined by any method known in the art, for example, but not limited to, angiography, ultrasound, fluoroscopy, magnetic resonance imaging, optical coherence tumography and histology.”

A Medical Preparation for Treating Arthrosis, Arthritis, and Other Diseases

In one embodiment of this invention, a novel medical preparation comprised of applicants' nanomagnetic particles is provided. This preparation is similar to the preparation described in U.S. Pat. No. 6,669,623.

U.S. Pat. No. 6,669,623, the entire disclosure of which is hereby incorporated by reference into this specification, discloses and claims “1. A medical preparation including nanoscalar particles that generate heat when an alternating electromagnetic field is applied, said nanoscalar particles comprising: a core containing iron oxide and an inner shell with groups that are capable of forming cationic groups, wherein the iron oxide concentration is in the range from 0.01 to 50 mg/ml of synovial fluid at a power absorption in the range from 50 to 500 mW/mg of iron and heating to a temperature in the range from 42 to 50° C.; and pharmacologically active species bound to said inner shell selected from the group consisting of thermosensitizers and thermosensitive chemotherapeutics or isotopes thereof; wherein said preparation is used for treating arthrosis, arthritis and rheumatic joint diseases by directly injecting said nanoscalar particles into the synovial fluid, said nanoscalar particles being absorbed by said fluid and transported to the inflamed synovial membrane where they are activated after a predefined period of time by applying said alternating electromagnetic field.”

Applicants' medical preparation is similar to the preparation of U.S. Pat. No. 6,669,623 but differs therefrom in that, instead of an iron oxide core, applicants' preparation is comprised of the nanomagnetic material described elsewhere in this specification.

As is disclosed in column 2 of U.S. Pat. No. 6,669,623, “The invention is based on the concept of using a suspension of nanoscalar particles designed based on the description given in DE 197 26 282 for treating rheumatic joint diseases, said particles comprising, in a first embodiment, a core containing iron oxide, an inner shell that encompasses said core and comprises groups capable of forming cationic groups, and an outer shell made of species comprising neutral and/or anionic groups, and radionuclides and cytotoxic substances bound to said inner shell. These nanoscalar particles may also be one-shelled, i.e. consist just of the core and the inner shell, designed as described above . . . . It has been found that despite the fact that phagocytic activity in the synovial fluid decreases as the patients' age increases, intracellular adsorption of the particles according to the invention in macrophages is increased even in pathologically changed macrophage titers in the joint cavity, and that the inflammatory process is controlled as said particles adhere to actively proliferating cells of the synovia. Due to these effects and the heat generated when applying an alternating electromagnetic field, the radionuclides show increased efficacy as compared to radiosynoviorthesis. Last but not least, success of treatment is increased beyond the additive effect of each component due to binding substances that have a cytotoxic effect when exposed to heat to the particles, as this efficiently combines radiotherapy, thermotherapy, and chemotherapy.”

As is disclosed at columns 2-3 of U.S. Pat. No. 6,669,623, “According to an embodiment that utilizes the invention, a suspension of nanoscalar particles formed by an iron oxide core and two shells, with doxorubicin as a heat-sensitive cytotoxic material and beta emitting radionuclides bound to said particles, is directly injected into the joint cavity to be treated. Depending on phagocytic activity in the synovia, the suspension will stay there without generating heat for a period of time that is determined before the therapy begins. This period can be from 1 hour to 72 hours. In this period, the two-shelled nanoparticles according to the invention are absorbed by the synovial fluid and flow into the inflamed synovial membrane. The therapist then ascertains using magnetic resonance tomography whether the nanoparticles are really deposited in the synovial membrane, the adjacent lymph nodes, and in the healthy tissue. If required, an extravasation to adjacent areas may be performed but this should not be necessary due to the high rate of phagocytosis . . . . Subsequently, the area is exposed to an alternating electromagnetic field with an excitation frequency in the range from 1 kHz and 100 MHz. Its actual value depends on the location of the diseased joint. While hands and arms are treated at higher frequencies, 500 kHz will be sufficient for back pain, the lower joints and the thigh joints. The alternating electromagnetic field brings out the localized heat; at the same time, the radionuclide and the cytotoxic substances (here: doxorubicin) are activated, and success of treatment beyond the added effects of its components is achieved due to the trimodal combinatorial effect of therapies and the differential endocytosis and high rate of phagocytosis of the nano-particles. This means that the synovial membrane shows increased and sustained sclerosing with this treatment as compared to other medical preparations and methods of treating rheumatic diseases . . . . The heat that can be generated by the alternating electromagnetic field applied to the nanoparticles, or, in other words, the duration of applying the alternating electromagnetic field to obtain a specific equilibrium temperature is calculated in advance based on the iron oxide concentration that is typically in the range from 0.01 to 50 mg/ml of synovial fluid and power absorption that is typically in the range from 50 to 500 mW/mg of iron. Then the field strength is reduced to keep the temperature on a predefined level of, for example, 45° C. However, there is a considerable temperature drop from the synovial layer treated to adjacent cartilage and bone tissue so that the cartilage layer and the bone will not be damaged by this heat treatment. The temperature in the cartilage layer is slightly increased as compared to normal physiological conditions (38° C. to 40° C.). The resulting stimulation of osteoblasts improves the reconstitution of degeneratively modified bone borders and cartilage. Repeated applications of the alternating electromagnetic field not only counteract recurring inflammation after the decline of radioactivity but—at an equilibrium temperature in the range from 38 to 40° C.—are also used to stimulate osteoblast division. When applying static magnetic field gradients, the particles can be concentrated in the treated joint (‘magnetic targeting’).” The iron-oxide core of the particles of this U.S. Pat. No. 6,669,223 may advantageously be replaced with the nanomagnetic material core of the present invention.

By way of further illustration, one may replace the iron-oxide containing core of the nanoparticles of published United States patent application US2003/0180370 with the nanomagnetic material of this invention. The entire disclosure of this published United States patent application is hereby incorporated by reference into this specification.

Claim 1 of published United States patent application 2003/0180370 describes “1. Nanoscale particles having an iron oxide-containing core and at least two shells surrounding said core, the (innermost) shell adjacent to the core being a coat that features groups capable of forming cationic groups and that is degraded by the human or animal body tissue at such a low rate that an association of the core surrounded by said coat with the surfaces of cells and the incorporation of said core into the inside of cells, respectively is possible, and the outer shell(s) being constituted by species having neutral and/or anionic groups which, from without, make the nanoscale particles appear neutral or negatively charged and which is (are) degraded by the human or animal body tissue to expose the underlying shell(s) at a rate which is higher than that for the innermost shell but still low enough to ensure a sufficient distribution of said nanoscale particles within a body tissue which has been punctually infiltrated therewith.” The particles of this published application comprise an iron-oxide-containing core with at least two shells (coats).

As is disclosed in paragraphs 0005 and 0006 of published United States patent application 2003/018370,” . . . such particles can be obtained by providing a (preferably superparamagnetic) iron oxide-containing core with at least two shells (coats), the shell adjacent to the core having many positively charged functional groups which permits an easy incorporation of the thus encased iron oxide-containing cores into the inside of the tumor cells, said inner shell additionally being degraded by the (tumor) tissue at such a low rate that the cores encased by said shell have sufficient time to adhere to the cell surface (e.g. through electrostatic interactions between said positively charged groups and negatively charged groups on the cell surface) and to subsequently be incorporated into the inside of the cell. In contrast thereto, the outer shell(s) is (are) constituted by species which shield (mask) or compensate, respectively, or even overcompensate the underlying positively charged groups of the inner shell (e.g. by negatively charged functional groups) so that, from without, the nanoscale particle having said outer shell(s) appears to have an overall neutral or negative charge. Furthermore the outer shell(s) is (are) degraded by the body tissue at a (substantially) higher rate than the innermost shell, said rate being however still low enough to give the particles sufficient time to distribute themselves within the tissue if they are injected punctually into the tissue (e.g. in the form of a magnetic fluid). In the course of the degradation of said outer shell(s) the shell adjacent to the core is exposed gradually. As a result thereof, due the outer shell(s) (and their electroneutrality or negative charge as seen from the exterior) the coated cores initially become well distributed within the tissue and upon their distribution they also will be readily imported into the inside of the tumor cells (and first bound to the surfaces thereof, respectively), due to the innermost shell that has been exposed by the biological degradation of the outer shell(s) . . . . Thus the present invention relates to nanoscale particles having an iron oxide-containing core (which is ferro-, ferri- or, preferably, superparamagnetic) and at least two shells surrounding said core, the (innermost) shell adjacent to the core being a coat that features groups capable of forming cationic groups and that is degraded by the human or animal body tissue at such a low rate that an association of the core surrounded by said coat with the surfaces of cells and the incorporation of said core into the inside of cells, respectively is possible, and the outer shell(s) being constituted by species having neutral and/or anionic groups which, from without, make the nanoscale particles appear neutral or negatively charged and which is (are) degraded by the human or animal body tissue to expose the underlying shell(s) at a rate which is higher than that for the innermost shell but still low enough to ensure a sufficient distribution of said nanoscale particles within a body tissue which has been punctually infiltrated therewith.”

Paragraph 0007 of published United States patent application US2003/0180370 indicates that the core of the particles of this patent application “ . . . consists of pure iron oxide . . . . ” Applicants advantageously substitute their nanomagnetic material of this invention for such “ . . . pure iron oxide . . . . ”

The shells of published United States patent application US2003/0180370 are discussed in paragraphs 0013 through 0016 of such patent application. As is disclosed in these paragraphs, “According to the present invention one or more (preferably one) outer shells are provided on the described innermost shell . . . the outer shell serves to achieve a good distribution within the tumor tissue of the iron oxide-containing cores having said inner shell, said outer shell being required to be biologically degradable (i.e., by the tissue) after having served its purpose to expose the underlying innermost shell, which permits a smooth incorporation into the inside of the cells and an association with the surfaces of the cells, respectively. The outer shell is constituted by species having no positively charged functional groups, but on the contrary having preferably negatively charged functional groups so that, from without, said nanoscale particles appear to have an overall neutral charge (either by virtue of a shielding (masking) of the positive charges inside thereof and/or neutralization thereof by negative charges as may, for example, be provided by carboxylic groups) or even a negative charge (for example due to an excess of negatively charged groups). According to the present invention for said purpose there may be employed, for example, readily (rapidly) biologically degradable polymers featuring groups suitable for coupling to the underlying shell (particularly innermost shell), e.g., (co)polymers based on α-hydroxycarboxylic acids (such as, e.g., polylactic acid, polyglycolic acid and copolymers of said acids) or polyacids (e.g., sebacic acid). The use of optionally modified, naturally occurring substances, particularly biopolymers, is particularly preferred for said purpose. Among the biopolymers the carbohydrates (sugars) and particularly the dextrans may, for example, be cited. In order to generate negatively charged groups in said neutral molecules one may employ, for example, weak oxidants that convert part of the hydroxyl or aldehyde functionalities into (negatively charged) carboxylic groups).”

Published United States patent application 2003/0180370 also discloses that: “ . . . in the synthesis of the outer coat one is not limited to carbohydrates or the other species recited above but that on the contrary any other naturally occurring or synthetic substances may be employed as well as long as they satisfy the requirements as to biological degradability (e.g. enzymatically) and charge or masking of charge, respectively . . . The outer layer may be coupled to the inner layer (or an underlying layer, respectively) in a manner known to the person skilled in the art. The coupling may, for example, be of the electrostatic, covalent or coordination type. In the case of covalent interactions there may, for example, be employed the conventional bond-forming reactions of organic chemistry, such as, e.g., ester formation, amide formation and imine formation. It is, for example, possible to react a part of or all of the amino groups of the innermost shell with carboxylic groups or aldehyde groups of corresponding species employed for the synthesis of the outer shell(s), whereby said amino groups are consumed (masked) with formation of (poly-)amides or imines. The biological degradation of the outer shell(s) may then be effected by (e.g., enzymatic) cleavage of said bonds, whereby at the same time said amino groups are regenerated.”

The particles of published United States patent application 2003/0180370 (and the related particles of the instant invention) may be used to deliver therapeutic agents to the inside of cells in the manner disclosed in paragraphs 0017 et seq. of published United States patent application 2003/0180370. As is disclosed in such published patent application, “Although the essential elements of the nanoscale particles according to the present invention are (i) the iron oxide-containing core, (ii) the inner shell which in its exposed state is positively charged and which is degradable at a lower rate, and (iii) the outer shell which is biologically degradable at a higher rate and which, from without, makes the nanoscale particles appear to have an overall neutral or negative charge, the particles according to the invention still may comprise other, additional components. In this context there may particularly be cited substances which by means of the particles of the present invention are to be imported into the inside of cells (preferably tumor cells) to enhance the effect of the cores excited by an alternating magnetic field therein or to fulfill a function independent thereof. Such substances are coupled to the -inner shell preferably via covalent bonds or electrostatic interactions (preferably prior to the synthesis of the outer shell(s)). This can be effected according to the same mechanisms as in the case of attaching the outer shell to the inner shell. Thus, for example in the case of using aminosilanes as the compounds constituting the inner shell, part of the amino groups present could be employed for attaching such compounds. However, in that case there still must remain a sufficient number of amino groups (after the degradation of the outer shell) to ensure the smooth importation of the iron oxide-containing cores into the inside of the cells. Not more than 10% of the amino groups present should in general be consumed for the importation of other substances into the inside of the cells. However, alternatively or cumulatively it is also possible to employ silanes different from aminosilanes and having different functional groups for the synthesis of the inner shell, to subsequently utilize said different functional groups for the attachment of other substances and/or the outer shell to the inner shell. Examples of other functional groups are, e.g., unsaturated bonds or epoxy groups as they are provided by, for example, silanes having (meth)acrylic groups or epoxy groups.”

Published United States patent application 2003/0180370 also discloses that “According to the present invention it is particularly preferred to link to the inner shell substances which become completely effective only at slightly elevated temperatures as generated by the excitation of the iron oxide-containing cores of the particles according to the invention by an alternating magnetic field, such as, e.g., thermosensitive chemotherapeutic agents (cytostatic agents, thermosensitizers such as doxorubicin, proteins, etc.). If for example a thermosensitizer is coupled to the innermost shell (e.g. via amino groups) the corresponding thermosensitizer molecules become reactive only after the degradation of the outer coat (e.g. of dextran) upon generation of heat (by the alternating magnetic field).”

Such “thermosensitive chemotherapeutic agents” are also referred to in claim 18 of U.S. Pat. No. 6,541,039 (“ . . . at least one pharmacologically active species is selected from the group consisting of thermosensitizers and thermosensitive chemotherapeutic agents), and in claim 6 of U.S. Pat. No. 6,669,623 (“thermosensitive cytotoxic agents bound to said inner shell); the entire disclosure of each of these United States patent applications is hereby incorporated by reference into this specification.

These “thermosensitive cytotoxic agents” are also referred to in paragraph 18 of published United States patent application US 2003/0180370, wherein it is disclosed that: “According to the present invention it is particularly preferred to link to the inner shell substances which become completely effective only at slightly elevated temperatures as generated by the excitation of the iron oxide-containing cores of the particles according to the invention by an alternating magnetic field, such as, e.g., thermosensitive chemotherapeutic agents (cytostatic agents, thermosensitizers such as doxorubicin, proteins, etc.). If for example a thermosensitizer is coupled to the innermost shell (e.g. via amino groups) the corresponding thermosensitizer molecules become reactive only after the degradation of the outer coat (e.g. of dextran) upon generation of heat (by the alternating magnetic field).”

The activity of the compositions of published United States patent application US2003/0180370 (and of applicants' derivative compositions) is described in paragraphs 0019-0020 of published United States patent application 2003/0180370. As is disclosed in these paragraphs, “For achieving optimum results, e.g. in tumor therapy, the excitation frequency of the alternating magnetic field applicator must be tuned to the size of the nanoscale particles according to the present invention in order to achieve a maximum energy yield. Due to the good distribution of the particle suspension within the tumor tissue, spaces of only a few micrometers in length can be bridged in a so-called “bystander” effect known from gene therapy, on the one hand by the generation of heat and on the other hand through the effect of the thermosensitizer, especially if excited several times by the alternating field, with the result that eventually the entire tumor tissue becomes destroyed . . . . Particles leaving the tumor tissue are transported by capillaries and the lymphatic system into the blood stream, and from there into liver and spleen. In said organs the biogenous degradation of the particles down to the cores (usually iron oxide and iron ions, respectively) then takes place, which cores on the one hand become excreted and on the other hand also become resorbed and introduced into the body's iron pool. Thus, if there is a time interval of at least 0.5 to 2 hours between the intralesional application of magnetic fluid and the excitation by the alternating field the surrounding environment of the tumor itself has “purged” itself of the magnetic particles so that during excitation by the alternating field indeed only the lesion, but not the surrounding neighborhood will be heated.”

When, however, the particles in question are nano-sized (as is the case with applicants' nanomagnetic particles), they do not leave the tissue in which they have been applied. Thus, as is disclosed in paragraph 0021 of published United States patent application 2003/0180370, “ . . . nanoparticles do not leave the tissue into which they have been applied, but get caught within the interstices of the tissue. They will get transported away only via vessels that have been perforated in the course of the application. High molecular weight substances, on the other hand, leave the tissue already due to diffusion and tumor pressure or become deactivated by biodegradation. Said processes cannot take place with the nanoscale particles of the present invention since on the one hand they are already small enough to be able to penetrate interstices of the tissue (which is not possible with particles in the μm range, for example, liposomes) and on the other hand are larger than molecules and, therefore cannot leave the tissue through diffusion and capillary pressure. Moreover, in the absence of an alternating magnetic field, the nanoscale particles lack osmotic activity and hardly influence the tumor growth, which is absolutely necessary for an optimum distribution of the particles within the tumor tissue . . . . If an early loading of the primary tumor is effected the particles will be incorporated to a high extent by the tumor cells and will later also be transferred to the daughter cells at a probability of 50% via the parental cytoplasm. Thus, if also the more remote surroundings of the tumor and known sites of metastatic spread, respectively are subjected to an alternating magnetic field individual tumor cells far remote from the primary tumor will be affected by the treatment as well. Particularly the therapy of affected lymphatic nodes can thus be conducted more selectively than in the case of chemotherapy. Additional actions by gradients of a static magnetic field at sites of risk of a subsequent application of an alternating field may even increase the number of hits of loaded tumor cells.”

The composition of published United States patent application US 2003/0180370, and also of applicants' related composition, also effect an anti-mitotic activity because of “selective embolization.” Thus, as is disclosed in paragraphs 24-25 of such United States patent application, “Due to the two-stage interlesional application a selective accumulation is not necessary. Instead the exact localization of the lesion determined in the course of routine examination and the subsequently conducted infiltration, in stereotactic manner or by means of navigation systems (robotics), of the magnetic fluid into a target region of any small (or bigger) size are sufficient . . . The combination with a gradient of a static magnetic field permits a regioselective chemoembolization since not only the cyctostatic agent preferably present on the particles of the invention is activated by heat but also a reversible aggregation of the particles and, thus a selective embolization may be caused by the static field.”

It is known that, when cancer cells are treated with hyperthermia, the survival levels of cells treated in the absence of nutrients is greatly reduced over those heat treated with nutrients; see, e.g., an article by G. M. Hahn, “Metabolic aspects of the role of hyperthermia in mammalian cell inactivation and their possible relevance to cancer treatment,” Cancer Res. 34:3117-3123, November, 1974. In this Hahn article, it was disclosed that “The sensitivity of cells to hyperthermia (as well as their ability to repair heat-induced damage after 43 degrees) is strongly related to their nutritional history. Chinese hamster cells chronically deprived of serum (and probably other medium components) become extremely heat sensitive.

In one embodiment of the instant invention, applicants' “two-shell nanomagnetic compositions” are incorporated into tumor cells and, with the use of an external electromagnetic field, used to cause a regioselective embolization. Thereafter, when the tumor cells have been deprived of serum, the nanomagnetic materials permanently disposed within the cells are caused to heat up and kill the cells, which are now more sensitive to hyperthermia.

Other applications for applicants' compositions (and the related compositions of published United States patent application 2003/0180370) are discussed in paragraphs 0026 and 0027 of such patent application, wherein it is disclosed that: “In addition to tumor therapy, further applications of the nanoscale particles according to the present invention (optionally without the outer shell(s)) are the heat-induced lysis of clotted microcapillaries (thrombi) of any localization in areas which are not accessible by surgery and the successive dissolution of thrombi in coronary blood vessels. For example thrombolytic enzymes which show an up to ten-fold increase in activity under the action of heat or even become reactive only on heating, respectively may for said purpose be coupled to the inner shell of the particles according to the invention. Following intraarterial puncture of the vessel in the immediate vicinity of the clogging the particles will automatically be transported to the “point of congestion” (e.g., under MRT control). A fiberoptical temperature probe having a diameter of, e.g., 0.5 mm is introduced angiographically and the temperature is measured in the vicinity of the point of congestion while, again by external application of an alternating magnetic field, a microregional heating and activation of said proteolytic enzymes is caused. In the case of precise application of the magnetic fluid and of MRT control a determination of the temperature can even be dispensed with on principle since the energy absorption to be expected can already be estimated with relatively high accuracy on the basis of the amount of magnetic fluid applied and the known field strength and frequency. The field is reapplied in intervals of about 6 to 8 hours. In the intervals of no excitation the body has the opportunity to partly transport away cell debris until eventually, supported by the body itself, the clogging is removed. Due to the small size of the particles of the invention the migration of said particles through the ventricles of the heart and the blood vessels is uncritical. Eventually the particles again reach liver and spleen via RES.”

Published United States patent application US 2003/0180370 also discloses that: “Apart from classical hyperthermia at temperatures of up to 46/47° C. also a thermoablation can be conducted with the nanoscale particles of the present invention. According to the state of the art mainly interstitial laser systems that are in part also used in surgery are employed for thermoablative purposes. A big disadvantage of said method is the high invasivity of the microcatheter-guided fiberoptical laser provision and the hard to control expansion of the target volume. The nanoparticles according to the present invention can be used for such purposes in a less traumatic way: following MRT-aided accumulation of the particle suspension in the target region, at higher amplitudes of the alternating field also temperatures above 50° C. can homogeneously be generated. Temperature control may, for example, also be effected through an extremely thin fiberoptical probe having a diameter of less than 0.5 mm. The energy absorption as such is non-invasive.”

The compositions described in published United States patent application US 2003/0180370 may be used in the processes described by the claims of U.S. Pat. No. 6,541,039, the entire disclosure of which is hereby incorporated by reference into this specification.

Claim 1 of U.S. Pat. No. 6,541,039 describes: “1. A method of hyperthermic treatment of a region of the body selected from the group consisting of hyperthermic tumor therapy, heat-induced lysis of a thrombus, and thermoablation of a target region, comprising: (a) accumulating in the region of the body a magnetic fluid comprising nanoscale particles suspended in a fluid medium, each particle having an iron oxide-containing core and at least two shells surrounding said core, (1) the innermost shell adjacent to the core being a shell that: (a) is formed from polycondensable silanes comprising at least one aminosilane and comprises groups that are positively charged or positively chargeable, and (b) is degraded by human or animal body tissue at such a low rate that adhesion of the core surrounded by the innermost shell with the surface of a cell through said positively charged or positively chargeable groups of the innermost shell and incorporation of the core into the interior of the cell are possible, and (2) the outer shell or shells comprising at least one species that: (a) is a biologically degradable polymer selected from (co)polymers based on .alpha.-hydroxycarboxylic acids, polyols, polyacids, and carbohydrates optionally modified by carboxylic groups and comprises neutral and/or negatively charged groups so that the nanoscale particle has an overall neutral or negative charge from the outside of the particle, and (b) is degraded by human or animal body tissue to expose the underlying shell or shells at a rate which is higher than that for the innermost shell but is still low enough to ensure a sufficient distribution of a plurality of the nanoscale particles within a body tissue which has been infiltrated therewith; and (b) applying an alternating magnetic field to generate heat in the region by excitation of the iron oxide-containing cores of the particles, thereby causing the hyperthermic treatment”

Claims 2-15 of U.S. Pat. No. 6,541,039 are dependent upon claim 1. Claim 3 describes “3. The method of claim 1 that is a method of heat-induced lysis of a thrombus, comprising accumulating in the thrombus the magnetic fluid, and applying an alternating magnetic field to generate heat by excitation of the iron oxide-containing cores of the particles to cause heat-induced lysis of the thrombus.” Claim 4 describes “4. The method of claim 1 that is a method of thermoablation of a target region, comprising accumulating in the target region the magnetic fluid, and applying an alternating magnetic field to generate heat by excitation of the iron oxide-containing cores of the particles to cause thermoablation of the target region.” Claim 10 describes “10. The method of claim 1 where the innermost shell is derived from aminosilanes.” Claim 11 describes “11. The method of claim 1 where the at least one species comprising the outer shell or shells is selected from carbohydrates optionally modified by carboxylic groups.” Claim 12 describes “12. The method of claim 11 where the at least one species comprising the outer shell or shells is selected from dextrans optionally modified by carboxylic groups.” Claim 13 describes “13. The method of claim 12 where the at least one species comprising the outer shell or shells is selected from dextrans modified by carboxylic groups.” Claim 14 describes “4. The method of claim 1 where at least one pharmacologically active species is linked to the innermost shell.” Claim 15 describes “15. The method of claim 14 where the at least one pharmacologically active species is selected from the group consisting of thermosensitizers and thermosensitive chemotherapeutic agents

The other independent claim in U.S. Pat. No. 6,541,039 is claim 16, which describes “16. A method of tumor therapy by hyperthermia, comprising: (a) accumulating in the tumor a magnetic fluid comprising nanoscale particles suspended in a fluid medium, each particle having a superparamagnetic iron oxide-containing core having an average particle size of 3 to 30 nm comprising magnetite, maghemite, or stoichiometric intermediate forms thereof and at least two shells surrounding said core, (1) the innermost shell adjacent to the core being a shell that:(a) is formed from polycondensable aminosilanes and comprises groups that are positively charged or positively chargeable, and (b) is degraded by human or animal body tissue at such a low rate that adhesion of the core surrounded by the innermost shell with the surface of a cell through said positively charged or positively chargeable groups of the innermost shell and incorporation of the core into the interior of the cell are possible, and (2) the outer shell or shells being a shell or shells comprising at least one species that: (a) is a biologically degradable polymer selected from dextrans optionally modified by carboxylic groups and comprises neutral and/or negatively charged groups so that the nanoscale particle has an overall neutral or negative charge from the outside of the particle, and (b) is degraded by human or animal body tissue to expose the underlying shell or shells at a rate which is higher than that for the innermost shell but is still low enough to ensure a sufficient distribution of a plurality of the nanoscale particles within a body tissue which has been infiltrated therewith; and (b) applying an alternating magnetic field to generate heat in the tumor by excitation of the iron oxide-contain cores of the particles, thereby causing hyperthermia of the tumor.”

Claims 17 and 18 of U.S. Pat. No. 6,541,039 are dependent upon claim 16. Claim 17 describes “17. The method of claim 16 where at least one pharmacologically active species is linked to the innermost shell.” Claim 18 describes “18. The method of claim 17 where the at least one pharmacologically active species is selected from the group consisting of thermosensitizers and thermosensitive chemotherapeutic agents.”

As will be apparent to those skilled in the art, all of the processes described in U.S. Pat. No. 6,541,039 may be conducted with a composition that contains applicants' nanomagnetic material rather than the iron oxide material of the Lesniak et al. patent.

The nanosize iron-containing oxide particles used in the process of U.S. Pat. No. 6,541,039 may be prepared by conventional means such as, e.g., the process described in U.S. Pat. No. 6,183,658. This latter patent claims “1. A process for producing an-agglomerate-free suspension of stably coated nanosize iron-containing oxide particles, comprising the following steps in the order indicated: (1) preparing an aqueous suspension of nanosize iron-containing oxide particles which are partly or completely present in the form of agglomerates; (2) adding (i) a trialkoxysilane which has a hydrocarbon group which is directly bound to Si and to which is bound at least one group selected from amino, carboxyl, epoxy, mercapto, cyano, hydroxy, acrylic, and methacrylic, and (ii) a water-miscible polar organic solvent whose boiling point is at least 10° C. above that of water; (3) treating the resulting suspension with ultrasound until at least 70% of the particles present have a size within the range from 20% below to 20% above the mean particle diameter; (4) removing the water by distillation under the action of ultrasound; and (5) removing the agglomerates which have not been broken up.”

An Anticancer Agent Releasing Microcapsule

In one embodiment of the invention, a microcapsule for hyperthermia treatment is made by coating nanomagnetic particles with cis-platinum diamine dichloride (CDDP), and then covering the layer of anticancer agent with a mixture of hydroxylpropyl cellulose and mannitol. This microcapsule is similar to the microcapsule described in an article by Tomoya Sato et al., “The Development of Anticancer Agent Releasing Microcapsule Made of Ferromagnetic Amorphous Flakes for Intratissue Hyperthermia,” IEEE Transactions on Magnetics, Volume 29, Number 6, November, 1993.

The “core” of the Sato et al. microcapsule was ferromagnetic amorphous flakes with an average size of about 50 microns and a Curie temperature of about 45 degrees Centigrade. In one embodiment of the instant invention, the Sato et al. ferromagnetic material is replaced with the nanomagnetic material of this invention.

The core of the Sato et al. microcapsule was then coated with an anticancer agent, such as Cis-platinum diamine dichloride (CDDP). Thereafter, the coated cores were then coated with a material that did not react with the anticancer agent. As is disclosed on page 3329 of the article, “A wide variety of anticancer agents and macromolecular compounds can be used for coating of amorphous flakes, but the absence of reaction between the anticancer agent and the macromolecular compound as the base is the primary condition for their selection. In this study, CDDP was used as the anticancer agent, and a mixture of hydroxypropyl cellulose (HPC-H) and mannitol, which do not react with CDDP, was used as the macromolecular coating material.”

The coating used in the Sato et al. microcapsule was designed to dissolve in bodily fluid when it was heated to a temperature greater than about 40 degrees Centigrade. Thus, as is disclosed at page 3329 of the Sato et al. article, “We noted the characteristics of HPC-H that it becomes a viscous gel in water at 38 degrees C. or below but loses its viscosity above 40 degrees C. Because of this property, we expected that it would remain a viscous gel and slowly release CDDP at body temperatures of 36 to 37 degrees C. but would lose its viscosity and release more CDDP when it is heated to 40 degrees C. or above, and we attempted to regulate the release of CDDP by hyperthermia.”

Mixtures of Nanomagnetic Material and a Clay Mineral

In one embodiment of this invention, a mixture is provided of the nanomagnetic material of this invention (described elsewhere in this specification) and a second material selected from the group consisting of a clay mineral material and an organic material. The nanomagnetic material is present in this composition at a concentration of from about 1 to about 99 percent, by weight of the nanomagnetic material and the second material. In one embodiment, nanomagnetic material is present at a concentration of from about 5 to about 95 weight percent, by total weight of the two materials. In another embodiment, the nanomagnetic material is present at a concentration of from about 10 to about 90 percent. In yet another embodiment, at least 50 weight percent of the mixture of the two materials is nanomagnetic material.

In one aspect of this embodiment, the second material is a mineral. As is known to those skilled in the art, a mineral is a native, nonorganic or fossilized organic substance having a definite chemical composition and formed by inorganic reactions. See, e.g., page 431 of Julius Grant's “Hackh's Chemical Dictionary,” Fourth Edition (McGraw-Hill Book Company, New York, N.Y., 1972).

In one embodiment, the mineral used is a clay mineral, i.e., a mineral found in clay. These materials are well known in the patent literature. Reference may be had, e.g., to U.S. Pat. Nos. 3,873,585; 3,915,731; 4,405,371(clay mineral color developer); U.S. Pat. Nos. 4,600,437; 4,798,630; 4,810,737; 4,839,221(gasket containing PTFE and clay mineral); U.S. Pat. No. 4,929,580(process for treating clay minerals); U.S. Pat. Nos. 4,990,544; 5,908,500(activated clay composition); U.S. Pat. Nos. 5,322,879; 5,936,023(clay mineral/rubber composition); U.S. Pat. No. 5,973,053(composite clay material); U.S. Pat. Nos. 6,103,817; 6,121,361(clay rubber); U.S. Pat. No. 6,416,573(pigment); U.S. Pat. No. 6,562,891(modified clay mineral); U.S. Pat. No. 6,737,166; and the like. The entire disclosure of each of these United States patents is hereby incorporated by reference into this specification.

In one embodiment, the clay mineral used is a fibrous clay mineral, as that term is described in U.S. Pat. No. 4,364,857, the entire disclosure of which is hereby incorporated by reference into this specification. This patent claims (in claim 1) “1. A porous composition of matter comprising codispersed rods of a first fibrous clay and a second fibrous clay, said first fibrous clay having predominantly rods with the length range of 0.5-2 microns and a diameter range of 0.04-0.2 microns and said second fibrous clay having predominantly rods with a length range of 1-5 microns and a diameter range of 50-100 Angstroms.” These “fibrous clays,” and their preparation and processing, are described at columns 2-4 of U.S. Pat. No. 4,365,857, wherein it is disclosed that “The clay halloysite is readily available from natural deposits. It can also be synthesized, if desired. In its natural state, halloysite often comprises bundles of tubular rods or needles consolidated or bound together in weakly parallel orientation. These rods have a length range of about 0.5-2 microns and a diameter range of about 0.04-0.2 microns. Halloysite rods have a central co-axial hole approximately 100-300 Angstroms in diameter forming a scroll-like structure.”

U.S. Pat. No. 4,364,857 also discloses that “It has been found that halloysite can make a suitable catalyst for use in demetalizing and hydroprocessing asphaltenes. The halloysite is processed to break up the bundles of rods so that each rod is freely movable with respect to the other rod. When substantially all the rods are freely movable with respect to all the other rods, the rods are defined herein as “dispersed”. When the dispersed rod clay is dried and calcined, the random orientation of the rods provides pores of an appropriate size for hydroprocessing and hydrodemetalizing asphaltene fractions.”

U.S. Pat. No. 4,364,857 also discloses that “When halloysite rods or other rods of similar dimensions are agitated in a fluid such as water to disperse the rods, the dispersion can be shaped, dried and calcined to provide a porous body having a large pore volume present as 200-700 Angstroms diameter pores. When the shaping is by extrusion, however, it has been found that mixtures of dispersed clay rods of the halloysite type, do not extrude well. The rods on the surface of the extruded bodies tend to realign, destroying the desirable pore structure at the surface of the catalyst. This is defined herein as a “skin effect”. It has been discovered, however, that if a second fibrous clay with longer, narrower and presumably more flexible, fibers is codispersed with the halloysite-type clay, the resulting composition is easily extrudible, and there is no significant skin effect. “Codispersed” is defined herein as having rod- or tube-like clay particles of at least two distinct types substantially randomly oriented to one another.”

U.S. Pat. No. 4,364,857 also discloses that “The second fibrous clay should have long slender fibers typically about 1-5 microns in length with a diameter range of about 50-100 Angstroms. Clays for use as the second component include attapulgite, crysotile, immogolite, palygorskite, sepiolite and the like.”

U.S. Pat. No. 4,364,857 also discloses that “The composition of the present invention is prepared by vigorously agitating a mix comprising the first fibrous clay and a second fibrous clay in a liquid dispersing medium. Water is a satisfactory dispersing agent. It is preferred that the slurry contain no more than 25 weight percent of total solids. The vigorous agitation can be accomplished in any suitable manner. In the laboratory, excellent codispersions are achieved with a Waring blender. It is observed that the slurry thickens with agitation, apparently due to the rods dispersing. Agitation is continued until the slurry maintains a constant thickness. Excess water is removed by slow evaporation at 110° C. until a workable plastic mass is formed. The mass can be shaped, using well known techniques such as extrusion, pelletizing, or spheredizing to form catalyst bodies. The shaped particles are then calcined at 500° C.”

U.S. Pat. No. 4,364,857 also discloses that “To increase the crush strength of the catalyst support, a refractory inorganic binder oxide such as alumina, silica, boria, titania, magnesia, or the like can be added to the composition. Preferably, the finished catalyst support contains less than about 15 weight percent binder oxide, based on the total weight of clay plus binder oxide. An especially preferable inorganic oxide range is about 3-7 percent by weight of the support.”

U.S. Pat. No. 4,364,857 also discloses that “If an inorganic oxide component is to be present into the composition of the present invention, codispersal of the rods of the fibrous clay is preferably carried out in the presence of an aqueous hydrogel or the sol precursor of the inorganic oxide gel component. The preferred inorganic oxide is alumina. Mixture of two or more inorganic oxides are suitable for the present invention for example, silica and alumina.”

U.S. Pat. No. 4,364,857 also discloses that “A function of the inorganic oxide gel component is to act as a bonding agent for holding or bonding the clay rods in a rigid, three-dimensional matrix. The resulting rigid skeletal framework provides a catalyst body with high crush strength and attrition resistance.”

U.S. Pat. No. 4,364,857 also discloses that “The catalyst may also include one or more catalytically active metals, such as transition metals. A first preferred group of catalytically active metals for use in catalysts of this invention, is the group of chromium, molybdenum, tungsten and vanadium. A second preferred group of catalytically active metals is the group of iron, nickel, and cobalt. Preferably, one or more of the metals of the first group is present in the catalyst at a total amount as metal of about 0.1-10 weight percent and one or more of the metals of the second group is present at a total amount as metal of from about 0.1-10 weight percent, based on the total catalyst weight. Especially preferred combinations include between 0.1 and 10 weight percent of at least one metal from both the first and second preferred groups, for example, molybdenum and cobalt, molybdenum and nickel, tungsten and nickel, and vanadium and nickel.”

U.S. Pat. No. 4,364,857 also discloses that “The metal component can be added to the catalyst composition at any stage of the catalyst preparation by any conventional metal addition step. For example, metals or metal compounds can be added to the slurry as solids or in solution, preferably before dispersion of the clay rods. Alternatively, an aqueous solution of metal can impregnate the dried or calcined bodies. The metals can be present in reduced form or as one or more metal compounds such as oxides or sulfides. One preferred method is impregnating the calcined catalyst bodies with a solution of phosphomolybdic acid and nickel nitrate.”

In one embodiment, the clay mineral used is a crystalline clay mineral, as that term is used in the claims of U.S. Pat. No. 5,624,544, the entire disclosure of which is hereby incorporated by reference into this specification. Claim 1 of this patent describes “1. A method for manufacturing ionized water comprising: a first step of dissolving crystalline clay minerals selected from the group consisting of montmorillonite and halloysite in water for electrolysis treatment, and a second step of further dissolving crystal clay minerals in alkaline ionized water and acidic ionized water obtained at the first step, and supplying respectively to the cathode side and anode side, and performing electrolysis treatment so as to produce strong alkaline and strong acidic ionized water maintaining a stable pH, respectively at the cathode side and anode side.”

The crystalline clay minerals of U.S. Pat. No. 5,624,544 are described in columns 3-4 of this patent, wherein it is disclosed that “By repeating dissolution of crystalline clay minerals and electrolysis plural times on the alkaline ionized water and acidic ionized water, the alkaline ionized water comes to have a further higher pH value and the acidic ionized water, a further lower pH value. As a result, finally, an alkaline ionized water at pH 12 or more, and an acidic ionized water at pH 3 or less are produced. Moreover, the obtained alkaline ionized water and acidic ionized water are hardly changed in the time course, and the initial pH value is maintained stably for a long period. The crystalline clay minerals are formed in a thin layer state by secondary growth by bonding of tetrahedron of silicic acid and octahedron of alumina. Structurally, crystalline clay minerals are classified into 2-1 type and 1--1 type.”

U.S. Pat. No. 5,624,544 also discloses that “The crystalline clay mineral of 2-1 type represented by montmorillonite is formed by 2:1 bonding of a tetrahedron layer of silicic acid and an octahedron layer of alumina, and a pair of tetrahedron layers of silicic acid are placed from both sides of the octahedron layer of alumina. The crystalline clay mineral of 2-1 type is higher in the content of silicic acid and lower in the content of alumina, as compared with the crystalline clay mineral of 1--1 type.”

U.S. Pat. No. 5,624,544 also discloses that “Among overlapped unit layers of crystalline clay mineral of 2-1 type such as montmorillonite, water molecules, Na ions, Ca ions and other cations are invading, and generally bonding between layers is weak, and a large amount of water molecules can be aspirated between the layers.”

U.S. Pat. No. 5,624,544 also discloses that “The crystalline clay mineral of 1--1 type is formed by 1:1 bonding of tetrahedron layer of silicic acid and octahedron layer of alumina, and kaolinite and halloysite belong to the crystalline clay mineral of 1--1 type. In kaolinite, the alumina plane of basic unit layer is bonded with silicic acid plane of other basic unit layer by hydrogen bond, and groups of 0.03 to 0.05 μm are formed. In halloysite, on the other hand, one water molecule layer is present between basic unit layers, and this unit is grouped into a proper size, and the shape is varied including hollow tube, sphere, and cabbage form.”

U.S. Pat. No. 5,624,544 also discloses that “In the tetrahedron layer of silicic acid of lamellar clay mineral generally recognized, usually, one silicon ion is surrounded by four oxygen atoms, and the coordination is stable, but in the process of formation of clay mineral, its silicon ion (valence of plus 4) may be sometimes replaced by an aluminum ion (valence of plus 3). At this time, the tetrahedron layer of silicic acid comes to have one unit of negative charge (1.6×10⁻¹⁹ coulombs). Similarly, the aluminum ion in the octahedron of alumina may be replaced by Mg ion or Fe ion, and this octahedron of alumina also possesses one unit of negative charge. The permanent electric charge generated in such clay mineral continues to exist regardless of the ambient conditions. In particular, the montmorillonite has this property very obviously, and its charging density is a negative charge of 10² units per ¹ cm³, and in spite of its very large charge density, its structure is stably chemically.”

U.S. Pat. No. 5,624,544 also discloses that “A pair of tetrahedrons of silicic acid or a pair of octahedrons of alumina share an oxygen atom, but at the terminal end (end face), silicon or aluminum is present only at one side, and the negative charge of oxygen is not satisfied. The clay mineral is very fine and large in specific surface area (for example, montmorillonite has a thickness of about 0.002 to 0.02 μm in the expanse of 0.1 μm class, and kaolinite has a length of 0.07 to 3.5 μm, width of 0.5 to 2.1 μm, and thickness of 0.03 to 0.05 μm), and even a trace diffuses sufficiently in water, and electric (electronic) effects are very large.”

U.S. Pat. No. 5,624,544 also discloses that “On the end face of the tetrahedron of silicic acid, a negative charge is exposed on the surface, and H+ ions are weakly taken in, and an electric neutrality is maintained. This bond is, however, very weak, and although it is stable when many H+ ions are present in the material water (ionized water) to be electrolyzed (acid and low in pH value), but when the pH value of the material water (ionized water) becomes large and the concentration of OH− ions is high, H+ ions pop out from the tetrahedron of silicic acid accordingly, and silicic acid is charged negatively. That is, when the pH of the material water (ionized water) is larger, it tends to charge negatively, and as the pH value is smaller, it approaches the neutrality.”

U.S. Pat. No. 5,624,544 also discloses that “By contrast, the octahedron of alumina is firmly bonded with OH− ions in the state of the positive charge of aluminum exposed on the surface, and as a result, electrically, it is minus and further attracts H+ ions to be charged positively. That is, through the intervening OH− ions, H+ is attracted. This reaction is progressed when the H+ concentration of material water becomes large (the pH value becomes lower), and it is likely to be charged positively when the pH value of the material water (ionized water) becomes lower.”

U.S. Pat. No. 5,624,544 also discloses that “Accordingly, on the end face of clay mineral, when the pH value of the water to be electrolyzed becomes higher, the negative charge (OH⁻) increases relatively, and when the pH becomes lower, the positive charge (H⁺) becomes dominant.”

In one preferred embodiment, the clay mineral is selected from the group consisting of smectite clay minerals (e.g., montmorillonite, saponite, hectolite, beidellite, stevensite, nontronite), vermiculite, halloysite or fluorine mica. Reference may be had, e.g., to U.S. Pat. No. 5,936,023, the entire disclosure of which is hereby incorporated by reference into this specification.

In one preferred embodiment, the clay mineral is halloysite, a hydrated aluminosilicate that contains alumina (Al₂O₃), silica (Si0₂), and water (H₂0). In one embodiment, the halloysite contains abut 3 moles of silica and 2 moles of water for each mole of Lumina, it has a molecular weight of 318.1, and it has a melting point above 1,500 degrees Celsius.

As is disclosed in U.S. Pat. No. 6,401,816, the entire disclosure of which is hereby incorporated by reference into this specification, “Several naturally occurring minerals will, under appropriate hydration conditions, form tubules and other microstructures suitable for use in the present invention. The most common of these is halloysite, an inorganic aluminosilicate belonging to the kaolinite group of clay minerals. See generally, Bates et al., “Morphology and structure of endellite and halloysite”, American Minerologists 35 463-85 (1950), which remains the definitive paper on halloysite. The mineral has the chemical formula Al₂O₃.2SiO₂.nH₂O. In hydrated form the mineral forms good tubules. In dehydrated form the mineral forms broken, collapsed, split, or partially unrolled tubules.” (See lines 46-57 of column 3)

The term “hydrated halloysite” is used in the claims of U.S. Pat. No. 4,019,934, the entire disclosure of which is hereby incorporated by reference into this specification. Claim 1 of this patent refers to an “inorganic gel.” Claim 4 of the patent recites that “4. The inorganic gel-ammonium nitrate composite material as claimed in claim 1 wherein said inorganic gel is prepared from a material selected from the group consisting of hydrated halloysite and montmorillonite.” As is disclosed in column 1 of such patent, “The purified and swollen inorganic gel prepared from a clay such as montmorillonite group, vermiculite, hydrated halloysite, etc., by the manner described hereinafter contains free water, bound water, and water of crystallization . . . . ”

As is also disclosed in U.S. Pat. No. 6,401,816(see lines 58-65 of column 3), “The nomenclature for this halloysite mineral is not uniform. In the United States, the hydrated tubule form of the mineral is called endellite, and the dehydrated form is called halloysite. In Europe, the hydrated tubule form of the mineral is called halloysite, and the dehydrated form is called is called meta-halloysite. To avoid confusion, mineralogists will frequently refer to the hydrated mineral as halloysite 10 .A., and the dehydrated mineral as halloysite 7 .A.”

As is also disclosed in U.S. Pat. No. 6,401,816 (see the paragraph commencing on line 66 of column 3), it was reported by Bates et al. that the tube diameter of halloysite ranges from 400 to 1900 angstroms with a median value of 700 angstroms, the hole diameter of halloyite ranges from 200 to 1000 angstroms with a median value of 400 angstroms, and the wall thickness of halloysite ranges from 100 to 700 angstroms with a median value of 200 angstroms.

As is also disclosed in U.S. Pat. No. 6,401,816(see the paragraph starting at line 9 of column 4),“Tube lengths range from 0.1 to about 0.75 μm. Morphologically, both hydrated and dehydrated halloysite comprise layers of single silica tetrahedral and alumina octahedral units. They differ in the presence or absence of a layer of water molecules between the silicate and alumina layers. The basal spacing of the dehydrated form is about 7.2 angstroms, and the basal spacing of the hydrated form is about 10.1 angstroms (hence the names halloysite 7 .A. and halloysite 10 .A). The difference, about 2.9 .A., is about the thickness of a monolayer of water molecules.”

As is also disclosed in U.S. Pat. No. 6,401,816 (see the paragraph beginning at line 19 of column 4), “A theory for the formation of hollow tubular microcrystals is presented in Bates et al. There is a lattice mismatch between the gibbsite (Al₂O₃) and silicate (SiO₂) layers. Water molecules interposed between the layers prevents “tetrahedral rotation” in the silicate layer. Halloysite 10 .angstroms dehydrates to halloysite 7 .angstroms at about 110° C. All structural water is lost at about 575° C. The interlayer water in halloysite 10 .angstroms may be replaced by organic liquids such as ethylene glycol, di- and triethylene glycol, and glycerine.”

In one embodiment, the clay mineral used in applicants' composition is endellite. As is disclosed in U.S. Pat. No. 6,401,816, endellite is the hydrated form of halloysite; see, e.g., column 3 of such patent. Reference may also be had to U.S. Pat. No. 3,956,140 (drilling fluids), U.S. Pat. No. 4,375,406 (fibrous clay composition), U.S. Pat. No. 4,150,099 (synthetic halloysites), U.S. Pat. No. 4,158,521 (method of stabilizing clay formations), U.S. Pat. No. 4,421,699 (method for producing a cordierite body),U.S. Pat. No. 4,505,833 (stabilizing clayey formations), U.S. Pat. No. 4,509,985 (early high-strength mineral polymers), U.S. Pat. Nos. 4,828 5,561,976(release of active agents using in,726 (stabilizing clayey formations), organic tubules), U.S. Pat. No. 5,820,302 microstructures is imogolite.” Reference also may be had, e.g., to United States patents (aggregate mixtures and structures), and the like. The entire disclosure of each of these United States patents is hereby incorporated by reference into this specification.

In another embodiment, the clay mineral used in applicants' composition is cylindrite. As is disclosed in U.S. Pat. No. 6,401,816 (see column 4), “Another mineral that will, under appropriate conditions, form tubules and other microstructures is cylindrite. Cylindrite belongs to the class of minerals known as sulfosalts.” Reference may also be had, e.g., to U.S. Pat. Nos. 4,415,711, 5,561,976 (controlled release of active agents with inorganic tubules), U.S. Pat. No. 5,701,191 (sustained delivery of active compounds from tubules), U.S. Pat. No. 5,753,736 (dimensionally stable fibers), and the like. The entire disclosure of each of these United States patents is hereby incorporated by reference into this specification.

In another embodiment, the clay mineral used a sulfosalt known as “Boulangerite.” Reference may be had, e.g., to column 4 of U.S. Pat. No. 6,401,816. Reference may also be had to U.S. Pat. Nos. 4,515,688; 4,626,279; 4,650,569; 5,182,014; 5,615,976 (inorganic tubules); U.S. Pat. No. 5,705,191 (sustained active delivery of compounds from tubules); U.S. Pat. No. 6,669,882 (process for making fiber having functional mineral powder), and the like. The entire disclosure of each of these United States patents is hereby incorporated by reference into this specification.

In another embodiment, the clay mineral used is imogolite. Reference may be had, e.g., to U.S. Pat. No. 6,401,816 (see column 4). Reference also may be had, e.g., to U.S. Pat. No. 4,152,404 (synthetic imogolite), U.S. Pat. No. 4,241,035 (synthetic imogolite), U.S. Pat. No. 4,252,799 (synthetic imogolite), U.S. Pat. No. 4,394,253 (imogolite catalyst), U.S. Pat. No. 4,446,244 (imogolite catalyst), and the like. The entire disclosure of each of these United States patents is hereby incorporated by reference into this specification.

In one preferred embodiment, and as is described in the claims of U.S. Pat. No. 5,651,976 (the entire disclosure of which is hereby incorporated by reference into this specification), the clay mineral is comprised of hollow mineral microtubules with an inner diameter of from about 200 angstroms to about 2000 angstroms and having lengths ranging from about 0.1 microns to about 2.0 microns. This patent claims (in claim 1) “1. A composition for use in the delivery of an active agent at an effective rate for a selected time, comprising: hollow mineral microtubules selected from the group consisting of halloysite. cylindrite, boulangerite, and imogolite, wherein said microtubules have inner diameters ranging from about 200 Angstroms to about 2000 .Angstroms, and have lengths ranging from about 0.1 μm to about 2.0 μm, wherein said active agent is selected from the group consisting of pesticides, antibiotics, antihelmetics, antifouling compounds, dyes, enzymes, peptides. bacterial spores, fungi, hormones, and drugs and is contained within the lumen of said microtubules, and wherein outer and end surfaces of said microtubules are essentially free of said adsorbed active agent.”

The incorporation of “active agents” into microtubules is described at columns 3-8 of U.S. Pat. No. 5,651,976. The process described in this patent may also be used to incorporate the nanomagnetic material of this invention into such microtubules. The entire disclosure of such United States patent is hereby incorporated by reference into this specification.

As is disclosed is U.S. Pat. No. 5,651,976 (see columns 3 et seq.), “Chemical agents, including the active agents of interest to the present invention, can enter or exit from the internal volume (lumen) of a cylindrical tubule by several mechanisms. For example, active agents can enter or exit tubules by capillary action, if the tubules are sufficiently wide. Capillary attraction and release occurs in tubules having inner diameters of at least about 0.2 μm. Capillary attraction is relatively weak: agents in tubules having inner diameters of at least about 10 μm. typically will be released in a matter of hours, without the use of other barriers to release.”

U.S. Pat. No. 5,651,976 also discloses that “In contrast to capillary action, adsorption/desorption processes occur over much smaller distance scales, typically on the order of about 1000 .Angstroms. Thus, for tubules in this size range, adsorption/desorption is the controlling process for the release of an active agent inside the interior volume of a microtubule. For a molecule of an active agent contained within the interior volume of a microtubule to reach the end of the tubule, so that the molecule can be released into the environment, the molecule must diffuse through the interior of the tubule while repeatedly being adsorbed and then desorbed by the inner surface of the tubule. This process, which may be conceptualized as a chromatography type of process, is much slower than capillary action, by several orders of magnitude.”

U.S. Pat. No. 5,651,976 also discloses that “Several naturally occurring minerals will, under appropriate hydration conditions, form tubules and other microstructures suitable for use in the present invention. The most common of these is halloysite, an inorganic aluminosilicate belonging to the kaolinite group of clay minerals. See generally, Bates et al., “Morphology and structure of endellite and halloysite”, American Minerologists 35 463-85 (1950), which remains the definitive paper on halloysite. The mineral has the chemical formula Al2O3.2SiO2.nH2O. In hydrated form the mineral forms good tubules. In dehydrated form the mineral forms broken, collapsed, split, or partially unrolled tubules.”

U.S. Pat. No. 5,651,976 also discloses that “The nomenclature for this mineral is not uniform. In the United States, the hydrated tubule form of the mineral is called endellite, and the dehydrated form is called halloysite. In Europe, the hydrated tubule form of the mineral is called halloysite, and the dehydrated form is called is called meta-halloysite. To avoid confusion, mineralogists will frequently refer to the hydrated mineral as halloysite 10 .Angstroms, and the dehydrated mineral as halloysite 7 .Angstroms.”

U.S. Pat. No. 5,651,976 also discloses that “Bates et al. present data on the tubes, which is summarized below . . . . Tube lengths range from 0.1 to about 0.75 μm. Morphologically, both hydrated and dehydrated halloysite comprise layers of single silica tetrahedral and alumina octahedral units. They differ in the presence or absence of a layer of water molecules between the silicate and alumina layers. The basal spacing of the dehydrated form is about 7.2 .Angstroms, and the basal spacing of the hydrated form is about 10.1 .Angstroms (hence the names halloysite 7 .A. and halloysite 10). The difference, about 2.9 .Angstroms, is about the thickness of a monolayer of water molecules.”

U.S. Pat. No. 5,651,976 also discloses that “A theory for the formation of hollow tubular microcrystals is presented in Bates et al. Water molecules interposed between the gibbsite (Al2O3) and silicate (SiO2) layers results in a mismatch between the layers, which is compensated by curvature of the layers. Halloysite 10 .A. dehydrates to halloysite 7 .A. at about 110° C. All structural water is lost at about 575° C. The interlayer water in halloysite 10 .A. may be replaced by organic liquids such as ethylene glycol, di- and triethylene glycol, and glycerine.”

U.S. Pat. No. 5,651,976 also discloses that “Another mineral that will, under appropriate hydration conditions, form tubules and other microstructures is imogolite.”

U.S. Pat. No. 5,651,976 also discloses that “Another mineral that will, under appropriate conditions, form tubules and other microstructures is cylindrite. Cylindrite belongs to the class of minerals known as sulfosalts.”

U.S. Pat. No. 5,651,976 also discloses that “Yet another mineral that will, under appropriate conditions, form tubules and other microstructures is boulangerite. Boulangerite also belongs to the class of minerals known as sulfosalts.”

U.S. Pat. No. 5,651,976 also discloses that “In preferred embodiments of the invention, an active agent is adsorbed onto the inner surface of the lumen of a mineral microstructure. Skilled practitioners will be able to employ known techniques for introducing a wide range of active agents into the lumen of a mineral microstructure according to the invention, thereby making a structure for the modulated release of the active agent. Such structures according to the invention may be used as-is, i.e., as free structures which may be dispensed as desired. Dispensing techniques include scattering, spreading, injecting, etc.”

U.S. Pat. No. 5,651,976 also discloses that “An important aspect of the microstructures is the size of the lumen. Preferred inner diameters range from about 200 .Angstroms to about 2000 .Angstroms. Preferred lengths range from about 0.1 μm. to about 2.0 μm. Lumen size selection is governed in part by the availability of ceramic or inorganic microstructures within the suitable size range. Lumen size selection is also governed by the choice of active agent, and the choice of any carrier, coating, or matrix (see infra). The physical and chemical properties (e.g., viscosity, solubility, reactivity, resistance to wear, etc.) of the active agent, any carrier, any coating and any matrix will be considered by a skilled practitioner. Lumen size selection is also governed by the desired release profile for the active agent.”

U.S. Pat. No. 5,651,976 also discloses that “Such structures may be included in a surrounding matrix, such as a paint or a polymer. After release from the mineral microstructures, the active agent then diffuses through the surrounding matrix to interface with the use environment. If the surrounding matrix is ablative in the use environment, then the diffusion distance through the matrix is mitigated or eliminated by this ablation.”

U.S. Pat. No. 5,651,076 also discloses that “Suitable surrounding matrices will typically be insoluble in the use environment. These matrices include paints (including marine paints), stains, lacquers, shellacs, wood treatment products, and all manner of applied coatings.”

U.S. Pat. No. 5,651,076 also discloses that “In another embodiment of the invention, the lumen of the microstructure contains both an active agent and a carrier. This carrier further modulates the release of the active agent from the lumen of the microstructure. The active agent may be soluble or mobile in the carrier. In this case, the release rate of the active agent will depend on the solubility and diffusion rate of the active agent through the carrier and any coating or matrix. The active agent may be insoluble or immobile in the carrier. In this case, the release rate of the active agent will depend on the release rate of the carrier from the tubule, and any coating or matrix.”

U.S. Pat. No. 5,651,076 also discloses that “In another embodiment of the invention, the microstructure is coated with a coating material. This coating further modulates the release of the active agent from the lumen of the microstructure. By carefully selecting a coating for its chemical and physical properties, very precise control of the release of the active agent from the lumen of the microstructure can be achieved.”

U.S. Pat. No. 5,651,076 also discloses that “For example, a thermoset polymer may be used as a coating in a preferred embodiment of the invention. By carefully selecting the degree of crosslinking in a thermoset polymer coating, and thus the porosity of the thermoset polymer coating, one can obtain a precise degree of control over the release of the active agent from the lumen of the microstructure. Highly crosslinked thermoset coatings will retard the release of the active agent from the lumen more effectively than less crosslinked thermoset coatings.”

U.S. Pat. No. 5,651,076 also discloses that “Likewise, the chemical properties of a coating may be used to modulate the release of an active agent from the lumen of a microstructure. For example, it may be desired to use a hydrophobic active agent in an aqueous use environment. However, if one were to load a highly hydrophobic active agent into the lumen of a microstructure according to the invention, and then place this loaded microstructure in an aqueous use environment, the active agent typically would release into the use environment unacceptably slowly, if at all.”

U.S. Pat. No. 5,651,076 also discloses that “This problem of active agents that are highly insoluble in an intended use environment is a common one. Many antibiotics are highly insoluble in the serum. This problem can be largely mitigated by coating the microstructures with a coating material in which the active agent has an intermediate solubility (i.e., a solubility somewhere between the solubility of the active agent in itself and the solubility of the active agent in the use environment).”

U.S. Pat. No. 5,651,076 also discloses that “A wide range of active agents will be suitable for use in the present invention. These suitable active agents include pesticides, antibiotics, antihelmetics, antifouling compounds, dyes, enzymes, peptides, bacterial spores, fungi, hormones, etc.”

U.S. Pat. No. 5,651,076 also discloses that “Suitable herbicides include tri-chloro compounds (triox, ergerol), isothiazoline, and chlorothanolil (tufficide). Suitable pesticides include malathion, spectricide, and rotenone. Suitable antibiotics include albacilin, amforol, amoxicillin, ampicillin, amprol, ariaprime, aureomycin, aziumycin, chloratetracycline, oxytetracycline, gallimycin, fulvicin, garacin, gentocin, liquamicin, lincomix, nitrofurizone, penicillin, sulfamethazine, sulfapyridine, fulfaquinoxaline, fulfathiozole, and sulkamycin. Suitable antihelmetics include ivermictin, vetisulid, trichorofon, tribrissen, tramisol, topazone, telmin, furox, dichlorovos, anthecide, anaprime, acepromazine, pyrantel tartrate, trichlofon, fanbentel, benzimidazoles, and oxibenzidole. Suitable antifouling agents include ergerol, triazine, decanolactone, angelicalactone, galactilone, any lactone compound, capsicum oil, copper sulphate, isothiazalone, organochlorine compounds, organotin compounds, tetracyclines, calcium ionophores such as 504, C23187, tetracycline. Suitable hormones include estrogen, progestin, testosterone, and human growth factor.”

U.S. Pat. No. 5,651,076 also discloses that “Carriers are selected in view of their viscosity and the solubility of the active agent in the carrier. The carrier typically should possess a sufficiently low viscosity to fill the lumen of the microstructure. Alternatively, a low viscosity carrier precursor may be used, and the carrier formed in situ. For example, the lumen may be filled with a low viscosity monomer, and this monomer subsequently may be polymerized inside the lumen. Accordingly, suitable carriers include low molecular weight polymers and monomers, such as polysaccharides, polyesters, polyamides, nylons, polypeptides, polyurethanes, polyethylenes, polypropylenes, polyvinylchlorides, polystyrenes, polyphenols, polyvinyl pyrollidone, polyvinyl alcohol, ethyl cellulose, gar gum, polyvinyl formal resin, water soluble epoxy resins, quietol 651/nma/ddsa, aquon/ddsa/nsa, urea-formaldehyde, polylysine, chitosan, and polyvinylacetate and copolymers and blends thereof.”

U.S. Pat. No. 5,651,076 also discloses that “Frequently, skilled practitioners may desire to select a carrier that has a very highly selective binding affinity for an active agent of interest. A carrier that has a highly selective binding affinity for an active agent will tend to release that active agent very slowly. Thus, very slow release rates may be achieved by the use of carriers with high binding affinities for the active agent to be released. Skilled practitioners will recognize that a consequence of the extensive research that has been done on surface acoustic wave (SAW) analysis is that a large number of polymers have been identified as selective adsorbents for particular organic analytes. See generally, D. S. Ballantine, Jr., S. L. Rose, J. W. Grate, H. Wohltjen, Analytical Chemistry 58 3058-66 (1986), and references therein, incorporated by reference herein. See also R. A. McGill et al., “Choosing Polymer Coatings for Chemical Sensors”, CHEMTECH 24 (9) 27-37, and references therein, incorporated by reference herein.”

U.S. Pat. No. 5,651,076 also discloses that “Preferred carriers include polylactate, polyglycolic acid, polysaccharides (e.g., alginate or chitosan), and mixtures thereof. Each of these carriers is biodegradable. When used in combination with a naturally occurring mineral microtubule, such biodegradable carriers provide an environmentally friendly delivery system.”

U.S. Pat. No. 5,651,076 also discloses that “Having described the invention, the following examples are given to illustrate specific applications of the invention, including the best mode now known to perform the invention. These specific examples are not intended to limit the scope of the invention described in this application.”

U.S. Pat. No. 5,651,076 also discloses that, in Example 1, “The halloysite was obtained as a crude sample of the lump clay deposit and was hydrated in distilled water, containing 5% by weight sodium metaphosphate. The clay was then crudely crushed by hand, using a metal hammer to break up the large lumps, and foreign material and rocks were sorted by hand. The sample was then transferred into a common kitchen blender adding 200 g of the sample to 1 liter of water. The mixture was allowed to agitate at a medium speed for a period of 30 minutes. The material in suspension was removed and fresh water containing 5% by weight Na metaphosphate was added and the process repeated until the clumps would no longer break down. Following this step the suspension was allowed to stand in a 3 L graduate cylinder for 10 minutes, and then the suspended portion of the sample was removed for further processing. The gravity settlement allowed further separation of quartz sand particles from the halloysite. The resultant suspension was spun in an IEC Model C-6000 centrifuge in 1 L bottles and the supernatant removed and replaced with fresh distilled water, and the process was repeated an additional two cycles. The resultant slurry was then filtered through a cloth paint filter cone to remove any remaining large clumps, which were then ground in a mortar and pestle and retreated as before. Once the halloysite sample was found to be substantially free of foreign material, it was spun out of the water suspension and allowed to air dry. This yielded a white cake of halloysite that was then powdered in a mortar and pestle, to yield a friable white powder.”

U.S. Pat. No. 5,651,076 also discloses that “The powder of dry halloysite microcylinders were treated by the following scheme. The active agent which is to be employed by the first method of entrapment should be a solid at or below 40° C. In this method both the halloysite and the agent are heated to a temperature just above the melting point of the agent. The best method should be a vacuum oven, if possible, under a partial vacuum to aid in removal of retained gasses within the core of the microcylinders.”

U.S. Pat. No. 5,651,076 also discloses that “The halloysite was observed to be “wet” with the active agent. Following this step the vacuum was released and the resultant agent/microcylinders complex was suspended in a dispersant that was not a solvent for the agent, and was at the same temperature as the agent/halloysite. With sufficient agitation, the temperature was lowered until the agent became a solid again. The agitation optionally may be stopped at this point and the suspension allowed to settle. The dispersant was removed and the resultant halloysite/agent complex was then suspended in a solvent for the agent. This resulted in the removal of the exogenous agent from the microcylinder.”

U.S. Pat. No. 5,651,076 also discloses that “The second method employed utilized a suspension of the halloysite and agent in solution of a suitable biodegradable polymer such as a poly-lactic/polyglycolic acid system, which was diluted in a suitable solvent such as methanol. The resultant suspension was then injected into a fluidized bed to flash off the solvent and yield a halloysite/agent mixture which had an outer coating of an environmentally benign coating of degradable polymer.”

U.S. Pat. No. 5,651,076 also discloses that “The third method required the active agent to be miscible with the polylactic/polyglycolic acid mixture, or that the active agent be very small particulates (nanoparticulates). This mixture was then entrapped in the central core of the microcylinders by a method similar to that in the original method, except that the agent was allowed to flash off in the vacuum at ambient temperatures.”

U.S. Pat. No. 5,651,076 also discloses that “To determine the encapsulation efficiency, the microcylinders were crushed and suspended in a suitable solvent. The suspension was agitated for several hours to ensure full dissolution of the active agent. The determination of concentration of active agents was made either by weight or by suitable chemical analysis.”

U.S. Pat. No. 5,651,076 also discloses that “Laboratory Determination of Release Rate The microtubules were added to a conical 50 ml disposable centrifuge, and 50 ml of deionized H2O was added. Concentration determinations were made based on absorption in a Perkin Elmer UV/Vis series 6000 spectrophotometer. A peristaltic pump was employed to pump the solution through a quartz flow cell where absorption measurements were made each half-hour. When necessary, the deionized H2O was changed to prevent saturation.”

U.S. Pat. No. 5,651,076 also discloses that “Additional modification of the release characteristics has been achieved through employment of a further layer of the degradable polymeric material, where the secondary layer was free of any active agent. This provides a barrier coating to protect against short term exposure to the entrapped agent during handling. This coating then degrades in the environment at a rate that is determinable by the degree of cross-linking of the co-polymers or by employment of an additional crosslinking agent. This allows for a delayed release product. By mixing the thickness of the overcoating, the delay has been tailored to initiate release over a considerable time period.”

U.S. Pat. No. 5,651,076 also discloses that “For shorter term release profiles (<300 hr) polysaccharides (including alginate and chitosan) have provided a carrier and a coating that was biodegradable. Due to the open nature of the gel, the release rate has been rather fast, depending on the agent.”

Synthetic Clay Minerals

In one preferred embodiment, the clay mineral used in the composition of this invention is a synthetic clay mineral, that is, a naturally occurring clay mineral that has been modified by one or more human operations.

In one embodiment, the synthetic clay mineral is a 2:1 layer-type clay mineral product, as that term is defined in U.S. Pat. No. 3,875,288. This patent claims (in claim 1) “1. The process of producing a 2:1 layer-type clay-like mineral product having the empirical formula: nSiO2:Al2O3:mAB:xH2O where the layer lattices comprise said silica, said alumina, and said B, and where n is from 1.7 to 3.0, m is from 0.2 to 0.6, A is one equivalent of an exchangeable cation chosen from the group consisting of ammonium, sodium, calcium, hydrogen, and mixtures thereof, and is external to the lattice, B is chosen from the group of anions which consists of F−, OH−, 1/2O2--, and mixtures thereof, and is internal in the lattice, and x is from 2.0 to 3.5 at 50 percent relative humidity, said mineral being characterized by a d001 spacing at said humidity within the range which extends from a lower limit of about 12.0 A. when A is monovalent, to about 14.7 A. when A is divalent, and to a value intermediate between 12.0 A. and 14.7 A. when A includes both monovalent and divalent cations which comprises the steps of forming a reaction mixture by bringing together a 1:1 clay chosen from the group consisting of calcined kaolinite, calcined halloysite, acid-washed calcined kaolinite, acid-washed calcined halloysite, and mixtures thereof; a cation or mixture of cations chosen from the group consisting of said A, together with an equivalent amount of an anion chosen from the group consisting of hydroxyl and fluoride and mixtures thereof; and water; the relative quantities of said reaction mixture components being selected so as to give a molar ratio of SiO2/Al2O3 of between about 1.9 and 3.2; of F−/SiO2 of between about 0.02 and 0.3; and of NH4+/Al2O3 of between about 0.1 and 2.0; and so as to give a pH of between about 4.5 and 11.5 and a solids/water weight ratio of between about 0.08 to about 0.6; and thereafter heating said reaction mixture under hermetically sealed conditions to a temperature within the range of about 275° C. to about 320° C. and maintaining said mixture within said range for a period of time long enough for said mineral product to form; and thereafter allowing said mineral product to cool and recovering said mineral product.” The process described in such claim 1 is described in more detail at columns 2-4 of such U.S. Pat. No. 3,875,288, wherein it is disclosed that “The relative quantities of the several reaction mixture components are selected so as to give a molar ratio of silica to alumina, i.e., SiO2/Al2O3, of between about 1.9 and 3.2; of fluoride ion to silica, i.e., F−/SiO2, of between about 0.02 and 0.3; and of ammonium ion to alumina, i.e., NH4+/Al2O3, of between about 0.1 and 2.0; and so as to give a pH of between about 4.5 and 11.5; and a solids/water weight ratio of between about 0.08 and about 0.6, i.e., from about 8 percent to about 60 percent solids.”

U.S. Pat. No. 3,875,288 also discloses that “The reaction mixture having been formed, it is then placed in a pressure vessel if indeed not already therein, which is then hermetically sealed and heated to a temperature within the range of about 275° C. to about 320° C., about 300° C. being generally preferred. This temperature is maintained until the 2:1 layer-type clay-like mineral product has formed. As will be seen from the examples which follow, typical times are of the order of three hours for batches of a kilogram or so. This may be compared with typical times set forth in the cited Granquist patent of about 1 to 2 days. We have found that in general as the size of the equipment and batch increases, the processing times decrease. Thus, in lots of the order of a ton or so, the Granquist product may often be made in as short a time as 4 or 5 hours; and for the same size batch the present invention permits a processing time as short as 1 hour.”

U.S. Pat. No. 3,875,288 also discloses that “The product having been formed as described, the vessel and contents are allowed to cool until the vessel may be safely opened, and the product is recovered. Any after treatment naturally depends upon the use to be made of the product. Simple draining of excess liquid with or without drying may be adequate. Or, the solids may be washed to any desired degree of freedom from excess salts, and may be base-exchanged with any desired cation or mixture of cations, and ultimately dried and ground if desired.”

U.S. Pat. No. 3,875,288 also discloses that “The product thus produced in accordance with the invention has the characteristics described for the product of Granquist U.S. Pat. No. 3,252,757, and discussed therein in considerable detail. In particular, quite remarkably the product upon x-ray diffraction no longer exhibits any content of the starting 1:1 clay, but shows itself to be comprised of the randomly alternating mixture of interstratified mica-like and montmorillonite-like layers, both of which are 2:1 type phyllosilicates. This terminology is well understood by those skilled in the art. Reference may be made to the text by Ralph Grim: Clay Mineralogy, Ed. 2, New York 1968, and in particular chapters 3, on nomenclature, and 4, on structure, which are hereby incorporated herein by reference.”

“An especial advantage of the present invention is that it permits the production of the Granquist-type mineral product with a wider range of silica-to-alumina ratios than originally disclosed. Thus, good syntheses may be made at SiO2/Al2O3 ratios of as small as 1.7. [It may be noted that the product in accordance with the invention generally has an SiO2/Al2O3 ratio about 0.2 to 0.3 less than that of the reaction mixture.] When this is desired, a kaolinite of suitably low silica/alumina ratio may be selected, since there is some variation in the natural clay. Alternatively, most halloysites have lower ratios than most kaolinites.”

U.S. Pat. No. 3,875,288 also discloses that “In the event that higher ratios are desired, reactive silica is included in the reaction mixture. This may be polysilicic acid, produced for example in accordance with Hoffman U.S. Pat. No. 3,649,556; or a fumed silica, several of which are commercially available and which are characterized by extremely fine particle size, made for example by the silicon monoxide or the silicon tetrachloride route as described in the book by Ralph Iler: The Colloid Chemistry of Silica and Silicates, Ithaca 1955, on pages 168-9 and 172-3 thereof; or diatomite; or silica-rich tripoli. These are all described in Chapter VI of the book by Iler just cited, which is hereby incorporated herein by reference. The quantity of reactive silica admixed may be relatively small or great, but of course should not be so great as to exceed the silica/alumina ratio for the reaction mixture already specified herein.”

U.S. Pat. No. 3,875,288 also discloses that “Alternatively, the calcined kaolinite or calcined halloysite may be acid-washed, which selectively removes alumina by dissolution, leaving a usable structure with a higher silica/alumina ratio than the starting clay. Any strong acid may be used, such as sulfuric or hydrochloric, followed by water-washing to remove the residual acid and dissolved alumina. In general it is more practical and more economical to add reactive silica.”

U.S. Pat. No. 3,875,288 also discloses that “As already stated, the kaolinite or halloysite or the mixture of both is calcined before use in accordance with the invention. Calcination is carried out within the range 600° to 700° C., preferably about 650° C. The time is not critical, a half-hour or hour sufficing at the preferred temperature. Such calcining fundamentally changes the x-ray diffraction pattern of these clays. If the 1:1 clay is not calcined first, but used as mined, then the conversion to the unique 2:1 Granquist-type clay does not take place.”

U.S. Pat. No. 3,875,288 also discloses that “It may be noted that many clay firms will supply kaolinite already calcined to order, so that this step need not be carried out by the operator of the inventive procedure.”

U.S. Pat. No. 3,875,288 also discloses that “As will be evident from the examples to be given hereinbelow, the cation-anion combinations used in the reaction mixture may quite simply comprise ammonium bifluoride, NH4F.HF, also written as NH4HF2; and ammonium hydroxide, NH4 OH, in preselected proportions to give the desired ratios. Calcium ion is conveniently added as calcium oxide, or, if included before calcining, as calcium carbonate. Sodium may be added as the hydroxide or the fluoride. In general, we prefer a fluoride/silica ratio of about 0.1; as this ratio diminishes, the reaction time tends to be prolonged.”

U.S. Pat. No. 3,875,288 also discloses that “A variation in procedure within the broad scope of the invention comprises the formation of pellets from all or most of the reaction mixture; or from all of the 1:1 clay and most of the other ingredients, with enough water to enable pellets to be readily formed using any commercial pelletizer, as is commonplace in the catalyst industry. A suitable size for the pellets is from about one-eighth to three-sixteenths inch in diameter, although this range may be exceeded. We have had excellent results at one-eighth inch. Kaolinites and halloysites from different sources tend to have different pelletizing characteristics, so that in some cases it may be desirable to include a binder in the mix fed to the pelletizer. A minor quantity of the mineral product made in accordance with the invention in a previous run serves admirably; 10 to 20 percent by weight of the calcined 1:1 clay may be used, for example. Alternatively, or additionally, some of the reactive silicas have binding properties and may be included for this purpose, especially polysilicic acid.”

U.S. Pat. No. 3,875,288 also discloses that “While the pellets so produced may be used forthwith, we prefer and find best to dry the pellets at about 105° C. to 110° C. and then calcine them at about 600° C. to 700° C., and preferably at about 650° C. Remarkably, even though in the preferred embodiment the pellets will have been made up with ammonium bifluoride and ammonium hydroxide as already mentioned, no additional fluoride ion need be incorporated in the final reaction mixture in spite of the high temperature of calcining. It appears that a semi-solid-state reaction occurs within the pellets during the drying and calcining, so that when the final conversion to the 2:1 phyllosilicate product is made in the autoclave, the conversion time is shortened even more so. The calcination of the pellets has the further advantage that they tend to retain their shape during the autoclaving, thus permitting ready access of the chemical solution surrounding them.”

In one embodiment, the synthetic clay mineral is a halloysite that has a surface area greater than 85 square meters per gram, as is described in U.S. Pat. No. 4,098,678, the entire disclosure of which is hereby incorporated by reference into this specification. This United States patent claims (in claim 1) “1. A process for the conversion of hydrocarbons, which comprises contacting said hydrocarbons at hydrocarbon converting conditions with a synthetic, non-acid treated halloysite containing less than 0.05 wt. % iron and having a surface area greater than 85 sq. meters/gram.” Claim 2 of this patent describes “2. A process for the conversion of hydrocarbons which comprises contacting said hydrocarbons and hydrocarbon converting conditions with a synthetic, non-acid treated halloysite having a surface area greater than 85 sq. meters/gram and having the empirical formula: [xAl+3/n(1−x)M]2O3. (2+y)SiO2.2H2O where M is a metal selected from Groups IIA, IIIB, VIB and VIII of the Periodic Table; n is valence of M; x is equal to or less than 1; and y=0 to 1.” The preparation of these synthetic halloysties is described at columns 2-4 of such patent, wherein it is disclosed that “Preparation of the synthetic halloysite of the invention involves the reaction of hydrous alumina gel, i.e., Al(OH)3, and a source of silica. The hydrous alumina gel is prepared in accordance with known techniques such as by the reaction of aqueous mixtures of aluminum chloride or aluminum sulfate and an inorganic base such as NH4OH, NaOH or NaAlO2, and the like. Preparation of alumina gel by use of ammonium hydroxide is preferable to the use of sodium hydroxide since it is desirable to maintain the soda (Na2O) content to a low level and because the more alkaline gels tend to form crystalline boehmite.”

U.S. Pat. No. 4,098,678 also discloses that “The silica source may include those sources which are conventionally used for the preparation of crystalline aluminosilicate zeolites. These include silicic acid, silica sol, silica gel, sodium silicate, etc. Silica sols are particularly useful. These are colloidal dispersions of discrete spherical particles of surface-hydroxylated silica such as is sold by E. I. du Pont de Nemours & Company, Inc. under the trademark “Ludox”.”

U.S. Pat. No. 4,098,678 also discloses that “The proportions of the reactants employed in the initial reaction mixture are determined from the following molar ratio of reactants . . . . The pH of the reaction mixture should be adjusted to a range of about 4 to 10, preferably 6 to 8. The temperature of the reaction mixture should preferably be maintained at between about 230° and 270° C., more preferably 240° to 250° C., for a period from about 2 hours to 100 hours or more. The time necessary for crystallization will depend, of course, upon the temperature of the reaction mixture. By way of example, the crystallization of the synthetic halloysite occurs in about 24 hours at a temperature of about 250° C.”

U.S. Pat. No. 4,098,678 also discloses that “The catalytic activity of the synthetic halloysites of the invention can be improved by incorporating therein metals selected from Groups IIA, IIIB, VIB, and VIII of the Periodic Table as given in “Websters Seventh New Collegiate Dictionary”, (1963) published by G. C. Merriam Company. Specific examples of such metals include, among others, magnesium, lanthanum, molybdenum, cobalt, nickel, palladium, platinum and rare earths. Particularly preferred metals include magnesium, nickel, cobalt and lanthanum. The metals are incorporated into the synthetic halloysite structure by adding soluble salts of the metal to the reaction mixture or by coprecipitation of the metal hydroxide with Al(OH)3. The metals are most conveniently added to the reaction mixture in the form of their hydroxides. The synthetic halloysite of the invention, particularly when substituted with the afore-described metals, is useful for catalytic cracking, hydrocracking, desulfurization, demetallization and other hydrocarbon conversion processes. For example, substituted halloysites of the invention containing metals such as magnesium, lanthanum and rare earths such as cerium, praseodymium, neodymium, gadolinium, etc. are useful in catalytic cracking of petroleum feedstocks. Synthetic halloysite containing nickel, cobalt, palladium, platinum, and the like are particularly useful for hydrocracking petroleum feedstocks.”

U.S. Pat. No. 4,098,678 also discloses that “The feedstocks suitable for conversion in accordance with the invention include any of the well-known feeds conventionally employed in hydrocarbon conversion processes. Usually they will be petroleum derived, although other sources such as shale oil are not to be excluded. Typical of such feeds are heavy and light virgin gas oils, heavy and light virgin naphthas, solvent extracted gas oils, coker gas oils, steam-cracked gas oils, middle distillates, steam-cracked naphthas, coker naphthas, cycle oils, deasphalted residua, etc.”

U.S. Pat. No. 4,098,678 also discloses that “The operating conditions to be employed in the practice of the present invention are well-known and will, of course, vary with the particular conversion reaction desired. The following table summarizes typical reaction conditions effective in the present invention . . . . ”

U.S. Pat. No. 4,098,678 also discloses that “The halloysite structure of the composition of this invention has been confirmed by X-ray diffraction and electron microscopy. However, there are a number of significant differences between naturally occurring halloysite and the synthetic halloysite of this invention. For example, the synthetic halloysites of the invention have surface areas ranging from about 85 sq. meters/gram to about 200 sq. meters/gram (BET Method as used, for example, in U.S. Pat. No. 3,804,741) as compared to naturally occurring halloysite which has a surface area generally within the range of 40-85 sq. meters/gram (BET Method). Further, the synthetic halloysite of the invention will be substantially iron-free, i.e., less than 0.05% iron, as compared to naturally occurring halloysite which contains significant amounts of iron. The synthetic and naturally occurring halloysites also differ in that the physical form of the synthetic halloysite is flakes, while the physical form of the natural halloysite has a tube-like configuration. Furthermore, it has been discovered that the synthetic halloysite has considerably better catalytic activity than natural halloysite under analogous hydrocarbon conversion conditions. Although the synthetic halloysite has the same empirical formula as naturally occurring halloysite, the higher surface area, the elimination of iron and the presence of selective metals makes the synthetic halloysite a more effective hydrocarbon conversion catalyst.”

In one embodiment, the synthetic clay mineral is the synthetic halloysite described in U.S. Pat. No. 4,150,099, the entire disclosure of which is hereby incorporated by reference into this specification. Claim 1 of this patent describes “1. A process for preparing halloysite which comprises forming a reaction mixture of aluminum hydroxide gel, silica sol and water having a Al(OH)3/SiO2 molar ratio in the range of 0.5 to 1.2 and a H2O/SiO2 molar ratio in the range of 20 to 60 and maintaining said reaction mixture at a pH in the range of 4 to 10 and a temperature of about between 230° and 270° C. for a time sufficient to permit crystallization of halloysite.”

In one embodiment, the synthetic clay mineral is a chlorinated clay mineral, such as a chlorinated halloysite, as that term is defined in U.S. Pat. No. 4,798,630, the entire disclosure of which is hereby incorporated by reference into this specification. Claim 1 of U.S. Pat. No. 4,798,630 describes a process for chlorinating an aluminosilicate clay mineral starting composition, describing “1. A method for chlorinating and functionalizing an aluminosilicate clay mineral starting composition, comprising: reacting a said clay mineral composition selected from one or more members of the group consisting of clays of the halloysite, illite, kaolinite, montmorillonite, and polygorskite groups in substantially dry particulate form with gaseous SiCl4 to activate the surface of said composition, thereby forming a reactive chloride intermediate, said reaction being conducted at temperatures in the range of from about 56° C. to below 300° C.; maintaining said intermediate in a substantially dry state until used for further reaction; and thereafter functionalizing said intermediate with an active organic group.”

In one preferred embodiment, the synthetic clay mineral is a layered kaolinitic mineral (such as halloysite)that has undergone cation exchange with a specified cation. Such a “cation halloysite” is described, e.g., in claims 22, 28, and 29 of U.S. Pat. No. 5,530,052, the entire disclosure of which is hereby incorporated by reference into this specification; reference also may be had, e.g., to U.S. Pat. No. 5,707,439. As is disclosed in column 1 of U.S. Pat. No. 5,530,052, “Efforts have been disclosed for preparing polymeric nanocomposites. In International Application WO 94/11430, nanocomposites having two essential components are described and the two essential components are gamma phase polyamides and layered and fibrillar inorganic materials which are treated with quaternary ammonium cations . . . . Still other efforts have been made to prepare composite materials containing a layered silicate. In U.S. Pat. No. 4,889,885, a composite material having high mechanical strength and heat resistance which is suitable for use in automotive parts, aircraft parts and building materials is described . . . . The instant invention is patentably distinguishable from the, above-described since, among other reasons, it is directed to novel layered minerals that have undergone a cation exchange with at least one heteroaromatic cation comprising a positively charged organo-substituted heteroatom and/or a positively charged heteroatom not part of an aromatic ring with at least one bond having a bond order greater than one, and compositions prepared therefrom. Additionally, the instant invention is directed to novel compositions prepared from low viscosity macrocyclic oligomers.”

In one embodiment, the synthetic clay mineral is the organophilic phylosilicate described by the claims of U.S. Pat. No. 6,197,849, the entire disclosure of which is hereby incorporated by reference into this specification. Claim 1 of this patent describes “1. An organophilic phyllosilicate which has been prepared by treating a naturally occurring or synthetic phyllosilicate, or a mixture of such silicates, with a salt of a quaternary or other cyclic amidine compound or with a mixture of such salts.” Claim 2 describes “2. An organophilic phyllosilicate according to claim 1, whose preparation uses naturally occurring or synthetic smectite clay minerals, bentonite, vermiculite and/or halloysite, and preferably montmorillonite, saponite, beidelite, nontronite, hectorite, sauconite or stevensite, and particularly preferably montmorillonite and/or hectorite.” Claim 3 describes “3. An organophilic phyllosilicate according to claim 1, which has a distance between layers of from about 0.7 nm-1.2 nm (nanometers) and a cation-exchange capacity in the range from 50-200 meq/100 g.”

The organophylosilicates of these claims are further described in column 1 of U.S. Pat. No. 6,197,849, wherein it is disclosed that “It is known that organophilic phyllosilicates prepared, for example, by ion exchange, can be used as fillers for thermoplastic materials and also for thermosets, giving nanocomposites. When suitable organophilic phyllosilicates are used as fillers, the physical and mechanical properties of the moldings thus produced are considerably improved. A particular interesting feature is the increase in stiffness with no decrease in toughness. Nanocomposites which comprise the phyllosilicate in exfoliated form have particularly good properties.”

U.S. Pat. No. 6,197,849 also discloses that “U.S. Pat. No. 4,810,734 has disclosed that phyllosilicates can be treated with a quaternary or other ammonium salt of a primary, secondary or tertiary linear organic amine in the presence of a dispersing medium. During this there is ion exchange or cation exchange, where the cation of the ammonium salt becomes embedded into the space between the layers of the phyllosilicate. The organic radical of the absorbed amine makes phyllosilicates modified in this way organophilic. When this organic radical comprises functional groups the organophilic phyllosilicate is able to enter into chemical bonding with a suitable monomer or polymer. However, the use of the linear amines mentioned in U.S. Pat. No. 4,810,734 has the disadvantage that they decompose thermally at the high temperatures of up to 300° C. usually used for thermoplastics processing and can discolor the product. The formation of decomposition products can lead to emissions and to impairment of mechanical properties, for example impact strength.”

U.S. Pat. No. 6,197,849 also discloses that “Surprisingly, it has now been found that organophilic phyllosilicates which have been prepared by treating phyllosilicates, i.e. using cation exchange with salts of quaternary or other cyclic amidine compounds, have greater thermal stability during processing combined with excellent dispersing effect and interfacial adhesion. When the amidinium compounds according to the invention are used in thermosets there is no change in the stoichiometry of the reactive components, in contrast to the use of linear ammonium salts, and this allows addition to the thermosetting materials of an increased proportion of tillers. If the cyclic amidines used contain reactive groups the organophilic phyllosilicates prepared therewith and used as fillers can be covalently linked to the matrix by grafting. Amidinium ions derived, for example, from hydroxystearic acid or hydroxyoleic acid have surprisingly good layer separation combined with excellent adhesion to a wide variety of polymers and fillers. In contrast to the prior art alkyl groups with nonterminal hydroxyl groups in particular are useful, as well as alkyl substituents with terminal hydroxyl groups. The hydroxyl groups in the alkyl side chain may easily be derivatized in order to tailor a system-specific property spectrum. The compounds also create excellent dispersing effect and interfacial adhesion. It is also surprising that, despite their bulk, the heterocyclic amidine salts according to the invention, with long substituted or unsubstituted alkyl radicals, exchange cations efficiently within the spaces between the layers of the phyllosilicates.”

In one preferred embodiment, the synthetic clay mineral is an acidified calcined halloysite, as that term is defined in U.S. Pat. No. 6,294,108, the entire disclosure of which is hereby incorporated by reference into this specification. Claim 1 of this patent refers to “1. A dry solid composition for generating chlorine dioxide gas consisting essentially of a combination of at least one dry metal chlorite and at least one dry solid hydrophilic material comprising at least one inorganic material selected from the group consisting of hydrous clays, calcined clays, acidified clays and acidified calcined clays, wherein said combination is one which passes both the Dry Air and Humid Air Tests.” Claim 6 of this patent refers to a “hydrous halloysite,” stating “6. The composition of claim 1 wherein the hydrous clay is selected from the group consisting of bentonite, kaolin, attapulgite and halloysite.” Claim 7 refers to “calcined halloysite,” stating “7. The composition of claim 1 wherein the calcined clay is selected from the group consisting of metakaolin, spinel phase kaolin, calcined bentonite, calcined halloysite and calcined attapulgite.” Claim 8 refers to “acidified halloysite,” stating “8. The composition of claim 1 wherein the acidified clay is selected from the group consisting of bentonite, kaolin, attapulgite and halloysite that have been contacted with one or more acidic solutions containing sulfuric acid, hydrochloric acid, nitric acid or other acidic compounds so that the pH of the resulting liquid phase of the mixture is below 10.5.” Claim 9 refers to “acidified, calcined halloysite,” stating “9. The composition of claim 1 wherein the acidified calcined clay is selected from the group consisting of metakaolin, spinel phase kaolin, calcined bentonite, calcined halloysite and calcined attapulgite that have been contacted with one or more acidic solutions containing sulfuric acid, hydrochloric acid, nitric acid or other acidic compounds so that the pH of the resulting liquid phase of the mixture is below 10.5.” Any of these forms of halloysite may be used in the composition of this invention.

In one embodiment, the synthetic clay mineral is selected from the group consisting of organosilicate clay and organophilic clay, as these terms are defined by U.S. Pat. No. 6,501,934, the entire disclosure of which is hereby incorporated by reference into this specification. Claim 1 of this patent describes “An electrophotographic transfer member having a substrate comprising a nanosize polymer filler material wherein said nanosize polymer material is selected from the group consisting of particulate organosilicate clay filler material and organophilic clays, wherein the amount of said filler in said substrate is lower than about 10% by weight.”

In the embodiment defined by claim 2 of U.S. Pat. No. 6,501,934, the “ . . . organosilicate clay filler material is an organically modified talc-type silica (OMTS) in nanosize particulate form.” In the embodiment defined by claim 3 of U.S. Pat. No. 6,501,934, “ . . . wherein said organophilic clay is an organically modified particulate organically modified mica, bentonite; allophane; kaolinite; halloysite; illite; chlorite; vermiculite; sepiolite; attapulgite; palygorskite; and mixed-layer clay minerals in nanosize particulate form.”

The organophilic clay is described at column 3 of U.S. Pat. No. 6,501,934, wherein it is disclosed that “The nanosize polymer material may be an organophilic clay. “Organophilic clay” includes layered minerals such as particulate organically modified mica, e.g., muscovite, lepidolite, phlogopite or glauconite; or organically modified bentonite, e.g., montmorillonite; allophane; kaolinite; halloysite; illite; chlorite; vermiculite; sepiolite; attapulgite; palygorskite; and mixed-layer clay minerals in nanosize particulate form which have been intercalated with organic cations. Exemplary cations include onium cations, e.g., higher (including C4 to C20 alkyl) alkylammonium ions like laurylammonium, palmitylammonium, and stearylammonium. Desirably the clay from which the organophilic clays are prepared have a cation exchange capacity from 50 to 300 milliequivalents per 100 grams of clay.”

U.S. Pat. No. 6,501,934 also discloses that “The intercalation of the layered minerals in the substrate is a consequence of replacing inorganic ions intercalated between mineral layers of the clay with organic ions. The presence of the intercalated organic cations is believed to advantageously finely disperse the mineral in the material from which the substrate material of the invention may be made, e.g., a solution of polyamic acid, which is a polyimide prepolymer. The small size, packing and orientation of the organophilic clay in the film is believed to increase the film strength and the films ability to act as a heat, gas and moisture barrier, which is not feasible with ordinary filler materials.”

The term “organophilic clay” is also described in the claims of U.S. Pat. No. 6,617,020, the entire disclosure of which is hereby incorporated by reference into this specification. Claim 1 of U.S. Pat. No. 6,617,020 describes “1. A composition comprising: at least one elastomer; organophilic clay plate-like particles; and at least one non-volatile organophilic exfoliating agent; wherein the composition is a hot melt processable pressure sensitive adhesive.” Claim 5 describes the “organophilic clay plate-like particles” as comprising “ . . . organophilically modified versions of hydrated aluminum silicate, kaolinite, atapulgite, illite, bentonite, halloysite, beidelite, nontronite, hectorite, hectite, saponite, montmorillonite, and combinations thereof.” Claim 6 describes the “organophilic exfoliating agent” as comprising “ . . . a resin having a number average molecular weight of less than about 20,000 g/mol.”

The term “organophilic clay” is defined at column 2 of U.S. Pat. No. 6,617,020 as including “ . . . a clay that has been surface-modified to convert at least a portion of its surface nature from an organophobic state to an organophilic state (preferably to a hydrophobic state). For example, in one embodiment, a clay may initially have both organophobic and organophilic sites. However, upon modification according to the present invention, at least a portion of the organophobic sites are converted to organophilic sites. In other embodiments, a clay initially contains essentially only organophobic sites and, upon modification according to the present invention, at least a portion of the organophobic sites are converted to organophilic sites. Preferably, at least about 50% of exchangeable cations on the unmodified organophilic clay are exchanged with organophilic modifying cations.”

The term “organophilic exfoliating agent” is defined in column 2 of U.S. Pat. No. 6,617,020 as inlucindg “ . . . an organophilic material capable of separating an organophilic clay sheet into plate-like particles and maintaining the clay in plate-like particles at the use temperature (typically room temperature, i.e., about 21° C.).”

“Organophilic clays” and “organophilic exfoliating agents” are also described at columns 5-6 of U.S. Pat. No. 6,617,020, wherein it is disclosed that “Organophilic clay is obtainable by modifying a hydrophilic clay such that the clay is organophilic. Conventional hydrophilic clays are generally not able to be adequately exfoliated according to the present invention. Thus, the present invention utilizes organophilic clays to achieve a higher degree of exfoliation in the clay.”

U.S. Pat. No. 6,617,020 also discloses that “The hydrophilic clay to be modified can be any phyllosilicate or other clay that has a sheet-like structure. Examples thereof include, but are not limited to, hydrated aluminum silicate, kaolinite, atapulgite, illite, halloysite, beidelite, nontronite, hectorite, hectite, bentonite, saponite, and montmorillonite. The smectite clays such as, for example, beidelite, nontronite, hectorite, hectite, bentonite, saponite, and montmorillonite are preferred.”

U.S. Pat. No. 6,617,020 also discloses that “The organophilic clays useful for the invention may be prepared from commercially available hydrophilic clays. The following is an example of a method of preparing organophilic clay: The hydrophilic clay is stirred and dissolved in water to form an exfoliated hydrophilic clay solution. Then, depending on the clay, exchangeable ions (e.g., sodium or calcium ions), for example, of the hydrophilic clay are exchanged with organophilic modifying cations. Typical organophilic modifying cations comprise onium cations. For example, such cations include, but are not limited to, C2 to C60 alkyl primary, secondary, tertiary, and quaternary ammonium cations and quaternary phosphonium cations. Examples thereof include, but are not limited to, (meth)acrylate ammonium cations, such as 2-(dimethylammonium)ethyl methacrylate cations, octadecylammonium cations, dimethyl dihydrogenated tallow ammonium cations, thiol group functionalized alkyl ammonium cations, and combinations thereof. Exchange of the hydrophilic clay ions with organophilic modifying cations causes the modified clay to precipitate from the water solution. The precipitated clay (which is no longer in an exfoliated state) is then dried to remove excess water.

U.S. Pat. No. 6,617,020 also discloses that “Some organophilic clays are commercially available. For example, organophilically-modified montmorillonite is available as SCPX CLOISITE 20A, SCPX CLOISITE 15A, SCPX CLOISITE 10A, SCPX CLOISITE 6A, SCPX CLOISITE 30b, and SCPX CLOISITE 2398 from Southern Clay Products; Gonzalez, Tex., and under the trade designation, NANOMER, from Nanocor Inc.; Arlington Heights, Ill.”

U.S. Pat. No. 6,617,020 also discloses that “The composition of the invention typically comprises any suitable amount of organophilic clay. Generally, the amount of organophilic clay present is such that the overall composition is a pressure sensitive adhesive. Preferably the composition includes about 1 to about 40 weight percent of the organophilic clay plate-like particles, more preferably about 1 to about 20 weight percent, and most preferably 1 to about 10 weight percent based on the total weight of the composition. The exact amount varies depending on, for example, the type of elastomer and the presence and amount of other components in the composition.”

U.S. Pat. No. 6,617,020 also discloses that “The composition of the invention typically comprises about 1 to about 75 weight percent of a non-volatile organophilic exfoliating agent based on the total weight of the composition. A non-volatile organophilic exfoliating agent is used to exfoliate the organophilic clay. It has been found that the organophilic clay can be easily exfoliated by exfoliating agents, that are low molecular weight resins. Examples of useful low molecular weight resins include, but are not limited to, tackifying agents and low molecular weight block copolymers such as styrene-isoprene block copolymers, styrene-butadiene block copolymers, and hydrogenated block copolymers. Such exfoliating agents typically have a number average molecular weight of less than about 20,000 g/mol, preferably less than about 10,000 g/mol, and most preferably less than about 5,000 g/mol.”

U.S. Pat. No. 6,617,020 also discloses that “Tackifying agents are the preferred exfoliating agents. However, not all tackifying agents will act as an exfoliating agent in any given system. For a tackifying agent to function as an exfoliating agent according to the present invention, it generally needs to be viscous enough to impart shear forces in the composition upon exfoliation in order to effectively exfoliate the organophilic clay. It is also preferred that such a tackifying agent would minimize or prevent substantial agglomeration of the exfoliated particles. Selecting a tackifying agent in which the organophilic clay is compatible helps to accomplish this preferred embodiment. Suitable tackifying agents can be found in the following groups: aliphatic, aromatic-modified aliphatic, aromatic, and at least partially hydrogenated versions and derivatives thereof.”

U.S. Pat. No. 6,617,020 also discloses that “Examples of tackifying agents that are useful as exfoliating agents include, but are not limited to, rosins, such as wood rosins and their hydrogenated derivatives; derivatives of rosins, such as FORAL 85, a stabilized rosin ester from Hercules Chemical Co.; Wilmington, Del., the SNOWTACK series of gum rosins from Tenneco Corp.; Greenwich, Conn., and the AQUATAC series of tall oil rosins from Arizona Chemical Co.; Panama City, Fla.; terpene resins of various softening points, such as .alpha.-pinene and β-pinene, available as PICCOLYTE A-115 and ZONAREZ B-100 from Arizona Chemical Co.; Panama City, Fla.; petroleum-based resins, such as the ESCOREZ 1300 series of aliphatic olefin-derived resins and the ESCOREZ 2000 series of aromatic/aliphatic olefin-derived resins from Exxon Chemical Co.; Houston, Tex.; and synthetic hydrocarbon resins, such as the PICCOLYTE A series of aromatic resins such as PICCOTEX LC-55WK; and aliphatic resins, such as PICCOTAC 95, available from Hercules Chemical Co.; Wilmington, Del.”

U.S. Pat. No. 6,617,020 also discloses that “Particularly preferred are resins derived by polymerization of C5 to C9 unsaturated hydrocarbon monomers, polyterpenes, synthetic polyterpenes and the like. Examples of such commercially available resins of this type are WINGTACK PLUS tackifying agents, available from Goodyear Tire and Rubber Co.; Akron, Ohio; REGALREZ 1126 tackifying agents, available from Hercules Chemical Co.; Wilmington, Del.; and ESCOREZ 180, ESCOREZ 1310, and ESCOREZ 2393 tackifying agents, all available from Exxon Chemical Co.; Houston, Tex.”

In one preferred embodiment, the synthetic clay mineral is clay bridged with a metal compound, as that term is defined in U.S. Pat. No. 6,674,009, the entire disclosure of which is hereby incorporated by reference into this specification. As is disclosed in such patent, and as is described in claim 3 thereof, the bridged clay may be selected from the group consisting of “ . . . montmorillonite, laponite, beidellite, nontronite, saponite, sauconite, hectorite, stevensite, kaolinite, halloysite, vermiculite, and sepiolite, or one of their synthetic or naturally interstratified mixtures . . . . ” As is disclosed at column 2 of this patent, “The starting clay treated with a solution of a salt of a metallic compound, preferably a solution of iron and/or aluminum salt. After drying and heat treatment, a bridged clay is obtained.”

In one preferred embodiment, the synthetic clay mineral used in the process of this invention is an organophilic layer silicate as that term is defined in U.S. Pat. No. 6,683,122, the entire disclosure of which is hereby incorporated by reference into this specification. Claim 1 of this patent describes “1. A filler mixture comprising an (a) organophilic layer silicate obtainable by treatment of a natural or synthetic layer silicate with a swelling agent selected from the group consisting of sulfonium, phosphonium and ammonium compounds (salts of melamine compounds and cyclic amidine compounds being excluded as ammonium compounds); and (b) a mineral filler different from component (a).” Claim 2 of this patent “2. A filler mixture according to claim 1, wherein the natural or synthetic layer silicate is selected from the group consisting of bentonite, vermiculite, halloysite, saponite, beidellite, nontronite, hectorite, sauconite, stevensite and montmorillonite.” This filler mixture is described at columns 1-3 of U.S. Pat. No. 6,683,122, wherein it is disclosed that “The preparation of organophilic layer silicates by treatment of layer silicates with onium salts, e.g. quaternary ammonium salts, in the presence of a dispersion medium is known from U.S. Pat. No. 4,810,734. In that treatment an exchange of ions takes place, the cation of the onium salt being inserted into the interlayer space of the layer silicate. Layer silicates modified in that manner become organophilic as a result of the organic radical of the inter-calated amine. When that organic radical contains functional groups, the organophilic layer silicate is capable of forming chemical bonds with suitable monomers or polymers.”

U.S. Pat. No. 6,683,122 also discloses that “WO 96/08526 describes the use of such organophilic layer silicates as filler materials for epoxy resins, there being obtained nanocomposites having improved physical and mechanical properties. It is of special interest that there is an increase in rigidity while the toughness at least remains the same. Especially good properties are exhibited by nano-composites that contain the layer silicate in exfoliated form. However, the addition of such organophilic layer silicates gives rise not only to an improvement in rigidity but also to a reduction in tensile strength.”

U.S. Pat. No. 6,683,122 also discloses that “It has been found, surprisingly, that a combination of organophilic layer silicates and mineral fillers can yield considerably better mechanical properties than the individual components. In thermosetting resins, the addition of the filler mixtures according to the invention results in a considerable increase in rigidity as compared with the use of pure mineral fillers at the same total filler content, while the substantial reduction in tensile strength which occurs when organophilic layer silicates are used alone is prevented. The filler mixtures according to the invention therefore allow the preparation of filled resins which, while having a relatively low filler content, have good mechanical properties and can be processed without problems. By varying the mixing ratio of mineral filler to organophilic layer silicate it is possible to obtain tailored system-specific property profiles.”

U.S. Pat. No. 6,683,122 also discloses that “The present invention relates to a filler mixture comprising an organophilic layer silicate obtainable by treatment of a natural or synthetic layer silicate with a swelling agent selected from sulfonium, phosphonium and ammonium compounds (salts of melamine compounds and cyclic amidine compounds being excluded as ammonium compounds) and a mineral filler different therefrom.”

U.S. Pat. No. 6,683,122 also discloses that “As layer silicates for the preparation of the organophilic layer silicates of the filler mixtures according to the invention there come into consideration especially natural and synthetic smectite clay minerals, more especially bentonite, vermiculite, halloysite, saponite, beidellite, nontronite, hectorite, sauconite, stevensite and montmorillonite. Montmorillonite and hectorite are preferred.”

U.S. Pat. No. 6,683,122 also discloses that “The layer silicate montmorillonite, for example, corresponds generally to the formula Al2[(OH)2/Si4O10].nH2O, it being possible for some of the aluminum to have been replaced by magnesium. The composition varies according to the silicate deposit. A preferred composition of the layer silicate corresponds to the formula (Al3.15Mg0.85)Si8.00O20(OH)4X11.8.nH2O, wherein X is an exchangeable cation, generally sodium or potassium, and some of the hydroxyl groups may have been replaced by fluoride ions. By exchanging hydroxyl groups for fluoride ions, synthetic layer silicates are obtained.”

U.S. Pat. No. 6,683,122 also discloses that “The sulfonium, phosphonium and ammonium compounds required as swelling agents for the preparation of the organophilic layer silicates are known and some of them are commercially available. They are generally compounds having an onium ion, for example trimethylammonium, trimethylphosphonium and dimethylsulfonium, and a functional group that is capable of reacting or bonding with a polymeric compound. Suitable ammonium salts can be prepared, for example, by protonation or quaternisation of corresponding aliphatic, cycloaliphatic or aromatic amines, diamines, polyamines or aminated polyethylene or polypropylene glycols (Jeffamine® M series, D series or T series).”

U.S. Pat. No. 6,683,122 also discloses that “Special preference is given to layer silicates in which the layers have a layer spacing of about from 0.7 nm to 1.2 nm and which have a cation exchange capacity in the region of 50 to 200 meq./100 g (milliequivalents per 100 grams). After treatment with the swelling agent (sulfonium, phosphonium or ammonium compound), the layer spacing in the organophilic layer silicates so obtained is preferably at least 1.2 nm. Such layer silicates are described, for example, in A. D. Wilson, H. T. Posser, Developments in Ionic Polymers, London, Applied Science Publishers, Chapter 2, 1986. Synthetic layer silicates can be obtained, for example, by reaction of natural layer silicates with sodium hexafluorosilicate and are commercially available inter alia from the CO-OP Chemical Company, Ltd., Tokyo, Japan.”

U.S. Pat. No. 6,683,122 also discloses that “For the preparation of the organophilic layer silicates, the swelling agent is first advantageously dispersed or dissolved, with stirring, in a dispersion medium, preferably at elevated temperature of about from 40° C. to 90° C. The layer silicate is then added and dispersed, with stirring. The organophilic layer silicate so obtained is filtered off, washed with water and dried. It is, of course, also possible to prepare the dispersion of the layer silicate as initial batch and then to add the solution or dispersion of the swelling agent.”

U.S. Pat. No. 6,683,122 also discloses that “Suitable dispersion media are water, methanol, ethanol, propanol, isopropanol, ethylene glycol, 1,4-butanediol, glycerol, dimethyl sulfoxide, N,N-dimethylformamide, acetic acid, formic acid, pyridine, aniline, phenol, nitrobenzene, acetonitrile, acetone, 2-butanone, chloroform, carbon disulfide, propylene carbonate, 2-methoxyethanol, diethyl ether, tetrachloromethane and n-hexane. Preferred dispersion media are methanol, ethanol and especially water.”

U.S. Pat. No. 6,683,122 also discloses that “The swelling agent brings about a widening of the interlayer spacing of the layer silicate, so that the layer silicate is able to take up monomers into the interlayer space. The subsequent polymerization, polyaddition or polycondensation of the monomer or monomer mixture results in the formation of a composite material, a nanocomposite.”

U.S. Pat. No. 6,683,122 also discloses that “In the filler mixtures according to the invention it is preferable to use layer silicates that have been pre-treated with a polymerizable monomer prior to swelling. When the swelling is complete, the compositions are polymerized. Such monomers are, for example, acrylate monomers, methacrylate monomers, caprolactam, laurinlactam, aminoundecanoic acid, aminocaproic acid or aminododecanoic acid. The resin component or the hardener component of an epoxy resin system or the components of a polyurethane system can likewise be such monomers.”

U.S. Pat. No. 6,683,122 also discloses that “Suitable mineral fillers that can be used in the filler mixtures according to the invention are, for example, glass powder, glass beads, semi-metal and metal oxides, e.g. SiO2 (aerosils, quartz, quartz powder, fused silica), corundum and titanium oxide, semi-metal and metal nitrides, e.g. silicon nitride, boron nitride and aluminum nitride, semi-metal and metal carbides (SiC), metal carbonates (dolomite, chalk, CaCO3), metal sulfates (barite, gypsum), powdered minerals and natural or synthetic minerals primarily from the silicate series, e.g. talcum, mica, kaolin, wollastonite etc. It is also possible to use the untreated layer silicates that are used for the preparation of organophilic layer silicates.”

U.S. Pat. No. 6,683,122 also discloses that “Preferred mineral fillers are quartz powder, mica, kaolin, wollastonite, chalk and talcum.”

U.S. Pat. No. 6,683,122 also discloses that “The quantity ratio of the components can vary within wide limits according to the property profile desired in the filler mixtures according to the invention.”

U.S. Pat. No. 6,683,122 also discloses that “Preference is given to filler mixtures in which the proportion of organophilic layer silicate is from 1.0 to 60.0% by weight and the proportion of mineral filler is from 40.0 to 99.0% by weight.”

U.S. Pat. No. 6,683,122 also discloses that “In especially preferred filler mixtures, the proportion of organophilic layer silicate is from 2.0 to 50.0% by weight, especially from 4.0 to 30.0% by weight, and the proportion of mineral filler is from 50.0 to 98.0% by weight, especially from 70.0 to 96.0% by weight.”

U.S. Pat. No. 6,683,122 also discloses that “The filler mixtures according to the invention can be prepared prior to application in customary manner by mixing the components together using known mixing apparatus (e.g. stirrers, rollers).”

U.S. Pat. No. 6,683,122 also discloses that “It is also possible, however, to incorporate one of the components into the resin or into one of the resin components and then to add the other component prior to the polymerization or curing.”

U.S. Pat. No. 6,683,122 also discloses that “In one embodiment, the synthetic clay mineral used is an organoclay, as that term is described in U.S. Pat. No. 6,831,123, the entire disclosure of which is hereby incorporated by reference into this specification. Claim 1 of this patent describes “A composition comprising at least one ionomeric polyester resin and at least one organoclay, wherein the organoclay is not preswollen before combination with ionomeric polyester resin.” The organoclay is further described in claim 10, which recites that “10. The composition of claim 1 wherein the organoclay comprises at least one member selected from the group consisting of kaolinite, hafloysite, dickite, nacrite, montmorillonite, nontronite, beidellite, hectorite, saponite, hydromicas, phengite, brammallite, glaucomite, celadonite, kenyaite, magadite, bentonite, stevensite, muscovite, sauconite, vermiculite, volkonskoite, laponite, mica, fluoromica, and smectite.” These organoclays are further described in column 1 of such United States patent, wherein it is disclosed that “Organoclays typically consist of particles comprised of several layers of alumino-silicate plates held together by electrostatic interactions with organic moieties containing metal cations or alkyl ammonium ions intercalated between the plates. Such clays have been used as fillers in resinous compositions. In certain cases they may increase properties such as heat resistance, and/or mechanical strength, or they may beneficially decrease properties such as electrical conductivity or permeability to gases such as oxygen or water vapor.”

U.S. Pat. No. 6,831,122 also discloses that “The benefit of organoclays over other mineral fillers in resinous compositions is obtained when the alumino-silicate plates comprising the clay are separated from one another and dispersed in the polymer matrix. Since these plates have a very high aspect ratio, they may provide property enhancement such as reinforcement and improvement in modulus compared to traditional mineral fillers on a per weight of total inorganic content. In order to separate the layers of the clay and obtain maximum reinforcement in a resinous composition, it is typically necessary that polymer adsorb between the layers of the clay causing exfoliation (separation) of the layers. Typically, hydrophilic polymers such as polyamides or water-soluble polymers have been used in compositions with organoclays since they may have an affinity for the clay surface promoting exfoliation. It has been found, however, that intimately mixing typically hydrophobic polyester resins and organoclays does not allow for full exfoliation of the clay. Thus properties of the compositions such as modulus may be only marginally better than those properties obtained when traditional fillers are used in typical polyester resins. There is a need to prepare compositions of normally hydrophobic polyester resins with organoclay fillers which achieve optimum beneficial property improvement.”

U.S. Pat. No. 6,831,123 also discloses that “PCT Patent Application WO 99/32403 suggests the preparation of an expanded organoclay using a sulfonated polyester as an expanding agent. Following the expansion step, the expanded organoclay is combined in a separate step with a non-ionomeric polyester resin to form a composition with up to 30 weight % expanded organoclay, the clay containing 20 to 80 weight % expanding agent.”

The organoclays of U.S. Pat. No. 6,831,123 are also described at columns 5-6 of such patent, wherein it is disclosed that “The compositions of the present invention contain at least one organoclay. As used herein, “organoclay” comprises a layered clay, usually a silicate clay, typically derived from a layered mineral and in which organic moieties have been chemically incorporated, ordinarily by ion exchange and especially cation exchange with organic-containing ions and/or onium compounds. Illustrative organic ions are mono- and polyammonium cations such as trimethyldodecylammonium and N,N′-didodecylimidazolium.”

U.S. Pat. No. 6,831,123 also discloses that “There is no particular limitation with respect to the layered clays that may be employed in this invention other than that they are capable of undergoing cation exchange with cations and/or onium compounds comprising organic moieties to produce organoclays, and in the form of organoclays they are capable of producing an increase in modulus in a composition containing an ionomeric polyester resin compared to a similar composition containing essentially the same non-ionomeric polyester resin. Illustrative of such layered clays that may be employed in this invention include, for instance, smectite and those of the kaolinite group such as kaolinite, halloysite, dickite, nacrite and the like.”

U.S. Pat. No. 6,831,123 also discloses that “The layered clays are preferably natural or synthetic phyllosilicates, particularly smectic clays. Illustrative examples include, for instance, halloysite, montmorillonite, nontronite, beidellite, saponite, volkonskoite, laponite, sauconite, magadite, kenyaite, bentonite, stevensite, and the like. It is also within the scope of the invention to employ organoclays comprising minerals of the illite group, including hydromicas, phengite, brammallite, glaucomite, celadonite and the like. Often, the preferred layered minerals include those often referred to as 2:1 layered silicate minerals, including muscovite, vermiculite, saponite, hectorite and montmorillonite, the latter often being most preferred. The clays may be synthetically produced, but most often they comprise naturally occurring minerals and are commercially available. Mixtures of clays for example as described above are also suitable. A more detailed description of suitable clays can be found in U.S. Pat. No. 5,530,052, the disclosure of which is incorporated by reference herein.”

U.S. Pat. No. 6,831,123 also discloses that “It is also within the scope of the instant invention to include layered minerals which are classified as layered double hydroxides, as well as layered minerals having little or no charge on their layers provided that they are capable of undergoing cation exchange with cations and/or onium compounds comprising organic moieties to produce organoclays, and in the form of organoclays they are capable of producing an increase in modulus in a composition containing an ionomeric polyester resin compared to a similar composition containing essentially the same non-ionomeric polyester resin.”

U.S. Pat. No. 6,831,123 also discloses that “In addition to the clays mentioned above, admixtures prepared therefrom may also be employed as well as accessory minerals including, for instance, quartz, biotite, limonite, hydrous micas, fluoromicas, feldspar and the like.”

U.S. Pat. No. 6,831,123 also discloses that “Preferred layered clays comprise particles containing a plurality of silicate platelets having a thickness of about 7-15 .Angstroms. bound together at interlayer spacings of about 4 .Angstroms. or less, and containing exchangeable cations such as Na+, Ca+2, K+, Al+3, and/or Mg+2 present at the interlayer surfaces. They typically have a cation exchange capacity of about 50-200 milliequivalents per 100 grams.”

U.S. Pat. No. 6,831,123 also discloses that “The layered clay is cation exchanged with organic-containing ions and/or onium compounds to produce organoclay. Suitable organic-containing ions and/or onium compounds include ammonium cations, pyridinium cations, phosphonium cations, or sulfonium cation represented, respectively, by the general formulas NHx Ry+, PyR+, PyR+, and SR2+, wherein R is an aromatic group, an alkyl group, an aralkyl group, or a mixture thereof, and the sum of x and y is 4; preferably R is an alkyl group. Other suitable organic-containing ions and/or onium compounds include protonated amino acids and salts thereof containing about 2-30 carbon atoms. Other examples of suitable organic-containing ions and/or onium compounds and processes for employing them are disclosed in U.S. Pat. Nos. 4,810,734; 4,889,885; and 5,530,052 which are incorporated herein by reference.”

U.S. Pat. No. 6,831,123 also discloses that “Suitable specific commercially available or easily prepared organoclays which are illustrative of those which may be employed include CLAYTONE HY, a montmorillonite which has been cation exchanged with dimethyldi(hydrogenated tallow)ammonium ion available from Southern Clay Products, and montmorillonite which has been cation exchanged with such ions as dodecylammonium, trimethyldodecylammonium, N,N′-didodecylimidazolium, N,N′-ditetradecylbenzimidazolium, methyl bis(hydroxyethyl)(hydrogenated tallow)ammonium, or methyl bis(2-hydroxyethyl)octadecylammonium.”

U.S. Pat. No. 6,831,123 also discloses that “The compositions of the invention may also contain conventional additives. Suitable additives include flame retardants, anti-drip agents, stabilizers, resinous impact modifiers, other fillers such as extending fillers, pigments, dyes, antistatic agents, crystallization aids and mold release agents. Since these are well known in the art, they will not be dealt with in detail herein.”

Preparation of a Composite Containing Nanomaenetic Material and Mineral Material

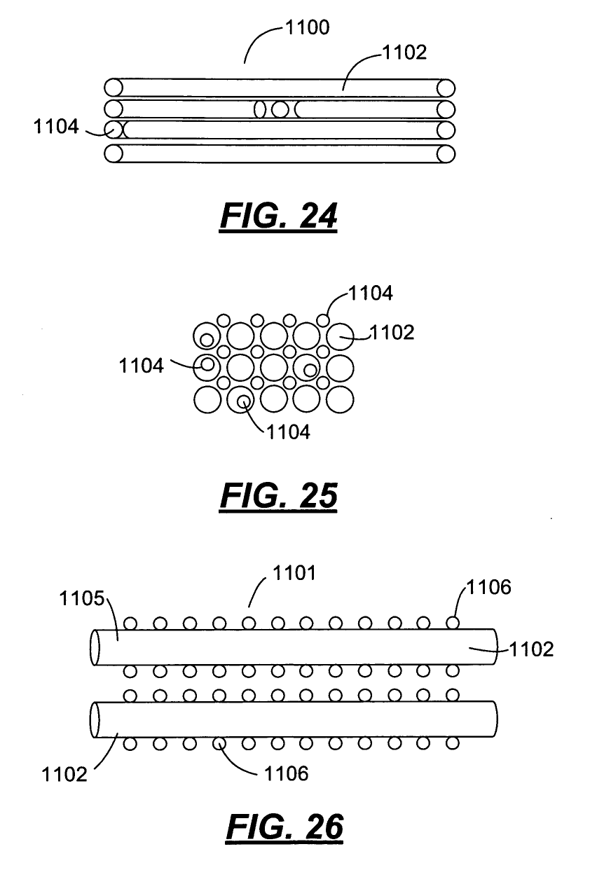

FIG. 24 is a schematic illustration of a nanocomposite assembly 1100 comprised of tubules 1102 and granular material 1104. These tubules 1102, and their properties, are described elsewhere in this specification and in U.S. Pat. Nos. 4,877,501 (process for fabrication of lipid microstrucutres), U.S. Pat. No. 4,911,981 (metal clad lipid microstrucutres), U.S. Pat. No. 5,049,382 (cating and composition containing lipid microstructure toxin dispenses), U.S. Pat. No. 5,492,696 (controlled release microstructures), U.S. Pat. No. 5,651,976 (controlled release of active agents using inorganic tubules), U.S. Pat. No. 5,705,191 (sustained delivery of active compounds from tubules, with rational control), U.S. Pat. No. 5,744,337 (internal gelation method for forming multilayer microspheres), U.S. Pat. No. 6,013,206 (formation of high aspect ratio lipid microtubules), U.S. Pat. No. 6,280,759 (method of controlled release and controlled release microstructures), and the like. The entire disclosure of each of these United States patent is hereby incorporated by reference into this specification.

In one preferred embodiment, the tubules 1102 are inorganic tubules, and the granular material 1104 is inorganic granular material. In one aspect of this embodiment, the inorganic tubules are halloysite tubules.

FIG. 25 is a sectional view of the nanocomposite assembly 1100 of FIG. 24, showing the granular material 1104 disposed both between the tubules 1102 as well as within the tubules 1102. In another embodiment, not shown, the granular material 1104 is disposed between the tubules 1102 but not within the tubules 1102. In yet another embodiment, not shown, the granular material 1104 is disposed within the tubules 1102 but not between the tubules 1102.

When the tubular material 1100 is mined (such as, e.g., when halloysite is ined), it generally contains from about 5 to about 95 weight of tubular material 1102; and it often contains from about 5 to about 50 weight percent of tubular material 1102. However, as is well-known to those skilled in the art, the as-mined mineral (such as, e.g., as mined halloysite) may be purified to increase its concentration of the tubular form 1102 of the mineral.

It is preferred that the as-mined mineral matter be purified by conventional means to concentrate the long tubules 1102. Such conventional means may include, e.g., electrostatic means, ultrasonic means, centrifugal means, and/or sieving.

As is known to those skilled in the art, halloysite has been obtained that contains at least 95 weight percent of the tubular form 1102. Reference may be had, e.g., to tubular halloysite from Yunnan China and, in particular, to a photograph thereof that appears in the “Mineral Gallery” of the Clay Minerals Group of the Mineralogical Society. This photograph was published on the website of the Mineralogical Society (www.minersoc.org). Information about it may be obtained from the Secretary of the Mineralogical Society, Dr. Steve Hillier, Secretary of the Clay Minerals Group, Environmental Science Group, Macaulay Institute, Craigi9ebuckler, Aberdeen, AB15 8QH. Scotland.

In one preferred embodiment, the composition 1100 (see FIGS. 24 and 25) contain at least 80 weight percent of the tubules 1102 and, more preferably, at least 90 weight percent of the tubules 1102. In one aspect of this embodiment, the composition 1100 contains at least 95 weight percent of tubules 1102.

FIG. 26 is a schematic illustration of a composition 1101 that is comprised of such tubules 1102 and, coated on the outer surfaces 1105 thereof, a multiplicity of particles of nanomagnetic material 1106; this nanomagnetic material 1106, and means for its preparation and coating onto the tubules 1102, are described elsewhere in this specification. In one aspect of this embodiment, the tubules 1102 are halloysite microtubules. In this aspect, it is preferred to incorporate the composition comprised of halloysite tubules into one or more of the polymeric, resinous, elastomeric, and/or ceramic compositions described elsewhere in this specification.

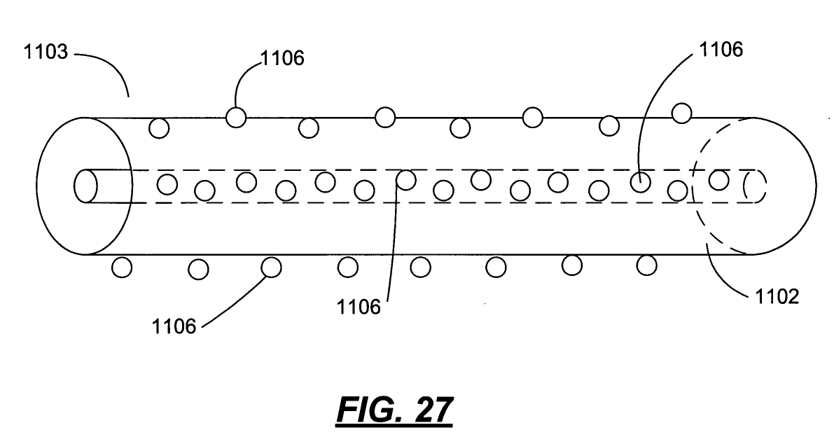

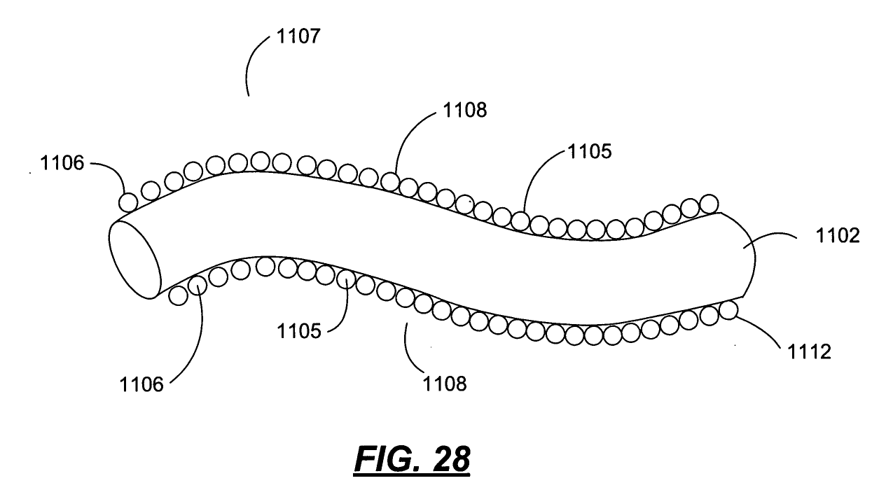

FIG. 27 is a schematic illustration of a tubule assembly 1103 comprising a tubules 1102 onto which and into which nanomagnetic material 1106 has been incorporated. Such incorporation of the nanomagnetic material into the microtubule 1102 may be done by conventional means. Reference may be had, e.g., to U.S. Pat. No. 4,877,501 (process for fabrication of lipid microstrucutres), U.S. Pat. No. 4,911,981 (metal clad lipid microstrucutres), U.S. Pat. No. 5,049,382(cating and composition containing lipid microstructure toxin dispenses), U.S. Pat. No. 5,492,696 (controlled release microstructures), U.S. Pat. No. 5,651,976 (controlled release of active agents using inorganic tubules), U.S. Pat. No. 5,705,191 (sustained delivery of active compounds from tubules, with rational control), U.S. Pat. No. 5,744,337 (internal gelation method for forming multilayer microspheres), U.S. Pat. No. 6,013,206 (formation of high aspect ratio lipid microtubules), U.S. Pat. No. 6,280,759 (method of controlled release and controlled release microstructures), and the like. The entire disclosure of each of these United States patent is hereby incorporated by reference into this specification.

In the embodiment depicted in FIG. 28, the tubule 1102 is coated with a multiplicity of nano-sized particles 1106 (such as, e.g., nanomagnetic particles that are smaller than about 100 nanometers and, more preferably, smaller than about 50 nanometers). In the embodiment depicted, the nanomagnetic particles 1106 adhere to both themselves and to the tubules 1102, thereby forming a continuous film 1108 on the outer surface of the tubule 1102.

The continuous film 1108 on the outer surface 1105 of the tubule 1102 provides several distinct advantages. In addition to providing adaptive shielding (discussed later in this specification) and potentially modifying the thermal characteristics of such tubule 1102, it also improves the mechanical properties of such tubule 1102.

The film 1108 of nanomagnetic particles 1106 preferably has a surface roughness of less than about 50 nanometers and, more preferably, less than about 10 nanometers. As is known to those skilled in the art, the average surface roughness of a thin film is preferably measured by an atomic force microscope (AFM). Reference may be had, e.g., to U.S. Pat. No. 5,420,796 (method of inspecting planarity of wafer surface), U.S. Pat. Nos. 6,610,004, 6,140,014, 6,548,139, 6,383,404, 6,586,322, 5,832,834, and 6,342,277. The entire disclosure of each of these United States patents is hereby incorporated by reference into this specification. Reference may also be had to the discussion of surface roughness that appears elsewhere in this specification.

Referring again to FIG. 28, a the coated tubules 1107 comprised of a continuous film 1108 of nanomagnetic particles 1106 on outer surface 1105 have improved compressive strength and flexural strength properties. When a composition comprised of at least 80 weight percent of such coated tubules 1107 (and, preferably, at least about 90 weight percent of such coated tubules 1107) is tested in accordance with the procedure described in U.S. Pat. No. 6,290,771, the compressive strength obtained is at least 2,000 kilograms per square centimeter, and the flexural strength obtained is at least about 200 kilograms per square centimeter.

U.S. Pat. No. 6,290,771 describes an “Activated kaolin powder compound for mixing with cement . . . ;” the entire disclosure of this United States patent is hereby incorporated by reference into this specification. In Examples 1 and 2 of this patent, a description is presented of a method for determining the compressive strength and the flexural strength of various mineral compositions.

The “Example 1” of U.S. Pat. No. 6,290,771 appears at column 7 of such patent. It discloses that “Cement of 450 g, activated kaolin of 50 g, sands of 1,500 g, water of 250 g and superplasticizer of 5 g were mixed together. Specimens of mortar of 40×40×160 mm were prepared from the mixture. The specimens were wet-cured in a 3-in-1 mold for 24 hours, and water-cured for 28 days. Three specimens (Specimens I, II and III) were prepared.”

The “Comparative Example 1” of U.S. Pat. No. 6,290,771 was also disclosed at such column 7. In such column 7, it was stated that “A conventional mortar was prepared. Cement of 500 g, sands of 1,500 g, water of 250 g and superplasticizer of 5 g were mixed together. Specimens of mortar of 40×40×160 mm were prepared from the mixture. The specimens were wet-cured in a 3-in-1 mold for 24 hours, and water-cured for 28 days. Three specimens (Specimens I, II and III) were prepared.”

The “Comparative Example 2” of U.S. Pat. No. 6,290,771 also appeared in such column 7, wherein it was disclosed that “Comparative Example 2 was performed as in Example 1 with the exceptions that unactivated kaolin of 50 g was employed instead of the activated kaolin of 50 g. Three specimens (Specimens I, II and III) were prepared . . . . Flexural strength, compressive strength and water permeability were measured for the specimens of Example 1 and Comparative Examples 1 and 2 . . . . Flexural strengths were measured according to Korean Industrial Standard KS L 5105. The distance of points was 100 mm and the applied force was 5 kg.multidot.force per second. The strengths of the specimens were shown in Table 6.”

The results of these experiments were discussed at columns 7-8 of U.S. Pat. No. 6,290,771, wherein it was disclosed that “As shown in Table 6, the mortar according to the present invention (Example 1) has an increase of 14.9% of the conventional mortar (Comparative Example 1) in flexural strength. The mortar using unactivated kaolin (Comparative Example 2) shows a decrease of 27.3% of the conventional mortar (Comparative Example 1) in flexural strength.”

U.S. Pat. No. 6,290,771 also discloses that (in column 7) “Compressive strengths were measured according to KS L 5105. The applied force was 80 kg.multidot.force per second. After measurement of the flexural strength, six specimens per Example were tested. The compressive strengths of the specimens were shown in Table 7 . . . . As shown in Table 7, the mortar according to the present invention (Example 1) has an increase of 25.8% of the conventional mortar (Comparative Example 1) in compressive strength. The mortar using unactivated kaolin (Comparative Example 2) shows a decrease of 8.9% of the conventional mortar (Comparative Example 1) in compressive strength.”

The best flexural strength obtainable in the experiments reported in U.S. Pat. No. 6,290,771 was 89.1 kilograms per square centimeter (see Table 6, Example 1). By comparison, when the experiments of U.S. Pat. No. 6,290,771 are repeated using 50 grams of a composition that contains at least 80 weight percent of the tubules 1107 (see FIG. 28), the flexural strength obtained is at least 200 kilograms per square centimeter. In one embodiment, the flexural strength so obtained is at least 300 kilograms per square centimeter. The term “flexural strength,” as used in this specification (and in the claims of this case), refers to the value obtained when 50 grams of the composition in question is used in the test specified in U.S. Pat. No. 6,290,771.

The best compressive strength obtainable in the experiments reported in U.S. Pat. No. 6,290,771 was 958 kilograms per square centimeter (see Table 7, Example 1). By comparison, when the experiments of U.S. Pat. No. 6,290,771 are repeated using 50 grams of a composition that contains at least 80 weight percent of the tubules 1107 (see FIG. 28), the compressive strength obtained is at least 2,000 kilograms per square centimeter. In one embodiment, the flexural strength so obtained is at least 3,000 kilograms per square centimeter. The term “compressive strength,” as used in this specification (and in the claims of this case), refers to the value obtained when 50 grams of the composition in question is used in the test specified in U.S. Pat. No. 6,290,771.

In addition to improving the physical properties of the tubules 1102, the coating/film 1108 also improves the shielding properties of a composition that contains at least 80 weight percent of the tubules 1107. Such a composition has shielding factor of at least 0.5 and, preferably, at least about 0.9.

As is discussed elsewhere in this specification, and referring again to FIG. 6 of U.S. Pat. No. 6,713,671 (the entire disclosure of which is hereby incorporated by reference into this specification), a film 104 is adapted to reduce the magnetic field strength at point 108 (which is disposed less than 1 centimeter above film 104) by at least about 50 percent. Thus, if one were to measure the magnetic field strength at point 108, and thereafter measure the magnetic field strength at point 110 (which is disposed less than 1 centimeter below film 104), the latter magnetic field strength would be no more than about 50 percent of the former magnetic field strength. Put another way, the film 104 has a magnetic shielding factor of at least about 0.5.

Referring again to FIG. 6 of U.S. Pat. No. 6,713,671, in one embodiment, the film 1108 (see FIG. 28) has a magnetic shielding factor of at least about 0.9, i.e., the magnetic field strength at “point 110” is no greater than about 10 percent of the magnetic field strength at “point 108”.

Referring again to FIG. 28, the film 1108 engages in “adaptive shielding,” i.e., it changes its electrical properties as it senses electromagnetic radiation.

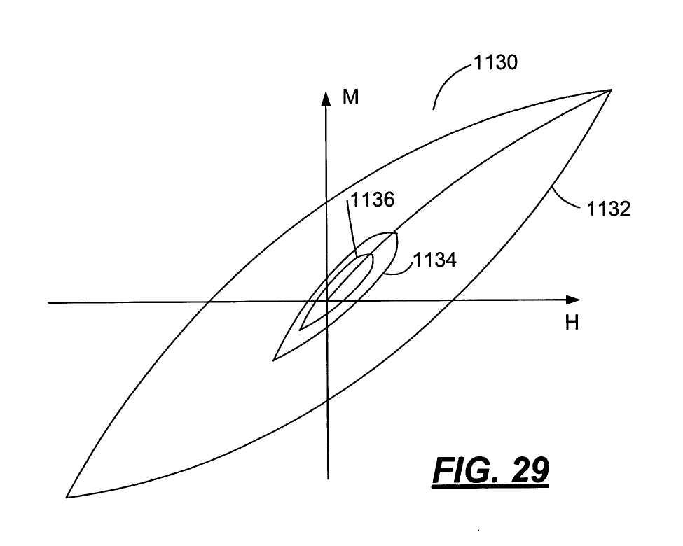

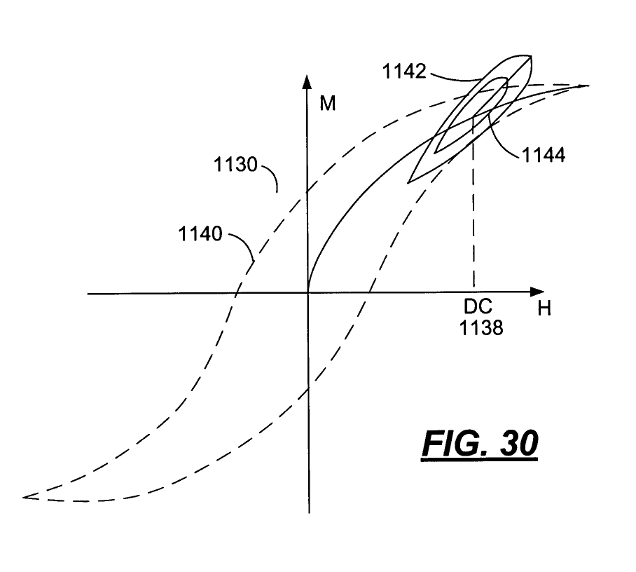

FIGS. 29 and 30 illustrate why this “adaptive shielding” occurs. FIG. 29 illustrates the response of a coating 1108 in response to an alternating current electromagnetic field. FIG. 30 illustrates the response of such coating 1108 to both an alternating current electromagnetic field and a direct current magnetic field 1138.

Referring to FIG. 29, when there is no direct current magnetic field 1138, you will produce a hysteresis loop 1130 that is comprised of a set of minor loops 1132, 1134, and 1136. When a d.c. magnetic field 1138 is also present (see FIG. 30), you will obtain a major loop 1140 and minor loops 1142 and 1144.

As will be apparent to those skilled in the art, the slope of the curve(s) obtained is the susceptibility, and it will vary depending upon the value of the applied alternating current field and the applied direct current field.

As the slopes of the curves change, as the susceptibility changes, the magnetization changes; and as the magnetization of the coating 1108 changes, the electromagnetic properties of the coating 1108 changes.

Thus, the electromagnetic properties of the coating 1108 will depend, at least in part, on the properties and intensity of the a.c. fields and/or d.c. fields to which it is exposed. It will also depend, in part, on the concentrations of the “A”, “B”, and “C” moieties discussed elsewhere in this specification and with reference to U.S. Pat. No. 6,765,144 (see FIG. 37), the entire disclosure of which is hereby incorporated by reference into this specification.

A Stent that can be Visualized by Magnetic Resonance Imaging

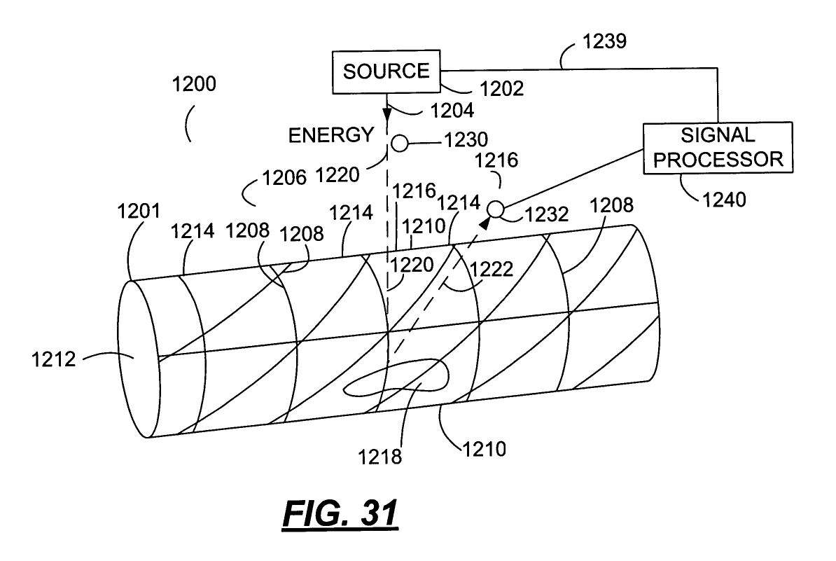

FIG. 31 is a schematic illustration of a stent assembly 1200 that can be readily visualized by magnetic resonance imaging. The stent assembly 1200 preferably contains a metallic stent 1201.

As used in this specification, the term “metallic stent” refers to a stent that is comprised of at least about 80 weight percent of metallic material and, preferably, at least about 90 weight percent of metallic material. Reference may be had, e.g., to U.S. Pat. Nos. 5,562,922; 5,665,103; 5,830,179 (urological stent therapy system); U.S. Pat. No. 5,843,172 (porous medicated stent); U.S. Pat. Nos. 6,027,811; 6,159,237 (implantable vascular and endoluminal stents); U.S. Pat. Nos. 6,174,305; 6,187,054; 6,238,421 (method for metallic implants in living beings); U.S. Pat. No. 6,403,635 (method of treating atherosclerosis or restenosis using microtubule stabilizing agent); U.S. Pat. No. 6,468,300 (stent covering heterologous tissue); U.S. Pat. No. 6,569,104 (Ni—Ti—W alloy); U.S. Pat. Nos. 6,605,109; 6,626,940; 6,679,980 (apparatus for electropolishing a stent); U.S. Pat. No. 6,712,844 (MRI compatible stent); U.S. Pat. Nos. 6,730,120; 6,753,071; 6,776,795 (Ni—Ti—W alloy), and the like. The entire disclosure of each of these United States patents is hereby incorporated by reference into this specification.

Metallic materials are described, e.g., at pages 522-523 of George S. Brady et al.'s “Materials Handbook,” Thirteenth Edition (McGraw-Hill, Inc., New York, New York, 1991). As is disclosed in this text, “About three-quarters of the elements available can be classified as metals . . . . Although the word metal, by strict definition, is limited to the pure metal elements, common usage gives it wider scope to include metal alloys. While pure metallic elements have a broad range of properties, they are quite limited in commercial use. Metal alloys, which are combinations of two or more elements, are far more versatile and for this reason are the form in which most metals are used by industry.”

As is also disclosed in the Brady et al. text, “Metallic materials are crystalline solids. Individual crystals are composed of unit cells repeated in a regular pattern to form a three-dimensional crystal lattice structure. A piece of metal is an aggregate of many thousands of interlocking crystals (grains) immersed in a cloud of negative valence electrons detached from the crystals' atoms. These loose electrons serve to hold the crystal structures together because of their electrostatic attraction to the positively charged metal atoms (ions). The bonding forces, being large because of the close-packed nature of metallic crystal structures, account for the generally good mechanical properties of metals. Also, the electron cloud makes most metals good conductors of heat and electricity.”

The Brady et al. work also discloses that “There are two families of metallic materials—ferrous and non-ferrous. The basic ingredient of all ferrous metals is the element iron. These metals range from cast irons and carbon steels, with over 90% iron, to specialty iron alloys, containing a variety of other elements that add up to nearly half the total composition.”

Several metallic stents are described in Patrick W. Serruys et al.'s “Handbook of Coronary Stents,” Fourth Edition (Martin Dunitz Ltd., London, England, 2002). These metallic stents may comprise stainless steel (ARTHOS stent), 316L stainless steel (ANTARES STARFLEX stent), 316L stainless steel coated with phosphorylcholine (BIODIVYSIO stent), 316 LVM stainless steel (SIRIUS stent), 316 L medical grade stainless steel coated with DYLYN(DYLYN stent), 316 stainless steel, polytetrafluoroethylene(JOSTENT stent), Nitinol (JOSTENT BIFLEX stent), niobium alloy coated with indium oxide (LUNAR stent), 316 LVM stainless steel (NEXUS stent), stainless steel plated with gold (NIROYAL stent), 316L stainless steel coated with hypothombogenenic a-SiC:H (RITHRON stent), and the like.

Referring to FIG. 31, and in the preferred embodiment depicted therein, it will be seen that stent assembly 1200 is comprised of a source 1202 of energy 1204.

In one preferred embodiment, the energy 1204 is energy typically emitted by a magnetic resonance imaging (MRI) apparatus and comprises both a static magnetic field with an MRI field strength of from about 0.1 Tesla to about 30 Tesla, a gradient magnetic field of from about 1 to about 200 kilohertz, and an alternating current electromagnetic field with a frequency of from about 1 megahertz to about 3 terahertz.

In one embodiment, the static magnetic field has a field strength of from about 0.5 Tesla to about 20 Tesla. In another embodiment, the static magnetic field has a field strength of from about 1 Tesla to about 10 Tesla. In yet another embodiment, the static magnetic field has a field strength of from about 1.5 Tesla to about 3.5 Tesla.

In one embodiment, the energy 1204 is comprised of an input alternating current electromagnetic field with a frequency of from about 1 megahertz to about 2 gigahertz and, more preferably, from about 50 megahertz to about 1 gigahertz. In one aspect of this embodiment, the input alternating current electromagnetic field has a frequency of from about 50 megahertz to about 300 megahertz.

Referring again to FIG. 31, and in the preferred embodiment depicted therein, a stent 1206 is comprised of a multiplicity of struts 1208 that define an exterior surface 1210 and an interior cavity 1212. A multiplicity of openings 1214 are defined are also defined by such struts; and these openings 1214 facilitate communication between the interior cavity 1212 and the areas 1216 disposed outside of such exterior surface 1210.

In the embodiment depicted in FIG. 31, biological material 1218 is disposed within the stent lumen 1212. In the prior art devices, this biological material would be screened from the energy 1204; and whatever energy did reach the interior area of the stent would not be retransmitted through such outer surface 1210.

Thus, and referring again to U.S. Pat. No. 6,712,844 (the entire disclosure of which is hereby incorporated by reference into this specification), “Because stents are constructed of electrically conductive materials, they suffer from a Faraday Cage effect when used with MRI's. Generically, a Faraday Cage is a box, cage, or array of electrically conductive material intended to shield its contents from electromagnetic radiation. The effectiveness of a Faraday Cage depends on the wave length of the radiation, the size of the mesh in the cage, the conductivity of the cage material, its thickness, and other variables. Stents do act as Faraday Cages in that they screen the stent lumen from the incident RF pulses of the MRI scanner. This prevents the proton spins of water molecules in the stent lumen from being flipped or excited.”Thus, and referring again to FIG. 31, in the prior art stent assemblies the input energy 1204 (and especially the input radio frequency energy) is substantially screened “ . . . from the incident RF pulses of the MRI scanner . . . ”; and very little, if any, of such incident RF pulses 1220 penetrate past the outer surface 1210 of the stent to reach the inner lumen 1212 and the biological material 1218.

To the extent that such incident RF pulses 1220 do penetrate the outer surface 1210 of the stent, they will interact with the biological material 1218 to produce an output signal 1222. This output signal 1222 generally does not have a fixed phase relationship with the input signal 1220 in the prior art stent assemblies. Thus, as is also disclosed in U.S. Pat. No. 6,712,844, “The stent's high magnetic susceptibility, however, perturbs the magnetic field in the vicinity of the implant. This alters the resonance condition of protons in the vicinity, thus leading to intravoxel dephasing with an attendant loss of signal” (see column 2 of such patent). This phenomenon of intravoxel dephasing is also discussed in U.S. Pat. No. 5,283,526 (method for performing single and multiple slice magnetic resonance spectroscopic imaging), U.S. Pat. No. 6,069,949 (gradient characterization using fourier-transform), U.S. Pat. No. 6,408,201 (method and apparatus for efficient stenosis identification in peripheral arterial vasculature using MR imaging), U.S. Pat. No. 6,472,872 (real-time shimming of polarizing field in magnetic resonance system), U.S. Pat. No. 6,587,708 (method for coherent steady-state imaging of constant-velocity flowing fluids), U.S. Pat. No. 6,618,607 (MRI imaging methods using a single excitation), and the like. Reference also may be had, e.g., to published U.S. Pat. No. applications US20020041833A1 (method of magnetic resonance imaging), US20020082497 (MRI imaging methods using a single excitation), and US20020188345A1 (MRI compatible stent). The entire disclosure of each of these United States patents, and of each of these published U.S. Pat. No. applications, is hereby incorporated by reference into this specification.

Referring again to FIG. 31, and in the preferred embodiment depicted therein, in the prior art stent assemblies the output signal 1222 has a difficult time in escaping the exterior surface 1210 of the stent. Thus, and referring again to U.S. Pat. No. 6,712,844 (see column 2), “ . . . the stent Faraday Cage likely impedes the escape of whatever signal is generated in the lumen. The stent's high magnetic susceptibility, however, perturbs the magnetic field in the vicinity of the implant. This alters the resonance condition of protons in the vicinity, thus leading to intravoxel dephasing with an attendant loss of signal. The net result with current metallic stents, most of which are stainless steel, is a signal void in the MRI images. Other metallic stents, such as those made from Nitinol, also have considerable signal loss in the stent lumen due to a combination of Faraday Cage and magnetic susceptibility effects.”

In applicants' stent assembly 1200, by comparison, the output signal 1222 is not “dephased,” i.e., it has a fixed phase relationship with the input signal 1220. The term “fixed phase relationship” is well known to those skilled in the art. Reference may be had, e.g., to U.S. Pat. Nos. 3,581,011; 3,594,738; 3,611,127; 3,611,144; 3,659,942; 3,669,209; 3,691,475; 3,774,115; 3,777,691; 3,784,930; 3,792,473; 3,851,247; 3,921,087; 3,932,811; 4,035,833; 4,038,756; 4,118,125; 4,142,489; 4,152,703; 4,164,577; 4,188,573; 4,204,151; 4,392,020; 4,499,534; 4,642,675; 4,700,359; 4,842,477; 4,872,164; 4,877,974; 4,914,421; 4,924,420; 4,965,810; 4,989,219; 5,315,232; 5,333,074; 5,337,040; 5,345,240; 5,528,112; 5,586,042; 5,722,744; 5,872,959; 6,047,808; 6,278,334; 6,348,826; 6,553,835; 6.583,645; and the like. The entire disclosure of each of these United States patents is hereby incorporated by reference into this specification.

Referring again to FIG. 31, and in the preferred embodiment depicted therein, the input alternating current electromagnetic field 1220 may be represented by the formula Acos (2πft+φ₀), wherein A is the magnitude of the input alternating current electromagnetic field (and is preferably from about 1×10⁻⁶ Tesla to about 100×10⁻⁶ Tesla), f is the frequency of the in alternating current electromagnetic field (and preferably is from about 1 megahertz to about 2 gigahertz), and φ₀ is the initial phase of the input alternating current electromagnetic field 1220 when t is 0 seconds.

By comparison, and referring again to FIG. 31, and in the preferred embodiment depicted therein, the output alternating current electromagnetic field 1222 may be represented by the formula B cos(2πft+φ₁), wherein B is the magnitude of the output alternating current electromagnetic field 1222, f is the frequency of the output alternating current electromagnetic field, and φ₁ is the phase of the output alternating current electromagnetic field 1222 when t₁ is measured in relation to t₀.

A fixed phase relationship exists between the input signal 1220 and the output signal 1222 when the following equation is satisfied: φ₁−φ₀=±C±2πn, wherein φ₁ is the phase of the output signal 1222, φ₀ is the phase of the input signal 1220, C is a number between 0 and 360 degrees, and n is an integer including 0.

Referring again to FIG. 31, and to the preferred embodiment depicted therein, it will be seen that implantable magnetic field detectors 1230 and 1232 may be used to detect input signal 1220 and output signal 1222. As will be apparent, one may also refer to the calibration of source 1202 to determine the characteristics of input signal 1230.

In one preferred embodiment, not shown, the magnetic field detectors 1230 and 1232 are omitted and external sources of radiation and detection are used in place of such omitted detectors 1230/1232. In one aspect of this embodiment, a set of coils is used to emit and receive radio frequency energy. In one aspect of this embodiment, such coils are phased array coils that are used to measure the energy 1204 that is supplied to the stent assembly, the energy that penetrates the stent assembly, and the energy that is retransmitted by the stent assembly.

In one embodiment, such set of coils are phased array coils. These coils, are their uses, are well known in the MRI art. Reference may be had, e.g., to U.S. Pat. No. 4,985,678 (horizontal field iron core magnetic resonance scanner), U.S. Pat. No. 5,394,087 (multiple quadrature surface coil system for simultaneous imaging in magnetic resonance imaging), U.S. Pat. No. 5,521,056 (orthogonal adjustment of magnetic resonance surface coils), U.S. Pat. No. 5,578,925 (vertical field quadrature phased array coil system), U.S. Pat. No. 6,097,186 (phased array coil, receive signal processing circuit, and MRI apparatus), U.S. Pat. No. 6,177,795 (spectral component imaging using phased array coils), U.S. Pat. No. 6,396,273 (magnetic resonance imaging receiver/transmitter coils), U.S. Pat. No. 6,411,090 (magnetic resonance imaging transmit coil), U.S. Pat. No. 6,469,406 (autocorrection of MR images acquired using phased array coils), U.S. Pat. No. 6,492,814 (self localizing receive coils for MR), U.S. Pat. No. 6,534,983 (multi-channel phased array coils having minimum mutual inductance for magnetic resonance systems), U.S. Pat. No. 6,604,697 (magnetic resonance imaging receiver/transmitter coils), U.S. Pat. No. 6,608,480 (RF coil for homogeneous quadrature transmit and multiple channel receive), U.S. Pat. No. 6,639,406 (apparatus for decoupling quadrature phased array coils), U.S. Pat. No. 6,714,013 (magnetic resonance imaging receiver/transmitter coils), U.S. Pat. No. 6,724,923 (automatic coil selection of multi-receiver MR data using fast prescan data analysis), U.S. Pat. No. 6,738,501 (adaptive data differentiation and selection from multi-coil receiver to reduce artifacts in reconstruction), U.S. Pat. No. 6,747,452 (decoupling circuit for magnetic resonance imaging local coils), U.S. Pat. No. 6,762,606 (retracting MRI head coil), U.S. Pat. No. 6,781,379 (cable routing and potential equalizing ring for magnetic resonance imaging coils), U.S. Pat. No. 6,788,057 (open architecture gradient coil set for magnetic resonance imaging apparatus), and the like. The entire disclosure of each of these United States patents is hereby incorporated by reference into this specification.

Referring again to FIG. 31, and to the embodiment depicted therein, the probes 1230 and 1232 may be conventional magnetic field detectors. One may use, e.g., conventional magnetic field detectors such as, e.g., the magnetic field detectors disclosed in U.S. Pat. No. 3,829,883 (magnetic field detector employing plural drain IGFET), U.S. Pat. No. 3,835,377 (three terminal magnetoresistive magnetic field detector), U.S. Pat. No. 4,064,453 (magnetic field detector), U.S. Pat. No. 4,210,083 (alternating magnetic field detector), U.S. Pat. Nos. 4,218,975, 4,714,880 (wide frequency pass band magnetic field detector), U.S. Pat. Nos. 4,767,989, 4,875,785, 5,187,437, 5,194,808 (magnetic field detector using a superconductor magnetoresistive element), U.S. Pat. No. 5,309,096 (magnetic field detector for a medical device implantable in the body of patient), U.S. Pat. No. 5,309,097 (video display terminal magnetic field detector), U.S. Pat. No. 5,317,251 (peak magnetic field detector with non-volatile storage), U.S. Pat. Nos. 5,365,391, 5,389,880 (hall analog magnetic field detector), U.S. Pat. No. 5,424,642 (magnetic field detector with a resiliently mounted electrical coil), U.S. Pat. No. 5,517,112 (magnetic field detector with noise blanking), U.S. Pat. No. 5,521,500 (thin-film magnetic field detector), U.S. Pat. No. 5,598,273 (highly sensitive magnetic field detector using low noise DC SQUID), U.S. Pat. No. 5,619,137 (chopped low power magnetic field detector with hysteresis memory), U.S. Pat. Nos. 5,662,694, 5,709,225 (combined magnetic field detector and activity detector employing a capacitive sensor for a medical implant), U.S. Pat. No. 6,005,383 (electrical current sensor with magnetic field detector), U.S. Pat. No. 6,144,196 (magnetic field measuring apparatus and apparatus for measuring spatial resolution of magnetic field detector), U.S. Pat. No. 6,396,264 (shielded loop magnetic field detector), U.S. Pat. Nos. 6,683,397, 6,750,648 (magnetic field detector having a dielectric looped face), and the like. The entire disclosure of each of these United States patents is hereby incorporated by reference into this specification.

In one preferred embodiment, each of the magnetic field detectors 1230/1232 is an implantable medical field detector such as, e.g., the “medical field detector and telemetry unit for implants” described and claimed in U.S. Pat. No. 5,545,187, the entire disclosure of which is hereby incorporated by reference into this specification. Claim 1 of this patent describes “1. A combination magnetic field detector and threshold unit for use in a medical implant, comprising: a telemetry circuit connected to a voltage source; control logic which generates control signals respectively for telemetry and magnetic field detection; a coil unit including a plurality of coil unit parts; switch means, controlled by said control logic for, when said control logic generates a control signal for telemetry, electrically connecting said coil unit into said telemetry circuit for forming means for receiving and transmitting telemetry signals and for, when said control logic generates a control signal for magnetic field detection, electrically connecting said coil unit parts for forming a primary side and a secondary side of a pulse transformer which generates an output signal having a characteristic which varies dependent on the presence of a magnetic field; and magnetic field indicator means, connected to said secondary side of said pulse transformer, for generating a signal indicating the presence of a magnetic field when said characteristic satisfies a predetermined condition.”

U.S. Pat. No. 5,545,187 contains an excellent discussion of some “prior art” magnetic field sensors. It discloses that “In a medical implant, such as a pacemaker, a magnetic field detector is used for non-invasive activation of different functions in the implant in combination with a permanent magnet placed in the vicinity of the implant at the outside of the patient's body. Some of the functions which can be activated in, e.g., a pacemaker are: disabling the pacemaker's demand function so the pacemaker adapts its operation to battery capacity and having the pacemaker operate in a special, temporary stimulation mode, e.g., in the case of tachycardia, and in conjunction with pacemaker programming . . . . Outside the implant art, the detection of magnetic fields in a number of different ways, e.g., with the aid of reed switches, by changing the resonance frequency or inductance, etc., is generally known.”

U.S. Pat. No. 5,545,187 also discloses that “One device for determining the strength of a magnetic field is described in an article by Lennart Grahm, “Elektrisk matteknik, Analoga instrument och matmetoder,” part 2, 1977, Elektrisk matteknik, Lund, pp. 543-545. As described therein, the voltage induced in a small test body made of ferromagnetic metal is examined with a Forster probe. The Forster probe consists of a small test body made of a ferromagnetic material with high permeability and provided with two windings, one of which is used for alternating current magnetization and the other is used for measuring the ensuing induced voltage. The larger the constant magnetic field, the greater the amplitude of even harmonics when the probe is placed in a constant magnetic field. Thus, a phase detector with a reference voltage equal to twice the frequency of the excitation current can be used for supplying a signal which increases with an increase in the constant magnetic field.”

U.S. Pat. No. 5,545,187 also discloses that “In the implant art, a conventional magnetic field detector consists of a reed switch. Reed switches, however, are sensitive and rather expensive components which also occupy a relatively large amount of space in the implant . . . In order to eliminate the need for a reed switch, therefore, recent proposals have suggested utilization of the implant's telemetry unit so that the unit can also be used for detecting the presence of a magnetic field, in addition to its telemetry function. U.S. Pat. No. 4,541,431 discloses one such proposal with a combined telemetry and magnetic field detector unit. This unit contains a conventional resonant circuit containing, e.g., a coil used in telemetry for transmitting and receiving data. The resonant circuit is also used for sensing the presence of a magnetic field whose strength exceeds a predefined value. The resonant frequency for the resonant circuit varies with the strength of the magnetic field. The resonant circuit is periodically activated, and the number of zero crossings of its signal with a sensing window with a predefined duration is determined. If a predetermined number of zero crossings occurs, this means that the strength of the magnetic field exceeds the predefined value.”

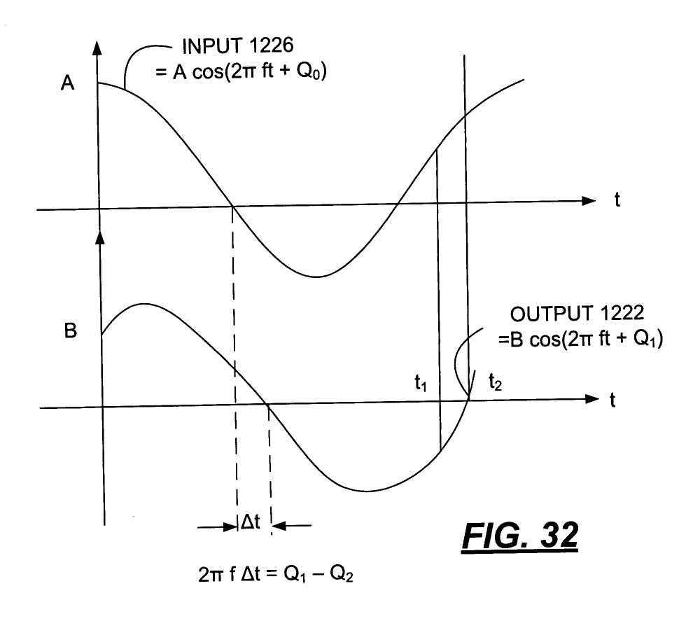

Referring again to FIG. 31, and in one preferred embodiment, the output from probe 1232 may be fed to a signal processor 1240 which, in addition, may also contain information about the input from source 1202. The signal processor 1240 may then be connected to a display (not shown) adapted to display graphs of the input field 1220 and the output field 1222, as illustrated in FIG. 32. From this display, one may determine the magnitude A of the input signal 1220, the magnitude B of the output signal 1222, and the difference in the phases (φ's) of the input and output signals.

As indicated elsewhere in this specification, it is preferred that the input signal 1220 and the output signal 122 have a fixed phase relationship. Furthermore, it is preferred that the ratio of B/A is at least 0.01 and, more preferably, at least about 0.1. In one embodiment, the ratio of B/A is at least 0.2. In yet another embodiment, the ratio of B/A is at least 0.3.

Parallel and Series Circuits in Material: A Preferred Coated Stent Assembly

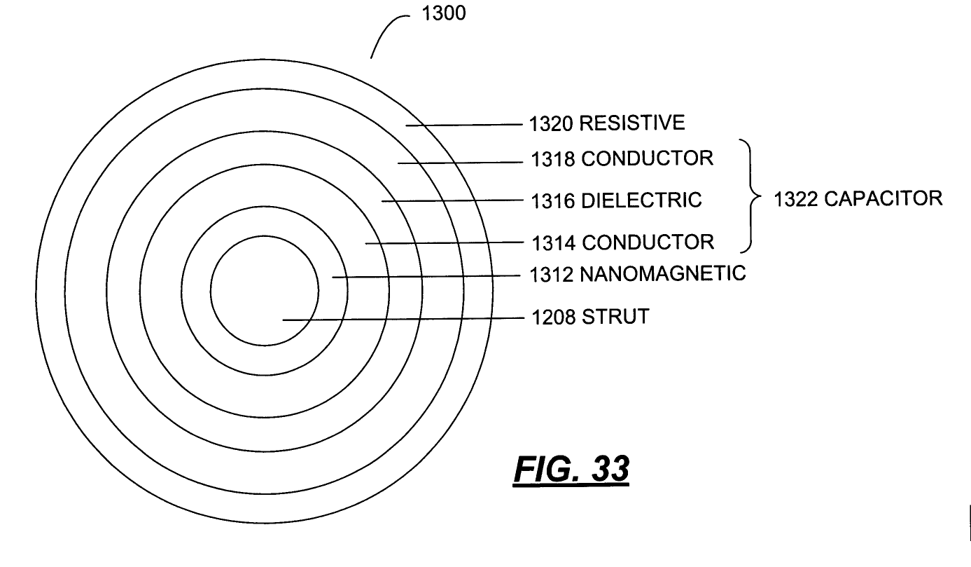

FIG. 33 is a sectional schematic view, not drawn to scale, of a section of the stent assembly 1200 (see FIG. 31) and, in particular, of a coated strut assembly 1300. Referring to FIG. 33, and in the preferred embodiment depicted therein, it will be seen that each of struts 1208 (see FIG. 31) is preferably coated with a first coating 1312 of nanomagnetic material.

In one preferred embodiment, the coating 1312 has a thickness of at least about 100 nanometers and, more preferably, at least about 500 nanometers. In one aspect of this embodiment, the thickness of coating 1312 is from about 800 nanometers to about 1200 nanometers.

In one preferred embodiment, the nanomagnetic coating 1312 has a magnetization, at a field strength of 2 Tesla, of less than about 100 electromagnetic units (emu) per cubic centimeter and, more preferably, of less than about 10 electromagnetic units per cubic centimeter. In one embodiment, the nanomagnetic coating 1312 has a magnetization, at a field strength of 2 Tesla, of less than about 1 electromagnetic units per cubic centimeters.

In one preferred embodiment, the nanomagnetic coating 1312 has a saturation magnetization of greater than about 1.5 Tesla and, more preferably, of greater than about 1.6 Tesla. In another embodiment, the saturation magnetization of the nanomagnetic coating 1312 is greater than about 2.0 Tesla. In another embodiment, the saturation magnetization of the nanomagnetic coating is greater than about 3.0 Tesla. Put another way, the nanomagnetic coating 1312 does preferably does not reach saturation magnetization at a field strength of 1.5 Tesla, or 1.6 Tesla, or 2.0 Tesla, or 3.0 Tesla, depending upon the embodiment in question.

As is discussed elsewhere in this specification, the nanomagnetic coating 1312 is comprised of nanomagnetic particles that, in one preferred embodiment, have an average particle size of from about 2 to about 100 nanometers and, preferably, from about 3 to about 10 nanometers.

In one embodiment, the nanomagnetic coating 1312 has a resistivity, at a temperature of 300 degrees Kelvin, of from about 1×10⁻² to 1×10⁻⁷ ohm-meters and, preferably, from about 8×10⁻⁵ to about 8×10⁻⁷ ohm-meters.

Referring again to FIG. 33, and in the preferred embodiment depicted therein, a coating 1314 of conductive material is preferably disposed above and contiguous with the coating 1312 of nanomagnetic material. The conductive coating 1314 preferably has a resistivity at a temperature of 300 degrees Kelvin of less than 10⁻⁷ ohm-meters. In one aspect of this embodiment, the conductive coating 1314 preferably has a resistivity of from about 1×10⁻⁸ to about 5×10⁻⁸ ohm-meters. Aluminum is one conductive material that may be used; copper is another conductive material that may be used; and other suitable conductive materials will be apparent to those skilled in the art.

The conductive coating 1314 preferably has a thickness of less than about 100 nanometers and, more preferably, less than about 60 nanometers. In one embodiment, the conductive coating 1314 has a thickness of from about 40 to about 55 nanometers.

Referring again to FIG. 33, and in the preferred embodiment depicted therein, disposed over coating 1314, and contiguous therewith, is dielectric coating 1316. Dielectric coating 1316, which preferably has a thickness of less than about 100 nanometers, also preferably has a dielectric constant larger than 1.0 and, more preferably, larger than 2.0. In one embodiment, the dielectric constant of coating 1316 is preferably greater than 3.0. The values of dielectric constant described are those measured at a temperature of 300 degrees Kelvin.

As is known to those skilled in the art, the dielectric constant for an isotropic medium is the ratio of the capacitance of a capacitor filled with a given dielectric to that of the same capacitor having only a vacuum as dielectric. See, e.g., page 531 of Sybil P. Parker's “McGraw-Hill Dictionary of Scientific and Technical Terms,” Fourth Edition (McGraw-Hill Book Company, New York, N.Y., 1989).

Referring again to FIG. 33, and in the embodiment depicted, disposed on top of dielectric layer 1316 is another coating 1318 of coating material. Conductive layer 1318 preferably has thickness and resistivity properties that are similar to the thickness and resistivity properties of conductive layer 1314.

The conductive layer 1318/dielectric layer 1316/conductive layer 1314 assembly form a capacitor 1322 that, exhibits capacitive reactance in the presence of a radio frequency field. The nanomagnetic layer 1312 enclosing the strut 1310 forms an inductor that exhibits inductive reactance in the presence of a radio frequency field. In one embodiment, the dielectric material used is chosen so that, in combination with the inductor assembly, one is near resonance at the frequency of the applied field.

The coatings illustrated in FIG. 33 act as a filter, with a specified inductive reactance and capacitive reactance, that presents minimal impedance to certain frequencies and maximum impedance to other frequencies. In order to “tune the bandwidth” and to allow a reasonable range of frequencies to pass through the filter around the resonant frequency, a resistive layer 1320 is deposited on top of the conductive layer 1318. In one embodiment, the resistive layer 1320 has a thickness less than about 100 nanometers and a resistivity of from about from about 1×10⁻² to 1×10⁻⁷ ohm-meters.

The construct illustrated in FIG. 33 is merely illustrative of many constructs that may be used to construct filter circuits utilizing strut 1208 and nanomagnetic coating 1312. In one embodiment, a combination of such conductor coatings 1314/1318 and dielectric coatings 1316 are used to construct other circuits.

In one preferred embodiment, one or more cancellation circuits are constructed so that the currents induced by the radio frequency field are out of phase with each other and tend to cancel each other. These (and other) cancellation circuits are well known to those skilled in the art. Reference may be had, e.g., to U.S. Pat. No. 3,720,941 (automatic monopulse clutter cancellation circuit); U.S. Pat. No. 3,715,488 (noise cancellation circuit); U.S. Pat. No. 3,932,713 (induction cancellation circuit); U.S. Pat. Nos. 3,947,848; 4,078,156 (drift cancellation circuit); U.S. Pat. No. 4,204,219 (noise cancellation circuit); U.S. Pat. No. 4,211,978 (cross-talk component cancellation circuit); U.S. Pat. No. 4,214,129 (sideband cancellation circuit); U.S. Pat. No. 4,245,202 (current cancellation circuit); U.S. Pat. No. 4,254,436 (noise cancellation circuit); U.S. Pat. No. 4,268,727 (echo cancellation circuit); U.S. Pat. No. 4,285,006 (ghost cancellation circuit); U.S. Pat. No. 4,341,990 (line ripple cancellation circuit); U.S. Pat. Nos. 4,525,683; 4,528,676 (echo cancellation circuit); U.S. Pat. No. 4,585,987 (sense current cancellation circuit); U.S. Pat. No. 4,629,996 (difference signal distortion cancellation circuit); U.S. Pat. No. 4,688,044 (multiple range interval clutter cancellation circuit); U.S. Pat. No. 4,827,161 (offset voltage cancellation circuit); U.S. Pat. No. 4,932,085 (pilot cancellation circuit); U.S. Pat. No. 5,001,773 (local oscillator feed thru cancellation circuit); U.S. Pat. No. 5,043,814 (adaptive ghost cancellation circuit); U.S. Pat. No. 5,046,133 (interference cancellation circuit); U.S. Pat. No. 5,051,704 (feed forward distortion cancellation circuit); U.S. Pat. No. 5,066,891 (magnetic field cancellation circuit); U.S. Pat. No. 5,161,017 (ghost cancellation circuit); U.S. Pat. No. 5,168,256 (distortion canceling circuit for audio peak limiting); U.S. Pat. No. 5,182,476 (offset cancellation circuit); U.S. Pat. No. 5,428,314 (odd/even order distortion generator and distortion cancellation circuit); U.S. Pat. No. 5,434,446 (parasitic capacitance cancellation circuit); U.S. Pat. No. 5,440,353 (display monitor including moire cancellation circuit); U.S. Pat. No. 5,561,288 (biasing voltage cancellation circuit); U.S. Pat. No. 5,563,587 (current cancellation circuit); U.S. Pat. No. 5,600,251 (induction noise cancellation circuit); U.S. Pat. No. 5,659,588 (filter leakage cancellation circuit); U.S. Pat. No. 5,719,907 (phase jitter cancellation circuit); U.S. Pat. No. 5,793,551 (differential input capacitance cancellation circuit); U.S. Pat. No. 5,796,301 (offset cancellation circuit); U.S. Pat. No. 5,929,692 (ripple cancellation circuit); U.S. Pat. No. 5,977,892 (offset cancellation circuit); U.S. Pat. No. 6,052,422 (analog signal offset cancellation circuit); U.S. Pat. No. 6,167,247 (leak cancellation circuit); U.S. Pat. No. 6,172,564 (intermodulation product cancellation circuit); U.S. Pat. No. 6,208,135 (inductive noise cancellation circuit); U.S. Pat. No. 6,211,724 (glitch cancellation circuit); U.S. Pat. No. 6,243,430 (noise cancellation circuit); U.S. Pat. No. 6,281,889 (Moire cancellation circuit); U.S. Pat. No. 6,333,947 (interference cancellation system); U.S. Pat. No. 6,344,756 (echo cancellation circuit); U.S. Pat. No. 6,429,749 (cancellation circuit that suppresses electromagnetic interference); U.S. Pat. No. 6,496,064 (intermodulation product cancellation circuit); U.S. Pat. No. 6,549,054 (DC offset cancellation circuit); U.S. Pat. No. 6,566,934 (charge cancellation circuit); U.S. Pat. No. 6,671,075 (offset voltage cancellation circuit); U.S. Pat. No. 6,693,805 (ripple cancellation circuit); U.S. Pat. No. 6,792,056 (cancellation circuit that suppresses electromagnetic interference using a function generator); and the like. The entire disclosure of each of these United States patents is hereby incorporated by reference into this specification. SEARCH NOTCH filter, also band PASS FILTER.

Coated strut 1208 assemblies, such as assembly 1300, may be constructed so as to include one or more of the cancellation circuits described in the patents in the prior paragraph of this specification. Such circuits may be constructed by using conductive and/or dielectric coatings. Alternatively, or additionally, one or more components of such circuits may be printed on the surface(s) of one or more of such coatings by conventional means.

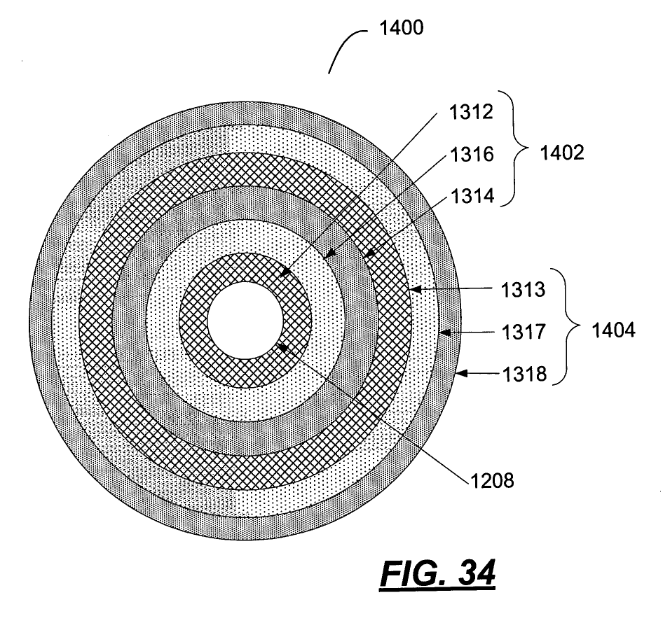

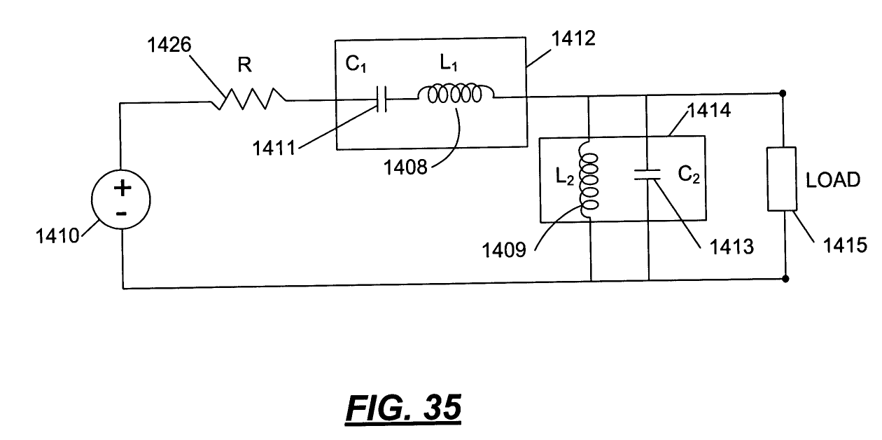

FIG. 34 is a sectional view of another preferred coated strut assembly 1400 that differs from the strut assembly 1300 in that, disposed about strut 1208, is a first coating 1312 of nanomagnetic material, a second coating 1316 of dielectric material, a third coating 1314 of conductive material, a fourth coating 1313 of nanomagnetic material (which may be the same as or different than coating 1312), a fifth coating 1317 of dielectric material (which may be the same as or different than coating 1316), and a sixth coating 1318 of conductive material (which may be the same as or different than coating 1314). The combination of coatings 1402 (which includes coatings 1314/1316/1312) is believed to form an equivalent circuit 1436 (see FIG. 35). The combination of coatings 1404 (which includes coatings 1313/1317/1318) is believed to form an equivalent circuit 1438.

Without wishing to be bound to any particular theory or theories, applicants believe that the circuit depicted in FIG. 35 is a reasonably accurate depiction of the equivalent circuit that exists in assembly 1400.

The strut 1208 contains both some resistance 1426 and inductance 1408 and inductance 1409. When strut 1208 is subjected to a radiofrequency field 1410 produced by the radio frequency generator of an MRI machine (not shown), a capacitance 1411 in series with inductance 1408 forms a series resonant circuit 1412 that preferably has a net reactance of zero at the frequency of the radiofrequency (which generally is either 64 megahertz or 128 megahertz, corresponding to d.c. field strengths of 1.5 Tesla and 3.0 Tesla, respectively).

The equivalent resistance 1426 is the resistive loss in the circuit caused by ohmic loss in the various coatings. This equivalent resistance 1426 is used in a well known manner to adjust the bandwidth of the series resonant circuit. The equation for a series resonant frequency is 1/(LC)^{0.5}. The equation for the bandwidth of such a circuit is R/L.

Referring again to FIG. 35, and in the preferred equivalent circuit depicted therein, there is another parallel resonant circuit 1414 comprised of inductance 1409 and capacitance 1413. The inductance 1409 comes from the inductive coatings that often contain nanomagnetic material; it also comes, in part, from the conductive substrate. The capacitance 1413 comes from the configuration of a dielectric coating between conductive materials; it also may come form interconnections (via vias) between various coating layers, as will be described in more detail later in this specification.

The resonant frequency of the parallel circuit 1414 is given by the equation 1/(LC)0.5. As will be apparent, in the parallel circuit configuration, the inductance is contributed by inductor 1409, and the capacitance is contributed by capacitor 1413.

At this parallel resonant frequency, the impedance is substantially infinite; and the input 1410 is thus coupled to the load 1415. The equivalent load 1415 is the interior of the metallic stent 1201 (see FIG. 31).

As will be apparent to those skilled in the art, and referring again to FIG. 34, modification of one or more of the coatings 1312, 1313, 1314, 1316, 1317, and/or 1318 will simultaneously modify both the values of the resistance, inductance, and capacitance presented by such coatings, and will also simultaneously modify the impedance of such coatings.

A Composition Comprised of Magnetic Material and Polymeric Material

As is disclosed elsewhere in this specification, one may prepare a composition comprised of both the nanomagnetic material of this invention and polymeric material.

The term polymer, as used herein, refers to a member of a series of polymeric compounds that are composed of very large molecules which consist essentially of recurring, long-chain structural units; these structural units distinguish polymers′from other types of organic molecules and confer on them tensile strength, deformability, elasticity, and hardness. See, e.g., page 534 of Julius Grant's “Hackh's Chemical Dictionary,” Fourth Edition (McGraw-Hill Book Company, New York, N.Y., 1972).

In one embodiment, the composition of this invention is comprised of such nanomagnetic material, such polymeric material, and one or more of the mineral materials described hereinabove. In the remainder of this section of the specification, various polymeric materials that may be used in such “magnetic mineral composition” will be described by way of illustration and not limitation.

The polymeric material used in the magnetic mineral composition of the instant invention may be comprised of one or more resins such as, e.g., the phenol-formaldehyde resin disclosed in U.S. Pat. No. 3,467,618, the entire disclosure of which is hereby incorporated by reference into this specification. This patent claims (in claim 1) a molded article comprised of a cured phenol-formaldehyde resin and about 10 to 75 weight percent of halloysite clay.”

The polymeric material used in the magnetic mineral composition of the instant invention may be a polyamide-containing resin, such as the polyamide material described in U.S. Pat. No. 4,894,411, the entire disclosure of which is hereby incorporated by reference into this specification. This polyamide resin is described in column 1 of such patent, wherein it is disclosed that “Various attempts have been made so far to incorporate an organic polymeric material with an inorganic material such as calcium carbonate, clay mineral, and mica for the improvement of its mechanical properties. As the result of such attempts, the present inventors developed a composite material composed of a resin containing a polyamide and a layered silicate having a layer thickness of 7-12 .ANG. uniformly dispersed therein, with the polymer chain of said polyamide being partly connected to said silicate through ionic bond. (See Japanese Patent Laid-open No. 74957/1987 (which corresponds to U.S. Pat. No. 4,739,007).) This composite material has a high elastic modulus and heat resistance because of its unique structure; that is, silicate having an extremely high aspect ratio are uniformly dispersed in and connected to a polyamide resin through ionic bond. This composite material, however, is subject to brittle fracture even at room temperature under a comparatively small load. Therefore, it is not necessarily satisfactory in mechanical strength.”

U.S. Pat. No. 4,894,411 also discloses that “In the meantime, the crystalline polyamide resin as a typical engineering plastics exemplified by nylon-6 and nylon-66 finds use as automotive parts and electric and electronic parts on account of its high melting point and high rigidity. A disadvantage of the crystalline polyamide resin is that it is opaque on account of its crystalline structure. This leads to a problem arising from the fact that automotive parts such as reservoir tanks, radiator tanks, and fuel tanks made of polyamide resin make the liquid level invisible from outside and the electronic parts such as connectors made of polyamide resin prevent the detection of conductor breakage therein. Unlike the crystalline polyamide resin, the amorphous polyamide resin having the aromatic skeleton structure is transparent. An example of the amorphous polyamide resin is “Trogamid” made by Dynamit Nobel Co., Ltd. Unfortunately, it is extremely expensive and cannot be a substitute for aliphatic nylons such as nylon-6 and nylon-66. Moreover, the aliphatic nylon extremely decreases in strength and heat resistance when it is made amorphous. Under these circumstances, there has been a demand for a polyamide resin which has high clarity without decrease in crystallinity.”

Another polyamide resin that may be used as the polymeric material in the magnetic mineral composition of this invention sideswiped in U.S. Pat. No. 5,164,440, the entire disclosure of which is hereby incorporated by reference into this specification. This patent claims “1. A polyamide resin composition comprising (A) at least one polyamide resin component selected from the group consisting of a polyamide resin and a resin composition comprising (i) at least 80 weight % of a polyamide resin and (ii) the remainder being another thermoplastic resin selected from the group consisting of polypropylene, an ABS resin, polycarbonate, polyethyleneterephthalate and polybutyleneterephthalate; (B) a layered silicate having a thickness of 6 to 20.ANG., a length of one side of 0.002 to 1 μm and being uniformly dispersed in the component (A) with a weight ratio of 0.05 to 30 parts by weight of (B) per 100 parts by weight of (A); and respective layers of silicate being positioned apart from each other by 20.ANG. or more on an average; and (C) an impact resistance improving material selected from the group consisting of: impact resistance improving materials comprising copolymers obtained from ethylene, unsaturated carboxylic acid and unsaturated carboxylic acid metal salt; impact resistance improving materials comprising olefin copolymers containing 0.01 to 10 mole % of acid groups; and impact resistance improving materials comprising block copolymers, containing 0.01 to 10 mole % of acid groups, obtained from vinyl aromatic compounds and conjugated diene compounds, hydrogenated products of said block copolymers or mixtures thereof, wherein there are 5 to 70 parts by weight (c) per 100 parts by weight of (A).”

The polymeric material used in the magnetic mineral composition of this invention may be a polyimide such as, e.g., the polyimide disclosed in U.S. Pat. No. 6,164,660, the entire disclosure of which is hereby incorporated by reference into this specification. Claim 1 of this patent describes “1. A polyimide composite material which comprises a polyimide-containing resin, organic monoonium ions and a layered clay mineral, said layered clay mineral being intercalated with the organic monoonium ions not bonding with said polyimide and uniformly dispersed in said polyimide.” The preparation of the polyimide material of this patent is described, e.g., in column 4 of such patent, wherein it is disclosed that “The polyimide in the present invention is produced from any dianhydride and diamine which are known as monomers for polyimide. Examples of the dianhydride include pyromellitic dianhydride, 3,3′,4,4′-biphenyltetracarboxylic dianhydride, and 3,3′,4,4′-benzophenonetetracarboxylic dianhydride. Examples of the diamine include 4,4′-diaminodiphenyl ether, 3,4′-diaminodiphenyl ether, and p-phenylenediamine. They may be used alone for homopolymerization or in combination with one another for copolymerization. They may be copolymerized with a dicarboxylic acid and a diol or their respective derivatives to give polyamideimide, polyesteramideimide, or polyesterimide.”

U.S. Pat. No. 5,164,460 also discloses that “The polyimide in the present invention is also produced from a prepolymer which is exemplified by poly(amic acid). Usually, a polyimide resin cannot be mixed in its molten state with the intercalated clay mineral because it decomposes at a temperature lower than the temperature at which it begins to flow. But, if the temperature of fluidization is lower than that of decomposition, the polyimide composite material can be produced by this melt-mixing method.”

The polymeric material used in the magnetic mineral composition of this invention may be a polypropylene material such as, e.g., the polypropylene thermoplastic resin composition disclosed in U.S. Pat. No. 5,206,284, the entire disclosure of which is hereby incorporated by reference into this specification. Claim 1 of this patent describes “1. A polypropylene thermoplastic resin composition comprising: 95-5% by weight of (a) a modified polypropylene obtained by grafting a crystalline polypropylene with 0.05 to 5% by weight of at least one compound selected from the group consisting of an unsaturated carboxylic acid, an unsaturated carboxylic acid anhydride, an unsaturated carboxylic ester, an unsaturated carboxylate salt and an unsaturated carboxylic acid amide, or a crystalline polypropylene comprising at least 5% by weight of said modified polypropylene, and 5-95% by weight of (b) a modified polyamide obtained by partially or wholly modifying a polyamide with 0.05 to 10% by weight of a clay mineral.” A discussion of crystalline polypropylenes is presented at column 1 of such patent, wherein it is disclosed that “Crystalline polypropylenes are superior in mechanical properties and moldability and used in wide applications, but are not satisfactory in heat resistance and impact resistance when used in industrial parts. It has conventionally been conducted to add an inorganic filler to a crystalline polypropylene to improve the heat resistance of the latter, or to add an ethylene-.alpha.-olefin copolymer rubber or a polyethylene to a crystalline polypropylene to improve the impact resistance of the latter; however, the addition of an inorganic filler significantly reduces the impact resistance of polypropylene and the addition of an ethylene-.alpha.-olefin copolymer rubber of a polyethylene reduces the rigidity, heat resistance and oil resistance of polypropylene. Even the combined addition of an inorganic filler and an ethylene-.alpha.-olefin copolymer rubber or a polyethylene to a polypropylene does not give an effect more than the sum of addition effects of respective additives, and accordingly provides no sufficient method for improvement of polypropylene in heat resistance and impact resistance.”

U.S. Pat. No. 5,206,284 also discloses that “Meanwhile, there was made an attempt of adding a polyamide to a polypropylene to improve the heat resistance, oil resistance, etc. of polypropylene without reducing the impact resistance or polypropylene. However, since there is no compatibility between polypropylene and polyamide, they cause delamination and no desired material can be obtained when they are melt mixed as they are. Hence, there was used, in place of a polypropylene, a modified polypropylene obtained by grafting a polypropylene with an unsaturated carboxylic acid or a derivative of an unsaturated carboxylic acid (Japanese Patent Publication No. 30945/1970). This approach makes a polypropylene and a polyamide to be compatible with each other and can improve the heat resistance of polypropylene without reducing the impact resistance of polypropylene.”

U.S. Pat. No. 5,206,284 also discloses that “However, even in the above improvement of polypropylene by addition of polyamide, the improvement effect is not satisfactory as long as there is used, as the polyamide, an ordinary polyamide such as nylon-6, nylon-6,6, nylon-112 or the like. Recently there has been made a proposal of adding an aromatic polyamide and a glass fiber to a polypropylene to obtain a material of high strength and low water absorbability [Japanese Patent Application Kokai (Laid-Open) No. 203654/1985]. This proposal is not sufficient when viewed from the improvement of polypropylene in both heat resistance and impact resistance. In order to significantly improve the heat resistance and impact resistance of polypropylene by addition of polyamide thereto, the dispersibility of polyamide particles in polypropylene and the cohesiveness among polyamide particles are very important. The improvement of polyamide particles in these properties has been necessary.”

The polymeric material used in the magnetic mineral composition of this invention may be a polyester, such as poly(ethylene terephthalate), as is disclosed in U.S. Pat. No. 5,876,812, the entire disclosure of which is hereby incorporated by reference into this specification. Claim 1 of such patent describes “1. A transparent container for a flowable food product having a decreased permeability for gases, the transparent container consisting essentially of a layer of polyethylene terephthalate integrated with a plurality of synthetic smectite particles between 0.1% and 10% weight of the layer of polyethylene terephthalate, each of the plurality of smectite particles having a thickness of between 9 Angstroms and 100 nanometers, and an aspect ratio of between 100 and 2000, the layer of polyethylene terephthalate having a thickness range of approximately 100 microns to approximately 2000 microns.”Reference may also be had to related U.S. Pat. No. 5,972,448, the entire disclosure of which is hereby incorporated by reference into this specification.

The polymeric material used in the magnetic mineral composition of this invention may be a melt processable polymer, as that term is defined in U.S. Pat. No. 5,962,53, the entire disclosure of which is hereby incorporated by reference into this specification. Claim 1 of U.S. Pat. No. 5,962,553 describes “1. A method of making a composite, comprising the steps of: (a) providing 100 parts by weight of melt processable polymer which is a fluoroplastic selected from the group consisting of ethylene-tetrafluoroethylene copolymer, perfluorinated ethylene-propylene copolymer, and tetrafluoroethylene-perfluoro(propyl vinyl ether) copolymer; (b) providing between 1 and 80 parts by weight of a modified layered clay, the modified layered clay being a layered clay having negatively charged layers and modified so as to have organophosphonium cations intercalated between the negatively charged layers, the organophosphonium cations having the structure R1P+(R2)3 wherein R1 is a C8 to C24 alkyl or arylalkyl group and each R2, which may be the same or different, is an aryl, arylalkyl, or a C1 to C6 alkyl group; and (c) melt-blending together the melt processable polymer and the modified layered clay to form the composite.”

Some of the “melt processable polymers” that may be used in the process of U.S. Pat. No. 5,962,553 are described at columns 4-5 of such patent, wherein it is disclosed that “Suitable melt processable polymers preferably have a melt processing temperature of at least about 250° C., preferably at least about 270° C. Typically, a melt processable polymer is melt processed at a temperature which is at least about 20 to 30° C. above a relevant transition temperature, which can be either a Tm or a Tg, in order to attain complete melting (or softening) of the polymer and to lower its viscosity. Further, even if a polymer is nominally melt-processed at a temperature such as 240° C., shear heating can increase the actual localized temperature experienced by the modified layered clay to rise above 250° C. for extended periods. Thus, a melt processable polymer having a melt processing temperature of at least about 250° C. will have a Tm or Tg of at least about 220° C.”

U.S. Pat. No. 5,962,553 also discloses that “One class of melt processable polymers which can be used are crystalline thermoplastics having a crystalline melting temperature (Tm) of at least about 220° C., preferably at least about 250° C., and most preferably at least about 270° C. Tm may be measured by the procedure of ASTM standard E794-85 (Reapproved 1989). For the purposes of this specification, Tm is the melting peak Tm as defined at page 541 of the standard. Either a differential scanning calorimeter (DSC) or a differential thermal analyzer (DTA) may be used, as permitted under the standard, the two techniques yielding similar results.”

U.S. Pat. No. 5,962,553 also discloses that “Another class of melt processable polymers which can be used are amorphous polymers having a glass transition temperature (Tg) of at least about 220° C., preferably at least about 250° C., and most preferably at least about 270° C. Tg may be measured according to ASTM E 1356-91 (Reapproved 1995), again using either DSC or DTA.”

U.S. Pat. No. 5,962,553 also discloses that “Turning now to specific types of melt processable polymers which can be used, these include fluoroplastics, poly(phenylene ether ketones), aliphatic polyketones, polyesters, poly(phenylene sulfides) (PPS), poly(phenylene ether sulfones) (PES), poly(ether imides), poly(imides), polycarbonate, and the like. Fluoroplastics are preferred. The organophosphonium modified clays of this invention can also be used to make nanocomposites with polymers having lower melting temperatures, such as aliphatic polyamides (nylons), but since the conventional quaternary ammonium salts can also be used, no special advantage is obtained in such instance.”

U.S. Pat. No. 5,962,553 also discloses that “A preferred fluoroplastic is ethylene-tetrafluoroethylene copolymer, by which is meant a crystalline copolymer of ethylene, tetrafluoroethylene and optionally additional monomers. Ethylene-tetrafluoroethylene copolymer is also known as ETFE or poly(ethylene-tetrafluoroethylene), and herein the acronym ETFE may be used synonymously for convenience. The mole ratio of ethylene to tetrafluoroethylene can be about 35-60:65-40. A third monomer may be present in an amount such that the mole ratio of ethylene to tetrafluoroethylene to third monomer is about 40-60:15-50:0-35. Preferably the third monomer, if present, is so in an amount of about 5 to about 30 mole %. The third monomer may be, e.g., hexafluoropropylene; 3,3,3-trifluoropropylene-1; 2-trifluoromethyl-3,3,3-trifluoropropylene-1; or perfluoro(alkyl vinyl ether). The melting point varies depending on the mole ratio of ethylene and tetrafluoroethylene and the presence or not of a third monomer. Commercially available ETFE's have melting points between 220 and 270° C., with the grades having melting points above 250° C. being most appropriate for this invention.”

U.S. Pat. No. 5,962,553 also discloses that “ETFE for use in this invention is available from various suppliers, including from E.I. du Pont de Nemours under the trade name Tefzel (e.g., grades 280, 2181 and 2129) and from Daikin Industries under the trade name Neoflon (e.g., grades 540, 610 and 620).”

U.S. Pat. No. 5,962,553 also discloses that “Another fluoroplastic suitable for use in this invention is perfluorinated ethylenepropylene copolymer (also known as FEP), by which is meant a copolymer of tetrafluoroethylene (TFE), hexafluoropropylene (HFP), and optionally additional monomers. Preferably, FEP is predominantly random and has a relatively low HFP content, between about 1 and about 15 weight % based on the total weight of TFE and HFP. Preferably the molecular weight is between about 100,000 and about 600,000. A preferred FEP is available from E.I. du Pont de Nemours under the trade name Teflon FEP. The melting point of FEP is about 260° C.”

U.S. Pat. No. 5,962,553 also discloses that “Yet another suitable fluoroplastic is tetrafluoroethylene-perfluoro(propyl vinyl ether) copolymer (also known as PFA), by which is meant a copolymer of tetrafluoroethylene, perfluoro(propyl vinyl ether), and optionally a third monomer. The third monomer, where present, is typically present in an amount of 5% or less by weight of the polymer and may be, for example, perfluoro(methyl vinyl ether), perfluoro(ethyl vinyl ether), perfluoro(butyl vinyl ether), or any other suitable monomer. A representative PFA has about 90 to 99 (preferably 96 to 98) weight % tetrafluoroethylene derived repeat units and about 1 to 10 (preferably 2 to 4) weight % perfluoro(propyl vinyl ether) derived repeat units. A representative crystalline melting point is about 302 to 305° C. PFA is available from E.I. du Pont de Nemours under the trade name Teflon PFA.”

U.S. Pat. No. 5,962,553 also discloses that “Suitable poly(phenylene ether ketones) are disclosed in Dahl, U.S. Pat. No. 3,953,400 (1976); Dahl et al., U.S. Pat. No. 3,956,240 (1976); Dahl, U.S. Pat. No. 4,111,908 (1978); Rose et al., U.S. Pat. No. 4,320,224 (1982); and Jansons et al., U.S. Pat. No. 4,709,007 (1987); the disclosures of which are incorporated herein by reference. Typically, they have Tm's in excess of 300° C. Exemplary poly(phenylene ether ketones) comprise one or more of the following repeat units: [Figure]”

U.S. Pat. No. 5,962,553 also discloses that “Suitable aliphatic polyketones have a repeat unit [Figure] alone or in combination with a repeat unit [Figure] An exemplary disclosure of such aliphatic polyketones is found in Machado et al., ANTEC '95, pp. 2335-2339 (1995), the disclosure of which is incorporated herein by reference. Aliphatic polyketones are believed to be crystalline with Tm's of 220° C. or above.”

U.S. Pat. No. 5,962,553 also discloses that “A suitable polyester is poly(ethylene terephthalate) (PET), having the repeat unit [Figure] PET is available commercially from a variety of suppliers. It is believed to be crystalline, with a Tm in the range of about 250 to about 265° C.”

U.S. Pat. No. 5,962,553 also discloses that “A suitable poly(phenylene sulfide) has the repeat unit [Figure] It has a Tm of about 285° C. and is available under the trade name Ryton from Phillips.”

U.S. Pat. No. 5,962,553 also discloses that “Suitable poly(phenylene ether sulfones) have the repeat units such as [Figure] or [Figure]”

U.S. Pat. No. 5,962,553 also discloses that “Suitable poly(ether imides) are disclosed in Wirth et al., U.S. Pat. No. 3,838,097 (1974); Heath et al., U.S. Pat. No. 3,847,867 (1974); and Williams, III et al., U.S. Pat. No. 4,107,147 (1978); the disclosures of which are incorporated herein by reference. Poly(ether imide) is available under the trade name Ultem from General Electric. A preferred poly(ether imide) has the repeat unit: [Figure]”

U.S. Pat. No. 5,962,553 also discloses that “A suitable polyimide is a thermoplastic supplied under the trade name Aurum by Mitsui Toatsu Chemical, Inc. It has a Tg of about 250° C. and a Tm of about 388° C.”

U.S. Pat. No. 5,962,553 also discloses that “A suitable polycarbonate has the repeat unit [Figure] and is available from General Electric Company.”

The polymeric material used in the magnetic mineral composition of this invention may be a mixture of two or more polymers such as, e.g., the mixture disclosed in U.S. Pat. No. 6,117,932, the entire disclosure of which is hereby incorporated by reference into this specification. Claim 1 of this patent describes “1. A resin composite comprising, an organophilic clay and a polymer, wherein said polymer comprises: component (a) two or more polymers, at least one of which is poly(phenylene oxide), or component (b) a copolymer comprising at least one oxazoline functional group.”

The polymeric material used in the magnetic mineral composition of this invention may be a polymerized aminoaryl lactam monomer, as is described in U.S. Pat. No. 6,136,908, the entire disclosure of which is hereby incorporated by reference into this specification. Claim 1 of this patent describes “1. A method for producing a thermoplastic nanocomposite, comprising the steps of: contacting a swellable layered silicate with a polymerizable N-aminoaryl substituted lactam monomer to achieve intercalation of said lactam monomer between adjacent layers of said layered silicate; and .” admixing the intercalated layered silicate with a thermoplastic polymer, and heating the admixture to provide for flow of said polymer and polymerization of the intercalated lactam monomer to cause exfoliation of the layered silicate, thereby forming a thermoplastic nanocomposite having exfoliated silicate layers dispersed in a thermoplastic polymer matrix.” The lactam monomer used in such process is described in column 2 of the patent as being “ . . . an N-aminoaryl substituted lactam monomer, which can be prepared via a one-step synthesis by coupling an aromatic amino acid with a lactam having a cyclic ring system containing 1 to 12 carbon atoms. Illustrative example of such aminoaryl lactams are N-(p-aminobenzoyl)caprolactam, N-(p-aminobenzoyl)butyrolactam, N-(p-aminobenzoyl)valerolactam, and N-(p-aminobenzoyl)dodecanelactam.”

The polymeric material used in the magnetic mineral composition may be a conducting polymer, as that term is described in U.S. Pat. No. 6,136,909, the entire disclosure of which is hereby incorporated by reference into this specification. Claim 1 of this patent describes a process for preparing a conductive polymeric nanocomposite, disclosing “1. A method for producing a conductive polymeric nanocomposite, comprising the steps of: (a) forming a reaction mixture comprising water, an aniline monomer, a protonic acid, an oxidizing agent, and a layered silicate which has been subjected to an acid treatment or is intercalated with polyethylene glycol; and (b) subjecting said reaction mixture to oxidative polymerization to form a conducive polymeric nanocomposite having said layered silicate dispersed in a polymeric matrix of polyaniline, wherein said nanocomposite has a conductivity of greater than 10⁻¹ S/cm.”

Conducting polymers are discussed in column 1 of U.S. Pat. No. 6,136,909, wherein it is disclosed that “In the past decade, conducting polymers have been used in many fields, such as batteries, displays, optics, EMI shielding, LEDs, sensors, and the aeronautical industry. High molecular weight polyaniline has emerged as one of the more promising conducting polymers because of its excellent chemical stability combined with respectable levels of electrical conductivity of the doped or protonated material. Processing of polyaniline high polymers into useful objects and devices, however, has been problematic. Melt processing is not possible, since the polymer decomposes at temperatures below a softening or melting point. In addition, major difficulties have been encountered in attempts to dissolve the high molecular weight polymer.”

U.S. Pat. No. 6,136,909 also discloses that “One known method to improve the processibility of polyaniline is by employing a protonic acid dopant containing a long-chain sulfonic group in the polymerization of aniline to form an emulsified colloidal dispersion. However, this method requires a large quantity of long-chain dopants, which decrease the conductivity and mechanical properties of polyaniline. In addition, high aspect ratios of polyaniline are unavailable through this method. In conventional guest-host methods for preparing polyaniline/layered inorganic composites, aniline monomers are interposed between layered hosts, and then subjected to oxidative polymerization to form composites with highly ordered polymer matrices. The polyaniline composite thus obtained, however, commonly has a conductivity lower than 10-2 S/cm. Moreover, they do not give nanoscale structures. The interlayer spacing (d-spacing) of the inorganic layers is less than 15 .Angstroms.”

The polymeric material used in the magnetic mineral composition of this invention may be a benzoaxazine polymer as described, e.g., in claim 1 of U.S. Pat. No. 6,323,270, the entire disclosure of which is hereby incorporated by reference into this specification. Claim 1 of this patent describes “1. A nanocomposite composition comprising clay and a benzoxazine monomer, oligomer, and/or polymer in amount effective to form nanocomposite.” The preparation of these polymers is described in column 5 of the patent, wherein it is disclosed that “Benzoxazines are prepared by reacting a phenolic compound with an aldehyde and an amine, desirably an aromatic amine. The conventional phenolic reactants for benzoxazines include, for instance, mono and polyphenolic compounds having one or more phenolic groups of the formula [Figure] in which R1 through R5 can independently be H; OH; halogen; linear or branched aliphatic groups having from 1 to 10 carbon atoms; mono, di, or polyvalent aromatic groups having from 6 to 12 carbon atoms; or a combination of said aliphatic groups and said aromatic groups having from 7 to 12 carbon atoms; mono and divalent phosphine groups having up to 6 carbon atoms; or mono, di and polyvalent amines having up to 6 carbon atoms. In one embodiment, at least one of the ortho positions to the OH is unsubstituted, i.e. at least one of R1 to R5 is hydrogen. In polyphenolic compounds, one or more of the R1 through R5 can be an oxygen, an alkylene such as methylene or other hydrocarbon connecting molecule, etc. Further nonhydrogen and nonhalogen R1 through R5 groups as described above less one or more hydrogens or a P═O can serve to connect two or more phenolic groups creating a polyphenolic compound which can be the phenolic compound. Example of mono-functional phenols include phenol; cresol; 2-bromo-4-methylphenol; 2-allyphenol; 1,4-aminophenol; and the like. Examples of difunctional phenols (polyphenolic compounds) include phenolphthalane; biphenol; 4-4′-methylene-di-phenol; 4-4′-dihydroxybenzophenone; bisphenol-A; 1,8-dihydroxyanthraquinone; 1,6-dihydroxnaphthalene; 2,2′-dihydroxyazobenzene; resorcinol; fluorene bisphenol; and the like. Examples of trifunctional phenols comprise 1,3,5-trihydroxy benzene and the like. Polyvinyl phenol is also a suitable component for the benzoxazine compounds that constitute the subject of the invention.”

The polymeric material used in the magnetic mineral composition may be a polyphenylene ether resin as is disclosed, e.g., in U.S. Pat. No. 6,350,804, the entire disclosure of which is hereby incorporated by reference into this specification. Claim 1 of this patent describes “1. A composition comprising: about 30 to about 70 parts by weight of a polyphenylene ether resin; about 20 to about 60 parts by weight of an alkenylaromatic compound, wherein the alkenylaromatic compound is a high impact polystyrene; and about 1 to about 10 parts by weight of an organoclay; wherein the parts by weight of the polyphenylene ether, the alkenylaromatic compound, and the organoclay sum to 100.”

The polymeric material used in the magnetic mineral composition may be a syndiotactic polystyrene, as that term is defined in the claims of U.S. Pat. No. 6,410,142, the entire disclosure of which is hereby incorporated by reference into this specification. Claim 1 of this patent describes “A syndiotactic polystyrene/clay nanocomposite comprising: a polymer matrix comprising syndiotactic polystyrene (sPS); and a layered clay material uniformly dispersed in the polymer matrix, said layered clay material being intercalated with an organic onium cation, and the interlayer distances of said layered clay material being at least 20 angstroms.” The nanocomposite described in such claim 1 is further described at columns 2-3 of U.S. Pat. No. 6,410,142, wherein it is disclosed that “The sPS/clay nanocomposite of this invention comprises a polymer matrix containing syndiotactic polystyrene (sPS), and a layered clay material uniformly dispersed in the polymer matrix, said layered clay material being intercalated with an organic onium cation, and the interlayer distances of said layered clay material being at least 20 .ANG. Optionally, the layered clay material may be intercalated with a polymer or oligomer which is compatible or partially compatible with sPS. The amount of the optionally intercalated polymer or oligomer is preferably in the range from 0.5 to 50 parts by weight per 100 parts by weight of the clay material.”

U.S. Pat. No. 6,410,142 also discloses that “The polymer matrix in the composite material of this invention is a resin containing sPS, namely, a sPS or a mixture thereof with other polymers. The molecular weight of the sPS to be used in the present invention is not specifically limited, but is preferably within the range from about of 15,000 to 800,000 in terms of weight-average molecular weight (Mw).”

U.S. Pat. No. 6,410,142 also discloses that “The layers of clay material in the composite material of this invention, which are intended to impart the polymeric material with high mechanical strength, have a thickness of about 7 to 12 .Angstroms. Also, it has been found that the nano-dispersed clay material unexpectedly increases the crystallization rate and crystallization temperature of sPS. The greater the proportion of the clay material in the sPS matrix, the more marked the effects achieved.”

U.S. Pat. No. 6,410,142 also discloses that “The amount of the clay material dispersed in the composite material of this invention is preferably in the range from about 0.1 to 40 parts by weight per 100 parts by weight of the polymer matrix. If this amount is less than 0.1 parts, a sufficient reinforcing effect cannot be expected. If the amount exceeds 40 parts, on the other hand, the resulting product is powdery interlayer compound which cannot be used as moldings. In addition, it is also preferable that the composite material of this invention be such that the interlayer distance is at least 30 Angstroms. The greater the interlayer distance is, the better the mechanical strength will be.”

U.S. Pat. No. 6,410,142 also discloses that “Next, the process for manufacturing composite material of this invention is described below. The first step is to bring a cation-type surfactant into contact with a clay material having a cation-exchange capacity of about 50 to 200 meq/100 g, thereby adsorbing the surfactant on the clay material. This can be accomplished by immersing the clay material in an aqueous solution containing the surfactant, followed by washing the treated clay material with water to remove excess ions, thereby effecting ion-exchange operation.”

U.S. Pat. No. 6,410,142 also discloses that “The clay material used in this invention can be any clay material (both natural and synthesized) having a cation exchange capacity of about 50 to 200 meq/100 g. Typical examples include smectite clays (e.g., montmorillonite, saponite, beidellite, nontronite, hectorite, and stevensite), vermiculite, halloysite, sericite, and mica. With a clay material whose cation-exchange capacity exceeds 200 meg/100 g, its interlayer bonding force is too strong to give intended composite materials of this invention. If the capacity is less than 50 meq/100 g, on the other hand, ion exchange or adsorption of surfactant will not be sufficient, making it difficult to produce composite materials as intended by this invention.”

U.S. Pat. No. 6,410,142 also discloses that “The cation-type surfactant serves to expand the interlayer distance in a clay material, thus facilitating the formation of polymer between the silicate layers. The surfactants used in the present invention are organic compounds containing onium ions which-are capable of forming a firm chemical bond with silicates through ion-exchange reaction. Particularly preferred surfactants are ammonium salts containing at least 12 carbon atoms, such as n-hexadecyl trimethylammonium bromide and cetyl pyridinium chloride.”

U.S. Pat. No. 6,410,142 also discloses that “Optionally, the surface modified clay material may be intercalated with a polymer or oligomer, which is compatible or partially compatible with sPS, as a subsequent modification. For example, this can be accomplished by admixing the modified clay material with a styrene monomer or 2,6-xylenol monomer, and polymerizing the monomer to obtain atactic polystyrene (aPS) or poly(2,6-dimethyl-1,4-phenylenen oxide) (PPO) intercalated in the modified clay material, respectively.”

U.S. Pat. No. 6,410,142 also discloses that “The next step in the process of this invention is to mix a styrene monomer with the modified clay material, which may be intercalated with a polymer or oligomer other than sPS and is compatible or partially compatible with sPS, and to polymerize the mixture by using a catalyst composition containing metallocene, thereby giving an intended composite material of this invention. Typically, the polymerization of syndiotactic polystyrene requires a catalyst composition containing a metallocene catalyst and a methyl aluminoxane (MAO) co-catalyst. The concerted action of the metallocene and the methyl aluminoxane allows syndiotactic polystyrene to be polymerized. Suitable polymerization time varies with the surfactant adopted, but is usually in the range from 15 to 40 minutes for reaching a weight-average molecular weight of 15,000 to 800,000.”

U.S. Pat. No. 6,410,142 also discloses that “Alternatively, the composite material of this invention can be obtained by directly blending the modified clay material with a syndiotactic polystyrene, wherein the clay material may be intercalated with a polymer or oligomer which is compatible or partially compatible with sPS. The blending can be accomplished by a variety of methods which are well-known in the art, such as melt blending or solution blending. In general, the blending can be accomplished by melt blending in a closed system. For example, this can be carried out in a single- or multi-screw extruder, a Banbury mill, or a kneader at a temperature sufficient to cause the polymer blend to melt flow. According to this invention, the blending is preferably carried out at a temperature ranging from about 290° to 310° C. Solution blending can be carried out by dispersing the modified clay in an organic solution of sPS, and thoroughly mixing the dispersion. The intended composite material of this invention can be therefore obtained after evaporation of the organic solvent.”

U.S. Pat. No. 6,410,142 also discloses that “The composite materials obtained according to the procedure detailed above may be directly injection-molded, extrusion-molded or compression-molded, or may be mixed with other types of polymers before molding.”

The polymeric material used in the magnetic mineral composition of this invention may be an epoxy resin such as, e.g., the epoxy resin described in U.S. Pat. No. 6,548,159, the entire disclosure of which is hereby incorporated by reference into this specification. Claim 1 of this patent describes “1. An epoxy/clay nanocomposite, comprising: a polymer matrix comprising an epoxy resin; and an exfoliated layered clay material uniformly dispersed in the polymer matrix, wherein the exfoliated layered clay material is present in an amount ranging from about 0.1% to 10% by weight based on the total weight of the nanocomposite and has been modified by ion exchange with (1) benzalkonium chloride and (2) dicyandiamide or tetraethylenepentamine.”

The polymeric material used in the magnetic mineral composition of this invention may be almost any kind of thermoplastic or thermosetting polymer, as is disclosed in U.S. Pat. No. 6,562,891, the entire disclosure of which is hereby incorporated by reference into this specification. Claim 1 of this patent describes “1. A method for producing a polymer/clay composite comprising a polymer matrix selected from the group consisting of polyethylene terephthalate (PET), epoxy resins and polyaniline and a layered clay mineral uniformly dispersed in said polymer matrix, said method comprising the steps of: (a) intercalating a layered clay mineral with a polymerization catalyst in a polar solvent selected from the group consisting of ethylene glycol and water; (b) admixing the intercalated clay mineral with monomers or oligomers of said polymer matrix; and (c) polymerizing said monomers or oligomers under the catalysis of said polymerization catalyst.” Some of the polymers that may be used in the process of such U.S. Pat. No. 6,562,891 are described in column 3 of the patent, wherein it is disclosed that “The modified clay mineral of the present invention can be admixed with almost any kind of thermoplastic or thermosetting polymers by way of melt blending or oligomer intercalating, followed by polymerization to form polymer/clay nanocomposites. If necessary, oligomers can be first included between the adjacent silicate layers before subjected to polymerization, which results in a better dispersibility of the exfoliated silicate layers in the polymer matrix. The matrix polymer suitable for use in the present invention includes, for example; conductive polymers such as polyaniline, polypyrrole, polythiphene; polyesters such as polyethylene terephthalate (PET), polybutylene terephthalate (PBT), polycarbonate (PC); silicones such as polydimethyl siloxane, silicone rubber, silicone resin; acrylic resins such as polymethylmethacrylate, polyacrylate; epoxy resins such as bisphenol-epoxy, phenolic-epoxy; and styrene polymers such as polystyrene, styrene-acrylonitrile copolymer, acrylonitrile-butadiene-styrene copolymer.”

The polymeric material used in the magnetic mineral composition of this invention may be one of more of the polyamides described in U.S. Pat. No. 6,627,324, the entire disclosure of which is hereby incorporated by reference into this specification. Claim 1 of this patent describes “1. A single or multi-layer film having at least one layer (I) of a polyamide composition having nanoscale nucleating particles dispersed therein wherein said nanoscale particles have an aspect ratio of at least 10 in two randomly selectable directions, and, on a number-weighted average a dimension of less than 100 nm in at least one direction that is randomly selectable, the amount by weight of the nanoscale particles, based on the total weight of the polyamide forming the layer (I), is between 10 ppm and 2000 ppm, and wherein the polyamide composition forming the layer (I) is selected from the group consisting of polyamide 6/66, partially aromatic copolyamides having mole-weighted aromatic monomer contents of between 3% and 15% and mixtures of at least one of polyamide 6/66 and polyamide 6 with partially aromatic homopolyamides or copolyamides having mole-weighted aromatic monomer contents of between 3% and 15% by weight of mixture.”

The polymeric material used in the magnetic mineral composition of this invention may be one or more of the epoxy resins disclosed in U.S. Pat. No. 6,683,122, the entire disclosure of which is hereby incorporated by reference into this specification. As is disclosed in such patent at columns 4 et seq., examples of suitable epoxy resins include “I) Polyglycidyl and poly(β-methylglycidyl) esters, obtainable by reaction of a compound having at least two carboxyl groups in the molecule with epichlorohydrin and β-methyl-epichlorohydrin, respectively. The reaction is advantageously carried out in the presence of bases. Aliphatic polycarboxylic acids can be used as the compound having at least two carboxyl groups in the molecule. Examples of such polycarboxylic acids are oxalic acid, succinic acid, glutaric acid, adipic acid, pimelic acid, sebacic acid, suberic acid, azelaic acid and dimerised or trimerised linoleic acid. It is also possible, however, to use cycloaliphatic polycarboxylic acids, for example tetrahydrophthalic acid, 4-methyltetrahydrophthalic acid, hexahydrophthalic acid or 4-methylhexahydrophthalic acid. Aromatic polycarboxylic acids, for example phthalic acid, isophthalic acid or terephthalic acid, may also be used.”

As is also disclosed in U.S. Pat. No. 6,683,122, to illustrate suitable epoxy resins, “II) Polyglycidyl or poly(β-methylglycidyl) ethers, obtainable by reaction of a compound having at least two free alcoholic hydroxy groups and/or phenolic hydroxy groups with epichlorohydrin or β-methylepichlorohydrin under alkaline conditions, or in the presence of an acidic catalyst and subsequent alkali treatment. The glycidyl ethers of this kind may be derived, for example, from acyclic alcohols, such as from ethylene glycol, diethylene glycol and higher poly(oxyethylene) glycols, propane-1,2-diol or poly(oxypropylene) glycols, propane-1,3-diol, butane-1,4-diol, poly(oxytetramethylene) glycols, pentane-1,5-diol, hexane-1,6-diol, hexane-2,4,6-triol, glycerol, 1,1,1-trimethylolpropane, pentaerythritol, sorbitol and also from polyepichlorohydrins, but they may also be derived, for example, from cycloaliphatic alcohols, such as 1,4-cyclohexanedimethanol, bis(4-hydroxycyclohexyl)methane or 2,2-bis(4-hydroxycyclohexyl)propane, or they may have aromatic nuclei, such as N,N-bis(2-hydroxyethyl)aniline or p,p′-bis(2-hydroxyethylamino)diphenylmethane. The glycidyl ethers may also be derived from mononuclear phenols, for example from resorcinol or hydroquinone, or they may be based on polynuclear phenols, for example bis(4-hydroxyphenyl)methane, 4,4′-di-hydroxybiphenyl, bis(4-hydroxyphenyl)sulfone, 1,1,2,2-tetrakis(4-hydroxyphenyl)ethane, 2,2-bis(4-hydroxyphenyl)propane, 2,2-bis(3,5-dibromo-4-hydroxyphenyl)propane and also on novolaks, obtainable by condensation of aldehydes, such as formaldehyde, acetaldehyde, chloral or furfuraldehyde, with phenols, such as phenol, or with phenols substituted in the nucleus by chlorine atoms or C1-C9 alkyl groups, for example 4-chlorophenol, 2-methylphenol or 4-tert-butylphenol, or by condensation with bisphenols, such as those of the kind mentioned above.”

As is also disclosed in U.S. Pat. No. 6,683,122, to further illustrate suitable epoxy resins, “III) Poly(N-glycidyl) compounds, obtainable by dehydrochlorination of the reaction products of epichlorohydrin with amines that contain at least two amine hydrogen atoms. Such amines are, for example, aniline, n-butylamine, bis(4-aminophenyl)methane, m-xylylene-diamine and bis(4-methylaminophenyl)methane. Poly(N-glycidyl) compounds also include, however, triglycidyl isocyanurate, N,N′-diglycidyl derivatives of cycloalkyleneureas, such as ethyleneurea or 1,3-propyleneurea, and diglycidyl derivatives of hydantoins, such as of 5,5-dimethylhydantoin.

As is also disclosed in U.S. Pat. No. 6,683,122, to further illustrate suitable epoxy resins, “IV) Poly(S-glycidyl) compounds, for example di-S-glycidyl derivatives, derived from dithiols, for example ethane-1,2-dithiol or bis(4-mercaptomethylphenyl) ether.”

As is also disclosed in U.S. Pat. No. 6,683,122, to further illustrate suitable epoxy resins, “V Cycloaliphatic epoxy resins, for example bis(2,3-epoxycyclopentyl) ether, 2,3-epoxycyclo-pentylglycidyl ether, 1,2-bis(2,3-epoxycyclopentyloxy)ethane or 3,4-epoxycyclohexylmethyl 3′,4′-epoxycyclohexanecarboxylate.”

As is also disclosed in U.S. Pat. No. 6,683,122, to further illustrate suitable epoxy resins, “VI) Epoxy resins in which the 1,2-epoxy groups are bonded to different hetero atoms or functional groups, for example the N,N,O-triglycidyl derivative of 4-aminophenol, the glycidyl ether glycidyl ester of salicylic acid, N-glycidyl-N′-(2-glycidyloxypropyl)-5,5-dimethyhydantoin or 2-glycidyloxy-1,3-bis(5,5-dimethyl-1-glycidylhydantoin-3-yl)propane.”

As is also disclosed in U.S. Pat. No. 6,683,122, to further illustrate suitable epoxy resins, “VII) Epoxidation products of unsaturated synthetic or natural oils or derivatives thereof; suitable natural oils are, for example, soybean oil, linseed oil, perilla oil, tung oil, oiticica oil, safflower oil, poppyseed oil, hemp oil, cottonseed oil, sunflower oil, rapeseed oil, walnut oil, beet oil, high oleic triglycerides, triglycerides from euphorbia plants, groundnut oil, olive oil, olive kernel oil, almond oil, kapok oil, hazelnut oil, apricot kernel oil, beechnut oil, lupin oil, maize oil, sesame oil, grapeseed oil, lallemantia oil, castor oil, herring oil, sardine oil, menhaden oil, whale oil, tall oil, palm oil, palm kernel oil, coconut oil, cashew oil and tallow oil and derivatives derived therefrom. Also suitable are higher unsaturated derivatives that can be obtained by subsequent dehydration reactions of those oils. Examples of suitable synthetic oils are polybutadiene oils, polyethylene oils, polypropylene oils, polypropylene oxide oils, polyethylene oxide oils and paraffin oils.”

U.S. Pat. No. 6,683,122 also discloses that “It is preferable to use as epoxy resin in the curable mixtures according to the invention a fluid or viscous polyglycidyl ether or ester, especially a fluid or viscous bisphenol diglycidyl ether. Especially preferred are bisphenol diglycidyl ethers, especially bisphenol A diglycidyl ether and bisphenol F diglycidyl ether.”

U.S. Pat. No. 6,683,122 also discloses that “The above-mentioned epoxy compounds are known and some of them are commercially available. It is also possible to use mixtures of epoxy resins. For example, cured products having a high tensile strength and a high modulus of elasticity can be obtained when the epoxy resin used is a mixture of a bisphenol diglycidyl ether and an epoxidised oil or an epoxidised rubber.”

U.S. Pat. No. 6,683,122 also discloses that “Preferably such mixtures comprise bisphenol A diglycidyl ether and epoxidised soybean oil or linseed oil. The amount of epoxidised oil or rubber is preferably from 0.5 to 30% by weight, especially from 1 to 20% by weight, based on the total amount of epoxy resin.”

U.S. Pat. No. 6,683,122 also discloses that “All customary hardeners for epoxides can be used; preferred hardeners are amines, carboxylic acids, carboxylic acid anhydrides and phenols. It is also possible to use catalytic hardeners, for example imidazoles. Such hardeners are described, for example, in H. Lee, K. Neville, Handbook of Epoxy Resins, McGraw Hill Book Company, 1982.”

U.S. Pat. No. 6,683,122 also discloses that “In a special embodiment of the invention the hardener is an amine, a carboxylic acid, a carboxylic acid anhydride or a phenol and additionally contains a maleinated oil, a maleinated rubber or an alkenyl succinate. Using those specific hardener mixtures it is possible to obtain cured products having a high tensile strength and a high modulus of elasticity.”

U.S. Pat. No. 6,683,122 also discloses that “Suitable maleinated oils are, for example, the reaction products of the above-mentioned synthetic or natural oils or rubbers with maleic acid anhydride. An example of an alkenyl succinate is dodecenyl succinate. The amount of maleinated oil or rubber or of alkenyl succinate is preferably from 0.5 to 30% by weight, more especially from 1 to 20% by weight, based on the total amount of hardener.”

U.S. Pat. No. 6,683,122 also discloses that “The amount of hardening agent used is governed by the chemical nature of the hardening agent and by the desired properties of the curable mixture and of the cured product. The maximum amount can readily be determined by a person skilled in the art. The preparation of the mixtures can be carried out in customary manner by mixing the components together by manual stirring or with the aid of known mixing apparatus, for example by means of stirrers, kneaders or rollers. Depending upon the application, conventionally used additives, for example fillers, pigments, colourings, flow agents or plasticisers, may be added to the mixtures.”

U.S. Pat. No. 6,683,122 also discloses that the polymeric material that may be mixed with the layer silicate material may be a polyurethane. U.S. Pat. No. 6,683,122 also discloses that “Further preferred components A are polyurethane precursors. Structural components for crosslinked polyurethanes are polyisocyanates, polyols and optionally polyamines, in each case having two or more of the respective functional groups per molecule.”

U.S. Pat. No. 6,683,122 also discloses that “The invention therefore relates also to compositions comprising as component A mixture of a polyisocyanate having at least two isocyanate groups and a polyol having at least two hydroxyl groups.”

U.S. Pat. No. 6,683,122 also discloses that “Aromatic and also aliphatic and cycloaliphatic polyisocyanates are suitable building blocks for polyurethane chemistry. Examples of frequently used polyisocyanates are 2,4- and 2,6-diisocyanatotoluene (TDI) and mixtures thereof, especially the mixture of 80% by weight 2,4-isomer and 20% by weight 2,6-isomer; 4,4′- and 2,4′- and 2,2′-methylenediisocyanate (MDI) and mixtures thereof and technical grades that, in addition to containing the above-mentioned simple forms having two aromatic nuclei, may also contain polynuclear forms (polymer MDI); naphthalene-1,5-diisocyanate (NDI); 4,4′,4″-triisocyanatotriphenylmethane and 1,1-bis(3,5-diisocyanato-2-methyl)-1-phenylmethane; 1,6-hexamethylene diisocyanate (HDI) and 1-isocyanato-3-(isocyanatomethyl)-3,5,5-trimethylcyclohexane (isophorone diisocyanate, IDPI). Such basic types of polyisocyanates may optionally also have been modified by dimerisation or trimerisation with the formation of corresponding carbodiimides, uretdiones, biurets or allophanates.”

U.S. Pat. No. 6,683,122 also discloses that “Especially preferred polyisocyanates are the various methylene diisocyanates, hexamethylene diisocyanate and isophorone diisocyanate.”

U.S. Pat. No. 6,683,122 also discloses that “As polyols there may be used in the polyurethane production both low molecular weight compounds and oligomeric and polymeric polyhydroxyl compounds. Suitable low molecular weight polyols are, for example, glycols, glycerol, butanediol, trimethylolpropane, erythritol, pentaerythritol; pentitols, such as arabitol, adonitol or xylitol; hexitols, such as sorbitol, mannitol or dulcitol, various sugars, for example saccharose, or sugar and starch derivatives. Low molecular weight reaction products of polyhydroxyl compounds, such as those mentioned, with ethylene oxide and/or propylene oxide are also frequently used as polyurethane components, as well as the low molecular weight reaction products of other compounds that contain sufficient numbers of groups capable of reaction with ethylene oxide and/or propylene oxide, for example the corresponding reaction products of amines, such as especially ammonia, ethylenediamine, 1,4-diaminobenzene, 2,4-diaminotoluene, 2,4′-diaminodiphenylmethane, 4,4′-diaminodiphenylmethane, 1-methyl-3,5-diethyl-2,4-diaminobenzene and/or 1-methyl-3,5-diethyl-2,6-diaminobenzene. Further suitable polyamines are given in EP-A-0 265 781.”

U.S. Pat. No. 6,683,122 also discloses that “As long-chain polyol components there are used chiefly polyester polyols, including polylactones, for example polycaprolactones, and polyether polyols. The polyester polyols are generally linear hydroxyl polyesters having molar masses of approximately from 1000 to 3000, preferably up to 2000. Suitable polyether polyols preferably have a molecular weight of about from 300 to 8000 and can be obtained, for example, by reaction of a starter with alkylene oxides, for example with ethylene, propylene or butylene oxides or tetrahydrofuran (polyalkylene glycols). Starters that come into consideration are, for example, water, aliphatic, cycloaliphatic or aromatic polyhydroxyl compounds having generally 2, 3 or 4 hydroxyl groups, such as ethylene glycol, propylene glycol, butanediols, hexanediols, octanediols, dihydroxybenzenes or bisphenols, e.g. bisphenol A, trimethylolpropane or glycerol, or amines (see Ullmanns Encyclopadie der technischen Chemie, 4 th edition, Vol. 19, Verlag Chemie GmbH, Weinheim 1980, pages 31-38 and pages 304, 305). Especially preferred kinds of polyalkylene glycols are polyether polyols based on ethylene oxide and polyether polyols based on propylene oxide, and also corresponding ethylene oxide/propylene oxide copolymers, it being possible for such polymers to be statistical or block copolymers. The ratio of ethylene oxide to propylene oxide in such copolymers may vary within wide limits. For example, only the terminal hydroxyl groups of the polyether polyols may have been reacted with ethylene oxide (end capping). The content of ethylene oxide units in the polyether polyols may also, however, have values of e.g. up to 75 or 80% by weight. It will frequently be advantageous for the polyether polyols to be at least end-capped with ethylene oxide, since in that case they will have terminal primary hydroxyl groups which are more reactive than the secondary hydroxyl groups originating from the reaction with propylene oxide. Special mention should also be made of polytetrahydrofurans which, like the polyalkylene glycols already mentioned above, are commercially available (trade name e.g. POLYMEG®). The preparation and properties of such polytetrahydrofurans are described in greater detail, for example, in Ullmanns Encyclopadie der technischen Chemie, 4 th edition, Vol. 19, Verlag Chemie GmbH, Weinheim 1980, pages 297-299.”

U.S. Pat. No. 6,683,122 also discloses that “Also suitable as components of polyurethanes are polyether polyols that contain solid organic fillers in disperse distribution and chemically partially bonded to the polyether, such as polymer polyols and polyurea polyols. Polymer polyols are, as is known, polymer dispersions that can be prepared by free-radical polymerisation of suitable olefinic monomers, especially acrylonitrile or styrene or mixtures of the two, in a polyether serving as graft base. Polyurea polyols (PHD polyethers), which can be prepared by reaction of polyisocyanates with polyamines in the presence of polyether polyols, are dispersions of polyureas in polyether polyols, there likewise taking place a partially chemical linkage of the polyurea material to the polyether polyols by way of the hydroxyl groups on the polyether chains. Polyols such as those mentioned in this section are described in greater detail, for example, in Becker/Braun “Kunststoffhandbuch”, Vol. 7 (Polyurethanes), 2 nd edition, Carl Hanser Verlag, Munich, Vienna (1983), pages 76, 77.”

U.S. Pat. No. 6,683,122 also discloses that “Polyamines also play an important role as components in the preparation of polyurethanes, especially because they exhibit greater reactivity than comparable polyols. As in the case of the polyols, both low molecular weight polyamines, e.g. aliphatic or aromatic di- and polyamines, and polymeric polyamines, e.g. poly(oxyalkylene)polyamines, can be used. Suitable poly(oxyalkylene)polyamines, which, for example, in accordance with U.S. Pat. No. 3,267,050 are obtainable from polyether polyols, preferably have a molecular weight of from 1000 to 4000 and are also commercially available, e.g. under the name JEFFAMINE®, such as JEFFAMINE® D2000, an amino-terminated polypropylene glycol of the general formula H2NCH(CH3)CH2-[OCH2CH(CH3)]x-NH2, wherein x has on average the value 33, resulting in a total molecular weight of about 2000; JEFFAMINE® D2001 having the formula H2NCH(CH3)CH2-[OCH2CH(CH3)]a-[OCH2CH2]b-[OCH2CH(CH3)]c-NH2, wherein b is on average about 40.5 and a+c is about 2.5; JEFFAMINE® BUD 2000, a urea-terminated polypropylene ether of formula H2N(CO)NH—CH(CH3)CH2-[OCH2CH(CH3)]n-NH(CO)NH2, wherein n is on average about 33, resulting in a molecular weight of about 2075; or JEFFAMINE® T 3000, a glycerol-started poly(oxypropylene)triamine having a molecular weight of about 3000.”

U.S. Pat. No. 6,683,122 also discloses that “For the preparation of polyurethanes there are often used mixtures of one or more polyols and/or one or more polyamines, as described, for example, in EP-A-0 512 947, EP-A-0 581 739 or the prior art cited in those documents.”

U.S. Pat. No. 6,683,122 also discloses that “Various process variants can be employed for the preparation of the nanocomposites according to the invention: The swelling agent can be inserted into the layer silicate by cation exchange and the resulting organophilic layer silicate can then be incorporated as part of the filler mixture together with the mineral filler into the resin mass or into one of the components of the resin mass.”

U.S. Pat. No. 6,683,122 also discloses that “It is also possible, however, firstly to adduct the swelling agent with a portion of the monomer or monomer mixture, insert the resulting product into the layer silicate and then process that mass with the remaining portion of the resin mixture and the mineral filler to form a moulding material.”

U.S. Pat. No. 6,683,122 also discloses that “The quantity ratio of components A and B in the compositions according to the invention may vary within wide limits. The proportion of component A is preferably from 30 to 95% by weight, more especially from 40 to 92% by weight, and the proportion of component B is preferably from 5 to 70% by weight, more especially from 8 to 60% by weight, based on the sum of components A and B.”

U.S. Pat. No. 6,683,122 also discloses that “In addition to components A and B, the compositions according to the invention may contain further customary additives, for example catalysts, stabilisers, propellants, parting agents, fireproofing agents, fillers and pigments, etc.”

U.S. Pat. No. 6,683,122 also discloses that “The invention relates also to a process for the preparation of a nanocomposite, wherein a composition comprising components A and B is solidified by curing or polymerisation of component A. Special preference is given to nanocomposites that contain the layer silicate in exfoliated form.”

U.S. Pat. No. 6,683,122 also discloses that “By virtue of the very good property profile of the nanocomposites, the compositions according to the invention have a wide variety of uses, inter alia as coatings, paints/varnishes or adhesives.”

U.S. Pat. No. 6,683,122 also discloses that “The nanocomposites according to the invention can be processed by customary methods of plastics processing, such as injection moulding or extrusion, or other methods of shaping to form finished mouldings. Epoxy resins can be used as casting resins.”

The polymeric material used in the magnetic mineral composition of this invention may be one or more of the polymers used in the “high molecular substrate” of U.S. Pat. No. 6,710,111, the entire disclosure of which is hereby incorporated by reference into this specification. Claim 1 of this patent describes “1. A polymer nanocomposite, comprising: 60˜99 wt % of high molecular substrate; 0.5˜30 wt % of layer structured inorganic, well dispersed, coated evenly on the high molecular substrate; and 0.5˜30 wt % of polyelectrolyte, which carries the opposite charge of the layer-structured inorganic material and it is attached onto the layer-structured inorganic material.” Claim 2 of this patent describes the “high molecular substrate” as being “ . . . selected from the group consisting of styrene-butadiene rubber, isopiperylene rubber, butadiene rubber, acrylonitrile-butadiene rubber, natural rubber, PVC, PS, PMMA, PU and combinations thereof.”

The composition of U.S. Pat. No. 6,710,111 is a “polymer nanocomposite,” and this type of material is discussed at columns 1-2 of U.S. Pat. No. 6,710,111, wherein it is disclosed that “Nanocomposites are the composites that the diameter of its dispersed particles are in the range of 1-100 nm. In particular, the nanocomposites contain layered inorganic material, such as clay, which has the characteristics of nanoscale layer thickness, a high aspect ratio, and ionic bonding between layers. As a result, the material has high strength, high rigidity, high resistance to heat, low moisture absorption, low gas permeability and can be multiple recycled for reuse. The currently available commercial product of this nano-composites material is Nylon 6/clay from Ube Company, Japan, which is used in vehicle parts and air-blocking wrapping films (1990); and from Unitika Company, Japan, which is used in vehicle parts and as an engineering plastic (1996).”

U.S. Pat. No. 6,710,111 also discloses that “Conventional methods to produce nanocomposites are: (1) in-situ polymerization, (2) kneading and (3) coagulation and sedimentation. Nylon 6 nanocomposite has been successfully commercialized by in-situ polymerization. However, this method is successful for Nylon 6 nanocomposites only until to now. Moreover, although kneading is convenient, the equipment is considerably expensive and the relative techniques are very complex. It has not been commercialized. As for coagulation and sedimentation, most research, such as Applied Clay Science volume 15 (1999), pages 1˜9, has shown that it is hard to avoid the re-coagulate of the layered inorganic material. For example, the preparation methods of nanocomposite of Styrene-Butadiene Rubber (SBR) as disclosed in the journal of Special Rubber Products, issued by Beijing-Univ-Chem-Technol in China, volume 19 (2), pages 6˜9, 1997, include: (1) Latex method: Vigorously stirring the aqueous to allow clay dispersed in water, SBR latex and antioxidant are then added and uniformly mixed. The mixture is coagulated with the addition of diluted hydrochloric acid. After it is washed with water and dried, clay/SBR nanocomposite is obtained. The lattice spacing of the clay is expanded from 0.98 nm of pure clay to 1.46 nm. This indicates that SBR molecules inserted between layers of clay to form intercalated nanocomposites. (2) Solution method: Modify the clay by organic chemicals and the obtained clay is vigorously stirred to disperse in toluene. A SBR-toluene solution is then added and the mixture is stirred vigorously to become a uniform mixture. After it is sedimented and dried, clay/SBR nanocomposite is obtained. The lattice spacing of clay is expanded from 0.98 nm of pure clay to 1.90 nm after it is organically modified, and further expanded from 1.90 nm to 4.16 nm in clay/SBR nanocomposite. This indicates that more SBR molecules are inserted into layers of clay than the above latex method. Nevertheless, this method uses a large amount of toluene, which causes the production cost to increase and the occurrence of environmental problems.”

The polymeric material used in the magnetic mineral composition of this invention may be a polymeric foam as is described, e.g., U.S. Pat. No. 6,750,264, the entire disclosure of which is hereby incorporated by reference into this specification. Claim 1 of this patent describes “1. A polymeric foam comprising a polymer having multiple cells defined therein and at least one absorbent clay dispersed within said polymer; wherein said foam has a multimodal cell size distribution and contains less than 0.2 parts by weight of bentonite based on 100 parts by weight of polymer.” Processes for the preparation of such foams are described in columns 3-5 of this patent, wherein it is disclosed that “Polymer resins useful for preparing polymeric foams of the present invention are desirably thermoplastic polymer resins. Suitable thermoplastic polymer resins include any extrudable polymer (including copolymers) including semi-crystalline, amorphous, and ionomeric polymers and blends thereof. Suitable semi-crystalline thermoplastic polymers include polyethylene (PE), such as high-density polyethylene (HDPE), and low-density polyethylene (LDPE); polyesters such as polyethylene terephthalate (PET); polypropylene (PP) including linear, branched and syndiotactic PP; polylactic acid (PLA); syndiotactic polystyrene (SPS); ethylene copolymers including ethylene/styrene copolymers (also known as ethylene/styrene interpolymers), ethylene/alpha-olefin copolymers such as ethylene/octene copolymers including linear low density polyethylene (LLDPE), and ethylene/propylene copolymers. Suitable amorphous polymers include polystyrene (PS), polycarbonate (PC), thermoplastic polyurethanes (TPU), polyacrylates (e.g., polymethyl-methacrylate), and polyether sulfone. Preferred thermoplastic polymers include those selected from a group consisting of polymers and copolymers of PS, PP, PE, PC and polyester. Suitable polymer resins include coupled polymers such as coupled PP (see, for example, U.S. Pat. No. 5,986,009 column 16, line 15 through column 18, line 44, incorporated herein by reference), coupled blends of alpha olefin/vinyl aromatic monomer or hindered aliphatic vinyl monomer interpolymers with polyolefins (see, for example, U.S. Pat. No. 6,284,842, incorporated herein by reference), and lightly crosslinked polyolefins, particularly PE (see, for example U.S. Pat. No. 5,589,519, incorporated herein by reference). Lightly crosslinked polyolefins desirably have a composition content of 0.01% or more, preferably 0.1% or more, and 5% or less, preferably 1% or less according to American Society for Testing and Materials (ASTM) method D2765-84.”

U.S. Pat. No. 6,750,264 also discloses that “Foams and processes of the present invention include at least one absorbent clay. An absorbent clay absorbs water into interlayer spacings and, when present in a foamable composition, releases at least a portion of that water as a polymer expands into a foam during foam manufacturing.”

U.S. Pat. No. 6,750,264 also discloses that “An absorbent clay for use in the present invention also desirably has a plasticity index (PI) of less than 500, preferably less than 200, more preferably less than 100, still more preferably less than 75, and greater than zero. A PI is the difference between the wt % of absorbed water necessary for a clay to change to a near liquid state (liquid limit) and the wt % of absorbed water necessary for a clay to become plastic (plastic limit). A PI is a measure of a clay's plastic range breadth. If a clay has a large PI (greater than 500), it can develop an undesirably high viscosity in the presence of water and hinder foam manufacturing.”

U.S. Pat. No. 6,750,264 also discloses that “Absorbent clays are distinct from clays that adsorb water. Clays that adsorb water only take up water onto their surface. Clays for use in the present invention absorb water by taking it up into interlayer spacings in the clay. Release of water absorbed into a clay can be controlled more ways than release of water adsorbed on the surface of a clay, providing absorbent clays an advantage over adsorbing clays. Controlling water release allows control over multimodal cell formation. Examples of clays that are not considered absorbent clays because they tend to adsorb rather than absorb water include mica-illite group three-layer-minerals such as pyrophylite, muskovite, dioktaedric illite, glaukonite, talc, biotite, and dioktaedric illite.”

U.S. Pat. No. 6,750,264 also discloses that “Examples of suitable absorbent clays for use in the present invention include two-layer-minerals of the kaolinite-group such as kaolinite, dickite, halloysite, nakrite, serpentine, greenalithe, berthrierine, cronstedtite, and amesite. Halloysite is a particularly desirable absorbent clay for use in the present invention. Two-layer minerals of the kaolinite group tend to absorb water into interlayer spacings without swelling the clay. Absorbent clays that absorb water without swelling are desirable because they tend to undergo minimal viscosity increase upon absorption of water.”

U.S. Pat. No. 6,750,264 also discloses that “Smectite-group three-layer minerals can also fall within the scope of an absorbent clay. Smectite-group three-layer minerals include dioktaedric vermiculite, dioktaedric smectite, montmorillonite, beidellite, nontronite, volkonskoite, trioctaedric vermiculite, trioctaedric smectite, saponite, hectorite, and saukonite. Smectite-group three-layer minerals tend to swell as they absorb water between their interlayer spaces.”

U.S. Pat. No. 6,750,264 also discloses that “Salt forms of minerals are also included within the scope of absorbent clays. Absorbent clay salts generally have potassium, calcium or magnesium counterions but can also have organic counterions. Certain salt forms of smectite-group three-layer minerals have a plasticity index outside the desired scope of an absorbent clay. For example, sodium montmorillonite has a plastic limit of 97, liquid limit of 700, and a PI of 603.”

U.S. Pat. No. 6,750,264 also discloses that “WO 01/51551 A1 discloses a process for forming bimodal polymeric foam using bentonite at a concentration of 0.2 to 10 parts by weight in 100 parts by weight of a thermoplastic resin. “Bentonite” is a rock whose principle components are montomorillonite salts, particularly sodium montmorillonite. WO 01/51551 A1 (incorporated herein by reference) includes in the definition of bentonite natural bentonite, purified bentonite, organic bentonite, modified montmorillonite such as montorillonite modified with an anionic polymer, montmorillonite treated with a silane, and montmorillonite containing a high polarity organic solvent. Herein, “bentonite” refers to the broad definition used in WO 01/51551 A1. In contrast to teachings in WO 01/51551 A1, multimodal foams of the present invention can be made using less than 0.2 weight parts, preferably less than 0.1 weight parts, more preferably less than 0.05 weight parts of bentonite, based on 100 weight parts of polymer. Foams and process for preparing foams of the present invention can be free of bentonite.”

U.S. Pat. No. 6,750,264 also discloses that “Polymeric foams of the present invention contain absorbent clays at a concentration of 0.01 wt % or more, preferably 0.1 wt % or more, more preferably 0.2 wt % or more and generally 10 wt % or less, preferably 5 wt % or less, and more preferably 3 wt % or less based on polymer resin weight. Generally, suitable absorbent clays have a particle size of 100 micrometers or less, preferably 50 micrometers or less, more preferably 20 micrometers or less. There is no known limit as to how small absorbent clay particles can be for use in the present invention, however the particles typically have a size of one micrometer or more, often 5 micrometers or more. Typically, particle clays having a particle size of 20 micrometers or less are useful for preparing close-celled foams while clays having a particle size of 50 micrometers or greater are useful for preparing open-celled foams. If an absorbent clay swells with water, determine particle size prior to swelling.”

U.S. Pat. No. 6,750,264 also discloses that “Cell-controlling agents (also known as nucleating agents) can be present, but are not necessary for preparing foams of the present invention. Nucleating agents are often useful for controlling cell sizes of smaller cells of a bimodal foam. Examples of typical nucleating agents include talc powder and calcium carbonate powder. Foams and processes of the present invention can be substantially free of nucleating agents apart from the absorbent clay. “Substantially free” means having less than 0.05 weight parts per 100 weight parts of polymer resin. Foams and foam preparation process of the present invention can include 0.02 weight parts or less, even 0.01 weight parts or less of nucleating agents other than the absorbent clay. Foams and foam preparation processes of the present invention can be free of nucleating agents other than the absorbent clay.”

U.S. Pat. No. 6,750,264 also discloses that “Prepare multimodal foams of the present invention, in general, by preparing a foamable polymer composition at an initial pressure and then expanding the foamable polymer composition at a foaming pressure, which is lower than the initial pressure, into a polymeric foam having a multimodal cell size distribution. The foamable polymer composition comprises a mixture of plasticized polymer resin, a blowing agent composition and an absorbent clay that is capable of expanding into a multimodal polymer foam when upon lowering the initial pressure to the foaming pressure. The initial pressure is a pressure sufficient to liquefy the blowing agent composition and to preclude foaming of the foamable polymer composition.”

U.S. Pat. No. 6,750,264 also discloses that “Prepare a foamable polymer composition by blending together components comprising foamable polymer composition in any order. Typically, prepare a foamable polymer composition by plasticizing a polymer resin, blending in an absorbent clay, and then blending in components of a blowing agent composition at an initial pressure. A common process of plasticizing a polymer resin is heat plasticization, which involves heating a polymer resin enough to soften it sufficiently to blend in a blowing agent composition, an absorbent clay, or both. Generally, heat plasticization involves heating a thermoplastic polymer resin to or near to its glass transition temperature (Tg), or melt temperature (Tm) for crystalline polymers.”

U.S. Pat. No. 6,750,264 also discloses that “Addition of an absorbent clay can occur at any point prior to foaming the foamable polymer composition. For example, an artisan can blend polymer resin and an absorbent clay together while polymerizing the polymer resin, during a melt-blending procedure with a polymer resin but prior to initiating a foaming process (e.g., making polymer pellets containing an absorbent clay), or during a foaming process.”

U.S. Pat. No. 6,750,264 also discloses that “Blowing agent compositions for use in the present invention comprise CO2 and water, and can contain additional blowing agent components. CO2 is present at a concentration of 0.5 wt % or more, preferably 10 wt % or more, more preferably 20 wt % or more and 99.5 wt % or less, preferably 98 wt % or less, and more preferably 95 wt % or less based on blowing agent composition weight. Water is present at a concentration of 0.5 wt % or more, preferably 3 wt % or more, and 80 wt % or less, more preferably 50 wt % or less, and more preferably 20 wt % or less based on blowing agent composition weight.”

U.S. Pat. No. 6,750,264 also discloses that “Additional blowing agents can be present at a concentration ranging from 0 wt % to 80 wt %, based on blowing agent composition weight. Preferably, less than 40 wt % of the blowing agent composition is selected from a group consisting of dimethyl ether, methyl ether, and diethyl ether. Suitable additional blowing agents include physical and chemical blowing agents. Suitable physical blowing agents include HFCs such as methyl fluoride, difluoromethane (HFC-32), perfluoromethane, ethyl fluoride (HFC-161), 1,1-difluoroethane (HFC-152a), 1,1,1-trifluoroethane (HFC-143a), 1,1,2,2-tetrafluoroethane (HFC-134), 1,1,1,2-tetrafluoroethane (HFC-134a), pentafluoroethane (HFC-125), perfluoroethane, 2,2-difluoropropane (HFC-272fb), 1,1,1-trifluoropropane (HFC-263fb), and 1,1,1,2,3,3,3-heptafluoropropane (HFC-227ea); liquid hydrofluorocarbons such as 1,1,1,3,3-pentafluoropropane (HFC-245fa), and 1,1,1,3,3-pentafluorobutane (HFC-365mfc); hydrofluoroether; inorganic gases such as argon, nitrogen, and air; organic blowing agents such as aliphatic hydrocarbons having from one to nine carbons (C1-C9) including methane, ethane, propane, n-butane, isobutane, n-pentane, isopentane, neopentane, cyclobutane, and cyclopentane; fully and partially halogenated aliphatic hydrocarbons having from one to four carbons (C1-C4) including aliphatic and cyclic hydrocarbons; and aliphatic alcohols having from one to five carbons (C1-C5) such as methanol, ethanol, n-propanol, and isopropanol; carbonyl containing compounds such as acetone, 2-butanone, and acetaldehyde. Suitable chemical blowing agents include azodicarbonamide, azodiisobutyronitrile, benzenesulfo-hydrazide, 4,4-oxybenzene sulfonyl semi-carbazide, p-toluene sulfonyl semi-carbazide, barium azodicarboxylate, N,N′-dimethyl-N,N′-dinitrosoterephthalamide, trihydrazino triazine and sodium bicarbonate.”

U.S. Pat. No. 6,750,264 also discloses that “CO2, water, and any additional blowing agents account for 100 wt % of a blowing agent composition for use in the present invention. A blowing agent composition is typically present at a concentration of 3 parts per hundred (pph) or more, preferably 4 pph or more, more preferably 5 pph or more and typically 18 pph or less, preferably 15 pph or less, and more preferably 12 pph or less based on polymer resin weight.”

U.S. Pat. No. 6,750,264 also discloses that “One desirable blowing agent composition for use in the present invention contains CO2 and water, and is essentially free of additional blowing agents, meaning that the blowing agent composition comprises 1 wt % or less, preferably 0.5 wt % or less, more preferably 0.1 wt % or less, still more preferably zero wt % of additional blowing agent based on blowing agent composition weight.”

U.S. Pat. No. 6,750,264 also discloses that “Another desirable blowing agent composition consists essentially of carbon dioxide, water, and ethanol. Ethanol is useful to reduce foam density and increase foam cell sizes over foams prepared with blowing agents without ethanol. Still another desirable blowing agent composition consists essentially of CO2, water, a C1-C5 hydrocarbon, and, optionally, ethanol. The hydrocarbon in this particular blowing agent composition can be halogen-free or can be a hydrofluorocarbon. Preferably, select the hydrocarbon from a group consisting of isobutane, cyclopentane, n-pentane, isopentane, HFC-134a, HFC-235fa, and HFC-365mfc. The hydrocarbon serves to reduce the thermal conductivity of a resulting foam over a foam prepared without such a hydrofluorocarbon. Examples of such blowing agent compositions include CO2, water, and at least one of cyclopentane, n-pentane, and isopentane, HFC-134a, HFC-245fa, and HFC-365mfc; and CO2, water, ethanol and at least one of isobutane, cyclopentane, n-pentane, isopentane, HFC-134a, HFC-245fa, and HFC-365mfc.”

U.S. Pat. No. 6,750,264 also discloses that “One hypothesis for how multimodal foams form according to the present invention is that the absorbent clay absorbs water in the blowing agent composition in such a manner so as to delay release (and subsequent expansion) of the water until after the CO2 has begun expanding. Delaying expansion of the water during foaming until after CO2 expansion begins effectively causes formation of multiple cells having smaller sizes than cells resulting from CO2 expansion. Water release from an absorbent clay is controllable by an absorbent clay's affinity for water (binding energy) as well as the size and tortuosity of the clay's interlayer spaces within which water absorbs.”

U.S. Pat. No. 6,750,264 also discloses that “A foamable polymer composition can contain additional additives such as pigments, fillers, antioxidants, extrusion aids, stabilizing agents, antistatic agents, fire retardants, acid scavengers, and thermally insulating additives. One desirable embodiment includes thermally insulating additives such as carbon black, graphite, silicon dioxide, metal flake or powder, or a combination thereof in the foamable polymer composition and foam of the present invention. Add additional additives to a polymer, polymer composition, or foamable polymer composition at any point in the foaming process prior to reducing a foamable polymer composition from an initial pressure to a foaming pressure, preferably after plasticizing a polymer and prior to adding a blowing agent.”

U.S. Pat. No. 6,750,264 also discloses that “Foam preparation processes of the present invention include batch, semi-batch, and continuous processes. Batch processes involve preparation of at least one portion of the foamable polymer composition in a storable state and then using that portion of foamable polymer composition at some future point in time to prepare a foam. For example, prepare a portion of a foamable polymer composition containing an absorbent clay and polymer resin by heat plasticizing a polymer resin, blending in an absorbent clay to form a polymer/clay blend, and then cooling and extruding the polymer/clay blend into pellets. Use the polymer/clay blend pellets later to prepare a foamable polymer composition and expand into a foam.”

U.S. Pat. No. 6,750,264 also discloses that “A semi-batch process involves preparing at least a portion of a foamable polymer composition and intermittently expanding that foamable polymer composition into a foam all in a single process. For example, U.S. Pat. No. 4,323,528, herein incorporated by reference, discloses a process for making polyolefin foams via an accumulating extrusion process. The process comprises: 1) mixing a thermoplastic material and a blowing agent composition to form a foamable polymer composition; 2) extruding the foamable polymer composition into a holding zone maintained at a temperature and pressure which does not allow the foamable polymer composition to foam; the holding zone has a die defining an orifice opening into a zone of lower pressure at which the foamable polymer composition foams and an openable gate closing the die orifice; 3) periodically opening the gate while substantially concurrently applying mechanical pressure by means of a movable ram on the foamable polymer composition to eject it from the holding zone through the die orifice into the zone of lower pressure, and 4) allowing the ejected foamable polymer composition to expand to form the foam.”

U.S. Pat. No. 6,750,264 also discloses that “A continuous process involves forming a foamable polymer composition and then expanding that foamable polymer composition in a non-stop manner. For example, prepare a foamable polymer composition in an extruder by heating a polymer resin to form a molten resin, blending into the molten resin an absorbent clay and blowing agent composition at an initial pressure to form a foamable polymer composition, and then extruding that foamable polymer composition through a die into a zone at a foaming pressure and allowing the foamable polymer composition to expand into a multimodal foam. Desirably, cool the foamable polymer composition after addition of the blowing agent and prior to extruding through the die in order to optimize foam properties. Cool the foamable polymer composition, for example, with heat exchangers.”

U.S. Pat. No. 6,750,264 also discloses that “Foams of the present invention can be of any form imaginable including sheet, plank, rod, tube, beads, or any combination thereof. Included in the present invention are laminate foams that comprise multiple distinguishable longitudinal foam members that are bound to one another. Laminate foams include coalesced foams that comprise multiple coalesced longitudinal foam members. Longitudinal foam members typically extend the length (extrusion direction) of a coalesced polymeric foam. Longitudinal foam members are strands, sheets, or a combination of strands and sheets. Sheets extend the full width or height of a coalesced polymeric foam while strands extend less than the full width and/or height. Width and height are orthogonal dimensions mutually perpendicular to the extrusion direction (length) of a foam. Strands can be of any cross-sectional shape including circular, oval, square, rectangular, hexagonal, or star-shaped. Strands in a single foam can have the same or different cross-sectional shapes. Longitudinal foam members can be solid foam or can be hollow, such as hollow foam tubes (see, for example, U.S. Pat. No. 4,755,408; incorporated herein by reference). The foam of one preferred embodiment of the present invention comprises multiple coalesced foam strands.”

U.S. Pat. No. 6,750,264 also discloses that “Preparing coalesced polymeric foams typically involves extruding a foamable polymer composition containing polymer resin and a blowing agent formulation through a die defining multiple holes, such as orifices or slits. The foamable polymer composition flows through the holes, forming multiple streams of foamable polymer composition. Each stream expands into a foam member. “Skins” form around each foam member. A skin can be a film of polymer resin or polymer foam having a density higher than an average density of a foam member it is around. Skins extend the full length of each foam member, thereby retaining distinguishability of each foam member within a coalesced polymeric foam. Foam streams contact one another and their skins join together during expansion, thereby forming a coalesced polymeric foam.”

U.S. Pat. No. 6,750,264 also discloses that “Other methods are available for joining longitudinal foam members together to form a foam including use of an adhesive between foam members and coalescing foam members together after they are formed by orienting the members and then applying sufficient heat, pressure, or both to coalesce them together. Similar processes are suitable for forming bead foam, which comprises multiple foam beads partially coalesced together. Bead foam is also within the scope of the present invention.”

U.S. Pat. No. 6,750,264 also discloses that “Foams of the present invention contain residual blowing agents, including CO2 and water, when fresh. Fresh, herein, means within one day, preferably within one hour, more preferably immediately after manufacturing. Foams of the present invention can also contain residuals of additional blowing agents if they were present during foam preparation.”

U.S. Pat. No. 6,750,264 also discloses that “Foams of the present invention typically have a density of 16 kilograms per cubic meter (kg/m3) or more, more typically 20 kg/m3 or more, and still more typically 24 kg/m3 or more and 64 kg/m3 or less, preferably 52 kg/m3 or less, and more preferably 48 kg/m3 or less. Determine foam density according to ASTM method D-1622.”

U.S. Pat. No. 6,750,264 also discloses that “Foams of the present invention can be open-celled or close-celled. Open-celled foams have an open cell content of 20% or more while close-celled foams have an open cell content of less than 20%. Determine open cell content according to ASTM method D-6226. Desirably, the present foams are close-celled foams.”

U.S. Pat. No. 6,750,264 also discloses that “Foams of the present invention are particularly useful as thermal insulating materials and desirably have a thermal conductivity of 30 milliwatts per meter-Kelvin (mW/m-K) or less, preferably 25 mW/m-K or less (according to ASTM method C-518 at 24° C.). Foams of the present invention also preferably include a thermally insulating additive. Articles, such as thermally insulating containers, that contain foams of the present invention “

The polymeric material used in the magnetic mineral composition of this invention may be, e.g., one or more of the copolymers disclosed in U.S. Pat. No. 6,767,951, the entire disclosure of which is hereby incorporated by reference into this specification. As is well known to those skilled in the art, polymers can be built of one, two, or even three different monomers and termed homopolymers, copolymers, and terpolymers, respectively. The claims of U.S. Pat. No. 6,767,951 describe clay intercalated with a block copolymer. Thus, e.g., claim 1 of this patent describes “1. An article comprising a matrix polymer and clay wherein said clay is intercalated with a block copolymer, wherein said block copolymer comprises a hydrophilic block capable of intercalating said clay and a matrix compatible block compatible with said matrix polymer wherein said block copolymer comprises three blocks.” Claim 2 of this patent describes the matrix polymers as “ . . . consisting of polyester.” Claim 3 of this patent describes the polyester as being “ . . . selected from the group comprising poly(ethylene terephthalate), poly(butylene terephthalate), poly(1,4-cyclohexylene dimethylene terephthalate), poly(ethylene naphthalate) and amorphous glycol modified poly(ethylene terepthalate).” Claim 4 describes the “hydrophilic block” as comprising “ . . . at least one member selected from the group consisting of poly(alkylene oxide), poly 6, (2-ethyloxazolines), poly(ethyleneimine), poly(vinylpyrrolidone), poly(vinyl alcohol), polyacrylamides, polyacrylonitrile, polysaccharides and dextrans.” Claim 5 describes the “hydrophilic block” as comprising “ . . . at least one member selected from the group consisting of poly(alkylene oxide), poly 6, (2-ethyloxazolines), polysaccharide, poly(vinylpyrrolidone), poly(vinyl alcohol) and poly(vinylacetate).” Claim 6 describes the “hydrophilic block” as comprising poly(ethylene oxide) In claim 7, the “hydrophilic block is described as being polysaccharide. In claim 8, the “ . . . .hydrophilic block comprises poly(vinyl pyrrolidone).” In claim 9, the “ . . . hydrophilic block comprises poly(vinyl acetate).”

The polymeric material used in the magnetic mineral composition of this invention may be the polyamide material of U.S. Pat. No. 6,780,522 that has non-scale nucleating particles dispersed therein; the entire disclosure of this United States patent is hereby incorporated by reference into this specification. Claim 1 of this patent describes “1. Multi-layer film having at least one layer (I) of polyamide with nano-scale nucleating particles dispersed therein, wherein said nano-scale nucleating particles have an aspect ratio of at least 10 in two randomly selectable directions, and, as a number-weighted average, a dimension no greater than 100 nm in at least one direction that is randomly selectable for each consent, having crystalline structures that emanate from the surface of the particles, the amount by weight of the particles, based on the total weight of the polyamide forming the layer (I), is from 10 ppm to 2000 ppm, the polyamide forming the layer (I) contains at least 90% polyamide 6, based on the total mass of the polyamide in that layer and comprising further polyamide-containing layers (II) containing no or less than 10 ppm nano-scale nucleating agent.”

The polymeric material used in the magnetic mineral composition of this invention may be an ionomeric polyester as described, e.g., in U.S. Pat. No. 6,831,123, the entire disclosure of which is hereby incorporated by reference into this specification. Claim 1 of this patent describes “1. A composition comprising at least one ionomeric polyester resin and at least one organoclay, wherein the organoclay is not preswollen before combination with ionomeric polyester resin.”

A .Composition that Contains Ceramic Material and Nanomagnetic Material.

In the preceding section of this specification, applicants described a composition that contains nanomagnetic material, polymeric material, and (optionally) one or more mineral materials. In this section of the specification, applicants will describe a comparable composition in which the polymeric material is replaced by a ceramic material.

As used in this specification, the term ceramic refers to any of a class of inorganic, nonmetallic products which are subjected to a temperature of 540 degrees Celsius and above during manufacture or use, including metallic oxides, borides, carbides, or nitrides, and mixtures or compounds of such materials. Reference may be had, e.g., to page 54 of Loran S. O'Bannon's “Dictionary of Ceramic Science and Engineering” (Plenum Press, New York, N.Y., 1984).

The ceramic material used in the magnetic mineral composition of this invention may be a calcined diatomaceous earth, as described, e.g., in U.S. Pat. No. 3,793,042, the entire disclosure of which is hereby incorporated by reference into this specification. Claim 1 of this patent describes “1. A plastic refractory composition suitable for ramming into place to form a monolithic refractory furnace lining consisting essentially of 20-70 parts by weight coarse graded alcined diatomaceous earth, said calcined diatomaceous earth being in the cristobalite form, 3-12 parts by weight finely ground plastic clay selected from the group consisting of bentonite, kaolinite, halloysite, illite and attapulgite, and 20-70 parts.” The calcined diatomaceous earth is also described at column 1 of this patent, which discloses that “The calcined diatomaceous earth is a coarse graded calcined diatomaceous silica aggregate that has been converted to the crystobalite form by calcining at not lower than 2,100° F. This calcining gives the diatomaceous earth maximum volume stability which prevents swelling during the heating cycles.”

The ceramic material used in the magnetic mineral composition of this invention may be a porous ceramic composition such as, e.g., the porous ceramic composition of matter described in U.S. Pat. No. 4,358,400, the entire disclosure of which is hereby incorporated by reference into this specification. Claim 1 of this patent describes “1. A porous composition of matter comprising: dispersed rods of halloysite, and 0-15 percent by weight of a binder oxide, based on the total weight of said halloysite and binder oxide having a pore volume of at least 0.35 cc/gm of which at least 70 percent of the pore volume is present as pores having a diameter of between 200-700 Angstroms and at least 70 percent of said pores have a diameter of 300-700 Angstroms.” The preparation of the “dispersed rods of halloysite” is discussed at columns 2-4 of this patent, wherein it is disclosed that “The tubular or rod form of halloysite is readily available from natural deposits. It frequently comprises bundles of tubular rods or needles consolidated or bonded together in a weakly parallel orientation. It has been discovered that if these bundles of rods are broken up by mechanical means and re-oriented in a substantially random orientation with respect to one another, a catalyst support with superior asphaltene hydroconversion properties results. Halloysite occurs naturally in tubular rods that are approximately 1 micron long and 0.1 micron in diameter with a centrally located hole penetrating the rod from about 100 Angstroms to about 300 Angstroms in diameter resulting in a scroll-like rod, in contrast to fibrous clays like attapulgite and sepiolite which are nontubular. The exact dimensions vary from rod to rod and are not critical. It is critical that the rod form, rather than the platy form, of halloysite be used.

U.S. Pat. No. 4,358,400 also discloses that “In addition to the halloysite component of the present catalyst, an inorganic binder oxide may be added. Inorganic binder oxides are defined as refractory inorganic oxide such as, silica and oxides of elements in Group 2a, 3b and 3a of the Periodic Table as defined in Handbook of Chemistry and Physics, 45th Edition. Preferable binder oxides include: silica, alumina, magnesia, zirconia, titania, boria and the like. An especially preferred binder oxide is alumina. It has been discovered that the amount of asphaltene adsorbed onto a catalyst support of dispersed rods of halloysite is related to the amount of binder oxide used. When the amount of binder oxide exceeds about 15 percent of the total weight of halloysite and binder oxide, the amount of asphaltenes adsorbed is severely reduced. It has been found that an especially preferable amount of binder oxide is about 5 percent. As more binder oxide is added to the catalyst support, the pore sizes tend to cluster around smaller distributions. A catalyst support with 25 percent alumina has substantially all of its pores less than 100 Angstroms in diameter.”

U.S. Pat. No. 4,358,400 also discloses that “A catalyst support made from halloysite can contain any catalytic reactive transition metal. The catalytic metal component can be added during any stage of preparation. Catalytic metals can be added as powdered salts or oxides during the agitation stage or by impregnation of the catalyst body by adding a metal containing solution after the catalyst bodies have been formed. Preferred catalytic metals are those of Groups VI-B and VIII of the Periodic Table. When preparing hydroprocessing catalysts, it is preferable that the composition include at least one metal of the group of chromium, molybdenum, tungsten and vanadium, and at least one metal of the group of iron, nickel and cobalt, such as cobalt-molybdenum, nickel-tungsten or nickel-molybdenum.” It should be noted that, in addition to the means described elsewhere in this specification, one may add the nanomagnetic material of this invention to the dispersed rods of halloysite by the means taught in U.S. Pat. No. 4,358,400.

U.S. Pat. No. 4,358,400 also discloses that “Preparation of the catalyst with dispersed rods is accomplished by creating a mixture of tubular halloysite and if desired, binder oxide and enough water to form a slurry of about 20 weight percent solid content. As the mixture is violently agitated the slurry is observed to thicken. Agitation is continued until the slurry stops getting thicker with continued agitation. This takes about 10 minutes of agitation. This thickening is indicative of dispersal of the rods. Excess water in the slurry is removed by evaporation until a moldable plastic mass is formed. The bodies are then shaped by spheridizing, pelletizing and similar procedures and then calcined. It has been observed that a catalyst body made of dispersed rods of halloysite tends not to extrude well. The rods tend to realign on the surface of the extruded mass, and this skin effect decreases the average pore diameter at the surface of the extruded mass. Alternatively, the halloysite mass can be dried and calcined; and the calcined mass broken up to produce catalyst bodies. The final product is a catalyst body with the characteristics of dispersed rods of halloysite. It is preferable that the binder oxide be added to the halloysite as the gel or the sol precursor to the gel at the agitation stage of the slurry.” This means may also be used to add nanomagnetic material to the dispersed rods of halloysite.

U.S. Pat. No. 4,358,400 also discloses that “Referring to Table I, the pore size distribution for unprocessed halloysite and pore size distribution for halloysite with dispersed rods are compared. It will be noted that in unprocessed halloysite most of the pore size is in the 200-400 Angstrom range. On the other hand, halloysite with dispersed rods has most of it pores distributed from 400-600 Angstroms. In halloysite with dispersed rods there is a substantial amount of pore volume provided by pores having diameters in the range of 100-300 Angstroms. It is believed that these pores are from the central hole present in halloysite rods. The presence of these smaller pores is not a gauge of the thoroughness of dispersion of the rods.”

U.S. Pat. No. 4,358,400 also discloses that “It will also be noted that the halloysite with dispersed rods has a substantially greater total pore volume than the natural halloysite. It is believed that the pores in the range of 200 Angstroms to about 700 Angstroms impart especially good deasphalting properties to the catalyst support. One explanation is that demetalation and desulfurization reactions tend to be fast, therefore, pores significantly larger than the molecules tend to allow rapid diffusion into and out of the pores. Large pores are preferable in demetalation catalysts since the metals removed from the feedstocks tend to deposit on the surface of the catalyst support, thereby rapidly plugging the mouths of the smaller pores. Since there is no substantial amount of pore volume in pores greater than 1000 Angstroms, there is less problem with mechanically weak catalyst bodies and attendant attrition.”

“Example II” of U.S. Pat. No. 4,358,400 discloses the preparation of halloysite with a binder support. As will be apparent to those skilled in the art, one may use the procedure of this Example to prepare a mixture of halloysite and magnetic material.

The experiment described in such “Example II” used naturally occurring halloysite from the Dragon Iron Mine in Utah; #13 powder was used. As is disclosed in this Example, “This example illustrates preparation of a catalyst support containing halloysite and a binder oxide. Dragon Halloysite #13 powder is placed in a blender. Enough 5 percent alumina by weight alumina hydrogel is added to form a mixture that is 5 percent by dry weight alumina. The alumina hydrogel is prepared conventionally, as by peptizing a commercially available alumina by a vigorous agitation with a peptizing agent such a nitric acid or formic acid, or by precipitation of the hydrogel from an aluminum nitrate solution with a base such as ammonium hydroxide. Enough water is then added to make a slurry that is no more than about 20 percent solid content. The mixture is then vigorously agitated in a Waring blender until the slurry no longer visibly thickens. Once the halloysite rods are adequately dispersed, the slurry will not get any thicker. Normally this takes about 10 minutes of agitation. Excess water is evaporated from the slurry to form a plastic, workable mass. The mixture is heated to 500° C. for three hours and the calcined mass is broken up into catalyst particles.”

By way of yet further illustration, the ceramic material used in the magnetic mineral composition of this invention may be cordierite as described, e.g., in U.S. Pat. No. 4,421,699, the entire disclosure of which is hereby incorporated by reference into this specification. Claim 1 of this patent describes “1. A method of producing a cordierite body having a coefficient of thermal expansion of less than 10.5×10⁻⁷/° C. comprising the steps of: (1) mixing together and kneading a batch raw material containing tubular-shaped halloysite particles and plate-shaped talc particles delaminated along the (001) plane thereof, said halloysite particles including at least one material selected from the group consisting of halloysite, metahalloysite, endellite and allophane; (2) anisostatically forming the mixed batch raw material into a formed body thereby imparting a planar orientation to said plate-shaped talc particles contained in said batch raw material; and (3) drying and firing the thus formed body.”

The use of a delaminated halloysite material is discussed at columns 2-3 of U.S. Pat. No. 4,421,699, wherein it is disclosed that “We inventors have made various studies and experiments to obtain a cordierite body exhibiting a more excellent low thermal expansion property and to promote the sintering in the firing step of the cordierite body. As a result, we have found that by mixing and kneading a batch raw material containing halloysite particles used as kaolin minerals, and talc particles which are delaminated like platelets along the (001) plane, by subjecting the mixed raw material to anisostatic forming such as extrusion forming so as to impart a planar orientation to the platelet shaped talc particles therein and by drying and firing the obtained green body, a cordierite body having high crystallinity can be obtained at a relatively low firing temperature.”

U.S. Pat. No. 4,421,699 also discloses that “And furthermore, we have found that by using the above described production method, a cordierite body of which the coefficient of thermal expansion is less than 10.0×10−7/° C. in a specific direction can be obtained.”

U.S. Pat. No. 4,421,699 also discloses that “The important points of the present invention are that plate-shaped talc particles contained within the batch raw material impart a low thermal expansion property to the obtained cordierite body, and that halloysite contained within the batch raw material promotes the sintering of the cordierite body.”

U.S. Pat. No. 4,421,699 also discloses that “Namely, when talc (3MgO.4SiO2.H2O) is broken, it is generally delaminated into plate-shaped particles along the (001) plane perpendicular to the C-crystal axis thereof. And when the batch raw material containing these plate-shaped talc particles is extruded by means of an extrusion die, the plate-shaped talc particles 1 align themselves while the batch raw material passes thin slits of the extrusion die, and the plate-shaped talc particles 1 are oriented in the plane along the surface of the sheet-shaped extruded green body 2.”

U.S. Pat. No. 4,421,699 also discloses that “The cordierite body obtained by drying and firing the extruded green body exhibits very excellent low thermal expansion property in a direction along the surface thereof. This result shows that the cordierite body exhibits a low thermal expansion property in the direction parallel with the (001) plane of the talc particles.”

U.S. Pat. No. 4,421,699 also discloses that “Next, halloysite is expressed by the chemical formula of Al2O3.2SiO2.4H2O which is similar to that of kaolinite (Al2O3.2SiO2.2H2O). However, crystallinity of halloysite is lower than that of kaolinite. And a typical form of a halloysite crystal is a tubular form. When the batch raw material containing halloysite is fired, the cordierite body having excellent crystallinity can be obtained at a relatively lower firing temperature as compared with the case wherein other kaolin minerals such as kaolinite are used. It is recognized that the weaker chemical bonding and the lower crystallinity of halloysite than those of kaolinite have a beneficial effect in the sintering reaction of the cordierite body.”

U.S. Pat. No. 4,421,699 also discloses that “In the present invention, halloysite includes metahalloysite and endellite, allophane and the like all of which are formed in the process that the halloysite crystals grow.” It should be noted that each of these clay minerals, or mixtures thereof, or different forms thereof, may be used in the magnetic mineral composition of the instant invention.

By way of yet further illustration, one may use a ceramic susceptor material in the magnetic mineral composition of this invention, as that term is described, e.g., in U.S. Pat. No. 4,818,831, the entire disclosure of which is hereby incorporated by reference into this specification. Claim 1 of this patent describes “1. 1. A package article for food to be heated by microwave energy in a microwave oven comprising:
a tray for holding a food item having a top and bottom surface, a substantially planar microwave heating susceptor disposed within said tray, said microwave heating susceptor fabricated from a ceramic composition, comprising: a ceramic binder; and
a ceramic susceptor material which absorbs energy and having a residual lattice charge, wherein the compound is unvitrified, and wherein the susceptor is in intimate physical contact with the food item and ranges in thickness from about 0.5 to 8 mm.” Similar ceramic susceptor compositions are described in U.S. Pat. Nos. 4,965,423; 4,965,427; and 5,183,787, the entire disclosure of each of which is hereby incorporated by reference into this specification

By way of yet further illustration, one may use the activated kaolin described in U.S. Pat. No. 6,290,771 in the magnetic mineral composition of this invention; the entire disclosure of such patent is hereby incorporated by reference into this specification.

Claim 1 of U.S. Pat. No. 6,290,771 describes “A method of preparing an activated kaolin powder compound for mixing with cement, which comprises:
heating natural kaolin to 480° C. for a time period up to one hour, wherein the natural kaolin is primarily composed of halloysite; calcinating the heated natural kaolin in the range of 800°-950° C. over at least 15 minutes; quenching the calcinated kaolin; and
pulverizing the quenched activated kaolin to form powder having particle sizes of 2 μm or less.” The halloysite described in this claim is referred to as “natural kaolin” in the specification of U.S. Pat. No. 6,290,771. Thus, and referring to columns 2-4 of such patent, “The present invention utilizes natural kaolin which is buried under the ground in an tremendous amount. The natural kaolin is activated for use mixing with cement in this invention. The natural kaolin has been used for manufacturing pottery, porcelain, china, etc. In this invention, an activated kaolin powder has been developed from the natural kaolin, which is capable of being used as one of composite materials for mortar or concrete. The activated kaolin powder is prepared by heating natural kaolin to a certain temperature, calcinating the heated kaolin at high temperature, quenching the calcinated kaolin with water or air, and pulverizing the quenched activated kaolin in a form of powder.”

As is also disclosed in U.S. Pat. No. 6,290,771, “Generally, activation of a mineral compound means a state wherein a large amount of crystallization energy is reserved in the molecular structure of the mineral compound when energy is applied to the mineral compound and then the mineral material is quenched, and wherein the mineral compound is in a free state having a strong chemical bonding ability due to the reserved crystallization energy when an external force is applied to the mineral compound.”

As is also disclosed in U.S. Pat. No. 6,290,771, “When the natural kaolin is calcinated at high temperature and then quenched, the kaolin reserves a large amount of crystallization energy in the molecular structure and has a latent hydraulicity, because the kaolin molecules are in a free state. In other words, although the activated kaolin has a high reaction activity and does not cause a hydration when the kaolin contacts with water, the kaolin shows a significant water-setting under a certain circumstance, for instance, in an alkali state. Such water-setting is called as “latent hydraulicity”. The present invention is to provide a natural kaolin with the latent hydraulicity by activation, and to cause a mechanism for hydration and pozzolan reaction of the activated kaolin under a certain circumstance such as in mortar or in concrete.”

As is also disclosed in U.S. Pat. No. 6,290,771, “The reactions intended to derive in the present invention are pozzolan reaction and straetlingite reaction, and the pozzolan reaction shown in the following reaction formula (I) is that a silica and Ca(OH)2 are reacted each other, and the straetlingite reaction shown in the following formula (II) is that a silica, a alumina and Ca(OH)2 are reacted each other. 3Ca(OH)2+2SiO2=3CaO.2SiO2.3H2O (I) 2.a(OH)2+Al2O3+SiO2+6H2O.fwdarw.2CaO.Al2O3.SiO2.8H2O (II)”

As is also disclosed in U.S. Pat. No. 6,290,771, The activated kaolin according to the present invention and Ca(OH)2 from cement cause a pozzolan reaction, and the mortar or concrete using the activated kaolin has excellent strength and water permeability due to the latent hydraulicity.” In one preferred embodiment, the activated kaolin material is mixed with nanomagnetic material, and this mixture is then formed into cement building blocks that, because of the presence of nanomagnetic material, provides shielding against electromagnetic radiation.

As is also disclosed in U.S. Pat. No. 6,290,771, “A bleeding or segregation phenomenon can be improved when a fine particle component is added to a mortar or concrete, which is called as “stabilizing effect”. In this invention, the activated kaolin powder causes the stabilizing effect. When the activated kaolin powder is added to a mortar or concrete, the bleeding or segregation phenomenon of the composition is reduced. Thus, the activated kaolin powder of this invention can cause an excellent stabilizing effect when the activated kaolin powder is together used with mortar or cement. This is believed because the activated kaolin powder fills the porosities of cement particles or reduces the porosity sizes, and because the activated kaolin powder increases the surface of cement paste and aggregate thereby increasing the bonding force of mortar or cement.” As will be apparent to those skilled in the art, when the activated kaolin also contains nanomagnetic particles, not only is the bonding force of the mortar or cement increased, but also the mortar or cement objects formed are capable of shielding against electromagnetic radiation.

As is also disclosed in U.S. Pat. No. 6,290,771, “The activated kaolin powder compound is prepared from natural kaolin. The activated kaolin powder compound is prepared by heating natural kaolin to 480° C. (for a time period up to one hour, calcinating the heated natural kaolin at 800˜950° C. over at least 15 minutes, quenching the calcinated kaolin with water or air, and pulverizing the quenched activated kaolin to give powder having particle sizes of 2 μm or less.”

As is also disclosed in U.S. Pat. No. 6,290,771, “A kaolin used in the present invention is primarily composed of halloysite(Al2O3.2SiO2.4H2O), and a composition thereof is Al2O3 of 36˜39%, SiO2 of 45˜47% and CaO of 1˜2%, and has Al2O3/2SiO2=0.76˜0.87. The kaolin without any treatment can be used in the present invention. A method of pulverizing the quenched activated kaolin comprises crushing by Crusher, and pulverizing by Air Jet Mill. The maximum particle size is 2 μm, and the average particle size is 0.1˜1.0 μm.”

As is also disclosed in U.S. Pat. No. 6,290,771, “FIG. 1 is a schematic graph showing the relationship of temperature with heating and calcinating time in the process of preparing an activated kaolin powder compound according to the present invention. As shown in FIG. 1, the natural kaolin which is dried at ambient temperature is heated to 480° C. It is preferable to heat the natural kaolin for a time period up to one hour in aspect of heat efficiency and energy consumption.”

As is also disclosed in U.S. Pat. No. 6,290,771, “The heated natural kaolin is calcinated in the range of 800˜950° C. In this calcinating step, it is preferable to calcinate the heated kaolin over at least 15 minutes. For excellent physical properties of the activated kaolin powder compound, the calcinating step should be conducted for more than 15 minutes in consideration of the heat efficiency and amount of energy used. In this calcinating step, the temperature should be lower than 950° C., because the physical properties can be adversely affected at the higher temperature than 950° C. The starting temperature for activation of kaolin is in the range of 450˜500° C., and the terminating temperature for that is 980° C. The optimum temperature to improve the compressive strength is in the range of 800˜950° C.”

As is also disclosed in U.S. Pat. No. 6,290,771, “The calcinated kaolin is quenched with water or air. A water-cooling method is more effective in cost than an air-cooling method. The air-cooling method is that the calcinated kaolin is quenched by using an air spray in the range of 20˜60° C., and the water-cooling method is that the calcinated kaolin is immersed in water ranging from 15 to 40° C. Through the quenching step, the kaolin is in an activation state which reserves crystallization energy therein.”

As is also disclosed in U.S. Pat. No. 6,290,771, “The quenched kaolin is pulverized in a form of powder to give particle sizes of 2 μm or less. Kaolin particles having about 1 μm are preferably used. The pulverized kaolin powder has a specific gravity of 1.5 to 3.0.”

As is also disclosed in U.S. Pat. No. 6,290,771, “The activated kaolin powder compound is employed in an amount of about 5 to 15% by weight of cement for preparing mortar or cement. It is preferable to employ the activated kaolin powder compound in an amount of about 10% by weight of the cement.”

A Magnetic Mineral Composition Comprised of an Elastomer

In one preferred embodiment of the invention, the magnetic mineral composition of this invention, in addition to containing magnetic material (such as nanomagnetic material), also contains an elastomer. One may use any of the elastomers that have been used together with clay minerals in the prior art.

By way of illustration, one may use the fibrillated polytetrafluorethylene resin described in U.S. Pat. No. 4,839,221, the entire disclosure of which is hereby incorporated by reference into this specification. Claim 1 of this patent describes “1. A gasket, comprising a sheet of a composition consisting essentially of a fibrillated polytetrafluoroethylene resin and a fine inorganic powder having an average particle size of not larger than 100 μm and containing at least 30% by weight of a clay mineral, based on the total weight of the fine inorganic powder, said composition characterized in that the polytetrafluoroethylene resin is at least 5% by weight and the fine inorganic powder is at least 40% by weight, based on the total amount of the polytetrafluoroethylene resin and the fine inorganic powder, the polytetrafluoroethylene resin and the fine inorganic powder are mutually uniformly dispersed and mixed with each other, and further comprising a metal support for said sheet.” Reference may also be had to U.S. Pat. No. 4,990,544 for a description of a similar material.

The elastomer may, e.g., be an adhesive composition, as described in U.S. Pat. No. 5,686,099, the entire disclosure of which is hereby incorporated by reference into this specification. Claim 1 of this patent describes “A dermal composition comprising a mixture of 0.1 to 50 by dry weight of a drug, a pressure sensitive adhesive, a liquid solvent for one or more of the components of the composition and about 0.1 to about 10% by dry/weight of the total composition of clay to increase the adhesiveness of the composition.” The term “pressure sensitive adhesive” is also described at columns 5-6 of the patent, wherein it is disclosed that “The term “pressure sensitive adhesive” as used herein means and refers to polymers, including but not limited to homopolymers, copolymers and mixtures of polymers, which are adhesive in the sense that they can adhere to the skin of an animal and which are pressure sensitive in the sense that adherence can be effected by the application of pressure. The pressure sensitive adhesive can function as a matrix for the drug. The adhesive is sufficiently resistant to chemical and/or physical attack by the environment of use so that it remains substantially intact throughout the period of use. The adhesive is biocompatible in the environment of use, plastically deformable and with limited water solubility solubility. The term “water” as used herein includes water containing biological fluids such as saline and buffers.”

U.S. Pat. No. 5,686,099 also discloses that “

A wide variety of polymers are known to be suitable for use in pressure sensitive adhesives. Suitable polymers include a natural or synthetic rubber, acryates, polycarboxylic acids or anhydrides thereof, vinyl acetate polymers and the like. A pressure sensitive adhesive can be composed of a single polymer or mixtures thereof. It is generally found that the preferred polymers for pressure sensitive applications have a glass transition temperature of between about −50 to +10 degrees Celsius (° C.). The glass transition temperature is related to the molecular weight of the adhesive.

A preferred dermal composition of this invention comprises a drug; a multipolymer comprising an ethylene/vinyl acetate polymer and an acrylate polymer; a rubber, a clay and, optionally, a tackifying agent. The multipolymer and rubber are preferably in a ratio by weight respectively from about 1:10 to about 30:1, more desirably about 1:5 to 20:1 and preferably about 1:2 to 15:1. The ratio of ethylene/vinyl acetate polymer to acrylate polymer is preferably about 20:1 to about 1:20 by weight. The clay is present in the composition in an amount by dry weight of less than about 50% and preferably from 0.1 to 20%.

By way of yet further illustration, the elastomer may be rubber as is described, e.g., in U.S. Pat. No. 5,936,023, the entire disclosure of which is hereby incorporated by reference into this specification. Claim 1 of this patent describes “1. A method of manufacturing a composite material comprising a rubber and a clay mineral comprising the steps of: exchanging an inorganic ion of a clay mineral with an organic onium ion to organize the clay mineral; mixing the organized clay mineral and a process oil and/or a plasticizer; and mixing a rubber material with the mixture of the organized clay mineral and the process oil and/or the plasticizer and dispersing the clay mineral uniformly in the rubber material.” The rubber material is described at column 3 of this patent as comprising “ . . . at least one rubber selected from natural rubber, isoprene rubber, chloroprene rubber, styrene rubber, nitrile rubber, ethylene-propylene rubber, butadiene rubber, styrene-butadiene rubber, butyl rubber, epichlorohydrin rubber, acrylic rubber, urethane rubber, fluorine rubber, and silicone rubber.”

By way of yet further illustration, one may use one or more of the elastomers described in U.S. Pat. No. 6,617,020, the entire disclosure of which is hereby incorporated by reference into this specification. Claim 1 of this patent describes”

Magnetic Mineral Compositions Comprised of Magnetic Minerals and Other Materials

In the prior sections of this specification, applicants have decribed combinations of a natural mineral and/or a synthetic mineral and/or a nanomagnetic material with either a polymeric material and/or a resin material and/or elastomer material and/or a ceramic material. In this section of the specification, applicants will describe compositions comprised of the natural mineral and/or a synthetic mineral and/or a nanomagnetic material with a material other than such polymeric material, such resin material, such elastomer material, or such ceramic material.

The other material may be an adhesive material, as is described in U.S. Pat. No. 5,686,099, the entire disclosure of which is hereby incorporated by reference into this specification. Claim 1 of such patent describes “A dermal composition comprising a mixture of 0.1 to 50 by dry weight of a drug, a pressure sensitive adhesive, a liquid solvent for one or more of the components of the composition and about 0.1 to about 10% by dry/weight of the total composition of clay to increase the adhesiveness of the composition.”

“The pressure sensistive adhesive” described in the such claim 1 of U.S. Pat. No. 5,686,099 is further described at columns 5-6 of such patent, wherein it is disclosed that “The term “pressure sensitive adhesive” as used herein means and refers to polymers, including but not limited to homopolymers, copolymers and mixtures of polymers, which are adhesive in the sense that they can adhere to the skin of an animal and which are pressure sensitive in the sense that adherence can be effected by the application of pressure. The pressure sensitive adhesive can function as a matrix for the drug. The adhesive is sufficiently resistant to chemical and/or physical attack by the environment of use so that it remains substantially intact throughout the period of use. The adhesive is biocompatible in the environment of use, plastically deformable and with limited water solubility solubility. The term “water” as used herein includes water containing biological fluids such as saline and buffers.”

U.S. Pat. No. 5,686,099 also discloses that “A wide variety of polymers are known to be suitable for use in pressure sensitive adhesives. Suitable polymers include a natural or synthetic rubber, acryates, polycarboxylic acids or anhydrides thereof, vinyl acetate polymers and the like. A pressure sensitive adhesive can be composed of a single polymer or mixtures thereof. It is generally found that the preferred polymers for pressure sensitive applications have a glass transition temperature of between about −50 to +10 degrees Celsius (° C.). The glass transition temperature is related to the molecular weight of the adhesive.”

U.S. Pat. No. 5,686,099 also discloses that “A preferred dermal composition of this invention comprises a drug; a multipolymer comprising an ethylene/vinyl acetate polymer and an acrylate polymer; a rubber, a clay and, optionally, a tackifying agent. The multipolymer and rubber are preferably in a ratio by weight respectively from about 1:10 to about 30:1, more desirably about 1:5 to 20:1 and preferably about 1:2 to 15:1. The ratio of ethylene/vinyl acetate polymer to acrylate polymer is preferably about 20:1 to about 1:20 by weight. The clay is present in the composition in an amount by dry weight of less than about 50% and preferably from 0.1 to 20%”

Certain Nanocomposite Materials Comprised of Mineral Matter and/or Nanomagnetic Material.

In the prior sections of this specification, applicants have described certain “magnetic mineral compositions” that contain mineral matter and/or nanomagnetic material and/or one or more other materials that may be, e.g., polymeric material, resinous material, elastomeric material, ceramic material, mixtures thereof, and the like. In this section of the specification, certain particular nanocomposite materials are described by way of further illustration.

In one embodiment, the halloysite nanotubules described elsewhere in this specification are used as a structural component in a composite material. Such a composite material may comprise a polymer, a polymer blend, or a copolymer into which the nanotubules are dispersed and blended.

Composites containing micron or nanometer scale particles, rods, needles, or tubules are well known. In recent years, polymer composites comprised of clay nanoparticles in particular have been prepared and made into or incorporated in products. Reference may be had to U.S. Pat. No. 6,767,952, “Article utilizing block copolymer intercalated clay,” of Dontula et al., the disclosure of which is incorporated herein by reference. In this patent, there is disclosed an intercalated clay comprising a clay intercalated with a block copolymer wherein said block copolymer comprises a hydrophilic block capable of intercalating said clay. An additional embodiment is an article comprising a matrix polymer and clay wherein said clay is intercalated with a block copolymer, wherein said block copolymer comprises a hydrophilic block capable of intercalating said clay and a matrix compatible block compatible with said matrix polymer. At column 6 of the '952 patent of Dontula et al., it is disclosed that, “The clay material suitable for this invention can comprise any inorganic phase desirably comprising layered materials in the shape of plates with significantly high aspect ratio. However, other shapes with high aspect ratio will also be advantageous, as per the invention. . . . Preferred clays for the present invention include smectite clay such as montmorillonite, nontronite, beidellite, volkonskoite, hectorite, saponite, sauconite, sobockite, stevensite, svinfordite, halloysite, magadiite, kenyaite and vermiculite as well as layered double hydroxides or hydrotalcites.”

Unique and superior properties are attained with nanocomposites comprising inorganic nanoparticles. At column 1 of the '952 patent of Dontula et al., it is further disclosed that, “These properties include improved mechanical properties, such as elastic modulus and tensile strength, thermal properties such as coefficient of linear thermal expansion and heat distortion temperature, barrier properties, such as oxygen and water vapor transmission rate, flammability resistance, ablation performance, solvent uptake, etc. Some of the related prior art is illustrated in U.S. Pat. Nos. 4,739,007; 4,810,734; 4,894,411; 5,102,948; 5,164,440; 5,16,460 5,248,720; 5,854,326; and 6,034,163.”

The '952 patent also discloses that “In general, the physical property enhancements for these nanocomposites are achieved with less than 20 vol. % addition, and usually less than 10 vol. % addition of the inorganic phase, which is typically clay or organically modified clay. Although these enhancements appear to be a general phenomenon related to the nanoscale dispersion of the inorganic phase, the degree of property enhancement is not universal for all polymers. It has been postulated that the property enhancement is very much dependent on the morphology and degree of dispersion of the inorganic phase in the polymeric matrix.

The '952 patent also discloses that “The clays in the polymer-clay nanocomposites are ideally thought to have three structures (1) clay tactoids wherein the clay particles are in face-to-face aggregation with no organics inserted within the clay lattice, (2) intercalated clay wherein the clay lattice has been expanded to a thermodynamically defined equilibrium spacing due to the insertion of individual polymer chains, yet maintaining a long range order in the lattice; and (3) exfoliated clay wherein singular clay platelets are randomly suspended in the polymer, resulting from extensive penetration of the polymer into the clay lattice and its subsequent delamination. The greatest property enhancements of the polymer-clay nanocomposites are expected with the latter two structures mentioned herein above.”

Further disclosures of polymer-clay nanocomposites, methods of preparation thereof, and articles made therefrom may be found in United States published application 2004/00593037, “Materials and method for making splayed layered materials,” of Wang et al.; U.S. Pat. No. 6,767,952, “Polyester nanocomposites,” of Nair et al.; United States published application 2003/0203989, “Article utilizing highly branched polymers to splay layered materials,” of Rao et al.; United States published application 2003/0191224, “Organically modified layered clay as well as organic polymer composition and tire inner liner containing same,” of Maruyama et al.; United States published application 2004/0233526, “Optical element with nanoparticles,” of Kaminsky et al.; United States published application 2004/0259999, “Polyester/clay nanocomposite and preparation method,” of Kim et al.; U.S. Pat. No. 6,832,037, “Waveguide and method for making same,” of Aylward et al.; United States published application 2004/0067033, “Waveguide with nanoparticle induced refractive index gradient,” of Aylward et al.; U.S. Pat. No. 6,728,456, “Waveguide with nanoparticle induced refractive index gradient,” of Aylward et al.; United States published application 2004/0242752, “Hydrophilized porous film and process for producing the same,” of Fujioka et al.; U.S. Pat. No. 6,770,697, “High melt-strength polyolefin composites and methods for making and using same,” of Drewniak et al.; U.S. Pat. No. 6,811,599, “Biodegrable thermoplastic material,” of Fischer et al.; United States published application 2004/0068038, “Exfoliated polystyrene-clay nanocomposite comprising star-shaped polymer,” of Robello et al.; U.S. Pat. No. 6,710,111, “Polymer nanocomposites and the process of preparing the same,” of Kuo et al.; U.S. Pat. No. 6,060,549, “Rubber toughened thermoplastic resin nano composites,” of Li et al.; U.S. Pat. No. 5,972,448, “Nanocomposite polymer container,” of Frisk et al.; United States published application 2002/0132875, “Solid nanocomposites and their use in dental applications,” of Stadtmueller; United States published application 2002/0110686, “Fibers including a nanocomposite material,” of Dugan; U.S. Pat. No. 6,117,541, “Polyolefin material integrated with nanophase particles,” of Frisk; U.S. Pat. No. 6,117,541, “Transparent high barrier multilayer structure,” of Frisk; U.S. Pat. No. 6,265,038, “Transfer/transfuse member having increased durability,” of Ahuja et al.; U.S. Pat. No. 6,190,775, “Enhanced dielectric strength mica tapes,” of Smith et al. The disclosures of each of these United States patents and published applications in its entirety is hereby incorporated herein by reference.

In the formulation of the nanocomposite materials of the present invention, nanotubules of halloysite clay are provided alternatively or additionally to the clay constituents of prior art nanocomposites. In such nanocomposite materials of the present invention, there is provided superior and improved mechanical properties as described in e.g., the '952 patent of Dontula et al. In addition, in certain embodiments, when such nanotubules are loaded with certain active agents and incorporated into the composite, these properties may be tuned by triggering or accelerating the release of such active agent into the polymer matrix of the composite.

In the present invention, and in one embodiment thereof, the halloysite nanotubules are preferably between about 40 nanometers and about 200 nanometers in outer diameter, about 20 nanometers and 100 nanometers in inside diameter, and about 100 to about 2000 nanometers in length. The preferred dimensional ranges and aspect ratio for the nanotubules may vary depending upon the particular application for the composite material.

In preparation of a polymer-halloysite nanotube composite (hereinafter abbreviated PHNT composite) comprised of halloysite nanotubules, the nanotubules are mixed with and blended into the polymer when such polymer is in a liquid state as a hot melt, or is dissolved in a suitable solvent. Alternatively, such polymer may be in an unpolymerized state, i.e., as an unreacted monomer or a partially polymerized resin. In another embodiment, the tubules may be mixed in with one component of a two component reactive system, such as an epoxy resin that is mixed and subsequently polymerized by the use of an “activator” or “hardener.” Both thermoset and thermoplastic polymers may be used in PHNT composites, including but not limited to nylons, polyolefins (e.g. polypropylene), polystyrene, ethylene-vinyl acetate copolymer, epoxies, polyurethanes, polyimides and poly(ethylene terephthalate) (PET).

The nanotubules may be provided as a powder, or as a liquid dispersion or slurry, with such liquid being mixed in with the liquid polymer, monomer resin, or polymer component by conventional means such as batch mixing by an impeller, or other rotational mixing agitator, in a vessel. In one embodiment, the halloysite nanotubules may be mixed in using a twin screw compounded as described at columns 12 and 13 of U.S. Pat. No. 6,767,952 of Dontula et al. Alternatively, the nanotubules may be provided as a dispersion or slurry, wherein a liquid stream of such dispersion flowing in a first tube or conduit is joined with a flowing liquid stream of liquid polymer, monomer resin, or polymer component in a second tube or conduit, and such combined streams in a third tube or conduit are immediately delivered through a motionless mixer, in order to thoroughly mix the nanotubules with the liquid polymer, monomer resin, or polymer component into a nanotube-containing liquid.

Subsequently, the nanotube-containing liquid is processed to make an intermediate PHNT product, or an end PHNT product. Intermediate products include films, sheets, rods, bars, and other elongated structural shapes that can be subsequently machined, molded, pressed, or otherwise formed into other shapes for use as or within a product. Many end products may be made from the halloysite nanotubule composites of the present invention, including but not limited to food packaging, dental implants, optical waveguides, woven fiber products, imaging films, tapes, and rubber goods.

The particular process used to make such intermediate products will depend upon the form of the intermediate product. Thin films of PHNT composite may be formed from the nanotube-containing liquid on a suitable substrate by conventional thin-film forming methods including but not limited to spray coating, dip coating, and roll coating. The latter method, roll coating, pertains to the coating of thin liquid films upon rolls of sheet substrate such as e.g., acetate polymer substrate used in photographic film, or metallized poly(ethylene terephthalate) substrate used in organic photoconductors. Film formation methods for roll coating include reverse roll coating, forward roll coating, gravure coating, slot die extrusion coating, and slide die coating. After formation of the PHNT composite thin film, such film may remain on the substrate in such cases where the substrate is an integral functional part of the product, or provides additional structural support to the product. In other embodiments, a substrate is provided that has poor adhesion to the PHNT composite thin film, thereby enabling the PHNT film to be delaminated from the substrate, and wound into a separate roll for subsequent use.

In other embodiments, intermediate PHNT product in the form of sheets, rods, bars, and other elongated structural shapes may be made by processes such as extrusion, molding, or pultrusion (wherein a long fiber constituent such as glass fibers is also provided in the product). In extrusion processes for the manufacture of such sheets, rods, bars, and other elongated structural shapes, the nanotube-containing liquid may contain a dissolved gas and may be delivered through an extrusion die at high pressure, such that an extruded PHNT foam is produced when the nanotube-containing liquid exits the extrusion die and is at the much lower pressure of the ambient atmosphere. The PHNT product may be comprise a thermoset polymer such as an epoxy or polyester, or a thermoplastic polymer such as polypropylene. When the PHNT product comprises a thermoplastic polymer, the PHNT product may be made using a process wherein the nanotube-containing polymer liquid is provided as a hot-melt polymer liquid.

In certain embodiments, the PHNT composite materials are formed with the nanotubules oriented in selected directions, so as to provide anisotropy in certain mechanical properties. If the nanotubules are preferentially oriented along the x-axis, for example, a PHNT composite will exhibit greater tensile and compressive strength along the x-axis than along the y- and z-axes and more resistance to bending and shear stress perpendicular to the x-axis. In certain manufacturing processes, the nanotubules may be “passively” aligned at least to a significant extent by certain effects inherent in the process. For example, in a process where a film of high viscosity nanotube-containing polymer liquid is extruded as a free-standing film, or onto a substrate, the flow of such liquid is laminar, and the nanotubes will tend to align preferentially along the streamlines of such flow. When the film is dried or cured to a final state, its mechanical properties will be anisotropic due to the directional alignment of the nanotubules.

In other embodiments, the nanotubules may be provided with a coating that allows such nanotubules to be “actively” aligned. For example, such tubules may be coated with a magnetic material such as, e.g., the nanomagnetic material described elsewhere in this specification. During the process when the intermediate or end product is fabricated, the product is preferably subjected to a magnetic field while still in a liquid state, thereby providing the nanotubules with an alignment with the field lines of the magnetic field. The product is subsequently dried or cured into a solid state, thereby retaining the alignment of the coated nanotubules.

Multiple layers of sheet or films of such directionally oriented PHNT composite may be laminated together, wherein the orientation of the nanotubules varies from layer to layer, thereby providing a laminated structure of high strength.

In another embodiment, the nanotubules are loaded with an active agent that can be released after the initial curing/drying and solidification of the product. The active agent is reactive with the polymer (or polymer matrix) in a manner that changes the mechanical properties of the polymer. Thus, when the active agent is released over time in a controlled matter into the solid polymer matrix, the active agent will react or otherwise interact with the polymer to result in a time dependent change in the overall PHNT composite properties. For example, in one embodiment, the nanotubules may be filled with a solvent that can soften the polymer. The nanotubules may also be provided with end caps to retard the release of such solvent during the formation of the PHNT product.

After initial curing or drying, the resulting product has a certain modulus of elasticity and stress vs. strain behavior. Subsequently, the solvent is released from the nanotubules, providing the PHNT product with a more elastic and/or plastic behavior. This effect may be temporary, in that such solvent will subsequently diffuse and evaporate from the PHNT product. In an alternative embodiment, the nanotubules are filled with a plasticizing agent that imparts a long term change in the structural properties of the polymer matrix.

In another embodiment, the nanotubules may be filled with an active agent that reacts with the polymer to render the polymer more rigid. When the active agent is released from the nanotubules, such active agent causes cross-linking of the polymer, thereby increasing the strength of such polymer, and of the PHNT product.

The controlled release of such active agents is described in detail in U.S. Pat. No. 5,705,191, “Sustained delivery of active compounds from tubules, with rational control,” of Price et al., the disclosure of which is incorporated herein by reference. In this patent, Price et al. disclose a method for releasing an active agent into a use environment, by disposing such active agent within the lumen of a population of tubules, and disposing such tubules into a use environment, either directly or in some matrix such as a paint in contact with the use environment. The tubules have a preselected release profile to provide a preselected release rate curve. The preselected release profile may be achieved by controlling the length or length distribution of the tubules, or by placing degradable endcaps over some or all of the tubules in the population, by mixing the active agent with a carrier, and filling the tubules with the carrier/agent, or by combinations of these methods.

In a further embodiment, the rate at which the active agent is released is accelerated and/or further controlled by subjecting the PHNT product/material to an energy source such as ultrasonic energy. For active agents that are volatile, or have a highly volatile component, the ultrasonic energy may result in localized cavitation within or at the ends of the tubules, thereby greatly accelerating the rate of discharge of active agent.

The description of PHNT composites of the present invention has heretofore been with regard to bulk composites, i.e. composites wherein the distribution of nanotubules through the polymer matrix is substantially homogeneous. In another embodiment, such nanotubules are provided to form a thin outer nanocomposite layer or “skin” on the external surface of a polymer or other material.

In one embodiment, a nanocomposite material comprised of halloysite nanotubules distributed through a matrix of polyvinylidene fluoride polymer. It is well known that polyvinylidene fluoride (PVDF) is a piezoelectric material. The application of a mechanical stress to a film of PVDF results in the generation of an electric potential across such film. Conversely, the application of an electric potential across a film of PVDF results in a mechanical stress in such film, and a deformation of such film. Such piezoelectric films have thus found utility in acoustic applications, sensors, microactuators, and the like.

In one preferred embodiment, a nanocomposite material comprising polyvinylidene fluoride polymer and halloysite nanotubules filled with an active agent to be released from the film. A high frequency AC voltage is applied to such film, resulting in a high frequency oscillation and increase in temperature of such film, with a corresponding accelerated release of active agent.

A Composition Comprised of a Biodegradable Material and Nanomagnetic Material.

In one embodiment of this invention, there is provided a biodegradable thermoplastic material comprised of a clay mineral. This composition is similar in to the composition described in U.S. Pat. No. 6,811,599, the entire disclosure of which is hereby incorporated by reference into this specification. However, in addition to the biodegradable thermoplastic material, it also contains the nanomagnetic material described elsewhere in this specification.

Claim 1 of U.S. Pat. No. 6,811,899 describes “1. A biodegradable thermoplastic material comprising a natural polymer, a plasticizer and an exfoliated clay having a layered structure, said clay having a cation exchange capacity of from 30 to 250 milliequivalents per 100 grams.” Some of these biodegradable thermoplastic materials are described at columns 2-3 of such patent, wherein it is disclosed that “Most of the known biodegradable thermoplastic materials are either also based on hydrocarbon sources, or based on natural raw materials (monomers) or even natural polymers, such as cellulose, starch, polylactic acid, keratin, and the like. These natural raw materials are, more or less intrinsically, biodegradable. Furthermore, they have the advantage that they originate from renewable sources and will therefore always be available. Natural polymers are, however, generally not thermoplastic. In order to achieve that property, the materials are typically processed (often extruded) in combination with a plasticizer. Of course, the biodegradable properties of a suitable plasticizer are to be considered in its selection.”

U.S. Pat. No. 6,811,599 also discloses that “Unfortunately, in practice there are not many choices for the plasticizer. Usually, either water, urea, glycerol or a low aliphatic or aromatic ester is selected. Problems that are encountered are that these plasticizers either are insufficiently compatible with the biodegradable polymer, or may leach out of the product, which in its turn will become brittle and may even fall apart. This problem is particularly encountered in applications wherein the product is used in a humid or aqueous environment, i.e. when it is brought into contact with water. This disadvantage puts a serious limitation on the applications of the biodegradable thermoplastic material. It moreover means that the (mechanical) properties of the material deteriorate rather fast, making it unsuitable for use long before its biodegradation takes effect.”

U.S. Pat. No. 6,811,599 also discloses that “The present invention seeks to overcome the problems associated with the known biodegradable thermoplastic materials from natural polymers. In particular, it is an object of the invention to provide a material, which is biodegradable and has good thermoplastic and mechanical properties, which material is highly compatible with biodegradable plasticizers. It is furthermore an object of the invention that the favorable properties of the biodegradable thermoplastic material remain apparent over a prolonged period of time, preferably at least until biodegradation affects said properties.”

U.S. Pat. No. 6,811,599 also discloses that “Surprisingly, it has been found that these objects can be reached by incorporating a specific clay into a biodegradable thermoplastic material. Accordingly, the invention relates to a biodegradable, thermoplastic material comprising a natural polymer, a plasticizer and a clay having a layered structure and a cation exchange capacity of from 30 to 250 milliequivalents per 100 gram.”

U.S. Pat. No. 6,811,599 also discloses that “Due to the presence of the clay, the plasticizer is substantially retained in the biodegradable thermoplastic material, thereby avoiding the problems with loss of plasticizer that were encountered with the known biodegradable thermoplastic materials. Hence, a material according to the invention has superior properties, and those properties are maintained over a prolonged period of time. In other words, the stability of a biodegradable thermoplastic material is significantly improved because of the presence of the clay. In accordance with the invention, a thermoplastic material is a material that is deformable upon increase of temperature.”

U.S. Pat. No. 6,811,599 also discloses that “In the prior art, a combination of a natural polymer, in this case a polysaccharide, and a clay has been disclosed in the German patent application 195 04 899. However, this combination is not a thermoplastic material as no plasticizer is present. Furthermore, the clay is used in combination with the polysaccharide merely in order to control the porosity of the material.”

U.S. Pat. No. 6,811,599 also discloses that “In the European patent application 0 691 381 a biodegradable resin is disclosed containing a biodegradable polymer, such as a polysaccharide, and an inorganic layered compound. In an embodiment for the production of a resin, the inorganic layered compound has been treated with a swelling agent, which is removed after formation of the product. The swelling agent helps to provide inorganic laminar compounds with a very high aspect ratio (i.e. particle size divided by particle thickness) more easily. The swelling agent is removed by drying the resin product at a high temperature (e.g. 2 hours at 80° C. or 10 min at 140° C.). Water is claimed to be a suitable swelling agent, because of its relatively low boiling point, which makes removal more easy.”

U.S. Pat. No. 6,811,599 also discloses that “The natural polymer on which the present biodegradable thermoplastic material is based, may be any natural polymer that is conventionally used to serve as bas for a biodegradable thermoplastic material. Examples include carbohydrates (polysacides) and proteins. Particular good results have been obtained using starch, cellulose, chitosan, alginic acid, inulin, pectin, casein and derivatives thereof. Derivatives that may be used are for example esters, such as acetylated starch, or carboxymethylated cellulose, and ethers, such as hydroxypropylated starch.”

U.S. Pat. No. 6,811,599 also discloses that “In accordance with the invention, it has further been found that some of these natural polymers may be used without plasticizer, leading, in combination with the clay, to a biodegradable thermoplastic or thermosetting material. Natural polymers that have been found suitable or preparing a thermoplastic or thermosetting material in accordance with this embodiment are the above mentioned derivatives having a high degree of substitution (DS), typically at least 1. Specific examples include acetylated starch and hydroxypropylated cellulose.”

U.S. Pat. No. 6,811,599 also discloses that “A suitable plasticizer is a compound that is compatible with the other constituents of the material and that is capable of imparting thermoplastic properties to the material. Suitable examples for the plasticizer include water, urea, glycerol, sorbitol ethylene glycol, oligomers of ethylene glycol and mixtures thereof. Preferably, the plasticizer is used in an amount of 15 to 60 wt. %, more preferably of 25 to 45 wt. %, based an the weight of the thermoplastic material. It is an important aspect of the present invention that the added plasticizer is substantially retained in the thermoplastic material after processing. In a preferred embodiment the thermoplastic material comprises a relative amount of at least 15 wt. %, more preferably of at least 20 wt. % and most preferably at least 25 wt. % of plasticizer based on the weight of the thermoplastic material.”

A Composition Comprised of Biological Material and Nanomagnetic Material

In one especially preferred embodiment, the nanomagnetic material of this invention, described elsewhere in this case, is used to construct the magnetic nanoparticles described in U.S. Pat. No. 6,767,635, the entire disclosure of which is hereby incorporated by reference into this specification. This patent describes, in claim 1 thereof, “1. Magnetic nanoparticles having biochemical activity, consisting of a magnetic core particle and an envelope layer fixed to the core particle, wherein the magnetic nanoparticles comprise a compound of general formula M-S-L-Z (I), the linkage sites between S and L and, L and Z further comprise covalently bound functional groups, wherein M represents said magnetic core particle; S represents a biocompatible substrate fixed to M; L represents a linker group, and Z represents a group comprised of nucleic acids, peptides or proteins or derivatives thereof, at least one of which binds to an intracellular biomacromolecule.” The magnetic core is further defined in claim 2 of the patent as consisting of “ . . . magnetite, maghemite, ferrites of general formula MeOxFe2O3 wherein Me is a bivalent metal selected from the group consisting of cobalt, manganese, iron, of cobalt, iron, nickel, iron carbide, and iron nitride.” By comparison, the preferred nanomagnetic particles of the instant invention are comprised of a trivalent metal (such as aluminum) and are substantially fully oxidized (unlike Fe203). Thus, the nanomagnetic particles of the instant invention are more stable and possess superior magnetic properties.

The size of the “core particles” of the magnetic nanoparticles of U.S. Pat. No. 6,767,635 is defined in claim 3 of such patent as being “ . . . from 2 to 100 nm.” Claim 4 of such patent describes the biocompatible substrate, S, as being “ . . . a compound selected from the group consisting of poly- or oligosaccharides or derivatives thereof, such as dextran, carboxymethyldextran, starch, dialdehyde starch, chitin, alginate, cellulose, carboxymethylcellulose, proteins or derivatives thereof, albumins, peptides, synthetic polymers, polyethyleneglycols, polyvinylpyrrolidone, polyethyleneimine, polymethacrylates, bifunctional carboxylic acids and derivatives thereof, mercaptosuccinic acid or hydroxycarboxylic acids.”Claim 5 of such patent describes the linker group, L, as being “ . . . formed by reaction of a compound selected from the group consisting of poly- and dicarboxylic acids, polyhydroxycarboxylic acids, diamines, amino acids, peptides, proteins, lipids, lipoproteins, glycoproteins, lectins, oligosaccharides, polysaccharides, oligonucleotides and alkylated derivatives thereof, and nucleic acids (DNA, RNA, PNA) and alkylated derivatives thereof, present either in single-stranded or double-stranded form, which compound includes at least two identical or different functional groups.” Claim 6 of such patent describes the functional groups as being “ . . . selected from the group consisting of —CHO, —COOH, —NH2, —SH, —NCS, —NCO, —OH, —COOR, wherein R represents an alkyl, acyl or aryl residue . . . . ” Claim 7 of such patent describes that “ . . . S and M are covalently linked to each other.” Claim 8 of such patent describes that “ . . . an electrostatic bond is formed between M and S.” Claim 9 of such patent describes “A dispersion, comprised of magnetic nanoparticles according to claim 1 and a carrier fluid.” This dispersion is further described in claim 10 of the patent as having wherein

Claim 13 of U.S. Pat. No. 6,767,635, the entire disclosure of which is hereby incorporated by reference into this specification, describes “13. A biochemically active compound of general formula S-L-Z (II), the linkage sites between S and L and L and Z having covalently bound functional groups, wherein S represents a biocompatible substrate fixed to M represents magnetic core particle; L represents a biocompatible linker group, and Z represents a group comprised of nucleic acids, peptides and/or proteins or derivatives thereof, which group has at least one structure that binds to an intracellular biomacromolecule.”

A will be apparent to those skilled in the art, the preferred nanomagnetic materials of the instant invention may be used to replace the nano-sized ferrites of U.S. Pat. No. 6,767,635 to produce improved magnetic nanoparticles. One may make such preferred nanomagnetic materials in accordance with the procedure described elsewhere in this specification and use them in accordance with the process of U.S. Pat. No. 6,767,635 to prepare the improved magnetic nanoparticles.

The process for producing the improved magnetic nanoparticles is described at columns 2-9 of U.S. Pat. No. 6,767,635, the entire disclosure of which is hereby incorporated by reference into this specification. As is disclosed in this portion of the patent, “The production of the magnetic nanoparticles is performed in steps. The magnetic core particles are produced in a per se known manner and, in a preferred variant, reacted directly with the biochemically active compound (II).”

U.S. Pat. No. 6,767,635 also discloses that “In another embodiment of the invention, the magnetic core particles are produced according to the following method: a. producing the magnetic core particles in a per se known manner; b. reacting the magnetic core particles with the biocompatible substrate S; and c. reacting the compound M-S having formed with a compound L-Z; wherein in order to produce L-Z, a compound such as poly- and dicarboxylic acids, polyhydroxycarboxylic acids, diamines, amino acids, peptides, proteins, lipids, lipoproteins, glycoproteins, lectins, oligosaccharides, polysaccharides, oligonucleotides and alkylated derivatives thereof, and nucleic acids (DNA, RNA, PNA) and alkylated derivatives thereof, present either in single-stranded or double-stranded form, which compound includes at least two identical or different functional groups, is reacted with nucleic acids, peptides and/or proteins or derivatives thereof having at least one functional group and including at least one structure capable of specifically binding to a binding domain of an intracellular biomacromolecule.”

U.S. Pat. No. 6,767,635 also discloses that “The procedure for producing the biochemically active compound (II) is such that compound L-Z is produced first, and L-Z subsequently is reacted with the substrate S.”

U.S. Pat. No. 6,767,635 also discloses, in Example 1 thereof, “0.5 mol FeCl2.multidot.4H2O and 1 mol FeCl3.multidot.6H2O are completely dissolved in 100 ml of water and added with concentrated ammonium hydroxide with stirring until a pH value of 9 is reached. The black particles in the dispersion are separated by magnetic means, and the supernatant is decanted. Thereafter, the dispersion is brought to pH 1-4 using half-concentrated HCl, thereby exchanging the particle charges. This process is repeated until the particles begin to redisperse. Subsequently, this is centrifuged (5,000 to 10,000 g), and the supernatant low in particles is decanted. The residue is taken up in HCl (3-10 N), and the complete process is repeated until an electric conductivity of 20-500 μS/cm at a pH value of 4-5 is reached, or, the residue is dialyzed against HCl (3-10 N) until the same values are reached.”

U.S. Pat. No. 6,767,635 also discloses, in Example 1 thereof, “The saturation polarization of the stable magnetite/maghemite sol having formed is 6 mT at maximum.”

U.S. Pat. No. 6,767,635 also discloses, in Example 2 thereof, “0.5 mol FeCl2.multidot.4H2O and 1 mol FeCl3.multidot.6H2O are completely dissolved in 100 ml of water and added with concentrated ammonium hydroxide with stirring until a pH value of 9 is reached. The black particles in the dispersion are separated by magnetic means, and the supernatant is decanted. Subsequently, this is added with some milliliters of hydrogen peroxide (30%), thereby oxidizing the particles to form maghemite. Thereafter, the particles are treated by adding half-concentrated HCl as described in Example 1. The saturation polarization of the stable maghemite sol having formed is 6 mT at maximum.”

U.S. Pat. No. 6,767,635 also discloses, in Example 3 thereof, “100.ml of the magnetite and/or maghemite sol described in Examples 1 and 2 is added with 6 g of CM-dextran (DS 0.4-2) dissolved in 20 ml of water, and the mixture is heated with stirring at 40-80° C., preferably 50-60° C., for 30 minutes. The stable sol being formed, consisting of magnetite/maghemite particles coated with CM-dextran, is subsequently purified using dialysis against water.”

U.S. Pat. No. 6,767,635 also discloses, in Example 4 thereof, ” To a solution of 0.6 g of CM-dextran (DS 0.4-2) in 25 ml of water, 13.1 ml of a 1 M Fe(III) chloride solution including 2.04 g of FeCl2.multidot.4H2O dissolved therein is slowly added drop wise at 70° C. with stirring. Thereafter, the reaction mixture is brought to pH 9-10 by adding dilute NaOH (2N), and this is subsequently neutralized with dilute HCl (2N) and stirred for 2 hours at 70° C., the pH value of the solution being maintained at about 6.5-7.5 by further addition of dilute NaOH or HCl. The reaction mixture is cooled, followed by removal of insolubles by centrifugation, and the magnetic fluid obtained is purified using dialysis against water. The saturation polarization of the nanoparticles coated with CM-dextran is 6 mT at maximum.”

U.S. Pat. No. 6,767,635 also discloses, in Example 5 thereof, “100.ml of the magnetite and/or maghemite sol described in Examples 1 and 2 is added with 2 g of dimercaptosuccinic acid dissolved in 20 ml of water, and the mixture is heated with stirring at 70° C. for 30 minutes. The stable sol being formed, consisting of magnetite/maghemite particles coated with dimercaptosuccinic acid, is subsequently purified using dialysis against water. The saturation polarization is 1-8 mT, preferably 3-6 mT.”

U.S. Pat. No. 6,767,635 also discloses, in Example 6 thereof, “100.ml of the magnetite and/or maghemite sol described in Examples 1 and 2 is added with 6 g of bovine albumin dissolved in 100 ml of water, and the mixture is heated with stirring at 70° C. for 30 minutes. The stable sol being formed, consisting of albumin-coated magnetite/maghemite particles, is subsequently purified using dialysis against water.”

U.S. Pat. No. 6,767,635 also discloses, in Example 7 thereof, “100.ml of the dispersion produced according to Example 1 or 2 is mixed up in an alkaline solution containing 7 g of N-oleoylsarcosine (Korantin SH from BASF) and stirred for 30 minutes at 50-80° C., preferably at 65° C. The particles agglomerate upon mixing, but re-stabilize when maintaining the pH value in the alkaline range, preferably between 8 and 9. The particles precipitate in the acidic range, but undergo redispersion in the alkaline range.”

U.S. Pat. No. 6,767,635 also discloses, in Example 8 thereof, “To 1 mg of succinic acid dissolved in 10 ml of water, an equimolar amount of a water-soluble carbodiimide (N-ethyl-N′-(3-dimethylaminopropyl)carbodiimide hydrochloride) is added with stirring, and this is stirred for 30 minutes at 5-10° C. Subsequently, 10 μg of an amino-functionalized oligonucleotide . . . dissolved in 50 μl of phosphate buffer (pH 7.0) is added, and the mixture is maintained at 5-10° C. for 24 hours. To remove byproducts and non-reacted starting materials, this is dialyzed against water, and the reaction product is lyophilized.”

U.S. Pat. No. 6,767,635 also discloses, in Example 9 thereof, “To 10 μg of the oligonucleotide functionalized according to Example 8 and dissolved in 100 μl of phosphate buffer (pH 7.0), 20 μg of a water-soluble carbodiimide (N-ethyl-N′-(3-dimethylaminopropyl)carbodiimide hydrochloride) is added with stirring, and this is maintained at 5-10° C. for 30 minutes. Subsequently, this solution is added to 200 mg of albumin dissolved in 20 ml of phosphate buffer, and the mixture is maintained at 5-10° C. for 24 hours. To remove byproducts and non-reacted starting materials, this is dialyzed against water, and the reaction product obtained is lyophilized.”

U.S. Pat. No. 6,767,635 also discloses, in Example 10 thereof, “1.ml of the magnetite and/or maghemite sol described in Examples 1 and 2 is diluted with water at a ratio of 1:10 and adjusted to pH 7 by adding dilute NaOH. Subsequently, 60 mg of albumin functionalized according to Example 9 and dissolved in 10 ml of phosphate buffer (pH 7.0) is added, and this is heated for about 30 minutes at 40° C. with stirring. The magnetic fluid thus obtained is subsequently centrifuged, and the solution is purified using dialysis against water.”

U.S. Pat. No. 6,767,635 also discloses, in Example 11 thereof, “To 10 μg of the oligonucleotide functionalized according to Example 8 and dissolved in 100 μl of phosphate buffer (pH 7.0), 20 μg of a water-soluble carbodiimide (N-ethyl-N′-(3-dimethylaminopropyl)carbodiimide hydrochloride) is added with stirring, and this is maintained at 5-10° C. for 30 minutes. Subsequently, this solution is added to 10 ml of the magnetic fluid prepared according to Example 6 and diluted with water at a ratio of 1:10, maintained at 5-10° C. for 24 hours and then purified using dialysis against water.”

U.S. Pat. No. 6,767,635 also discloses, in Example 12 thereof, “1.ml of the magnetic fluid prepared according to Example 3 or 4 is diluted with water at a ratio of 1:10, added with 20 mg of a water-soluble carbodiimide (N-ethyl-N′-(3-dimethylaminopropyl)carbodiimide hydrochloride), and this is stirred at 5-10° C. for about 30 minutes. Thereafter, 10 mg of a peptide (H-Ala-Ala-Ala-Ala-OH) is added, and the mixture is maintained at 5-10° C. for 24 hours. To remove byproducts and non-reacted starting materials, this is dialyzed against water.”

U.S. Pat. No. 6,767,635 also discloses, in Example 13 thereof, “To 10 ml of the solution described in Example 12, 20 mg of a water-soluble carbodiimide (N-ethyl-N′-(3-dimethylaminopropyl)carbodiimide hydrochloride) is added, and this is stirred at 5-10° C. for 30 minutes and added with 10 μg of an amino-functionalized oligonucleotide (see Example 7) dissolved in 50 μl of phosphate buffer (pH 7.0). The mixture is maintained at 5-10° C. for 24 hours and subsequently dialyzed against water.”

A Time-Release Composition Comprised of a Clay Mineral, a Drug, and Nanomagnetic Material.

In one embodiment of this invention, there is provided a time-release composition comprised of a drug, a clay mineral, and nanomagnetic material. This composition is similar in some respects to the dermal compositions described in the claims of U.S. Pat. No. 5,686,099, the entire disclosure of which is hereby incorporated by reference into this specification; but, in addition to the materials described in such patent, it also contains the nanomagnetic material described elsewhere in this specification.

Claim 1 of U.S. Pat. No. 5,686,099 describes “1. A dermal composition comprising a mixture of 0.1 to 50 by dry weight of a drug, a pressure sensitive adhesive, a liquid solvent for one or more of the components of the composition and about 0.1 to about 10% by dry/weight of the total composition of clay to increase the adhesiveness of the composition.” As is described in claim 2 of such patent, the clay may be selected from the group consisting of “ . . . hydrated aluminum silicate, kaolinite, montmorillonite, atapulgite, illite, bentonite, and halloysite.” The composition may also contain a “multipolymers” as is disclosed, e.g., in claim 7 of such patent, which describes a composition that includes “ . . . 17-beta-estradiol, a multipolymer containing acrylate and ethylene vinyl acetate monomers, a natural or synthetic rubber and a clay.”

Some of the drugs that may be used in the composition of U.S. Pat. No. 5,686,099 are described at columns 4-5 of such patent and include, by way of illustration and not limitations, “ . . . 1. Anti-infectives, such as antibiotics, including penicillin, tetracycline, chloramphenicol, sulfacetamide, sulfamethazine, sulfadiazine, sulfamerazine, sulfamethizole and sulfisoxazole; antivirals, including idoxuridine; and other anti-infectives including nitrofurazone and the like . . . . 2. Anti-allergenics such as antazoline, methapyrilene, chlorpheniramine, pyrilamine and prophenpyridamine; 3. Anti-inflammatories such as hydrocortisone, cortisone, dexamethasone, fluocinolone, triamcinolone, medrysone, prednisolone, piroxicam, oxicam and the like; 4. Decongestants such as phenylephrine, naphazoline, and tetrahydrozoline; 5. Miotics and anticholinesterases such as pilocarpine, carbachol, and the like; 6. Mydriatics such as atropine, cyclopentolate, homatropine, scopolamine, tropicamide, ecuatropine and hydroxyamphetamine; 7. Sympathomimetics such as epinephrine; 8. Sedatives, hypnotics, analgesics and anesthetics such as chloral, pentobarbital, phenobarbital, secobarbital, codeine, lidocaine, fentanyl and fentanyl analogs, opiates, opioids, agonists and antagonists therefor; 9. Psychic energizers such as 3-(2-aminopropyl)indole, 3-(2-aminobutyl)indole, and the like; 10. Tranquilizers such as reserpine, chlorpromazine, thiopropazate and benzodiazepines such as alprazolam, triazolam, lorazepam and diazepam; 11. Androgenic steroids such as methyltestosterone and fluoxymesterone; 12. Estrogens such as estrone, 17-beta-estradiol, ethinyl estradiol, and diethylstilbestrol; 13. Progestational agents, such as progesterone, 19-norprogesterone, norethindrone, megestrol, melengestrol, chlormadinone, ethisterone, medroxyprogesterone, norethynodrel 17 alpha-hydroxyprogesterone dydrogesterone, and nomegesterol acetate; 14. Other steroids or steroid like substances such as androgens; 15. Humoral agents such as the prostaglandins, for example PGE1, PGE2 alpha, and PGF2 alpha; 16. Antipyretics such as aspirin, salicylamide, and the like; 17. Antispasmodics such as atropine, methantheline, papaverine, and methscopolamine; 18. Anti-malarials such as the 4-aminoquinolines, alpha-aminoquinolines, chloroquine, and pyrimethamine; 19. Antihistamines such as diphenhydramine, dimenhydrinate, perphenazine, and chloropenazine; 20. Cardiovascular agents such as nitroglycerin, isosorbide dinitrate, isosorbide mononitrate, quinidine sulfate, procainamide, flumethiazide, chlorothiazide, calcium antagonists such as nifedipine, verapamil and diltiazem and selective and non-selective beta blockers such as timolol, salbutamol, terbutaline and propranolol, ACE inhibitors such as captopril and various other agents such as clonidine and prazosin; 21. Nutritional agents such as essential amino acids and essential fats.”

In the preferred embodiment disclosed in U.S. Pat. No. 5,686,099, one or more of the aforementioned drugs is disposed within a pressure sensitive adhesive and is rapidly released therefrom. Thus, as is disclosed at columns 7-9 of such patent, “With the present invention, drugs incorporated into the pressure sensitive adhesive are rapidly released to the skin. The fact that the drug is rapidly released to the skin and may be in a liquid that functions as a solvent, does not in fact negatively affect the rate of permeation through the skin and the resulting blood levels of the drug. Rather, the system permits even delivery of the drug to the blood, particularly a steroidal drug, and with less percent fluctuation of blood levels of drug, namely peak to trough variation, than when controlled diffusion is used. When the device of this invention is placed on the skin, the drug will permeate to and through the skin.

As is also disclosed in U.S. Pat. No. 5,686,099, “The dermal composition according to the present invention can be prepared, for example, by mixing the adhesive, for example the multipolymer including the acrylate polymer, drug, the rubber, the optional solvent, clay and optional tackifying agent in an appropriate lower molecular weight liquid. Appropriate liquids are preferably volatile polar and non-polar organic liquids, such as an alcohol, such as isopropyl alcohol or ethanol, a benzene derivative such as xylene or toluene, alkanes and cycloalkanes such as hexane, heptane and cyclohexane and an alkanoic acid acetate such as an ethyl acetate. The liquid mixture is cast at ambient pressure and all lower molecular weight liquids removed; for example by evaporation, to form a film. The higher boiling solvents such as lower molecular weight alkane diols used in the composition remain therein.”

As is also disclosed in U.S. Pat. No. 5,686,099, “The ethylene/vinyl acetate polymers can be either a copolymer or a terpolymer. Thus a copolymer of vinyl acetate and ethylene can be used. A terpolymer of an acrylic acid/ethylene/vinyl acetate can also be used. Thus the third monomer of the terpolymer can be an acrylic acid such as acrylic acid or methacrylic acid or copolymers thereof. The acrylate polymer can be any of the various homopolymers, copolymers, terpolymers and the like of various acrylic acids. The acrylic polymer constitutes preferably from about 5% to about 95% the total weight of the multipolymer, and preferably 25% to 92%, the amount of the acrylate polymer being chosen being dependent on the amount and type of the drug used. Thus the smaller the amount of the drug used, the greater amount of the acrylate polymer can be used.”

As is also disclosed in U.S. Pat. No. 5,686,099, “The acrylate polymers of this invention are polymers of one or more acrylic acids and other copolymerizable functional monomers. The acrylate polymer is composed of at least 50% by weight of an acrylate or alkylacrylate, from 0 to 20% of a functional monomer copolymerizable with the acrylate and from 0 to 40% of other monomers.”

As is also disclosed in U.S. Pat. No. 5,686,099, “Acrylates which can be used include acrylic acid, methacrylic acid and esters thereof, including N-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decyl methacrylate, dodecyl acrylate, dodecyl methacrylate, tridecyl acrylate, and tridecyl methacrylate. Functional monomers copolymerizable with the above alkyl acrylates or methacrylates which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate.”

As is also disclosed in U.S. Pat. No. 5,686,099, “Ethylene/vinyl acetate copolymers and terpolymers are well known, commercially available materials. Typically such polymers have a vinyl acetate content of about 4 percent to 80 percent by weight and an ethylene content of 15 to 90 percent of the total. Preferably the ethylene/vinyl acetate copolymer or terpolymer has a vinyl acetate content of about 4 percent to 50 percent by weight, with a melt index of about 0.5 to 250 grams per ten minutes, and a density having a range of about 0.920 to 0.980. More preferably the polymer has a vinyl acetate content of about 40 percent by weight and a melt index of about 0.5 to 25 grams per ten minutes. Melt index is the number of grams of polymer which can be forced through a standard cylindrical orifice under a standard pressure at a standard temperature and thus is inversely related to molecular weight. As is used in the specification, melt index is determined in accordance with the standard ASTM D 1238-65T condition E.”

As is also disclosed in U.S. Pat. No. 5,686,099, “In addition to varying the percentage of vinyl acetate in the ethylene/vinyl acetate polymer, the properties of the multipolymer can be changed by varying the amount of acrylate.”

As is also disclosed in U.S. Pat. No. 5,686,099, “From the foregoing it can be understood that the multipolymer can be composed of an ethylene/vinyl acetate polymer containing at least about from 15 to 90 percent by weight of ethylene monomer and from about 4 to 80 percent by weight of vinyl acetate monomer, and from about 5 to 95% of an acrylate polymer. The selection of the particular ethylene/vinyl acetate and acrylate multipolymer, along, with the rubber and other agents will be dependent on the particular drug used and the form in which it is added, drug alone or drug plus solvent. By varying the composition, the release rate can be modified, as will be apparent to one skilled in the art.”

As is also disclosed in U.S. Pat. No. 5,686,099, “Selection of the particular multipolymer is governed in large part by the drug to be incorporated in the device, as well as the desired rate of delivery of the drug. Those skilled in the art can readily determine the rate of delivery of drugs from the polymers and select suitable combinations of polymer and drug for particular applications.”

While preferred embodiments of this invention have been shown and described, modifications thereof can be made by one skilled in the art without departing from the spirit or teaching of this invention. The embodiments described herein are exemplary only and are not limiting. Many variations of the method are possible and are within the scope of the invention.

1. A material that enhances the electromagnetic transparency of an electromagnetic energy absorbing object.

2. The material of claim 1, wherein the material exhibits the property of induced current cancellation in response to electromagnetic energy transmission through the material.

3. The material of claim 1, wherein the material is super paramagnetic.

4. The material of claim 3, arranged in at least one layer.

5. The material of claim 4, wherein the at least one layer has a thickness of less than 4 microns.

6. The material of claim 2, in the form of at least one layer surrounding the electromagnetic energy absorbing object.

7. The material of claim 2, wherein the object is a biocompatible implantable object.

8. The material of claim 1, selected from the group consisting of super paramagnetic nanoparticles, semiconductor, conducting plastic, piezoelectric crystal and nanomagnetic material.

9. The material of claim 1, wherein the material exhibits multiple parallel resonance in response to irradiation with electromagnetic radiation.

10. The material of claim 9, wherein the material comprises discrete circuit components that provide the multiple parallel resonance.

11. The material of claim 9, wherein the multiple parallel resonance provides a wide bandwidth that cancels out at least some loading effect.

12. The material of claim 1, wherein the material provides a bulk property of series resonance in response to irradiation of an electromagnetic field.

13. The material of claim 1, comprising grain oriented regions with characteristic properties of magnetic permeability.

14. The material of claim 1, wherein the electromagnetic energy is focused within the electromagnetic energy absorbing object.

15. The material of claim 14, wherein the material forms a hysteresis loop.

16. A device comprising an electromagnetic energy absorbing object and the material of claim 1, wherein the material covers the outside of at least a portion of the electromagnetic energy absorbing object.

17. A device as described in claim 16, wherein the electromagnetic energy absorbing object is a stent.

18. A method making an implantable device that is at least partially shielded to electromagnetic penetration, comprising the step of adding one or more current cancellation circuits to one or more electromagnetic energy responsive substrates of the device, thereby diminishing induced current in the device.

19. The method of claim 18, wherein the one or more current cancellation circuits comprise discrete circuit components that resonate in response to irradiation with electromagnetic energy.

20. The method of claim 18, wherein the one or more current cancellation circuits comprise bulk material that covers the one or more electromagnetic energy responsive substrates, and wherein the bulk material exhibits multiple parallel or series resonances.

21. The method of claim 18, further comprising the step of adding one or more voltage cancellation circuits to the one or more substrates.

22. The method of claim 18, wherein the one or more current cancellation circuits are positioned perpendicular to a long axis of the one or more electromagnetic energy responsive substrates.

23. The method of claim 18, wherein the cancellation circuits comprise a first type of material that is closer to the electromagnetic energy responsive substrate and that chokes signal, and a second type of material that is further away from the electromagnetic energy responsive substrate and that leads the electromagnetic signal in.

24. The method of claim 23, wherein the first and second types of material comprise nanomagnetic material in a layer having a thickness that is less than 4 microns.

25. The method of claim 24, wherein the nanomagnetic material comprises at least one of a polymer, paramagnetic metal, a powder, a suspension, an alloy, and super-paramagnetic material.

26. A shielded implantable device, comprising a first electromagnetic energy responsive substrate that forms an induced voltage in opposition to an applied electromagnetic energy, and a second set of multiple voltage cancellation circuits thereby diminishing the substrate's induced voltage.

27. An MRI compatible implantable device having enhanced transparency to an electromagnetic energy beam, comprising a first material opaque to the electromagnetic energy beam and a second material that enhances transparency of the first material to the electromagnetic energy beam wherein the device, after implantation, presents a direct current magnetic susceptibility that is plus or minus 0.001 centimeter-gram-seconds.

28. The device of claim 27, comprising one or more voltage cancellation circuits.

29. A method of enhancing transparency of an implantable device to radio frequency energy, comprising adding a material to the device, wherein the material has at least one of the properties of induced current cancellation and induced voltage cancellation.

30. The method of claim 29, wherein the material is selected from the group consisting of, a thin film, a piezoelectric film, a discrete circuit, multiple discrete circuits, and nanomagnetic material with a saturation magnetization of at least 2,000 electromagnetic units per cubic centimeter.

31. An implantable device prepared by the process of claim 29.

32. An implantable device coated with material that enhances the electromagnetic transparency of the device.

33. The device of claim 32, wherein the material exhibits the property of induced current cancellation in response to electromagnetic energy transmission through the material.
